(19) United States
(12) Patent Application Publication
(10) Pub. No.: US 2005/0130941 A1
(43) Pub. Date: Jun. 16, 2005

(54) Methods of treating alzheimer's disease

(76) Inventors: Heinrich Schostarez, Potrage, MI (US); Robert Allan Chrusciel, Portage, MI (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/480,967

(22) PCT Filed: Jun. 7, 2002
(86) PCT No.: PCT/US02/18262

Related U.S. Application Data
(60) Provisional application No. 60/296,941, filed on Jun. 8, 2001.

(51) Int. Cl. A61K 31/66; A61K 31/44; A61K 31/40; A61K 31/137; A61K 31/165

(52) U.S. Cl. 514/114; 514/352; 514/426; 514/602; 514/616; 514/649

(57) ABSTRACT

Disclosed are methods for treating Alzheimer's disease, and other diseases, and/or inhibiting beta-secretase enzyme, and/or inhibiting deposition of A beta peptide in a mammal, by use of hydrazine compounds of formula (I) wherein the variables R₁-R₉ are defined herein.

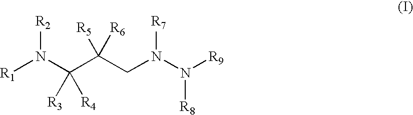

This application claims the benefit of U.S. Provisional Application No. 60/296,941, led Jun. 8, 2001.

### FIELD OF THE INVENTION

The present invention relates to the treatment of Alzheimer's disease and other similar diseases, and more specifically to the use of compounds that inhibit beta-secretase, an enzyme that cleaves amyloid precursor protein to produce A beta peptide, a major component of the amyloid plaques found in the brains of Alzheimer's sufferers, in such methods.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD. Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp, and Memapsin. See, for example, Sindha et al., 1999, Nature 402:537-554 (p501) and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6:487. Release of A beta from neuronal cells grown in culture and the presence of A beta in cerebrospinal fluid (CSF) of both normal individuals and AD patients has been demonstrated. See, for example, Seubert et al., 1992, Nature 359:325-327.

It has been proposed that A beta peptide accumulates as a result of APP processing by beta-secretase, thus inhibition of this enzyme's activity is desirable for the treatment of AD. In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that over express APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

U.S. Pat. No. 5,753,652 discloses hydrazine compounds of the formula 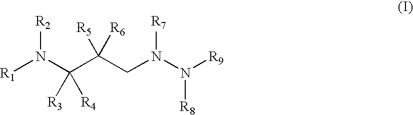
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉;
or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen, or R₅ and R₆ together are oxo; and
R₇ is unsubstituted or substituted alkyl or cycloalkyl; aryl; heterocyclyl; or unsubstituted or substituted alkenyl; and salts of the mentioned compounds where salt-forming groups are present.

U.S. Pat. No. 5,753,652 discloses how to make the above compounds and how to use them in treating HIV and AIDS; the disclosure of U.S. Pat. No. 5,753,652 is incorporated herein by reference in its entirety.

### SUMMARY OF INVENTION

The present invention relates to methods of treating a patient who has, or in preventing a patient from developing, a disease or condition selected from the group consisting of Alzheimer's disease, for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I): 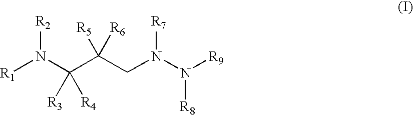
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉;
or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen, or R₅ and R₆ together are oxo; and
R₇ is unsubstituted or substituted alkyl or cycloalkyl; aryl; heterocyclyl; or unsubstituted or substituted alkenyl; and salts of the mentioned compounds where salt-forming groups are present.

### DETAILED DESCRIPTION OF THE INVENTION

In one aspect, the present invention relates to methods of treating a patient who has, or in preventing a patient from developing, a disease or condition selected from the group consisting of Alzheimer's disease, for helping prevent or delay the onset of Alzheimer's disease, for helping to slow the progression of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I): 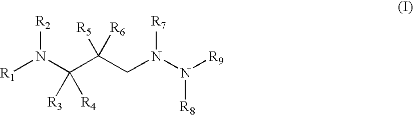
where R₁, R₂, R₃, R₄, R₅, R₆, R₇, R₈, and R₉ are as defined above.
   Preferred compounds of formula (I) include the following:
1-[2(S)-(2-pyridylcarbonyl)oxy-3(S)-(N-quinoline-2-carbonyl)-(L)-asparaginyl)amino4-phenylbutyl-1-[phenylmethyl]-2-[N-(methoxycarbonyl)-(L)-valyl]hydrazine;
1-[2(S)-butyryloxy-3(S)-(N-quinoline-2-carbonyl)-(L)-asparaginyl)amino-4-phenylbutyl-1-[phenylmethyl]-2-[N-(methoxycarbonyl)-(L)-valyl]hydrazine;
1-[2(S)-(2-pyridylcarbonyl)oxy-3(S)-(N-quinoline-2-carbonyl)-(L)-asparaginyl)amino4-phenylbutyl-1-[4-methoxyphenylmethyl]-2-[N-(benzyloxycarbonyl)-(L)-valyl]hydrazine;
1-[2(S)-(methoxy-acetyl)oxy-3(S)-(N-quinoline-2-carbonyl)-(L)-asparaginyl)amino-4-phenylbutyl-1-[4-methoxyphenylmethyl]-2-[N-(benzyloxycarbonyl)-(L)-valyl]hydrazine;
1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-octanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-furylcarbonyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-imidazolylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-pyridylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]hydrazine;
1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-benzyloxycarbonyl-N-methyl-aminoacetyl)oxy-3(S)-(N-(methoxycarbonyl)(L)-valyl)amino-4-phenyl-butyl]-1-[cyclohexylmethyl]-2-[N-(methoxycarbonyl)(L)-valyl]hydrazine;
1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl)hydrazine;
1-[2(S)-(4-morpholinomethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl)hydrazine;
1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(3-carboxypropionyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-(benzyloxycarbonyl)-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butoxy-carbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-methoxy-carbonyl-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[3,3-dimethylbutyryl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butylamino-carbonyl)hydrazine;
1-[2(S)-hydroxy-3(S)-(N-(quinoline-2-carbonyl)-(L)-asparaginyl)amino-4-phenyl-butyl]-1-[cyclohexylmethyl]-2-[benzylamino-carbonyl]hydrazine;
1-[2(S)-hydroxy-3(S)-(N-(quinoline-2-carbonyl)-(L)-asparaginyl)amino-4-phenyl-butyl]-1-[benzyl]-2-[N-methoxy-carbonyl)-(L)-valyl]hydrazine;
1-2(S)-hydroxy-3(S)-(N-(quinoline-2-carbonyl)-(L)-asparaginyl)amino-4-phenyl-butyl]-1-[benzyl]-2-[N-ethoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-allyloxy-carbonyl)-(L)-valyl]hydrazine;
1-[2(S)-hydroxy-3(S)-(N-(quinoline-sup.2-carbonyl)-(L)-asparaginyl)amino-4-phenyl-butyl]-1-[4-methoxyphenylmethyl]-2-[N-methoxy-carbonyl)-(L)valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-(4-methoxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-methoxyphenyl)-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-benzyloxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[2(S)-hydroxy-3(S)-(N-quinoline-2-carbonyl)-(L)-asparaginyl)amino-4-phenylbutyl-1-[phenylmethyl]-2-[N-(2-(2-methoxyethoxy)ethoxycarbonyl)-(L)-valyl]hydrazine;
1-[2 (S)-hydroxy-3 (S)-(N-quinoline-2-carbonyl)-(L)-asparaginyl)amino-4-phenylbutyl-1-[4-methoxyphenylmethyl]-2-[tert-butoxy-carbonyl]hydrazine;
1-[2(S)-hydroxy-3(S)-(N-quinoline-2-carbonyl)-(L)-asparaginyl)amino-4-phenylbutyl-1-[4-methoxyphenylmethyl]-2-[N-(ethoxycarbonyl)-(L)-valyl]hydrazine;
1-[2(S)-hydroxy-3(S)-(N-(2-methoxyethoxy)acetyl-(L)-valyl)amino-4-phenylbutyl]-1-[cyclohexylmethyl]-2-[N-(2-methoxyethoxy)acetyl-(L)-valyl]hydrazine;
1-[2(S)-hydroxy-3(S)-(N-(2-(2-methoxyethoxy)ethoxy)carbonyl-(L)-valyl)amino-4-phenylbutyl]-1-[cyclohexylmethyl]-2-[N-(2-(2-methoxyethoxy)ethoxy)carbonyl-(L)-valyl]hydrazine;
1-[2(S)-hydroxy-3(S)-(N-acetyl-(L)-valyl)amino-4-phenylbutyl]-1-[thien-2-ylmethyl]-2-[N-acetyl-(L)-valyl]hydrazine;
1-[2 (S)-hydroxy-3 (S)-(N-methoxycarbonyl-(L)-valyl)amino-4-phenylbutyl]-1-[thien-2-yl-methyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[thien-2-yl-methyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[thien-2-ylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenyl-butyl]-1-[thien-2-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[thien-2-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[thien-2-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[2,3,5,6-tetrahydropyran4-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[2,3,5,6-tetrahydropyran-4-ylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[2,3,5,6-tetrahydropyran-4-ylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[2,3,5,6-tetrahydropyran-4-ylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[2,3,5,6-tetrahydropyran-4-ylmethyl]-2-[N-(N-(2-methoxyethyl)aminocarbonyl)-(L)-valyl)hydrazine;
1-[2(S)-hydroxy-3(S)-(N-(N-(2-(morpholin-4-yl)ethyl)aminocarbonyl)-(L)-valyl)amino-4-phenylbutyl]-1-[2,3,5,6-tetrahydropyran-4-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[2,3,5,6-tetrahydropyran-4-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-hydroxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-hydroxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-hydroxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-hydroxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-hydroxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-hydroxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-hydroxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-methoxyphenylmethyl]-2-[N-acetyl-(L)-valyl]hydrazine
1-[2(S)-hydroxy-3(S)-(N-methoxycarbonyl-(L)-valyl)amino-4-phenylbutyl]-1-[4-methoxyphenylmethyl]-2-[N-methoxycarbonyl-(L)-valyl]hydrazine;
1-[2 (S)-hydroxy-3 (S)-(N-ethoxycarbonyl-(L)-valyl)amino-4-phenylbutyl]-1-[4-methoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-methoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-methoxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-methoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-methoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-benzyloxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-benzyloxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-benzyloxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-benzyloxyphenylmethyl)-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-benzyloxyphenylmethyl]-2-[N-(N-(2-methoxyethyl)-aminocarbonyl)(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-benzyloxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-benzyloxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(3,4-dimethoxybenzyl)oxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(3,4-dimethoxybenzyl)oxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(3,4-dimethoxybenzyl)oxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(3,4-dimethoxybenzyl)oxyphenylmethyl]-2-[N-(N,N-dimethylaminocarbonyl)(L)-valyl]hydrazine;
1-[2(S)-hydroxy-3(S)-(N-(N-(2-methoxyethyl)aminocarbonyl)-(L)-valyl)amino-4-phenylbutyl]-1-[4-(3,4-dimethoxybenzyl)oxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-isobutoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-isobutoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-isobutoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-isobutoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-isobutoxyphenylmethyl]-2-[N-(N-(2-methoxyethyl)aminocarbonyl)-(L)-valyl]-hydrazine;
1-[2(S)-hydroxy-3(S)-(N-(N-(2-(morpholin-4-yl)ethyl)aminocarbonyl)-(L)-valyl)amino-4-phenylbutyl]-1-[4-isobutoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-isobutoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(2-methoxyethoxy)phenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(2-methoxyethoxy)phenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(2-methoxyethoxy)phenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(2-methoxyethoxy)phenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-(2-methoxyethoxy)phenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(2-methoxyethoxy)phenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(2-methoxyethoxy)phenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[methylene-3,4-dioxyphenylmethyl]-2-[N-acetyl-(L)-valyl)hydrazine;
1-[2(S)-hydroxy-3(S)-(N-methoxycarbonyl-(L)-valyl)amino-4-phenylbutyl]-1-[methylene-3,4-dioxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[methylene-3,4-dioxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[methylene-3,4-dioxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[methylene-3,4-dioxyphenylmethyl]-2-[N— (N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[methylene-3,4-dioxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[methylene-3,4-dioxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[3,4-dimethoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[3,4-dimethoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[3,4-dimethoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[3,4-dimethoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[3,4-dimethoxyphenylmethyl]-2-[N-(N-(2-methoxyethyl)aminocarbonyl)-(L)-valyl]hydrazine:
1-[2(S)-hydroxy-3(S)-(N-(N-(2-(morpholin-4-yl)ethyl)aminocarbonyl)-(L)-valyl)amino-4-phenylbutyl]-1-[3,4-dimethoxyphenylmethyl]-2-[N-(N-(2-(morpholin-4-yl)ethyl)aminocarbonyl)-(L)-valyl)hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[3,4-dimethoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-biphenylylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-biphenylmethyl]-2-N-methoxycarbonyl-(L)-valyl]hydrazine;
1-[2(S)-hydroxy-3(S)-(N-ethoxycarbonyl-(L)-valyl)amino-4-phenylbutyl]-1-[4-biphenylylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-biphenylylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-biphenylylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-biphenylylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-biphenylylmethyl]-2-[N-(N-(2-(morpholin-4-yl)ethyl)N-methylaminocarbonyl)-(L)-valyl]hydrazine;
1-[2(S)-hydroxy-3(S)-(N-ethoxycarbonyl-(L)-valyl)amino-4-phenylbutyl]-1-[cyclohexylmethyl]-2-[N-ethoxycarbonyl-(L)-valyl]hydrazine;
1-[2(S)-hydroxy-3(S)-(N-trifluoroacetyl-(L)-valyl)amino-4-phenylbutyl]-1-[cyclohexylmethyl]-2-[N-trifluoroacetyl-(L)-valyl]hydrazine;
1-[2(S)-hydroxy-3(S)-(N-(methoxy-carbonyl)-(L)-valyl)amino-4-cyclohexyl-butyl]-1-[cyclohexylmethyl]-2-[N-methoxy-carbonyl-(L)-valyl]hydrazine;
1-[2(S)-hydroxy-3(S)-(N-(n-propoxy-carbonyl)-(L)-valyl)amino-4-phenyl-butyl]-1-[cyclohexylmethyl]-2-[N-(n-propyl)oxy-carbonyl-(L)-valyl]hydrazine;
1-[2(R)-hydroxy-3(S)-(N-(methoxy-carbonyl)-(L)-valyl)amino-4-phenyl-butyl]-1-[cyclohexylmethyl]-2-1N-methoxy-carbonyl-(L)-valyl]hydrazine;
1-[2(R)-hydroxy-3(R)-(N-(methoxy-carbonyl)-(L)-valyl)amino-4-phenyl-butyl]-1-[cyclohexylmethyl]-2-[N-methoxy-carbonyl-(L)-valyl]hydrazine;
1-[2(S)-hydroxy-3(S)-(benzyloxy-carbonyl-amino)-4-phenyl-butyl]-1-[phenylmethyl]-2-[N-methoxy-carbonyl-(L)-valyl]hydrazine;
1-[2(S)-hydroxy-3(S)-(N-(methoxy-carbonyl)-(L)-valyl)amino-4-cyclohexyl-butyl]-1-[phenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-methoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-isobutoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-ethoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-benzyloxyphenylmethyl]-2-[N-methoxy-carbonyl)-(L)-valyl]hydrazine;
1-[2(S)-hydroxy-3(S)-(N-methoxycarbonyl-(L)-valyl)amino-4-phenyl-butyl]-1-[cyclohexylmethyl]-2-[2-pyridylcarbonyl]hydrazine;
N-[(1S,2R)-3-(2-benzoyl-1-ethylhydrazino)-1-benzyl-2-hydroxypropyl]-2-[(methylsulfonyl)amino]-1,3-thiazole-4-carboxamide;
N-{(1S, 2R)-1-benzyl-3-[1-ethyl-2-(4-methylpentanoyl)hydrazino]-2-hydroxypropyl}-2-[(methylsulfonyl)amino]-1,3-thiazole-4-carboxamide;
N-{(1S, 2R)-1-benzyl-3-[1-ethyl-2-(4-methylpentanoyl)hydrazino]-2-hydroxypropyl}-4-methylpentanamide;
N¹-{(1S,2S)-1-benzyl-3-[1-ethyl-2-(4-methylpentanoyl)hydrazino]-2-hydroxypropyl}-5-methyl-N³,N³-dipropylisophthalamide,
N¹-{(1S,2S)-1-(3,5-difluorobenzyl)-3-[1-ethyl-2-(4-methylpentanoyl)hydrazino]-2-hydroxypropyl}-5-methyl-N³,N³-dipropylisophthalamide;
N¹-{(1S,2S)-1-(3,5-difluorobenzyl)-3-[1-ethyl-2-(4-methylbutanoyl)hydrazino]-2-hydroxypropyl}-5-methyl-N³,N³-dipropylisophthalamide;
   or pharmaceutically acceptable salts thereof.

In one aspect, this method of treatment can be used where the disease is Alzheimer's disease.

In another aspect, this method of treatment can help prevent or delay the onset of Alzheimer's disease.

In another aspect, this method of treatment can help slow the progression of Alzheimer's disease.

In another aspect, this method of treatment can be used where the disease is mild cognitive impairment.

In another aspect, this method of treatment can be used where the disease is Down's syndrome.

In another aspect, this method of treatment can be used where the disease is Hereditary Cerebral Hemorrhage with Amyloidosis of the Dutch-Type.

In another aspect, this method of treatment can be used where the disease is cerebral amyloid angiopathy.

In another aspect, this method of treatment can be used where the disease is degenerative dementias.

In another aspect, this method of treatment can be used where the disease is diffuse Lewy body type of Alzheimer's disease.

In another aspect, this method of treatment can treat an existing disease, such as those listed above.

In another aspect, this method of treatment can prevent a disease, such as those listed above, from developing or progressing.

The methods of the invention employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In a preferred aspect, the therapeutically effective amounts for oral administration is from about 1 mg/day to about 100 mg/day; and for parenteral administration from about 5 to about 50 mg daily.

In a more preferred aspect, the therapeutically effective amounts for oral administration is from about 5 mg/day to about 50 mg/day.

The present invention also includes the use of a compound of formula (I), or a pharmaceutically acceptable salt thereof for the manufacture of a medicament for use in treating a patient who has, or in preventing a patient from developing, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, diffuse Lewy body type of Alzheimer's disease and who is in need of such treatment.

In one aspect, this use of a compound of formula (I) can be employed where the disease is Alzheimer's disease.

In another aspect, this use of a compound of formula (I) can help prevent or delay the onset of Alzheimer's disease.

In another aspect, this use of a compound of formula (I) can help slow the progression of Alzheimer's disease.

In another aspect, this use of a compound of formula (I) can be employed where the disease is mild cognitive impairment.

In another aspect, this use of a compound of formula (I) can be employed where the disease is Down's syndrome.

In another aspect, this use of a compound of formula (I) can be employed where the disease is Hereditary Cerebral Hemorrhage with Amyloidosis of the Dutch-Type.

In another aspect, this use of a compound of formula (I) can be employed where the disease is cerebral amyloid angiopathy.

In another aspect, this use of a compound of formula (I) can be employed where the disease is degenerative dementias.

In another aspect, this use of a compound of formula (I) can be employed where the disease is diffuse Lewy body type of Alzheimer's disease.

In a preferred aspect, this use of a compound of formula (I) is a pharmaceutically acceptable salt of an acid selected from the group consisting of acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)ₙ—COOH where n is as defined above, HOOC—CH═CH—COOH, and phenyl-COOH.

The present invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. These methods each include administration of a therapeutically effective amount of a compound of formula (I), or a pharmaceutically acceptable salt thereof.

The present invention also includes a method for inhibiting beta-secretase activity, including exposing said beta-secretase to an effective inhibitory amount of a compound of formula (I), or a pharmaceutically acceptable salt thereof.

In one aspect, this method includes exposing said beta-secretase to said compound in vitro.

In another aspect, this method includes exposing said beta-secretase to said compound in a cell.

In another aspect, this method includes exposing said beta-secretase to said compound in a cell in an animal.

In another aspect, this method includes exposing said beta-secretase to said compound in a human.

The present invention also includes a method for inhibiting cleavage of amyloid precursor protein (APP), in a reaction mixture, at a site between Met596 and Asp597, numbered for the APP-695 amino acid isotype; or at a corresponding site of an isotype or mutant thereof, including exposing said reaction mixture to an effective inhibitory amount of a compound of formula (I), or a pharmaceutically acceptable salt thereof.

In one aspect, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671 and Asp672 of the APP-770 Swedish Mutation.

In another aspect, this method exposes said reaction mixture in vitro.

In another aspect, this method exposes said reaction mixture in a cell.

In another aspect, this method exposes said reaction mixture in an animal cell.

In another aspect, this method exposes said reaction mixture in a human cell.

The present invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The present invention also includes a method for inhibiting the production of beta-amyloid plaque in an animal, including administering to said animal an effective-inhibitory amount of a compound of formula (I), or a pharmaceutically acceptable salt thereof.

In one embodiment of this aspect, this method includes administering to a human.

The present invention also includes a method for treating or preventing a disease characterized by beta-amyloid deposits in the brain including administering to a patient an effective therapeutic amount of a hydrazine compound of formula (I), or a pharmaceutically acceptable salt thereof.

In one aspect, this method employs a compound at a therapeutic amount in the range of from about 0.1 to about 1000 mg/day.

In another aspect, this method employs a compound at a therapeutic amount in the range of from about 15 to about 1500 mg/day.

In another aspect, this method employs a compound at a therapeutic amount in the range of from about 1 to about 100 mg/day.

In another aspect, this method employs a compound at a therapeutic amount in the range of from about 5 to about 50 mg/day.

In another aspect, this method can be used where said disease is Alzheimer's disease.

In another aspect, this method can be used where said disease is Mild Cognitive Impairment, Down's Syndrome, or Hereditary Cerebral Hemorrhage with Amyloidosis of the Dutch Type.

The present invention also includes a composition including beta-secretase complexed with a compound of formula (I), or a pharmaceutically acceptable salt thereof.

The present invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent kit including a compound of formula (I), or a pharmaceutically acceptable salt thereof; and one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta antibody.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds of the invention possess beta-secretase inhibitory activity. The inhibitory activities of the compounds of the invention are readily demonstrated, for example, using one or more of the assays described herein or known in the art.

The compounds of formula (I) can form salts when reacted with acids. Pharmaceutically acceptable salts are preferred over the corresponding amines of formula (I) since they frequently produce compounds which are generally more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. The preferred pharmaceutically acceptable salts include salts of the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, succinic, sulfamic, sulfanilic, sulfonic, sulfuric, tannic, tartaric, teoclic and toluenesulfonic. For other acceptable salts, see Int. J. Pharm., 33, 201-217 (1986) and J. Pharm. Sci., 66(1), 1, (1977).

The present invention provides kits, and methods for inhibiting beta-secretase enzyme activity and A beta peptide production. Inhibition of beta-secretase enzyme activity halts or reduces the production of A beta from APP and reduces or eliminates the formation of beta-amyloid deposits in the brain.

Methods of the Invention

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating, preventing, or slowing the progression of Alzheimer's disease. When treating or preventing these diseases, the compounds of the invention can either be used individually or in combination, as is best for the patient.

With regard to these diseases, the term “treating” means that compounds of the invention can be used in humans with existing disease. The compounds of the invention will not necessarily cure the patient who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease, such as a known genetic mutation of APP or APP cleavage products in brain tissues or fluids. By delaying the onset of the disease, compounds of the invention have prevented the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to be predisposed to the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used and the route of administration, as is known to those skilled in the art.

In treating a patient displaying any of the diagnosed above conditions a physician may administer a compound of the invention immediately and continue administration indefinitely, as needed. In treating patients who are not diagnosed as having Alzheimer's disease, but who are believed to be at substantial risk for Alzheimer's disease, the physician should preferably start treatment when the patient first experiences early pre-Alzheimer's symptoms such as, memory or cognitive problems associated with aging. In addition, there are some patients who may be determined to be at risk for developing Alzheimer's through the detection of a genetic marker such as APOE4 or other biological indicators that are predictive for Alzheimer's disease. In these situations, even though the patient does not have symptoms of the disease, administration of the compounds of the invention may be started before symptoms appear, and treatment may be continued indefinitely to prevent or delay the onset of the disease.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used with each other or with other agents used to treat or prevent the conditions listed above. Such agents include gamma-secretase inhibitors, anti-amyloid vaccines and pharmaceutical agents such as donepezil hydrochloride (ARICEPT Tablets), tacrine hydrochloride (COGNEX Capsules) or other acetylcholine esterase inhibitors and with direct or indirectneurotropic agents of the future.

In addition, the compounds of the invention can also be used with inhibitors of P-glycoproten (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of the invention. To that end the P-gp inhibitor and the compounds of the invention can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that do the same function and are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a patient may be started on one dose, that dose may have to be varied over time as the patient's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the patient one thru four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ. The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the path is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art. The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

The compounds employed in the methods of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds employed in the methods of the invention administered, the particular condition being treated, the severity of the condition being treated, the age, weight, general physical condition of the particular patient, and other medication the individual may be taking as is well known to administering physicians who are skilled in this art.

Inhibition of APP Cleavage

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Preferred methods employ compounds that are effective to inhibit 50% of beta-secretase enzymatic activity at a concentration of less than about 50 micromolar, preferably at a concentration of less than about 10 micromolar, more preferably less than about 1 micromolar, and most preferably less than about 10 nanomolar.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In Vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a patient at risk for the disease.

Unless defined otherwise, all scientific and technical terms used herein have the same meaning as commonly understood by one of skill in the art to which this invention belongs. All patents and publications referred to herein are hereby incorporated by reference for all purposes.

Definitions

In the description of this invention, the term “lower” used in the definition of groups or radicals, for example lower alkyl, lower alkoxy, lower alkanoyl etc., means that, unless expressly otherwise defined, the groups or radicals so defined contain up to and including 7, and preferably up to and including 4, carbon atoms.

Unless indicated to the contrary, the radicals R₁, R₂, R₃, R₄, R₇, R₈ and/or R₉ may be mono- to poly-substituted, especially mono- to tri-substituted, for example mono-substituted, by identical or different substituents.

The carbon atoms in compounds of formula I substituted by R₃ and R₄ and by R₅ and R₆ may, if they are asymmetric, be in the (R)-, (S)- or (R,S)-configuration, as may also any other asymmetric carbon atoms present. Accordingly, the present compounds may be in the form of isomeric mixtures or in the form of pure isomers, especially in the form of diastereoisomeric mixtures, pairs of enantiomers or pure enantiomers. Preferred compounds of formula I are those wherein the carbon atom substituted by R₃ or by hydroxy R₅ has the (S)-configuration and any other asymmetric carbon atoms that may be present are, independently of one another, in the (R)-, (S)- or (R,S)-configuration.

The general terms and names used in the description of this invention preferably have the following meanings, and within the different levels of meanings of the radicals listed hereinbefore and hereinbelow it is possible to use any combinations or individual radicals instead of the general definitions:

Acyl R₁, R₂, R₈ or R₉ has, for example, up to 25, preferably up to 19, carbon atoms and is especially the acyl group of a carboxylic acid, of a semiester of carbonic acid, of an unsubstituted or N-substituted carbamic acid, of an unsubstituted or N-substituted oxalamide or of an unsubstituted or substituted amino acid, it being possible for there to be thiocarbonyl groups instead of carbonyl groups in each of the acyl radicals mentioned. Preferably not more than one of the radicals R₁ and R₂ and not more than one of the radicals R₈ and R₉ is acylated.

Preferred acyl groups R₁, R₂, R₈ and R₉ of a carboxylic acid are unsubstituted or substituted alkanoyl having up to 19 carbon atoms, for example n-decanoyl, or preferably lower alkanoyl, such as formyl, acetyl, propionyl, butyryl or pivaloyl, or substituted lower alkanoyl, especially in the form of cycloalkyl-lower alkanoyl wherein cycloalkyl has, for example, from 3 to 7 carbon atoms and lower alkanoyl is as defined above, for example cycloalkylcarbonyl, especially having a total of from 4 to 8 carbon atoms, such as cyclopropyl-, cyclobutyl-, cyclopentyl- or cyclohexyl-carbonyl, or 2-cyclohexyl- or 2-cyclopentyl-acetyl, cycloalkenyl-lower alkanoyl where in cycloalkenyl has, for example, from 3 to 7 carbon atoms, such as cycloalkenylcarbonyl, preferably having from 4 to 8 carbon atoms, such as 1-cyclohexenylcarbonyl, 1,4-cyclohexadienylcarbonyl or 1-cyclohexenylacetyl or 1,4-cyc [symbol] adienylacetyl,
bicycloalkyl-lower alkanoyl wherein bicycloalkyl contains, for example, from 5 to 10 carbon atoms, for example bicycloalkylcarbonyl, preferably having from 8 to 11 carbon atoms, such as decahydronaphthyl-2-carbonyl, endo- or exo-norbornyl-2-carbonyl, bicyclo[2.2.2]oct-2-ylcarbonyl or bicyclo[3.3.1]non-9-ylcarbonyl, and also bicyclo-hexyl-, -heptyl-, -octyl-, -nonyl- or -decyl-acetyl or -3-propionyl, such as bicyclo[3.1.0]hexyl-, -2- or -3-yl-, bicyclo[4.1.0]hept-1- or -7-yl-, bicyclo[2.2.1]hept-2-yl-, such as endo- or exonorbornyl-, bicyclo[3.2.1]oct-2-yl-, bicyclo[3.3.0]oct-3-yl- or bicyclo[3.3.1]non-9-yl-, and also α- or β-decahydronaphthyl-acetyl or -3-propionyl,
bicycloalkenylcarbonyl, preferably having from 8 to 12 carbon atoms, such as 5-norbornen-2-ylcarbonyl or bicyclo[2.2.2]octen-2-ylcarbonyl, tricycloalkyl-lower alkanoyl wherein tricycloalkyl contains, for example, from 8 to 10 carbon atoms, for example tricycloalkylcarbonyl, preferably having from 8 to 11 carbon atoms, such as 1- or 2-adamantylcarbonyl, and also tricyclo[5.2.1.0^{2,6}]dec-8-yl- or adamantyl-acetyl, such as 1-adamantyl-acetyl,
aryl-lower alkanoyl wherein aryl has from 6 to 14 carbon atoms, such as in phenyl, indenyl, indanyl, naphthyl, anthryl, phenanthryl, acenaphthyl or fluorenyl, and may be unsubstituted or especially mono- to tri-substituted by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, —C(O)NH₂, —C(O)NH(C₁-C₆ alkyl), —C(O)N(C₁-C₆ alkyl) (C₁-C₆ alkyl), carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, for example in diphenyl-, dibenzyl- or triphenyl-lower alkanoyl, such as diphenyl-, dibenzyl- or triphenyl-acetyl, and wherein lower alkanoyl may be unsubstituted or substituted, for example by lower alkyl, for example methyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, hydroxy, lower alkoxy, lower alkanoyloxy, for example acetoxy, propionyloxy, butyroxy, isobutyroxy or pivaloyloxy, acetoacetoxy, amino- or benzyloxycarbonylamino-lower alkanoyloxy, for example 2-amino- or 2-benzyloxycarbonylamino-2-methylpropionyloxy, aryl-lower alkanoyloxy wherein aryl has from 6 to 10 carbon atoms, for example in benzoyloxy, phenylacetoxy, 1- or 2-naphthoyloxy, lower alkoxycarbonyloxy, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyloxy, mono- or di-lower alkylaminocarbonyloxy, for example ethylaminocarbonyloxy or diethylaminocarbonyloxy, aryloxycarbonyloxy wherein aryl has from 6 to 10 carbon atoms, for example phenoxycarbonyloxy or 1- or 2-naphthyloxycarbonyloxy, aryl-lower alkoxycarbonyloxy wherein aryl has from 6 to 14 carbon atoms, especially phenyl-lower alkoxycarbonyloxy, for example benzyloxycarbonyloxy, and also 1- or 2-naphthylmethoxycarbonyloxy or 9-fluorenylmethoxycarbonyloxy, sulfonyloxy, lower alkylsulfonyloxy, for example methyl-, ethyl-, propyl-, isopropyl-, n-butyl-, isobutyl-, sec-butyl-, tert-butyl-, n-pentyl-, isopentyl-, neopentyl-, tert-pentyl-, n-hexyl-, isohexyl- or n-heptyl-sulfonyloxy, phenylsulfonyloxy, 2- or 4-toluenesulfonyloxy, 1- or 2-naphthylsulfonyloxy, carboxy, esterified carboxy selected from lower alkoxycarbonyl, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyl, aryloxycarbonyl wherein aryl has from 6 to 10 carbon atoms, for example phenoxycarbonyl or 1- or 2-naphthyloxycarbonyl, aryl-lower alkoxycarbonyl wherein aryl has from 6 to 12 carbon atoms, for example benzyloxycarbonyl, 1- or 2-naphthylmethoxycarbonyl or 2-fluorenylmethoxycarbonyl, lower alkanoyl, lower alkylsulfonyl, for example methyl- or tert-butyl-sulfonyl, hydroxy-lower alkoxyphosphoryl or di-lower alkoxyphosphoryl, carbamoyl, carbamoyl substituted by one or two radicals selected from lower alkyl, such as methyl, ethyl, propyl, isopropyl, n-butyl, isobutyl, sec-butyl, tert-butyl, n-pentyl, isopentyl, neopentyl, tert-pentyl, n-hexyl, isohexyl or n-heptyl, for example in N-methylcarbamoyl, N-n-butylcarbamoyl or N,N-dimethylcarbamoyl, from carboxy-lower alkyl or lower alkoxycarbonyl-lower alkyl, for example in the form of carboxymethylcarbamoyl (glycinylcarbonyl) or in the form of tert-butoxycarbonylmethylcarbamoyl, from di-lower alkylamino-lower alkyl, for example 2-dimethylaminoethyl, aminocarboxy-lower alkyl, for example 5-amino-5-carboxypentyl, from hydroxy-lower alkyl, for example hydroxymethyl or hydroxyethyl, and from di-lower alkoxy-lower alkyl, for example 2-(2,2-dimethoxyethyl), or carbamoyl substituted by one radical selected from ethylene, trimethylene, tetramethylene and pentamethylene in which a carbon atom may have been replaced by nitrogen, oxygen, sulfur or by sulfur mono- or di-substituted by oxygen and which may be unsaturated, such as piperidin-1-yl-, pyrazin-1-yl-, piperazin-1-yl-, pyrimidin-1-yl-, pyridazin-1-yl-, morpholino-, thiomorpholino- or S,S-dioxothiomorpholino-carbonyl; sulfamoyl, phosphono, benzofuranyl, oxo and/or by cyano and is unbranched or branched, for example selected from benzoyl that is unsubstituted or mono- or poly-substituted by lower alkyl, for example methyl, phenyl, halogen, for example fluorine or chlorine, hydroxy, lower alkoxy, for example methoxy, and/or by nitro, such as 4-chloro-, 4-methoxy- or 4-nitro-benzoyl, naphthylcarbonyl, such as α- or β-naphthylcarbonyl or 1,8-naphthalenedicarbonyl bonded to the amino group via both carbonyl groups, indenylcarbonyl, such as 1-, 2- or 3-indenylcarbonyl, indanylcarbonyl, such as 1- or 2-indanylcarbonyl, phenanthrenylcarbonyl, such as 9-phenanthrenylcarbonyl, phenylacetyl, α-naphthylacetyl, β-naphthylacetyl, lower alkylphenylacetyl, such as 4-methylphenylacetyl, lower alkoxyphenylacetyl, such as 4-methoxyphenylacetyl, 3-phenylpropionyl, 3-(p-hydroxyphenyl)-propionyl, diphenylacetyl, di-(4-methoxyphenyl)-acetyl, triphenylacetyl, 2,2-dibenzylacetyl, anilinophenylacetyl substituted in the phenyl radical by one or two radicals selected from lower alkyl, for example methyl or ethyl, hydroxy, lower alkoxy, for example methoxy, amino, mono- or di-lower alkylamino, for example ethylamino or dimethylamino, halogen, for example fluorine or chlorine, carboxy, sulfo, carbamoyl, sulfamoyl and cyano and/or at the amino group by one or two radicals selected from lower alkyl and benzyl, such as 2-(o,o-dichloroanilino)-phenylacetyl or 2-(o,o-dichloro-N-benzylanilino)-phenylacetyl, 3-α- or 3-β-naphthylpropionyl, 2-benzyl-3-(1-pyrazolyl)-propionyl, 3-phenyl- or 3-α-naphthyl-2-hydroxypropionyl, 3-phenyl- or 3-α-naphthyl-2-lower alkoxypropionyl, such as 3-phenyl- or 3-α-naphthyl-2-neopentyloxypropionyl, 3-phenyl- or 3-α-naphthyl-2-lower alkanoyloxypropionyl, such as 3-phenyl-2-pivaloyloxy- or -2-acetoxy-propionyl, 2-benzyl- or 1- or 2-naphthyl-3-(N-methoxy-N-methylamino)-propionyl, 3-α-naphthyl-2-acetoacetoxypropionyl, 3-α-naphthyl-2-ethylaminocarbonyloxy-propionyl or 3-α-naphthyl-2-(2-amino- or 2-benzyloxycarbonylamino-2-methylpropionyloxy)-propionyl, 3-phenyl- or 3-α-naphthyl-2-carboxymethylpropionyl, 3-phenyl- or 3-α-naphthyl-2-lower alkoxycarbonyl-propionyl, such as 3-α-naphthyl-2-ethoxycarbonyl-propionyl, 3-phenyl- or 3-α-naphthyl-2-benzyloxycarbonyl-methyl-propionyl, 2-(S)benzyl-3-tert-butylsulfonylpropionyl, 3-phenyl-2-phosphono- or -phosphonomethylpropionyl, 3-phenyl-2-dimethoxyphosphoryl- or -dimethoxyphosphorylmethyl-propionyl, 3-phenyl-2-diethoxyphosphoryl- or -diethoxyphosphorylmethyl-propionyl, 3-phenyl-2-ethoxy- or -methoxy-hydroxyphosphorylpropionyl, phenyl-lower alkanoyl wherein the lower alkanoyl radical is substituted by carbamoyl, such as 2(R,S)-carbamoyl-3-phenylpropionyl, 3-phenyl- or 3-α-naphthyl-2-carbamoylpropionyl, 3-phenyl- or 3-α-naphthyl-2-tert-butylcarbamoyl-propionyl, 3-phenyl- or 3-α-naphthyl-2-(2-dimethylaminoethyl)carbamoylpropionyl, 3-α-naphthyl-2-(carboxy- or tert-butoxy-carbonyl)methylcarbamoyl-propionyl, 3-phenyl- or 3-α-naphthyl-2-(3-hydroxy-2-propyl)carbamoyl-propionyl, 3-phenyl- or 3-α-naphthyl-2-(2,2-dimethoxyethyl)carbamoylpropionyl, 3-phenyl- or 3-α-naphthyl-2-(5-amino-5-carboxypentyl)-carbamoylpropionyl, 3-phenyl- or 3-α-naphthyl-2-cyanopropionyl, 3-phenyl- or 3-α-naphthyl-2-cyanomethylpropionyl, 3-phenyl- or 3-α-naphthyl-2-acetonyl-propionyl, 4-hydroxyphenylbutyryl, 4-phenyl- or 4-α-naphthyl-3-carboxybutyryl, 4-phenyl- or 4-(α-naphthyl-3-benzyloxycarbonyl-butyryl, 2-benzyl- or 2-α-naphthylmethyl-4-cyanobutyryl, 2-benzyl-4-(2-benzofuranyl)-4-oxobutyryl, 2-benzyl- or 2-α-naphthylmethyl-5-dimethylamino-pentanoyl, 2-benzyl- or 2-α-naphthylmethyl-4-oxapentanoyl, 2-benzyl- or 2-α-naphthylmethyl-4,4-dimethyl-3-oxo-pentanoyl, 2-benzyl- or 2-α-naphthylmethyl-5-dimethylamino-4-oxo-pentanoyl, and 2-benzyl- or 2-α-naphthylmethyl-5,5-dimethyl-4-oxo-hexanoyl, especially phenyl-lower alkanoyl, such as phenylacetyl, or phenyl-lower alkanoyl wherein the lower alkanoyl radical is substituted by carbamoyl, such as 2(R,S)-carbamoyl-3-phenylpropionyl,
phenyl-lower alkenoyl, such as β-phenylacryloyl or β-phenylvinylacetyl,
heterocyclyl-lower alkanoyl wherein lower alkanoyl is unsubstituted or substituted as defined above under aryl-lower alkanoyl R₁, R₂, R₈ and R₉ and wherein heterocyclyl is preferably a single or double ring system having from 3 to 10 ring atoms, is bonded via a carbon atom or, especially, via a nitrogen atom and contains up to 3 further hetero atoms selected from oxygen, nitrogen, sulfur, selenium, and sulfur linked to 1 or 2 oxygen atoms, the mentioned ring system may also be fused with 1 or 2 phenyl or naphthyl radicals, it being possible for naphthyl also to be fused-on on both sides, or with 1 or 2 cycloalkyl radicals, cycloalkyl preferably having from 5 to 7 ring atoms; and may be unsaturated or partially or fully saturated, for example thienyl-, furyl-, pyranyl-, pyrrolyl-, imidazolyl-, pyrazolyl-, oxazolyl-, isoxazolyl-, thiazolyl-, furazanyl-, tetrazolyl-, pyridyl-, pyrazinyl-, pyrimidinyl-, pyridazinyl-, azepinyl-, indolyl-, benzimidazolyl-, 1H-indazolyl-, quinolyl-, isoquinolyl-, quinoxalinyl-, quinazolinyl-, cinnolyl-, purinyl-, pteridinyl-, naphthyridinyl-, 4H-quinolizinyl-, 3,1-benzofuranyl-, benz[e]indolyl-, 4,1-benzoxazinyl-, 4,1-benzothiazinyl-, carbazolyl-, β-carbolinyl-, phenazinyl-, phenanthridyl-, acridinyl-, phenoxazinyl-, phenothiazinyl-, 1-azaacenaphthenyl-, cyclohexa[b]pyrrolyl-, cyclohepta[b]-pyrrolyl-, cyclohexa[d]pyrazolyl-, cyclohexa[b]pyridyl-, cyclohexa[b)pyrazinyl-, cyclohexa[b]pyrimidinyl-, cyclohexa[b]-1,4-oxazinyl-, cyclohexa[b]-1,4-thiazinyl-, pyrrolidinyl-, pyrrolinyl-, imidazolidyl-, 2-imidazolinyl-, 2,3-dihydropyridyl-piperidyl-, piperazinyl-, 2,3,5,6-tetrahydropyrazinyl-, morpholinyl-, thiomorpholinyl-, S,S-dioxo-thiomorpholinyl-, indolinyl-, isoindolinyl-, 4,5,6,7-tetrahydroindolyl-, 1,2,3,4-tetrahydroquinolyl-, 1,2,3,4-tetrahydroisoquinolyl-, chromanyl-, thiochromanyl, 1,2,3,4-tetrahydro-3,1-benzodiazinyl-, 3,4-dihydro-3H-4,1-benzoxazinyl-, 3,4-dihydro-3H-4,1-benzothiazinyl-, 2,3,4,5-tetrahydro-1H-5,1-benzazepinyl- or 5,6-dihydrophenanthridinyl-lower alkanoyl, the mentioned heterocyclyl radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthyloxy, amino, lower alkylamino, for example methyl-, ethyl- or tert-butyl-amino, di-lower alkylamino, for example dimethyl- or diethyl-amino, —NH—SO₂—(C₁-C₆ alkyl), —N(C₁-C₆ alkyl)-SO₂—(C₁-C₆ alkyl), carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxyphosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, with heterocyclyl-lower alkanoyl being selected especially from pyrrolylcarbonyl that is unsubstituted or substituted by lower alkyl or by phenyl, for example 2- or 3-pyrrolylcarbonyl, 4- or 5-methylpyrrolylcarbonyl or 4- or 5-phenylpyrrolyl-2-carbonyl, thienylcarbonyl, such as 2-thienylcarbonyl, furylcarbonyl, such as 2-furylcarbonyl, pyridylcarbonyl, such as 2-, 3- or 4-pyridylcarbonyl, pyrimidin-1-ylcarbonyl, indolylcarbonyl that is unsubstituted or substituted by lower alkyl, such as methyl, phenyl-lower alkyl, such as benzyl, lower alkoxy, such as methoxy, phenyl-lower alkoxy, such as benzyloxy, or by halogen, such as chlorine, such as 2-, 3- or 5-indolylcarbonyl, 1-methyl-, 5-methyl-, 5-methoxy-, 5-benzyloxy-, 5-chloro- or 4,5-dimethyl-indolyl-2-carbonyl, 1-benzylindolyl-2- or -3-carbonyl, 4,5,6,7-tetrahydroindolyl-2-carbonyl, unsubstituted or hydroxy-substituted quinolyl-lower alkanoyl, for example quinolylcarbonyl, such as 2-, 3- or 4-quinolylcarbonyl or 4-hydroxyquinolyl-2-carbonyl, unsubstituted or hydroxy-substituted isoquinolylcarbonyl, such as 1-, 3- or 4-isoquinolylcarbonyl or 1-oxo-1,2-dihydroisoquinolyl-3-carbonyl, 2-quinoxalinylcarbonyl, 2-(3,1-benzofuranyl)-carbonyl, benz[e]indolyl-2-carbonyl, β-carbolinyl-3-carbonyl, cyclohepta[b]pyrrolyl-5-carbonyl, 3-chromanylcarbonyl, 3-thiochromanylcarbonyl, pyrrolidinyl-3-carbonyl, hydroxypyrrolidinylcarbonyl, such as 3- or 4-hydroxypyrrolidinyl-2-carbonyl, oxopyrrolidinylcarbonyl, such as 5-oxopyrrolidinyl-2-carbonyl, piperidylcarbonyl, such as piperidinocarbonyl or 2-, 3- or 4-piperidylcarbonyl, pyrazinylcarbonyl, such as pyrazin-1-ylcarbonyl, piperazinylcarbonyl, such as piperazin-1-ylcarbonyl, morpholinyl-lower alkanoyl, for example morpholinylcarbonyl, such as morpholinocarbonyl, thiomorpholinyl-lower alkanoyl, for example thiomorpholinylcarbonyl, such as thiomorpholinocarbonyl, S,S-dioxothiomorpholinylcarbonyl, such as S,S-dioxothiomorpholinocarbonyl, indolinylcarbonyl, such as 2- or 3-indolinylcarbonyl, 1,2,3,4-tetrahydroquinolylcarbonyl, such as 1,2,3,4-tetrahydroquinolyl-2-, 3- or 4-carbonyl, 1,2,3,4-tetrahydroisoquinolylcarbonyl, such as 1,2,3,4-tetrahydroisoquinolyl-1-, -3- or -4-carbonyl or 1-oxo-1,2,3,4-tetrahydroisoquinolyl-3-carbonyl, tetrazolyl-lower alkanoyl, such as 3-(tetrazol-1-yl)propionyl, and pyridyl-lower alkanoyl, for example pyridylacetyl, such as 2-, 3- or 4-pyridylacetyl, heterocyclyl-lower alkanoyl being selected more especially from morpholinocarbonyl, thiomorpholinocarbonyl, S,S-dioxothiomorpholinocarbonyl, pyridylacetyl, indolylacetyl, benzofuranylacetyl, 4-pyrrolidinylacetyl, 1-imidazolylacetyl, quinolin-2-ylacetyl, indol-2-ylacetyl, 2-morpholino-2-isopropylacetyl and 2-(S,S-dioxothiomorpholino)-2-isopropylacetyl, most especially morpholinocarbonyl, thiomorpholinocarbonyl, quinolin-2-ylcarbonyl, 3-(tetrazol-1-yl)-propionyl or 2- or 3-pyridylacetyl,
heterocyclyl-lower alkenoyl wherein heterocyclyl is selected especially from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, for example pyrrolidyl-lower alkenoyl, such as N-pyrrolidylacryloyl,
hydroxy-lower alkanoyl, such as 3-hydroxypropionyl or 2-hydroxy-2-methylpentanoyl,
hydroxy-lower alkoxy-lower alkanoyl, such as 3-hydroxy-n-propoxycarbonyl,
lower alkoxy-lower alkanoyl, for example lower alkoxyacetyl or lower alkoxypropionyl, such as methoxyacetyl, ethoxyacetyl or 3-methoxypropionyl,
lower alkoxy-lower alkoxy-lower alkanoyl, such as 2-methoxymethoxy-3-methylpentanoyl,
phenoxy-lower alkanoyl or nitrophenoxy-lower alkanoyl, such as phenoxyacetyl or 4-nitrophenoxyacetyl,
naphthyloxy-lower alkanoyl, such as α- or β-naphthyloxyacetyl,
lower alkanoyloxy-lower alkanoyl wherein lower alkanoyloxy is, for example, acetoxy, propionyloxy, butyroxy, isobutyroxy or pivaloyloxy, such as acetoxyacetyl or 3-acetoxypropionyl,
acetoacetoxy-lower alkanoyl, such as 3-acetoacetoxy-propionyl
amino- or benzyloxycarbonylamino-lower alkanoyloxy-lower alkanoyl, for example 2-amino- or 2-benzyloxycarbonylamino-2-methylpropionyloxy-lower alkanoyl, such as -acetyl or -3-propionyl,
aryl-lower alkanoyloxy-lower alkanoyl wherein aryl has from 6 to 10 carbon atoms, such as in benzoyloxy-, phenylacetoxy- or 1- or 2-naphthoyloxy-lower alkanoyl,
lower alkoxycarbonyloxy-lower alkanoyl, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyloxy-lower alkanoyl, such as -acetyl or -3-propionyl,
mono- or di-lower alkyl-aminocarbonyloxy-lower alkanoyl, for example ethylaminocarbonyloxy-lower alkanoyl or diethylaminocarbonyloxy-lower alkanoyl, such as -acetyl or -3-propionyl,
aryloxycarbonyloxy-lower alkanoyl wherein aryl has from 6 to 10 carbon atoms, for example phenoxycarbonyloxy- or 1- or 2-naphthyloxycarbonyloxy-lower alkanoyl, such as -acetyl or -3-propionyl,
aryl-lower alkoxycarbonyloxy-lower alkanoyl wherein aryl has from 6 to 12 carbon atoms, for example phenyl-lower alkoxycarbonyloxy-lower alkanoyl, such as benzyloxycarbonyloxy-acetyl or -3-propionyl, and also 1- or 2-naphthylmethoxycarbonyloxy-lower alkanoyl or 9-fluorenylmethoxycarbonyloxy-lower alkanoyl, such as -acetyl or -3-propionyl,
sulfonyloxy-lower alkanoyl, such as -acetyl or -3-propionyl,
lower alkylsulfonyloxy-lower alkanoyl, for example methyl-, ethyl-, propyl-, isopropyl-, n-butyl-, isobutyl-, sec-butyl-, tert-butyl-, n-pentyl-, isopentyl-, neopentyl-, tert-pentyl-, n-hexyl-, isohexyl- or n-heptyl-sulfonyloxy-lower alkanoyl, such as -acetyl or -3-propionyl,
phenylsulfonyloxy-, 2- or 4-toluenesulfonyloxy- or 1- or 2-naphthylsulfonyloxy-lower alkanoyl,
arylmercapto-lower alkanoyl wherein aryl has from 6 to 10 carbon atoms and is preferably phenyl or naphthyl, for example phenylmercapto-lower alkanoyl, such as -acetyl or -3-propionyl,
amino-lower alkanoyl wherein the amino group is not in the in α- or β-position, such as 5-aminopentanoyl,
lower alkanoylamino-lower alkanoyl wherein the amino group is not in the α- or β-position of the lower alkanoyl radical, such as 5-amino-pentanoyl,
lower alkoxycarbonylamino-lower alkanoyl wherein the amino group is not in the α- or β-position of the lower alkanoyl radical, such as 5-(tert-butoxycarbonylamino)-pentanoyl,
phenyl-lower alkoxycarbonylamino-lower alkanoyl wherein the amino group is not in the α- or β-position of the lower alkanoyl radical, such as 5-benzyloxycarbonylaminopentanoyl or 6-benzyloxycarbonylaminohexanoyl,
amino-lower alkanoyl substituted at the amino nitrogen atom by heterocyclyl-lower alkanoyl as defined above for heterocyclyl-lower alkanoyl R₁, R₂, R₈ or R₉, especially by N-morpholino- or N-thiomorpholino-carbonyl, for example N-morpholino- or N-thiomorpholino-carbonylamino-lower alkanoyl, such as N-morpholino- or N-thiomorpholinocarbonylamino-acetyl,
halo-lower alkanoyl preferably containing up to 3 halogen atoms, for example α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl,
carboxy-lower alkanoyl, for example carboxyacetyl or β-carboxypropionyl,
lower alkoxycarbonyl-lower alkanoyl, for example lower alkoxycarbonylacetyl or lower alkoxycarbonylpropionyl, such as methoxycarbonylacetyl, 3-methoxycarbonylpropionyl, ethoxycarbonylacetyl, 3-ethoxycarbonylpropionyl or 3-tert-butoxycarbonylpropionyl,
lower alkylcarbonyl-halo-lower alkanoyl, such as 3-ethoxycarbonyl-2-difluoromethylpropionyl,
2-halo-lower alkoxycarbonyl-lower alkanoyl, such as 2-chloro-, 2-bromo-, 2-iodo- or 2,2,2-trichloro-ethoxycarbonyl-acetyl or -3-propionyl,
phenyl- or naphthyl-lower alkoxycarbonyl-lower alkanoyl, for example benzyloxycarbonyl-lower alkanoyl, such as 3-benzyloxycarbonyl-2,2-dimethylpropionyl,
heterocyclyl-lower alkoxycarbonyl-lower alkanoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl and β-carbolinyl and a benzo-fused, cyclopenta-, cyclohexa- or cyclohepta-fused derivative of those radicals, it also being possible for the mentioned radicals to be fully or partially saturated, such as in 4-pyridylmethoxycarbonyl-acetyl or -3-propionyl or 2-morpholinocarbonyloxy-4-methylpentanoyl,
sulfonyl-lower alkanoyl, such as 3-sulfonylpropionyl,
lower alkylsulfonyl-lower alkanoyl, such as 2-ethylsulfonyl- or 2-tert-butylsulfonylacetyl,
arylsulfonyl-lower alkanoyl wherein aryl preferably has from 6 to 10 carbon atoms, for example phenyl or naphthyl, such as phenylsulfoacetyl,
carbamoyl-lower alkanoyl, such as carbamoylacetyl or 3-carbamoylpropionyl,
lower alkylcarbamoyl-lower alkanoyl, for example lower alkylcarbamoylacetyl or methylcarbamoyl-lower alkanoyl, such as methylcarbamoylacetyl,
di-lower alkylcarbamoyl-lower alkanoyl, for example di-lower alkylcarbamoylacetyl or dimethylcarbamoyl-lower alkanoyl, such as dimethylcarbamoylacetyl,
hydroxy-lower alkylcarbamoyl- or di(hydroxy-lower alkyl)carbamoyl-lower alkanoyl, such as hydroxymethylcarbamoyl- or di(hydroxymethyl)carbamoyl-acetyl or -propionyl,
N-lower alkoxy-lower alkoxy-lower alkylcarbamoyl-lower alkanoyl, such as 2-isobutyl-3-(2-(2-methoxyethoxy)ethylaminocarbonyl)-propionyl,
carboxy-lower alkylcarbamoyl- or di(carboxy-lower alkyl)carbamoyl-lower alkanoyl, such as carboxymethyl- or di(carboxymethyl)carbamoyl-acetyl or -propionyl,
carbamoyl-lower alkanoyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, pyrimidin-1-yl-, pyridazin-1-yl-, morpholino-, thiomorpholino- or S,S-dioxothiomorpholinocarbonyl-lower alkanoyl, such as in morpholinocarbonyl-acetyl, 3-(morpholinocarbonyl)-propionyl or 3-(morpholinocarbonyl)-2-isobutyl-propionyl,
N-heterocyclyl-lower alkylcarbamoyl-lower alkanoyl or N-lower alkyl-N-heterocyclyl-lower alkylcarbamoyl-lower alkanoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and thiomorpholinyl, such as N-methyl-2-(N-2-pyridylmethyl)-carbamoylacetyl, 2-(N-morpholino-lower alkylcarbamoyl)-lower alkanoyl, such as 2(R,S)-(N-(2-morpholinoethyl)-carbamoyl-3-methyl-butyryl or 2-(N-(pyridyl-lower alkyl)-carbamoyl)-lower alkanoyl, such as (2(R,S)-(N-(2-pyridylmethyl)-carbamoyl)-3-methyl)-butyryl, sulfamoyl-lower alkanoyl, such as 2-sulfamoylacetyl,
N-(phenyl- or naphthyl-lower alkyl)sulfamoyl-lower alkanoyl, such as 3-benzylaminosulfonyl-2-isopropyl-propionyl,
sulfamoyl-lower alkanoyl substituted at the nitrogen atom by a radical selected from ethylene, trimethylene, tetramethylene and pentamethylene wherein a carbon atom may have been replaced by nitrogen, lower alkyl-substituted, such as methyl-substituted, nitrogen, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, 4-methylpiperazin-1-yl, pyrimidin-1-yl-, pyridazin-1-yl-, morpholino-, thiomorpholino- or S,S-dioxothiomorpholinosulfonyl-lower alkanoyl, such as 3-(4-methylpiperazinylsulfonyl)-2-isopropylpropionyl or 3-(morpholinosulfonyl)-2-isopropyl-propionyl,
oxo-lower alkanoyl, such as acetoacetyl or propionylacetyl,
cyano-lower alkanoyl, such as cyanoacetyl, 2- or 3-cyanopropionyl or 2-, 3- or 4-cyanobutyryl,
hydroxy-carboxy-lower alkanoyl, such as 2-hydroxy-2-carboxy-acetyl or 2-hydroxy-3-carboxypropionyl,
(α-naphthyloxy-carboxy-lower alkanoyl, such as 2-α-naphthyloxy-4-carboxy-butyryl,
hydroxy-lower alkoxycarbonyl-lower alkanoyl, for example hydroxy-lower alkoxycarbonyl-acetyl or -propionyl or hydroxy-ethoxy- or hydroxy-methoxy-carbonyl-lower alkanoyl, such as 2-hydroxy-2-ethoxy- or -methoxy-carbonylacetyl or 2-hydroxy-3-ethoxy- or -methoxy-carbonyl-propionyl,
α-naphthyloxy-lower alkoxycarbonyl-lower alkanoyl, for example α-naphthyloxy-lower alkoxycarbonyl-acetyl, -propionyl or -butyryl or α-naphthyloxy-ethoxycarbonyl-lower alkanoyl, such as α-naphthyloxy-ethoxycarbonylacetyl, 2-α-naphthyloxy-3-ethoxycarbonylpropionyl or 2-α-naphthyloxy-4-tert-butoxycarbonylbutyryl,
α-naphthyloxy-benzyloxycarbonyl-lower alkanoyl, such as 2-α-naphthyloxy-3-benzyloxycarbonyl-propionyl,
esterified hydroxy-lower alkoxycarbonyl-lower alkanoyl wherein the hydroxy group is esterified by lower alkanoyl, for example acetyl, propionyl or pivaloyl; by cycloalkyl-lower alkanoyl wherein cycloalkyl has from 3 to 7 carbon atoms and lower alkanoyl is preferably as last defined, for example cyclohexylcarbonyl or 2-cyclohexyl- or 2-cyclopentyl-acetyl; by bicycloalkyl-lower alkanoyl wherein bicycloalkyl has, for example, from 5 to 10, especially from 6 to 9, carbon atoms, such as in bicyclohexyl-, -heptyl-, -octyl-, -nonyl- or -decyl-acetyl or -3-propionyl, for example bicyclo[3.1.0]hexyl-, -2- or -3-yl-, bicyclo[4.1.0]hept-1- or -7-yl-, bicyclo[2.2.1]hept-2-yl-, such as endo- or exo-norbornyl-, bicyclo[3.2.1]oct-2-yl-, bicyclo[3.3.0]oct-3-yl- or bicyclo[3.3.1]non-9-yl-, and also α- or β-decahydronaphthyl-acetyl or -3-propionyl; by tricycloalkyl-lower alkanoyl wherein tricycloalkyl has, for example, from 8 to 10 carbon atoms, for example in tricyclo-[5.2.1.0.sup.2,6]dec-8-yl- or adamantyl-, such as 1-adamantyl-acetyl; by aryl-lower alkanoyl wherein aryl has from 6 to 14 carbon atoms, for example phenyl, indenyl, indanyl, naphthyl, anthryl, phenanthryl, acenaphthyl or fluorenyl, which may be unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano; by lower alkoxycarbonyl, for example tert-butoxycarbonyl; by 2-halo-lower alkoxycarbonyl as defined above; or by phenyl- or fluorenyl-lower alkoxycarbonyl, for example benzyloxycarbonyl or 9-fluorenylmethoxycarbonyl, such as α-acetoxy-α-methoxycarbonyl-acetyl, α-benzoyloxy-, α-(1- or 2-naphthoyloxy)-, α-(phenyl-2-acetoxy)-, α-(1- or 2-naphthyl-2-acetoxy)-, α-(4-methylphenyl-2-acetoxy)-, α-(4-methoxyphenyl-2-acetoxy)- or α-(2-(o,o-dichlorophenyl)-2-acetoxy)-α-methoxycarbonyl-acetyl,
dihydroxy-carboxy-lower alkanoyl, such as 2,3-dihydroxy-3-carboxy-propionyl,
dihydroxy-lower alkoxycarbonyl-lower alkanoyl, such as 2,3-dihydroxy-3-ethoxy- or -methoxy-carbonyl-propionyl,
dihydroxy-lower alkoxycarbonyl-lower alkanoyl esterified by lower alkanoyl, such as acetyl, propionyl or butyryl, lower alkoxycarbonyl, for example tert-butoxycarbonyl, phenyl- or fluorenyl-lower alkoxycarbonyl, such as benzyloxycarbonyl or 9-fluorenylmethoxycarbonyl, lower alkylsulfonyl or by toluenesulfonyl, for example di-lower alkanoyloxy-lower alkoxy-propionyl, such as 2,3-diacetoxy-3-methoxycarbonylpropionyl,
α-naphthyloxy-di-lower alkylamino-lower alkanoyl, such as 2-α-naphthyloxy-5-dimethylamino-pentanoyl,
α-naphthyloxy-carbamoyl-lower alkanoyl, such as 2-α-naphthyloxy-4-carbamoyl-butyryl,
α-naphthyloxy-oxo-lower alkanoyl, such as 2-α-naphthyloxy-4-oxo-pentanoyl,
α-naphthyloxy-cyano-lower alkanoyl, such as α-naphthyloxy-cyano-acetyl or 2-α-naphthyloxy-4-cyanobutyryl,
lower alkenoyl having from 3 to 7 carbon atoms, preferably having 3 or 4 carbon atoms, lower alkenoyl being unsubstituted or substituted by the same substituents as lower alkanoyl, especially by phenyl, hydroxy, lower alkoxy, such as methoxy, phenyl-lower alkoxy, such as benzyloxy, lower alkanoyloxy, such as acetoxy, lower alkanoylamino, such as acetylamino, lower alkoxycarbonyloxy, such as tert-butoxycarbonyloxy, phenyl- or naphthyl-lower alkoxycarbonyloxy, such as benzyloxycarbonyloxy, carboxy, lower alkoxycarbonyl, such as tert-butoxycarbonyl, phenyl- or naphthyl-lower alkoxycarbonyl, halogen, such as chlorine or bromine, carbamoyl and/or by mono- or di-lower alkyl-carbamoyl, such as in acryloyl, vinylacetyl, crotonoyl or 3- or 4-pentenoyl, 3-phenylacryloyl, 3-phenylvinylacetyl or 5-phenyl-4-acetylaminopenten-2-oyl,
cycloalkyl-lower alkenoyl wherein cycloalkyl preferably has from 3 to 7 carbon atoms, for example cyclohexylacryloyl, or
lower alkynoyl having from 3 to 7, preferably 3 or 4, carbon atoms, for example propioloyl or 2- or 3-butynoyl.

Preferred acyl groups R₁, R₂, R₈ and R₉ of a semiester of carbonic acid are
lower alkoxycarbonyl, for example methoxy-, ethoxy-, isopropoxy-, isobutoxy- or tert-lower alkoxy-carbonyl, such as tert-butoxycarbonyl or isobutoxycarbonyl,
2-halo-lower alkoxycarbonyl, such as 2-chloro-, 2-bromo-, 2-iodo- or 2,2,2-trichloroethoxycarbonyl,
aryloxycarbonyl wherein aryl has from 6 to 14 carbon atoms and is, for example, phenyl, biphenylyl, 1- or 2-naphthyl, fluorenyl, or phenyl mono- or poly-substituted by lower alkyl, for example methyl or tert-butyl, hydroxy, lower alkoxy, for example methoxy, ethoxy or tert-butoxy, halogen, for example chlorine or bromine, and/or by nitro, such as phenoxycarbonyl,
aryl-lower alkoxycarbonyl, for example arylmethoxy-carbonyl, wherein aryl has from 6 to 14 carbon atoms and is, for example, phenyl, biphenylyl, 1- or 2-naphthyl, fluorenyl, or phenyl mono- or poly-substituted by lower alkyl, for example methyl or tert-butyl, hydroxy, lower alkoxy, for example methoxy, ethoxy or tert-butoxy, halogen, for example chlorine or bromine, and/or by nitro, for example phenyl-lower alkoxycarbonyl, such as benzyloxycarbonyl, 4-methoxybenzyloxycarbonyl, 4-nitrobenzyloxycarbonyl, diphenyl-lower alkoxycarbonyl, such as diphenylmethoxycarbonyl, di-(4-methoxyphenyl)-methoxycarbonyl, trityloxycarbonyl or fluorenyl-lower alkoxycarbonyl, such as 9-fluorenylmethoxycarbonyl, especially benzyloxycarbonyl,
heterocyclyl-lower alkoxycarbonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and from thiomorpholinyl and may be unsubstituted or substituted, especially by lower alkyl, such as methyl, such as 1-methylpyrrolidin-2-yl-methoxycarbonyl, 2-furylmethoxycarbonyl, 2-tetrahydrofuranyl-lower alkoxycarbonyl, such as 2-tetrahydrofuranyl-methoxycarbonyl, 1-methyl-2-piperidylmethoxycarbonyl or 2-morpholino-ethoxycarbonyl, or 2-, 3- or 4-pyridylmethoxycarbonyl,
2-tri-lower alkylsilyl-lower alkoxycarbonyl, such as 2-trimethylsilyloxycarbonyl, or
2-triarylsilyl-lower alkoxycarbonyl wherein aryl is phenyl or 1- or 2-naphthyl, such as triphenylsilylethoxycarbonyl.

Preferred acyl groups R₁, R₂, R₈ and R₉ of an unsubstituted or substituted carbamic acid, in addition to suitable radicals already mentioned as preferred acyl groups R₁, R₂, R₈ and R₉, are carbamoyl or
unsubstituted or substituted N-alkyl- or N,N-dialkylcarbamoyl wherein the alkyl radical has up to 12 carbon atoms, preferably unsubstituted or substituted lower alkyl- or di-lower alkyl-carbamoyl, such as methyl-, ethyl-, propyl-, tert-butyl-, dimethyl-, diethyl- or di-n-propyl-carbamoyl, the substituents being selected from phenyl, for example in benzylcarbamoyl, N-phenyl-lower alkyl-N-lower alkylcarbamoyl, such as N-benzyl-N-methylcarbamoyl, or dibenzylcarbamoyl, heterocyclyl as defined under heterocyclyl-lower alkanoyl R₁, R₂, R₈ and R₉, preferably selected from thienyl, furyl, pyranyl, pyrrolyl, imidazolyl, pyrazolyl, oxazolyl, isoxazolyl, thiazolyl, furazanyl, pyridyl, pyrazinyl, pyrimidinyl, pyridazinyl, azepinyl, indolyl, benzimidazolyl, 1H-indazolyl, quinolyl, isoquinolyl, quinoxalinyl, quinazolinyl, cinnolyl, purinyl, pteridinyl, naphthyridinyl, 4H-quinolizinyl, 3,1-benzofuranyl, benz[e]indolyl, 4,1-benzoxazinyl, 4,1-benzothiazinyl, carbazolyl, β-carbolinyl, phenazinyl, phenanthridyl, acridyl, phenoxazinyl, phenothiazinyl, 1-azaacenaphthenyl, cyclohexa[b]pyrrolyl, cyclohepta[b]pyrrolyl, cyclohexa[d]pyrazolyl, cyclohexa[b]pyridyl, cyclohexa[b]pyrazinyl, cyclohexa[b]pyrimidinyl, cyclohexa[b]-1,4-oxazinyl, cyclohexa[b]-1,4-thiazinyl, pyrrolidinyl, pyrrolinyl, imidazolidinyl, 2-imidazolinyl, 2,3-dihydropyridyl, piperidyl, piperazinyl, 2,3,5,6-tetrahydropyrazinyl, morpholinyl, thiomorpholinyl, S,S-dioxothiomorpholinyl, indolinyl, isoindolinyl, 4,5,6,7-tetrahydroindolyl, 1,2,3,4-tetrahydroquinolyl, 1,2,3,4-tetrahydroisoquinolyl, chromanyl, thiochromanyl, -1,2,3,4-tetrahydro-3,1-benzodiazinyl, 3,4-dihydro-3H-4,1-benzoxazinyl, 3,4-dihydro-3H-4,1-benzothiazinyl, 2,3,4,5-tetrahydro-1H-5,1-benzazepinyl and 5,6-dihydrophenanthrid the mentioned radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthylmethoxy, amino, lower alkylamino, for example methyl-, ethyl- or tert-butyl-amino, di-lower alkyl-amino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxy-phosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, especially pyridyl, such as 2-, 3- or 4-pyridyl, more especially in N-heterocyclyl-lower alkyl-N-lower alkylcarbamoyl, for example N-pyridyl-lower alkyl-N-lower alkylcarbamoyl, such as N-(2-, 3- or 4-pyridylmethyl)-N-methyl-carbamoyl, or in N-heterocyclyl-lower alkylcarbamoyl, for example 2- or 3-pyridyl-lower alkylaminocarbonyl, such as 2- or 3-pyridylmethylaminocarbonyl, hydroxy, for example in hydroxymethyl, 2-hydroxyethyl, 3-hydroxypropyl, 2-hydroxypropyl, N-dihydroxy-lower alkyl, such as 2,3-dihydroxy-n-propyl or 2-hydroxy-2,2-dimethylethyl, lower alkoxy, preferably in lower alkoxy-lower alkyl, for example methoxymethyl or 2-methoxyethyl, lower alkanoyloxy, preferably in lower alkanoyloxy-lower alkyl, for example lower alkanoyloxymethyl or lower alkanoyloxyethyl, such as acetoxymethyl, 2-acetoxyethyl, 3-propionyloxymethyl, 2-propionyloxyethyl, 4-butyroxymethyl or 2-butyroxyethyl, aryloxy or aryloxy and hydroxy wherein aryl has from 6 to 14 carbon atoms, such as phenyl, naphthyl or fluorenyl, preferably in aryloxy-lower alkyl or aryloxyhydroxy-lower alkyl, such as phenoxymethyl, 2-phenoxyethyl, 1- or 2-naphthyloxymethyl or 1- or 2-naphthyloxyethyl, or 2-phenyl-2-hydroxyethyl, aryl being unsubstituted or mono- or di-substituted, for example by lower alkyl, for example methyl, ethyl, propyl, isopropyl, butyl, sec-butyl or tert-butyl, by hydroxy, by lower alkoxy, for example methoxy, ethoxy, propoxy, isopropoxy, butoxy, sec-butoxy or tert-butoxy, by carboxy, by lower alkoxycarbonyl, for example isopropoxycarbonyl, sec-butoxycarbonyl or tert-butoxycarbonyl, or by carbamoyl, lower alkyl- or di-lower alkyl-carbamoyl and/or by mono- or di-(hydroxy- or carboxy-lower alkyl)carbamoyl and it being possible for the mentioned substituents to be present in different ring positions, for example in the form of 4-methylphenoxy, 2,4,5-trimethylphenoxy, 4-hydroxyphenoxy, 4-methoxyphenoxy, 3,5-dimethoxyphenoxy, 2-carboxyphenoxy, 2-tert-butoxycarbonylphenoxy, 2- or 4-carbamoylphenoxy, carbamoyl, carboxy-lower alkylcarbamoyl or hydroxy-lower alkylcarbamoyl, such as in 4-carbamoyl-n-butyl, 7-carbamoyl-n-heptyl, 2-hydroxyethylcarbamoyl-n-butyl or 4-(tris(hydroxymethyl]methyl)-carbamoyl-n-butyl, and also amino, for example in 2-aminoethyl or 3-aminopropyl, lower alkylamino, for example in methyl- or ethyl-aminomethyl, di-lower alkylamino, for example in dimethylaminomethyl, halogen, especially fluorine, chlorine or bromine, for example in 2,2,2-trichloroethyl, sulfo, for example in sulfomethyl or 4-sulfobutylamino, and sulfamoyl, for example in 2-sulfamoylethyl.

Preferred acyl groups R₁, R₂, R₈ and R₉ of an unsubstituted or substituted N-substituted oxalamide are oxamoyl or lower alkyloxamoyl, such as methyl- or ethyl-oxamoyl.

Preferred acyl groups R₁, R₂, R₈ and R₉ of an unsubstituted or substituted amino acid are formed by the amino acid residues of an α- or β-amino acid, especially a natural α-amino acid having the L-configuration, such as those normally occurring in proteins, or an epimer of such an amino acid, that is to say having the unnatural D-configuration, or a D,L-isomeric mixture thereof, a homologue of such an amino acid, for example wherein the amino acid side chain has been lengthened or shortened by one or two methylene groups, wherein the amino group is in the β-position and/or wherein a methyl group has been replaced by hydrogen, a substituted aromatic amino acid wherein the aromatic radical has from 6 to 14 carbon atoms, for example a substituted phenylalanine or phenylglycine wherein the phenyl may be mono- or poly-substituted by lower alkyl, for example methyl, hydroxy, lower alkoxy, for example methoxy, lower alkanoyloxy, for example acetoxy, amino, lower alkylamino, for example methylamino, di-lower alkylamino, for example dimethylamino, lower alkanoylamino, for example acetylamino or pivaloylamino, lower alkoxycarbonylamino, for example tert-butoxycarbonylamino, arylmethoxycarbonylamino wherein aryl preferably has from 6 to 14 carbon atoms, for example benzyloxycarbonylamino or 9-fluorenylmethoxycarbonylamino, halogen, for example fluorine, chlorine, bromine or iodine, carboxy and/or by nitro, a benzo-fused phenylalanine or phenylglycine, such as α-naphthylalanine, or a hydrogenated phenylalanine or phenylglycine, such as cyclohexylalanine or cyclohexylglycine.

Those amino acids can be substituted at free amino or hydroxy functions, preferably at a free amino function, by one of the radicals mentioned above under acyl R₁ as the acyl group of a carboxylic acid, a semiester of carbonic acid, an unsubstituted or N-substituted carbamic acid or an unsubstituted or N-substituted oxalamide or by one of the radicals mentioned below under unsubstituted or substituted alkyl; aryl-lower alkyl; heterocyclyl; heterocyclyl-lower alkyl; sulfo; sulfonyl substituted by alkyl, aryl, aryl-lower alkyl, heterocyclyl-lower alkyl, alkoxy, aryloxy, aryl-lower alkoxy or by heterocyclyl-lower alkoxy; phosphoryl R₁, R₂, R₈ or R₉ substituted by one or two identical or different radicals selected from alkyl, cycloalkyl, cycloalkyl-lower alkyl, aryl, aryl-lower alkyl, hydroxy, alkoxy, cycloalkoxy, cycloalkyl-lower alkoxy, aryloxy and aryl-lower alkoxy; and sulfamoyl R₁, R₂, R₈ or R₉, which is unsubstituted or substituted at the nitrogen atom; or by one of the radicals mentioned as protecting groups in the section relating to processes.

Especially preferred is the radical, bonded via a carboxy group, of an amino acid selected from glycine (H-Gly-OH), alanine (H-Ala-OH), valine (H-Val-OH), norvaline (α-amino-valeric acid), leucine (H-Leu-OH), isoleucine (H-Ile-OH), norleucine (α-aminohexanoic acid, H-Nle-OH), serine (H-Ser-OH), homoserine (α-amino-γ-hydroxybutyric acid), threonine (H-Thr-OH), methionine (H-Met-OH), cysteine (H-Cys-OH), proline (H-Pro-OH), trans-3- and trans-4-hydroxyproline, phenylalanine (H-Phe-OH), tyrosine (H-Tyr-OH), 4-aminophenylalanine, 4-chlorophenylalanine, 4-carboxyphenylalanine, β-phenylserine (β-hydroxyphenylalanine), phenylglycine, α-naphthylalanine (H-Nal-OH), cyclohexylalanine (H-Cha-OH), cyclohexylglycine, tryptophan (H-Trp-OH), indoline-2-carboxylic acid, 1,2,3,4-tetrahydroisoquinoline-3-carboxylic acid, aspartic acid (H-Asp-OH), asparagine (H-Asn-OH), aminomalonic acid, aminomalonic acid monoamide, glutamic acid (H-Glu-OH), glutamine (H-Gin-OH), histidine (H-His-OH), arginine (H-Arg-OH), lysine (H-Lys-OH), .delta.-hydroxylysine, ornithine (α,.delta.-diaminovaleric acid), 3-aminopropanoic acid, α,γ-diaminobutyric acid and α,β-diaminopropionic acid, especially preferably the radical of an aliphatic amino acid selected from valine, alanine, leucine and isoleucine, or an amino acid selected from glycine, glutamic acid and asparagine, it being possible for each of the mentioned amino acids (with the exception of glycine) to be in the D-, L- or (D,L)-form, preferably in the L-form (with the exception of Val which may also be in the (D)- or (D,L)-form), and
wherein the α-amino group may be unsubstituted or mono- or di-N-alkylated, for example by lower alkyl, such as methyl or n-propyl, by amino-lower alkyl, such as 3-aminopropyl, by phenyl- or naphthyl-amino-lower alkyl, such as 3-phenylaminopropyl, or by piperazinylcarbonyl-lower alkyl substituted at the nitrogen atom by lower alkyl, such as methyl, such as 4-methylpiperazinylcarbonylmethyl, or may be N-acylated, for example by lower alkanoyl, such as acetyl;
by aryl-lower alkanoyl wherein aryl is selected from phenyl, indenyl, indanyl, naphthyl, anthryl, phenanthryl, acenaphthyl and fluorenyl and may be unsubstituted or especially mono- to tri-substituted by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, for example in diphenyl-, dibenzyl- or triphenyl-lower alkanoyl, such as diphenyl-, dibenzyl- or triphenyl-acetyl, and wherein lower alkanoyl may be unsubstituted or substituted, for example by lower alkyl, for example methyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-, cyclopenta-, cyclohexa- or cyclohepta-fused derivative of those radicals, which may also be fully or partially saturated, hydroxy, lower alkoxy, lower alkanoyloxy, for example acetoxy, propionyloxy, butyroxy, isobutyroxy or pivaloyloxy, acetoacetoxy, amino- or benzyloxycarbonylamino-lower alkanoyloxy, for example 2-amino- or 2-benzyloxycarbonylamino-2-methylpropionyloxy, aryl-lower alkanoyloxy wherein aryl has from 6 to 10 carbon atoms, for example in benzoyloxy, phenylacetoxy, 1- or 2-naphthoyloxy, lower alkoxycarbonyloxy, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyloxy, mono- or di-lower alkyl-aminocarbonyloxy, for example ethylaminocarbonyloxy or diethylaminocarbonyloxy, aryloxy-carbonyloxy wherein aryl has from 6 to 10 carbon atoms, for example phenoxycarbonyloxy or 1- or 2-naphthyloxycarbonyloxy, aryl-lower alkoxycarbonyloxy wherein aryl has from 6 to 14 carbon atoms, especially phenyl-lower alkoxycarbonyloxy, for example benzyloxycarbonyloxy, and also 1- or 2-naphthylmethoxycarbonyloxy or 9-fluorenylmethoxycarbonyloxy, sulfonyloxy, lower alkylsulfonyloxy, for example methyl-, ethyl-, propyl-, isopropyl-, n-butyl-, isobutyl-, sec-butyl-, tert-butyl-, n-pentyl, isopentyl-, neopentyl-, tert-pentyl-, n-hexyl-, isohexyl- or n-heptyl-sulfonyloxy, or phenylsulfonyloxy, 2- or 4-toluenesulfonyloxy or 1- or 2-naphthylsulfonyloxy, carboxy, esterified carboxy selected from lower alkoxycarbonyl, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyl, aryloxycarbonyl wherein aryl has from 6 to 10 carbon atoms, for example phenoxycarbonyl or 1- or 2-naphthyloxycarbonyl, aryl-lower alkoxycarbonyl wherein aryl has from 6 to 12 carbon atoms, for example benzyloxycarbonyl, 1- or 2-naphthylmethoxycarbonyl or 9-fluorenylmethoxycarbonyl, lower alkanoyl, lower alkylsulfonyl, for example methyl- or tert-butyl-sulfonyl, hydroxy-lower alkoxyphosphoryl and di-lower alkoxyphosphoryl, carbamoyl, carbamoyl substituted by one or two radicals selected from lower alkyl, such as methyl, ethyl, propyl, isopropyl, n-butyl, isobutyl, sec-butyl, tert-butyl, n-pentyl, isopentyl, neopentyl, tert-pentyl, n-hexyl, isohexyl or n-heptyl, for example in N-methylcarbamoyl, N-n-butylcarbamoyl or N,N-dimethylcarbamoyl, from carboxy-lower alkyl or lower alkoxycarbonyl-lower alkyl, for example in the form of carboxymethylcarbamoyl (glycinylcarbonyl) or tert-butoxycarbonylmethylcarbamoyl, from di-lower alkylamino-lower alkyl, for example 2-dimethylaminoethyl, aminocarboxy-lower alkyl, for example 5-amino-5-carboxypentyl, from hydroxy-lower alkyl, for example hydroxymethyl or hydroxyethyl, and from di-lower alkoxy-lower alkyl, for example 2-(2,2-dimethoxyethyl), or carbamoyl substituted by one radical selected from ethylene, trimethylene, tetramethylene and pentamethylene in which a carbon atom may have been replaced by nitrogen, oxygen, sulfur or by sulfur mono- or di-substituted by oxygen and which may be unsaturated, for example in the form of piperidino-, pyrazin-1-yl-, piperazin-1-yl-, pyrimidin-1-yl-, pyridazin-1-yl-, morpholino-, thiomorpholino- or S, S-dioxothiomorpholino-carbonyl; sulfamoyl, phosphono, benzofuranyl, oxo and/or by cyano and is unbranched or branched, preferably by aryl-lower alkanoyl wherein aryl has from 6 to 14 carbon atoms and is unsubstituted or substituted, for example by lower alkanoyl, such as in 2-benzyl-3-pivaloylpropionyl, or by lower alkylsulfonyl, such as in 2-benzyl-3-tert-butylsulfonylpropionyl, with phenyl-lower alkanoyl, for example phenylacetyl, being especially preferred;
by heterocyclyl-lower alkanoyl selected from thienyl-, furyl-, pyranyl-, pyrrolyl-, imidazolyl-, pyrazolyl-, oxazolyl-, isoxazolyl-, thiazolyl-, furazanyl-, tetrazolyl-, pyridyl-, pyrazinyl-, pyrimidinyl-, pyridazinyl-, azepinyl-, indolyl-, benzimidazolyl-, 1H-indazolyl-, quinolyl-, isoquinolyl-, quinoxalinyl-, quinazolinyl-, cinnolyl-, purinyl-, pteridinyl-, naphthyridinyl-, 4H-quinolizinyl-, 3,1-benzofuranyl-, benz[e]indolyl-, 4,1-benzoxazinyl-, 4,1-benzothiazinyl-, carbazolyl-, β-carbolinyl-, phenazinyl-, phenanthridyl-, acridyl-, phenoxazinyl-, phenothiazinyl-, 1-azaacenaphthenyl-, cyclohexa[b]pyrrolyl-, cyclohepta[b]pyrrolyl-, cyclohexa[d]pyrazolyl-, cyclohexa[b]pyridyl-, cyclohexa[b]pyrazinyl-, cyclohexa[b]pyrimidinyl-, cyclohexa[b]-1,4-oxazinyl-, cyclohexa[b]-1,4-thiazinyl-, pyrrolidinyl-, pyrrolinyl-, imidazolidinyl-, 2-imidazolinyl-, 2,3-dihydropyridyl-, piperidyl-, piperazinyl-, 2,3,5,6-tetrahydropyrazinyl-, morpholinyl-, thio-morpholinyl-, S,S-dioxothiomorpholinyl-, indolinyl-, isoindolinyl-, 4,5,6,7-tetrahydro-indolyl-, 1,2,3,4-tetrahydroquinolyl-, 1,2,3,4-tetrahydroisoquinolyl-, chromanyl-, thiochromanyl, 1,2,3,4-tetrahydro-3,1-benzodiazinyl-, 3,4-dihydro-3H-4,1-benzoxazinyl-, 3,4-dihydro-3H-4,1-benzothiazinyl-, 2,3,4,5-tetrahydro-1H-5,1-benzazepinyl- and 5,6-dihydrophenanthridinyl-lower alkanoyl, the mentioned heterocyclyl radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthylmethoxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxy-phosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, for example pyridylcarbonyl, such as 2-, 3- or 4-pyridylcarbonyl, 3,4-dihydroxypyrrolidinylcarbonyl, N-benzyloxycarbonyl-piperidin-4-ylcarbonyl, 1-methylpiperazin-4-ylcarbonyl, morpholinocarbonyl, thiomorpholinocarbonyl, S,S-dioxothiomorpholinocarbonyl, indol-2-ylcarbonyl, quinol-2-ylcarbonyl, pyridylacetyl, such as 2- or 3-pyridylacetyl, imidazolylacetyl, such as imidazol-1-ylacetyl, morpholinylacetyl, such as morpholinoacetyl, pyridylpropionyl, such as 3-(2- or 3-pyridyl)propionyl, pyrrolidinylpropionyl, such as 3-(4-pyrrolidinyl)propionyl, morpholinylpropionyl, such as 3-morpholinopropionyl, or tetrazolylpropionyl, such as 3-(tetrazol-1-yl)-propionyl;
by heterocyclyl-lower alkenoyl wherein heterocyclyl is selected especially from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, for example pyrrolidyl-lower alkenoyl, such as N-pyrrolidyl-acryloyl, halo-lower alkanoyl containing up to 3 halogen atoms, for example α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl;
by lower alkoxycarbonyl, such as tert-butoxycarbonyl;
by aryl-lower alkoxycarbonyl wherein aryl has from 6 to 14 carbon atoms and is selected, for example, from phenyl, naphthyl and fluorenyl, for example phenyl-lower alkoxycarbonyl, such as benzyloxycarbonyl, or 9-fluorenylmethoxycarbonyl;
by heterocyclyl-lower alkoxycarbonyl wherein heterocyclyl is selected especially from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated and unsubstituted or substituted especially by lower alkyl, such as methyl, for example 1-methylpyrrolidin-2-yl-methoxycarbonyl, 2-furylmethoxycarbonyl, tetrahydrofuranyl-lower alkoxycarbonyl, such as 2-tetrahydrofuranyl-methoxycarbonyl, 1-methyl-2-piperidyl-methoxycarbonyl or 2-morpholinoethoxycarbonyl;
by carboxy-lower alkanoyl, such as 3-carboxypropionyl, 5-carboxypentanoyl or 6-carboxyhexanoyl;
by lower alkoxycarbonyl-lower alkanoyl, such as 5-methoxycarbonylpentanoyl or 6-methoxycarbonylhexanoyl;
by hydroxy-lower alkoxy-lower alkanoyl, such as 3-hydroxy-n-propoxycarbonyl;
by amino-lower alkanoyl wherein the amino group is not in the α- or β-position of the lower alkanoyl radical, such as 5-aminopentanoyl;
by phenyl-lower alkoxycarbonylamino-lower alkanoyl wherein the amino group is not in the α- or β-position of the lower alkanoyl radical, such as 5-benzyloxycarbonylaminopentanoyl or 6-benzyloxycarbonylaminohexanoyl;
by amino-lower alkanoyl substituted at the amino nitrogen atom by heterocyclyl-lower alkanoyl, preferably as defined above for heterocyclyl-lower alkanoyl R₁, R₂, R₈ or R₉, especially by N-morpholino- or N-thiomorpholino-carbonyl, for example N-morpholino- or N-thiomorpholino-carbonylamino-lower alkanoyl, such as N-morpholino- or N-thiomorpholino-carbonylamino-acetyl;
by carbamoyl;
by phenyl-lower alkylaminocarbonyl, such as benzylaminocarbonyl;
by N-di-lower alkylamino-lower alkyl-N-lower alkylaminocarbonyl, such as N-(2-dimethylamino)ethyl-N-methylaminocarbonyl;
by N-dihydroxy-lower alkyl-N-lower alkylaminocarbonyl, such as N-(2,3-dihydroxy-n-propyl)-N-methylaminocarbonyl;
by N-heterocyclyl-lower alkyl-N-lower alkylcarbamoyl wherein heterocyclyl is selected from thienyl, furyl, pyranyl, pyrrolyl, imidazolyl, pyrazolyl, oxazolyl, isoxazolyl, thiazolyl, furazanyl, pyridyl, pyrazinyl, pyrimidinyl, pyridazinyl, azepinyl, indolyl, benzimidazolyl, 1H-indazolyl, quinolyl, isoquinolyl, quinoxalinyl, quinazolinyl, cinnolyl, purinyl, pteridinyl, naphthyridinyl, 4H-quinolizinyl, 3,1-benzofuranyl, benz[e]indolyl, 4,1-benzoxazinyl, 4,1-benzothiazinyl, carbazolyl, β-carbolinyl, phenazinyl, phenanthridyl, acridyl, phenoxazinyl, phenothiazinyl, 1-azaacenaphthenyl, cyclohexa[b]pyrrolyl, cyclohepta[b]pyrrolyl, cyclohexa[d]pyrazolyl, cyclohexa[b]pyridyl, cyclohexa[b]pyrazinyl, cyclohexa[b]pyrimidinyl, cyclohexa[b]-1,4-oxazinyl, cyclohexa[b]-1,4-thiazinyl, pyrrolidinyl, pyrrolinyl, imidazolidinyl, 2-imidazolinyl, 2,3-dihydropyridyl, piperidyl, piperazinyl, 2,3,5,6-tetrahydropyrazinyl, morpholinyl, thiomorpholinyl, S,S-dioxothiomorpholinyl, indolinyl, isoindolinyl, 4,5,6,7-tetrahydroindolyl, 1,2,3,4-tetrahydroquinolyl, 1,2,3,4-tetra-hydroisoquinolyl, chromanyl, thiochromanyl, 1,2,3,4-tetrahydro-3,1-benzodiazinyl, 3,4-dihydro-3H-4,1-benzoxazinyl, 3,4-dihydro-3H-4,1-benzothiazinyl, 2,3,4,5-tetrahydro-1H-5,1-benzazepinyl and 5,6-dihydrophenanthridinyl, the mentioned radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or, naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthylmethoxy, amino, lower alkylamino, for example methyl-, ethyl- or tert-butyl-amino, di-lower alkyl-amino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxy-phosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, especially pyridyl, such as 2-, 3- or 4-pyridyl, for example 2- or 3-pyridyl-lower alkylaminocarbonyl, such as 2- or 3-pyridylmethylaminocarbonyl;
by N-2-, N-3- or N-4-pyridyl-lower alkyl-N-lower alkylaminocarbonyl, such as N-2-, N-3- or N-4-pyridylmethyl-N-methylaminocarbonyl;
by heterocyclyl-lower alkylcarbamoyl-lower alkanoyl, such as defined above for heterocyclyl-lower alkylcarbamoyl-lower alkanoyl R₁, R₂, R₈ or R₉, for example 2-(N-morpholino-lower alkylcarbamoyl)-lower alkanoyl, such as 2(R,S)-(N-(2-morpholinoethyl)-carbamoyl)-3-methyl-butyryl, or 2-(N-(pyridyl-lower alkyl)-carbamoyl)-lower alkanoyl, such as (2(R,S)-(N-(2-pyridylmethyl)-carbamoyl)-3-methyl)-butyryl;
by sulfonyl; by lower alkylsulfonyl, such as methyl- or ethyl-sulfonyl; by arylsulfonyl wherein aryl has from 6 to 10 carbon atoms and, for example, is selected from phenyl and naphthyl and is unsubstituted or especially substituted by lower alkyl, such as methyl, or by lower alkoxy, such as methoxy, such as p-toluenesulfonyl; by heterocyclylsulfonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and from thiomorpholinyl and may be unsubstituted or substituted, especially by lower alkyl, such as methyl, such as morpholinosulfonyl, thiomorpholinosulfonyl, piperidinosulfonyl, 4-methylpiperazinylsulfonyl or piperazinosulfonyl (heterocyclylsulfonyl not being a substituent in preferred forms); by sulfamoyl or by sulfamoyl substituted by heterocyclyl-lower alkyl, wherein heterocyclyl is as last defined, and/or by lower alkyl, such as N-2-pyridylmethyl-N-methylaminosulfonyl,
a carboxy group of the side chain is present in esterified or amidated form, for example in the form of a lower alkyl ester group, such as methoxycarbonyl or tert-butoxycarbonyl, an aryl ester group or an aryl-lower alkyl ester group, aryl being phenyl, 4-nitrophenyl, naphthyl or biphenylyl, for example in the form of a 4-nitrophenoxycarbonyl, benzyloxycarbonyl or 9-fluorenylmethoxycarbonyl group, or in the form of a carbamoyl, a lower alkylcarbamoyl, such as methylcarbamoyl, a di-lower alkylaminocarbamoyl, such as dimethylcarbamoyl, a mono- or di-(hydroxy-lower alkyl)carbamoyl, such as hydroxymethylcarbamoyl or di(hydroxymethyl)carbamoyl, or mono- or di-(carboxy-lower alkyl)carbamoyl group, such as a carboxymethylcarbamoyl or di-(carboxymethyl)carbamoyl group,
an amino group of the side chain is present in alkylated form, for example in the form of mono- or di-lower alkylamino, such as n-butylamino or dimethylamino, or in acylated form, for example in the form of lower alkanoylamino, such as acetylamino or pivaloylamino, amino-lower alkanoylamino, such as 3-amino-3,3-dimethylpropionylamino, aryl-lower alkanoylamino wherein aryl has from 6 to 14 carbon atoms, for example phenyl, naphthyl or fluorenyl, and is unsubstituted or substituted by lower alkyl, hydroxy, lower alkoxy, carboxy, carbamoyl or by sulfamoyl, such as 4-hydroxyphenylbutyryl, lower alkoxycarbonylamino, such as tert-butoxycarbonylamino, arylmethoxycarbonylamino wherein aryl has from 6 to 14 carbon atoms, such as benzyloxycarbonylamino or 9-fluorenylmethoxycarbonylamino, piperidyl-1-carbonyl, morpholinocarbonyl, thiomorpholinocarbonyl or S,S-dioxothiomorpholinocarbonyl, and/or
a hydroxy group of the side chain is present in etherified or esterified form, for example in the form of a lower alkoxy, such as methoxy or tert-butoxy, aryl-lower alkoxy, such as benzyloxy, lower alkanoyloxy, such as acetoxy, or lower alkoxycarbonyloxy group, for example a tert-butoxycarbonyloxy group.

Special preference is given to acyl groups R₁, R₂, R₈ and R₉ of an unsubstituted or substituted amino acid selected from phenylalanine, N-(benzyloxycarbonyl)-phenylalanine, N-(2(R,S)-benzyl-3-pivaloyl-propionyl)-phenylalanine, N-(9-fluorenylmethoxycarbonyl)-phenylalanine, tyrosine, N-propyltyrosine, tyrosine-O-methyl ether, N-(3-amino-3,3-dimethylpropionyl)-tyrosine-O-methyl ether, N-(2(S)-benzyl-3-tert-butylsulfonylpropionyl)-tyrosine-O-tert-butyl ether, N-(9-fluorenyl-methoxycarbonyl)-tyrosine-O-methyl ether, N-(9-fluorenylmethoxycarbonyl)-tyrosine-O-tert-butyl ether, N-morpholinocarbonyl-glycine, N-(N-(2-, 3- or 4-pyridyl)methyl-N-methylaminocarbonyl)glycine, valine, N-(3-phenylaminopropyl)-valine, N-(4-methylpiperazinylcarbonylmethyl)-valine, N-(trifluoroacetyl)-valine, N-phenylacetyl-valine, N-acetyl-valine, N-(3-phenylpropionyl)-valine, N-(2(R,S)- or -(2S)-benzyl-3-pivaloyl-propionyl)-valine, N-(2-carbamoyl-3-phenyl-propionyl)-valine, N-(2(S)-benzyl-3-tert-butylsulfonylpropionyl)valine, N-(2(R,S)-carbamoyl-3-phenyl-propionyl) valine, N-(2-, 3- or 4-pyridylcarbonyl)-valine, N-(1-imidazolylacetyl)-valine, N-(2- or 3-pyridylacetyl)-valine, N-(morpholino-acetyl)-valine, N-(3-(2- or 3-pyridyl)-propionyl)-valine, N-(3-(4-pyrrolidinyl)-propionyl)-valine, N-(3-(morpholino)-propionyl)-valine, N-(N-benzyloxycarbonylpiperidin-4-yl-carbonyl)-valine, N-tetrahydrofurylmethoxycarbonyl-valine, N-3-(tetrazol-1-yl)-propionyl-valine, N-(indol-2-ylcarbonyl)-valine, N-(quinoline-2-carbonyl)-valine, N-(1-methylpiperazin-4-ylcarbonyl)-valine, N-(3,4-dihydroxypyrrolidinylcarbonyl)-valine, N-methoxycarbonyl-valine, N-isobutoxycarbonyl-valine, N-tert-butoxycarbonyl-valine, N-benzyloxycarbonyl-valine, N-(2-furylmethoxycarbonyl)-valine, N-(1-methylpyrrolidin-2-yl-methoxycarbonyl)-valine, N-(1-methyl-2-piperidyl methoxycarbonyl)valine, N-(1-methyl-3-piperidyl-methoxycarbonyl)-valine, N-(2-(morpholino)ethoxycarbonyl)-valine, N-(3-carboxypropionyl)-valine, N-(5-carboxypentanoyl)-valine, N-(6-carboxyhexanoyl)-valine, N-(5-methoxycarbonylpentanoyl)-valine, N-(6-methoxycarbonylhexanoyl)-valine, N-(3-aminopropionyl)-valine, N-(4-aminobutyryl)-valine, N-(5-benzyloxycarbonyl-aminopentanoyl)-valine, N-(6-benzyloxycarbonylaminohexanoyl)-valine, N-(morpholinocarbonyl)-valine, N-(thiomorpholinocarbonyl)-valine, N-(S,S-dioxothio-morpholinocarbonyl)-valine, N-(N-benzylaminocarbonyl)-valine, N-(N-2-pyridylmethyl-N-methylaminocarbonyl)-valine, N-(N-3-pyridylmethyl-aminocarbonyl) valine, N-(N-2-pyridylmethyl-aminocarbonyl)-valine, N-morpholino-carbonylamino-acetyl-valine, N-(2-pyrrolidylacryloyl)-valine, N-methylsulfonyl-valine, N-morpholinosulfonyl-valine, N-(p-toluenesulfonyl)-valine, N-(4-methylpiperazinyl-sulfonyl)-valine, N-(N-(2-pyridylmethyl)-N-methyl-sulfamoyl)-valine, N-(N-2-pyridyl-methyl-N-methyl-aminocarbonyl)-valine, N-(3-aminopropyl)-leucine, N-acetyl-leucine, N-(3-aminopropionyl)-leucine, N-(2(R,S)- or N-(2S)-benzyl-3-pivaloyl-propionyl)-leucine, N-(2(S)-benzyl-3-tert-butylsulfonylpropionyl)-leucine, N-(2-, 3- or 4-pyridylcarbonyl)-leucine, N-(4-thiomorpholinocarbonyl)-leucine, N-(4-(S,S-dioxothiomorpholino)carbonyl)-leucine, N-(4-aminobutyryl)-leucine, N-(3-hydroxy-n-propoxycarbonyl)-leucine, N-(benzyloxycarbonyl)-leucine, N-(N-(2-(dimethylamino) ethyl)-N-methyl-aminocarbonyl)-leucine, N-(N-(2,3-dihydroxy-n-propyl)-N-methyl-aminocarbonyl)-leucine, N-acetyl-isoleucine, N-propionyl-isoleucine, N-(benzyloxycarbonyl)isoleucine, N-(2(R,S)-benzyl-3-pivaloyl-propionyl)-norleucine, N-(2 (S)-benzyl-3-tert-butylsulfonylpropionyl)-norleucine, N-(tert-butoxycarbonyl)-norleucine, N-(tert-butoxycarbonyl)-serine, N-(benzyloxycarbonyl)-serine, N-acetyl-serine O-methyl ether, N-(benzyloxycarbonyl)-serine O-methyl ether, N-(2(R,S)-benzyl-3-pivaloyl-propionyl)serine, N-benzyloxycarbonyl-glutamic acid, asparagine, N-benzyloxycarbonyl-asparagine, quinoline-2-carbonyl-asparagine, and N-(morpholinocarbonyl)-asparagine, the amino acid residues preferably being in the (L)- or (D,L)-form, and in the case of valine also in the (D)-form.

Unsubstituted or substituted alkyl R₁, R₂, R₈ or R₉ contains an alkyl radical having from 1 to 20, preferably up to 10, carbon atoms, is branched or unbranched, may contain instead of a methylene group a hetero atom selected from thia, aza and selena and is, for example, methyl, ethyl, n-propyl, isopropyl, n-butyl, isobutyl, sec-butyl, tert-butyl, n-pentyl, isopentyl, neopentyl, tert-pentyl, n-hexyl, isohexyl, n-heptyl, n-octyl, n-nonyl or n-decyl. Preference is given to lower alkyl, for example methyl, ethyl, n-propyl, isopropyl, n-butyl, isobutyl, sec-butyl, tert-butyl, n-pentyl, isopentyl, neopentyl, tert-pentyl, n-hexyl, isohexyl or n-heptyl, which is unsubstituted or substituted.

Radicals suitable as substituents in substituted alkyl, preferably substituted lower alkyl, are the radicals mentioned for lower alkanoyl R₁, R₂, R₈ and R₉.

Substituted lower alkyl is preferably cycloalkyl-lower alkyl wherein cycloalkyl has from 3 to 7 carbon atoms and lower alkyl is as defined above, for example cycloalkyl-methyl or -ethyl, preferably having a total of from 4 to 13 carbon atoms, for example cyclopropyl-, cyclobutyl-, cyclopentyl- or cyclohexyl-lower alkyl, such as -methyl or -ethyl,
cycloalkenyl-lower alkyl, for example cycloalkenylmethyl, wherein cycloalkyl preferably has from 4 to 8 carbon atoms, such as 1-cyclohexenylmethyl, 1,4-cyclohexadienylmethyl or 1-cyclohexenylethyl or 1,4-cyclohexadienylethyl,
bicycloalkyl-lower alkyl wherein bicycloalkyl has, for example, from 5 to 10 carbon atoms, for example bicycloalkyl-methyl or -ethyl, preferably having from 8 to 11 carbon atoms, such as decahydronaphthyl-2-methyl, endo- or exo-norbornyl-2-methyl, bicyclo[2.2.2]oct-2-ylmethyl or bicyclo[3.3.1]non-9-ylmethyl, and also bicyclo-hexyl-, -heptyl-, -octyl-, -nonyl- or -decyl-ethyl or -3-propyl, for example bicyclo[3.1.0]hex-1-, -2- or -3-yl-, bicyclo[4.1.0]hept-0.1- or -7-yl-, bicyclo[2.2.1]hept-2-yl-, for example endo- or -exo-norbornyl-, bicyclo[3.2.1]oct-2-yl-, bicyclo[3.3.0]oct-3-yl- or bicyclo[3.3.1]non-9-yl-, and also α- or β-decahydronaphthyl-ethyl or -3-propyl,
tricycloalkyl-lower alkyl wherein tricycloalkyl contains, for example, from 8 to 10 carbon atoms, for example tricycloalkyl-methyl or -ethyl, preferably having from 8 to 11 carbon atoms, such as 1- or 2-adamantylmethyl, and also tricyclo[5.2.1.0^{2,6}]dec-8-yl- or adamantyl-, such as 1-adamantyl-ethyl,
aryl-lower alkyl wherein especially aryl has from 6 to 14 carbon atoms, such as in phenyl, indenyl, indanyl, naphthyl, anthryl, phenanthryl, acenaphthyl or fluorenyl, which may be unsubstituted or especially mono- to tri-substituted by lower alkyl, for example methyl, ethyl or isopropyl, halo-lower alkyl, such as trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine or chlorine, carboxy, lower alkoxycarbonyl, benzyl-, naphthyl- or fluorenyl-lower alkoxycarbonyl, lower alkanoyl, sulfo, lower alkylsulfonyl, phosphono, hydroxy-lower alkoxy-phosphoryl or di-lower alkoxyphosphoryl, carbamoyl, mono- or di-lower alkylcarbamoyl, sulfamoyl, mono- or di-lower alkylsulfamoyl, nitro and/or by cyano, wherein phenyl may be present up to three times, such as in diphenyl-, dibenzyl- or triphenyl-lower alkyl, for example diphenyl-, dibenzyl- or triphenyl-2-ethyl, and
wherein lower alkyl is unsubstituted or substituted, for example by lower alkyl, such as methyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, hydroxy, lower alkoxy, lower alkanoyloxy, such as acetoxy, propionyloxy, butyroxy, isobutyroxy or pivaloyloxy, acetoacetoxy, amino- or benzyloxycarbonylamino-lower alkanoyloxy, such as 2-amino- or 2-benzyloxycarbonylamino-2-methylpropionyloxy, aryl-lower alkanoyloxy wherein aryl has from 6 to 10 carbon atoms, such as in benzoyloxy, phenylacetoxy, 1- or 2-naphthoyloxy, lower alkoxycarbonyloxy, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy, tert-pentyloxy-, n-hexyloxy-, isohexyloxy- or n-heptyloxy-carbonyloxy, mono- or di-lower alkyl-aminocarbonyloxy, such as ethylaminocarbonyloxy or diethylaminocarbonyloxy, aryloxycarbonyloxy wherein aryl has from 6 to 10 carbon atoms, for example phenoxycarbonyloxy or 1- or 2-naphthyloxycarbonyloxy, aryl-lower alkoxycarbonyloxy wherein aryl has from 6 to 12 carbon atoms, for example phenyl-lower alkoxycarbonyloxy, such as benzyloxycarbonyloxy, and also 1- or 2-naphthylmethoxycarbonyloxy or 9-fluorenylmethoxycarbonyloxy, sulfonyloxy, lower alkylsulfonyloxy, for example methyl-, ethyl-propyl-, isopropyl-, n-butyl-, isobutyl-, sec-butyl-, tert-butyl-, n-pentyl-, isopentyl-, neopentyl-, tert-pentyl-, n-hexyl-, isohexyl- or n-heptyl-sulfonyloxy, phenylsulfonyloxy, 2- or 4-toluenesulfonyloxy, 1- or 2-naphthylsulfonyloxy, amino, mono- or di-lower alkylamino, N-lower alkoxy-N-lower alkylamino, such as N-methoxy-N-methylamino, mono- or di-(phenyl- or -naphthyl-lower alkyl)amino, such as benzylamino, lower alkanoylamino, such as pivaloylamino, carboxy, esterified carboxy selected from lower alkoxycarbonyl, for example methoxy-ethoxy-, n-propoxy-, isopropoxy-, n-butoxy-, isobutoxy-, sec-butoxy-, tert-butoxy-, n-pentyloxy-, isopentyloxy-, neo-pentyloxy-, tert-pentyloxy-, n-hexyloxy-, isohexyloxy- or n-, heptyloxy-carbonyl, aryloxycarbonyl wherein aryl has from 6 to 10 carbon atoms, for example phenoxycarbonyl or 1- or 2-naphthyloxycarbonyl, aryl-lower alkoxycarbonyl wherein aryl has from 6 to 12 carbon atoms, for example benzyloxycarbonyl, 1- or 2-naphthylmethoxycarbonyl or 9-fluorenylmethoxycarbonyl, lower alkanoyl, such as pivaloyl or acetyl, lower alkylsulfonyl, such as tert-butylsulfonyl, hydroxy-lower alkoxyphosphoryl and di-lower alkoxyphosphoryl, carbamoyl, carbamoyl substituted by one or two radicals selected from lower alkyl, for example methyl, ethyl, propyl, isopropyl, n-butyl, isobutyl, sec-butyl, tert-butyl, n-pentyl, isopentyl, neopentyl, tert-pentyl, n-hexyl, isohexyl or n-heptyl, such as in N-methylcarbamoyl, N-n-butylcarbamoyl or N,N-dimethylcarbamoyl, from carboxy-lower alkyl or lower alkoxycarbonyl-lower alkyl, for example in the form of carboxy-methylcarbamoyl (glycinylcarbonyl) or tert-butoxycarbonylmethylcarbamoyl, from di-lower alkylamino-lower alkyl, for example 2-dimethylaminoethyl, from aminocarboxy-lower alkyl, such as 5-amino-5-carboxypentyl, from hydroxy-lower alkyl, for example hydroxymethyl or hydroxyethyl, and from di-lower alkoxy-lower alkyl, for example 2-(2,2-dimethoxyethyl), or carbamoyl substituted by one radical selected from ethylene, trimethylene, tetramethylene and pentamethylene in which a carbon atom may have been replaced by nitrogen, oxygen, sulfur or by sulfur mono- or di-substituted by oxygen and which may be unsaturated, for example in the form of piperidino-, pyrazin-1-yl-, piperazin-1-yl-, pyrimidin-1-yl-, pyridazin-1-yl-, morpholino-, thiomorpholino- or S,S-di-oxothiomorpholino-carbonyl; sulfamoyl, phosphono, benzfuranyl, oxo (which is not present at the carbon atom bonded to the amino nitrogen atom linked to R₁, R₂, R₈ or R₉) and/or by cyano and is unbranched or branched, is especially selected from phenyl-lower alkyl, such as benzyl that is unsubstituted or mono- or poly-substituted in the benzyl radical by lower alkyl, for example methyl, phenyl, hydroxy, lower alkoxy, for example methoxy, halogen, for example chlorine, nitro, and/or by cyano, such as 4-methoxy-, 4-fluoro-, 4-chloro-, 4-nitro- or 4-cyano-benzyl, naphthylmethyl, such as α- or β-naphthylmethyl, indenylmethyl, for example 1-, 2- or 3-indenylmethyl, indanylmethyl, such as 1- or 2-indanylmethyl, and phenanthrenylmethyl, such as 9-phenanthrenylmethyl, 2-phenylethyl, 2-α-naphthylethyl, 2-p-naphthylethyl, 2-lower alkylphenyl-ethyl, such as 2-(4-methylphenyl)ethyl, 2-lower alkoxyphenylethyl, such as 2-(4-methoxyphenyl)ethyl, 2,2-diphenylethyl, 2,2-di(4-methoxyphenyl)ethyl, 2,2,2-triphenylethyl and 2,2-dibenzylethyl, from phenyl-lower alkyl substituted in the 2- and p-positions by two radicals selected from phenyl-lower alkoxycarbonyl, such as benzyloxycarbonyl, and lower alkanoylamino, such as pivaloylamino, such as 2,p-dibenzyloxycarbonylamino-phenylethyl or 2-pivaloylamino-p-benzyloxycarbonyl-amino-phenylethyl, 2,p-diamino-phenylethyl, 3-phenylpropyl, 3-(p-hydroxyphenyl)-propyl, 3-α- or 3-β-naphthylpropyl, 2-benzyl-3-(1-pyrazolyl)-propyl, 3-phenyl- or 3-α-naphthyl-2-hydroxy-propyl, 3-phenyl- or 3-α-naphthyl-2-lower alkoxypropyl, such as 3-phenyl- or 3-α-naphthyl-2-neopentyloxy-propyl, 3-phenyl- or 3-(α-naphthyl-2-lower alkanoyloxy-propyl, such as 3-phenyl-2-pivaloyloxy- or -2-acetoxypropyl, 2-benzyl- or 1- or 2-naphthyl-3-(N-methoxyl-N-methylamino)-propyl, 3-phenyl- or 3-α-naphthyl-2-dimethylaminomethyl-propyl, 3-α-naphthyl-2-pivaloyloxy- or -2-acetoxy-propyl, 3-α-naphthyl-2-acetoacetoxy-propyl, 3-α-naphthyl-2-ethylamino-carbonyloxy-propyl or 3-α-naphthyl-2-((2-amino- or 2-benzyloxycarbonylamino)-2-methylpropionyloxy)-propyl, 3-phenyl- or 3-α-naphthyl-2-carboxymethylpropyl, 3-phenyl- or 3-α-naphthyl-2-lower alkoxycarbonyl-propyl, such as 3-α-naphthyl-2-ethoxycarbonyl-propyl, 3-phenyl- or 3-at-naphthyl-2-benzyloxycarbonylmethyl-propyl, 2-(S)-benzyl-3-tert-butylsulfonyl-propyl, 3-phenyl-2-phosphono- or -phosphonomethylpropyl, 3-phenyl-2-dimethoxyphosphoryl- or -dimethoxyphosphorylmethyl-propyl, 3-phenyl-2-diethoxyphosphoryl- or -diethoxyphosphorylmethyl-propyl, 3-phenyl-2-ethoxy- or -methoxyhydroxyphosphoryl-propyl, 3-phenyl- or 3-α-naphthyl-2-carbamoyl-propyl, 3-phenyl- or 3-α-naphthyl-2-tert-butylcarbamoyl-propyl, 3-phenyl- or 3-α-naphthyl-2-(2-dimethylaminoethyl)carbamoyl-propyl, 3-α-naphthyl-2-(carboxy- or tert-butoxycarbonyl)methylcarbamoyl-propyl, 3-phenyl- or 3-α-naphthyl-2-(3-hydroxy-2-propyl)carbamoyl-propyl, 3-phenyl- or 3-α-naphthyl-2-(2,2-dimethoxyethyl) carbamoylpropyl, 3-phenyl- or 3-α-naphthyl-2-(5-amino-5-carboxypentyl)-carbamoylpropyl, 3-phenyl- or 3-α-naphthyl-2-cyano-propyl, 3-phenyl- or 3-α-naphthyl-2-cyanomethyl-propyl, 3-phenyl- or 3-α-naphthyl-2-acetonyl-propyl, 4-hydroxyphenylbutyl, 4-phenyl- or 4-α-naphthyl-3-carboxy-butyl, 4-phenyl- or 4-α-naphthyl-3-benzyloxycarbonyl-butyl, 2-benzyl-4-(2-benzofuranyl)-4-oxobutyl, 2-benzyl- or 2-α-naphthylmethyl-4-cyano-butyl, 2-benzyl- or 2-α-naphthylmethyl-5-dimethylamino-pentyl, 2-benzyl- or 2-α-naphthylmethyl-4-oxo-pentyl, 2-benzyl- or 2-α-naphthylmethyl-4,4-dimethyl-3-oxo-pentyl, 2-benzyl- or 2-α-naphthylmethyl-5-dimethylamino-4-oxo-pentyl or 2-benzyl- or 2-α-naphthylmethyl-5,5-dimethyl-4-oxo-hexyl, preferably phenyl-lower alkyl, such as benzyl, 2-phenylethyl or 3-phenylpropyl, 4-hydroxybenzyl, 1- or 2-naphthylmethyl or 1- or 2-naphthyl-2-ethyl, especially phenyl-lower alkyl as last defined,
heterocyclyl-lower alkyl containing especially unsubstituted or substituted heterocyclyl as mentioned under heterocyclyl-lower alkanoyl R₁, R₂, R₈ and R₉ and lower alkyl that is unsubstituted or substituted in the same manner as lower alkanoyl in heterocyclyl-lower alkanoyl R₁, R₂, R₈ and R₉ (oxo not being present at the carbon atom bonded to the nitrogen atom carrying the radical R₁, R₂, R₈ or R₉), for example methyl, 2-ethyl or 3-propyl bonded to unsubstituted or lower alkyl- or phenyl-substituted pyrrolyl, such as 2- or 3-pyrrolyl, 4- or 5-methylpyrrolyl or 4- or 5-phenylpyrrolyl, thienyl, such as 2-thienyl, furyl, such as 2-furyl, pyrazolyl, such as 1-pyrazolyl, tetrazolyl, such as tetrazol-1-yl, pyridyl, such as 2-, 3- or 4-pyridyl, indolyl that is unsubstituted or substituted by lower alkyl, for example methyl, phenyl-lower alkyl, for example benzyl, lower alkoxy, for example methoxy, phenyl-lower alkoxy, for example benzyloxy, or by halogen, for example by chlorine, such as 2-, 3- or 5-indolyl, 1-methyl-, 2-methyl-, 5-methoxy-, 5-benzyloxy-, 5-chloro- or 4,5-dimethyl-indol-2-yl, 1-benzylindol-2-yl or -3-yl, 4,5,6,7-tetrahydroindol-2-yl, cyclohepta[b]pyrrol-5-yl, unsubstituted or hydroxy-substituted quinolyl, such as 2-, 3- or 4-quinolyl or 4-hydroxyquinol-2-yl, unsubstituted or hydroxy-substituted isoquinolyl, such as 1-, 3- or 4-isoquinolyl or 1-oxo-1,2-dihydroisoquinol-3-yl, 2-quinoxalinyl, 3,1-benzfuran-2-yl, benz[e]indol-2-yl, β-carbolin-3-yl, 3-chromanyl, 3-thiochromanyl, 3-pyrrolidinyl, hydroxypyrrolidinyl, such as 3- or 4-hydroxypyrrolidin-2-yl, oxopyrrolidinyl, such as 5-oxopyrrolidin-2-yl, piperidinyl, such as 2-, 3- or 4-piperidinyl, morpholinyl, such as 2- or 3-morpholinyl, thiomorpholinyl, such as 2- or 3-thiomorpholinyl, S,S-dioxothiomorpholinyl, such as S,S-dioxothiomorpholin-2- or -3-yl, indolinyl, such as 2- or 3-indolinyl, 1,2,3,4-tetrahydroquinolyl, such as 1,2,3,4-tetrahydroquinol-2-, -3- or -4-yl, 1,2,3,4-tetrahydroisoquinolyl, such as 1,2,3,4-tetrahydroisoquinol-1-, -2- or -3-yl, or 1-oxo-1,2,3,4-tetrahydroisoquinol-3-yl, or 4-pyrrolidinylmethyl or 1-imidazolylmethyl,
hydroxy-lower alkyl, such as 3-hydroxypropyl or 2-hydroxy-3-methylpentyl,
lower alkoxy-lower alkyl, for example lower alkoxyethyl or lower alkoxypropyl, such as 2-methoxyethyl, 2-ethoxyethyl or 3-methoxypropyl,
lower alkoxy-lower alkoxy-lower alkyl, such as 2-methoxymethoxy-3-methyl-pentyl,
phenoxy-lower alkyl or nitrophenoxy-lower alkyl, such as phenoxymethyl, phenoxyethyl or 4-nitrophenoxymethyl,
naphthyloxy-lower alkyl, for example α- or β-naphthyloxyethyl,
lower alkanoyloxy-lower alkyl, for example lower alkanoyloxyethyl or lower alkanoyloxypropyl, such as acetoxyethyl or 3-acetoxypropyl,
acetoacetoxy-lower alkyl,
arylmercapto-lower alkyl wherein aryl has from 6 to 10 carbon atoms, for example phenyl or naphthyl, such as phenylmercaptomethyl,
amino-lower alkyl, such as 3-aminopropyl or 5-aminopentyl,
mono- or di-lower alkylamino-lower alkyl, such as dimethylaminoethyl or 2-dimethyl-amino-2-isopropylethyl,
phenyl- or naphthyl-amino-lower alkyl, such as 3-phenylaminopropyl,
lower alkanoylamino-lower alkyl, such as 4-acetylaminopentyl,
piperazinylcarbonyl-lower alkyl substituted at the nitrogen atom by lower alkyl, such as methyl, such as 4-methylpiperazinylcarbonylmethyl,
lower alkoxycarbonylamino-lower alkyl, such as 5-(tert-butoxycarbonylamino)-pentyl or 3-ethoxycarbonylamino-2-isobutyl-propyl,
phenyl-lower alkoxycarbonylamino-lower alkyl, such as 5-(benzyloxycarbonylamino)pentyl,
aminocarbonylamino-lower alkyl, such as aminocarbonylamino-ethyl,
N-phenyl-lower alkyl-N-lower alkylaminocarbonylamino-lower alkyl, for example 2-isobutyl-3-(N-benzyl-N-methylaminocarbonylamino)propyl,
halo-lower alkyl, for example 2-haloethyl, such as 2-fluoro-, 2-chloro-, 2-bromo-, 2-iodo-, 2,2,2-trifluoro- or 2,2,2-trichloro-ethyl, trifluoro-lower alkyl, such as trifluoromethyl, or halopropyl, such as 3-chloro- or 3-bromopropyl,
carboxy-lower alkyl, for example carboxyethyl or 3-carboxypropyl,
lower alkoxycarbonyl-lower alkyl, for example lower alkoxycarbonylethyl or lower alkoxycarbonylpropyl, such as methoxycarbonylethyl, 3-methoxycarbonylpropyl, ethoxycarbonylethyl or 3-ethoxycarbonylpropyl,
2-halo-lower alkoxycarbonyl-lower alkyl, such as 2-chloro-, 2-bromo-, 2-iodo- or 2,2,2-trichloro-ethoxycarbonyl-2-ethyl or -3-propyl,
phenyl- or naphthyl-lower alkoxycarbonyl-lower alkyl, for example benzyloxycarbonyl-lower alkyl, such as 3-benzyloxycarbonyl-2,2-dimethylpropyl,
heterocyclyl-lower alkoxycarbonyl-lower alkyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, it also being possible for the mentioned radicals to be fully or partially saturated, such as in 4-pyridylmethoxycarbonyl-2-ethyl or -3-propyl or 2-morpholinocarbonyloxy-4-methylpentyl,
lower alkylsulfonyl-lower alkyl, such as 2-ethylsulfonyl- or 2-tert-butylsulfonyl-methyl,
arylsulfonyl-lower alkyl wherein aryl preferably has from 6 to 10 carbon atoms, for example phenyl or naphthyl, such as phenylsulfonylmethyl,
carbamoyl-lower alkyl, such as carbamoylethyl or 3-carbamoylpropyl,
lower alkylcarbamoyl-lower alkyl, for example lower alkylcarbamoylethyl or methylcarbamoyl-lower alkyl, such as 2-methylcarbamoylethyl,
di-lower alkylcarbamoyl-lower alkyl, for example 2-di-lower alkylcarbamoylethyl or dimethylcarbamoyl-lower alkyl, such as 2-dimethylcarbamoylethyl,
hydroxy-lower alkylcarbamoyl- or di(hydroxy-lower alkyl)carbamoyl-lower alkyl, such as 2-hydroxymethylcarbamoyl- or di(hydroxymethyl)carbamoyl-2-ethyl or -3-propyl,
N-lower alkoxy-lower alkoxy-lower alkylcarbamoyl-lower alkyl, such as 2-isobutyl-3-(2-(2-methoxyethoxy)ethylaminocarbonyl)-propyl, carboxy-lower alkylcarbamoyl- or di(carboxy-lower alkyl)carbamoyl-lower alkyl, such as carboxymethyl- or di(carboxymethyl)carbamoyl-2-ethyl or -3-propyl,
carbamoyl-lower alkyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it being possible for the radical so formed also to be fully or partially unsaturated, for example in the form of piperidino-, pyrazin-1-yl-, piperazin-1-yl-, pyrimidin-1-yl-, pyridazin-1-yl-, morpholino-, thiomorpholino- or S,S-dioxothiomorpholino-carbonyl-lower alkyl, such as in 2-morpholinocarbonyl-ethyl, 3-(morpholinocarbonyl)-propyl or 3-(morpholinocarbonyl)-2-isobutyl-propyl,
N-lower alkyl-N-heterocyclyl-lower alkylcarbamoyl-lower alkyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, such as 2-(N-2-pyridylmethyl)-N-methylcarbamoyl-ethyl,
sulfamoyl-lower alkyl, such as 2-sulfamoylethyl, N-(phenyl- or naphthyl-lower alkyl)sulfamoyl-lower alkyl, such as 3-benzylamino-sulfonyl-2-isopropyl-propyl, or sulfamoyl-lower alkyl substituted at the nitrogen atom by a radical selected from ethylene, trimethylene, tetramethylene and pentamethylene wherein a carbon atom may have been replaced by nitrogen, lower alkyl-substituted, such as methyl-substituted, nitrogen, oxygen, sulfur or by sulfur mono- or di-substituted by oxygen, it being possible for the radical so formed also to be fully or partially unsaturated, for example in the form of piperidino-, pyrazin-1-yl-, piperazin-1-yl-, 4-methylpiperazin-1-yl-, pyrimidin-1-yl-, pyridazin-1-yl-, morpholino-, thiomorpholino- or S,S-dioxothiomorpholino-sulfonyl-lower alkyl, such as 3-(4-methylpiperazinylsulfonyl)-2-isopropyl-propyl or 3-(4-morpholinysulfonyl)-2-isopropyl-propyl,
oxo-lower alkyl (wherein oxo is not present at the carbon atom bonded to the nitrogen atom carrying the radical R₁, R₂, R₈ or R₉), such as 3-oxo-n-butyl or 3-oxo-n-pentyl,
cyano-lower alkanoyl, such as cyanomethyl, 2-cyanoethyl, 2- or 3-cyano-n-propyl or 2-, 3- or 4-cyano-n-butyl,
hydroxy-carboxy-lower alkyl, such as 2-hydroxy-2-carboxy-ethyl or 2-hydroxy-3-carboxypropyl,
α-naphthyloxy-carboxy-lower alkyl, such as 2-α-naphthyloxy-4-carboxy-n-butyl,
hydroxy-lower alkoxycarbonyl-lower alkyl, for example 2-hydroxy-2-lower alkoxycarbonyl-ethyl or -propyl or hydroxy-ethoxy- or hydroxy-methoxy-carbonyl-lower alkyl, such as 2-hydroxy-2-ethoxy- or -methoxy-carbonylethyl or 2-hydroxy-3-ethoxy- or -methoxy-carbonylpropyl,
α-naphthyloxy-lower alkoxycarbonyl-lower alkyl, for example α-naphthyloxy-lower alkoxycarbonyl-2-ethyl, -2-propyl or -2-butyryl or α-naphthyloxyethoxycarbonyl-lower alkyl, such as α-naphthyloxy-ethoxycarbonyl-2-ethyl, 2-α-naphthyloxy-3-ethoxycarbonylpropyl or 2-α-naphthyloxy-4-tert-butoxycarbonylbutyl,
α-naphthyloxy-benzyloxycarbonyl-lower alkyl, such as 2-o-naphthyloxy-3-benzyloxycarbonyl-propyl,
esterified hydroxy-lower alkoxycarbonyl-lower alkyl wherein the hydroxy group is esterified by lower alkanoyl, for example acetyl, propionyl or pivaloyl, cycloalkyl-lower alkanoyl wherein cycloalkyl has from 3 to 7 carbon atoms and lower alkanoyl is as last defined, for example cyclohexylcarbonyl or 2-cyclohexyl- or 2-cyclopentyl-acetyl, bicycloalkyl-lower alkanoyl wherein bicycloalkyl has, for example from 5 to 10, especially from 6 to 9, carbon atoms, such as in bicyclo-hexyl-, -heptyl-, -octyl-, -nonyl- or -decyl-acetyl or -3-propionyl, for example bicyclo[3.1.0]hexyl-, -2- or -3-yl-, bicyclo-[4.1.0]hept-1- or -7-yl-, bicyclo[2.2.1]hept-2-yl-, for example endo- or exo-norbornyl-, bicyclo[3.2.1]oct-2-yl-, bicyclo[3.3.0]oct-3-yl- or bicyclo[3.3.1]non-9-yl-, and also α- or β-decahydronaphthyl-acetyl or -3-propionyl, tricycloalkyl-lower alkanoyl wherein tricycloalkyl contains, for example, from 8 to 10 carbon atoms, for example in tricyclo[5.2.1.0.sup.2,6]dec-8-yl- or adamantyl-, such as 1-adamantyl-acetyl, aryl-lower alkanoyl wherein aryl has from 6 to 14 carbon atoms, for example phenyl, indenyl, indanyl, naphthyl, anthryl, phenanthryl, acenaphthyl or fluorenyl, which may be unsubstituted or mono- to tri-substituted by lower alkyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, sulfamoyl, nitro and/or by cyano, lower alkoxycarbonyl, for example tert-butoxycarbonyl, 2-halo-lower alkoxycarbonyl as defined above, or by phenyl- or fluorenyl-lower alkoxycarbonyl, for example benzyloxycarbonyl or 9-fluorenylmethoxycarbonyl, such as 2-acetoxy-2-methoxycarbonyl-ethyl, 2-benzoyloxy-, 2-(1- or 2-naphthoyloxy)-, 2-(phenyl-2-acetoxy)-, 2-(1- or 2-naphthyl-2-acetoxy)-, 2-(4-methylphenyl-2-acetoxy)-, 2-(4-methoxyphenyl-2-acetoxy)- or 2-(2-(o,o-dichlorophenyl)₂-acetoxy)-2-methoxycarbonyl-ethyl or -3-propyl,
dihydroxy-carboxy-lower alkyl, such as 2,3-dihydroxy-3-carboxy-propyl,
dihydroxy-lower alkoxycarbonyl-lower alkyl, such as 2,3-dihydroxy-3-ethoxy- or -methoxy-carbonyl-propyl,
dihydroxy-lower alkoxycarbonyl-lower alkyl esterified by lower alkanoyl, such as acetyl, propionyl or butyryl, lower alkoxycarbonyl, for example tert-butoxycarbonyl, phenyl- or fluorenyl-lower alkoxycarbonyl, such as benzyloxycarbonyl or 9-fluorenylmethoxycarbonyl, lower alkylsulfonyl or by toluenesulfonyl, for example di-lower alkanoyloxy-lower alkoxy-propyl, such as 2,3-diacetoxy-3-methoxycarbonyl-propyl,
α-naphthyloxy-di-lower alkylamino-lower alkyl, such as 2-(X-naphthyloxy-5-dimethylaminopentyl,
α-naphthyloxy-carbamoyl-lower alkyl, such as 2-α-naphthyloxy-4-carbamoyl-butyl,
α-naphthyloxy-oxo-lower alkyl (wherein oxo is not present at the carbon atom bonded to the nitrogen atom carrying the radical R₁, R₂, R₈ or R₉), such as 2-α-naphthyloxy-4-oxo-pentyl, or
α-naphthyloxy-cyano-lower alkyl, such as 2-α-naphthyloxy-cyano-ethyl or 2-α-naphthyloxy-4-cyanobutyl.

Lower alkyl, such as methyl, ethyl, propyl, isopropyl, n-butyl, isobutyl or tert-butyl, is especially preferred.

Alkenyl R₁, R₂, R₈ or R₉ preferably contains from 2 to 10 carbon atoms, is preferably lower alkenyl having from 2 to 7, especially from 2 to 4, carbon atoms and is, for example, vinyl, allyl or 2- or 3-butenyl. Lower alkenyl R₁, R₂, R₈ or R₉ may be substituted by the same substituents as may lower alkyl, for example by cycloalkyl, as defined in the case of cycloalkyl-lower alkanoyl R₁, R₂, R₈ or R₉, that is unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methyl-sulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded, especially at the terminal carbon atom, to lower alkenyl, such as in cyclobutyl-, cyclopentyl-, cyclohexyl- or cycloheptyl-methyl-2-vinyl, -2- or -3-allyl or -2-, -3- or -4-but-2-enyl; aryl, as defined under aryl-lower alkanoyl R₁, R₂, R₈ or R₉, that is unsubstituted or substituted and is bonded, preferably terminally, to lower alkenyl, such as in styryl, 3-phenylallyl (cinnamyl), 2-(α-naphthyl)-vinyl or 2-(β-naphthyl)-vinyl; unsubstituted or substituted heterocyclyl, as defined under heterocyclyl-lower alkanoyl R₁, R₂, R₈ or R₉, preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-, cyclopenta-, cyclohexa- or cyclohepta-fused derivative of those radicals, which may also be fully or partially saturated and are unsubstituted or substituted as indicated above and are bonded via a nitrogen or carbon atom to lower alkenyl, preferably to the terminal carbon atom of the lower alkenyl radical, which is selected, for example, from vinyl, allyl and 2- or 3-butenyl, for example in the form of pyrimidin-1-yl-, piperidino-, pyrazin-1-yl-, piperazin-1-yl-, pyridazin-1-yl-, morpholino-, thiomorpholino- or S,S-dioxothiomorpholino-lower alkenyl, such as in 2-morpholino-vinyl, 3-morpholino-allyl or 4-morpholino-2- or -3-butenyl, pyrrolyl-lower alkenyl that is unsubstituted or substituted by lower alkyl or by phenyl, such as 2- or 3-pyrrolyl-vinyl or -allyl, 4- or 5-methylpyrrolyl-vinyl or -allyl or 4- or 5-phenylpyrrolyl-vinyl or -allyl, thienyl-lower alkenyl, such as 2-thienyl-vinyl or -allyl, furyl-lower alkenyl, such as 2-furyl-vinyl or -allyl, pyridyl-lower alkenyl, such as 2-, 3- or 4-pyridyl-vinyl or -allyl, indolyl-lower alkenyl that is unsubstituted or substituted by lower alkyl, for example methyl, phenyl-lower alkyl, for example benzyl, lower alkoxy, for example methoxy, phenyl-lower alkoxy, for example benzyloxy, or by halogen, for example chlorine, such as 2-, 3- or 5-indolyl-vinyl or -allyl, 1-methyl-, 2-methyl-, 5-methoxy-, 5-benzyloxy-, 5-chloro- or 4,5-dimethyl-indol-2-yl-, 1-benzylindol-2-yl- or -3-yl-vinyl or -allyl, 4,5,6,7-tetrahydroindol-2-yl-methyl, -ethyl or -n-propyl, cyclohepta-[b]pyrrol-5-yl-vinyl or -allyl, unsubstituted or hydroxy-substituted quinolyl-lower alkenyl, for example 2-, 3- or 4-quinolyl- or 4-hydroxyquinol-2-yl-vinyl or -allyl, unsubstituted or hydroxy-substituted isoquinolyl-lower alkenyl, such as 1-, 3- or 4-isoquinolyl- or. 1-oxo-1,2-dihydroisoquinol-3-yl-vinyl or -allyl, 2-quinoxalinyl-vinyl or -allyl, 3,1-benzofuran-2-yl-vinyl or -allyl, benz[e]indol-2-yl-vinyl or -allyl, β-carbolin-3-yl-vinyl or -allyl, 3-chromanyl-vinyl or -allyl, 3-thiochromanyl-vinyl or -allyl, 3-pyrrolidinyl-vinyl or -allyl, hydroxypyrrolidinyl-lower alkenyl, such as 3- or 4-hydroxypyrrolidin-2-yl-vinyl or -allyl, oxopyrrolidinyl-lower alkenyl, such as 5-oxopyrrolidin-2-yl-vinyl or -allyl, piperidinyl-lower alkenyl, such as 2-, 3- or 4-piperidinyl-vinyl- or -allyl, morpholinyl-lower alkenyl, such as 2- or 3-morpholinyl-vinyl or -allyl, thiomorpholinyl-lower alkenyl, such as 2- or 3-thiomorpholinyl-vinyl or -allyl, S,S-dioxothiomorpholinyl-lower alkenyl, such as S,S-dioxothiomorpholin-2- or -3-yl-vinyl or -allyl, indolinyl-lower alkenyl, such as 2- or 3-indolinyl-vinyl or -allyl, 1,2,3,4-tetrahydroquinolyl-lower alkenyl, such as 1,2,3,4-tetrahydroquinol-2-, -3- or -4-yl-vinyl or -allyl, 1,2,3,4-tetrahydroisoquinolyl-lower alkenyl, such as 1,2,3,4-tetrahydroisoquinol-1-, -2- or -3-yl-vinyl or -allyl, or 1-oxo-1,2,3,4-tetrahydroisoquinol-3-yl-vinyl or -allyl; also hydroxy; etherified hydroxy selected from lower alkoxy, such as methoxy or ethoxy, phenoxy or naphthyloxy, phenyl- or naphthyl-lower alkoxy, such as benzyloxy, and heterocyclyl-lower alkoxy wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, for example 4-pyrrolidinylmethoxy, 1-imidazolylmethoxy, 2-pyridylmethoxy, 3-pyridylmethoxy, quinolin-2-yl-methoxy or indol-2-ylmethoxy; esterified hydroxy selected from lower alkanoyloxy, such as acetoxy, propionyloxy, butyroxy, isobutyroxy or -pivaloyloxy, acetoacetoxy, amino- or benzyloxycarbonylamino-lower alkanoyloxy, such as 2-amino- or 2-benzyloxycarbonylamino-2-methylpropionyloxy, aryl-lower alkanoyloxy wherein aryl contains from 6 to 10 carbon atoms, such as in benzoyloxy, phenylacetoxy, 1- or 2-naphthoyloxy, lower alkoxycarbonyloxy, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyloxy, mono- or di-lower alkylaminocarbonyloxy, such as ethylaminocarbonyloxy or diethylaminocarbonyloxy, aryloxycarbonyloxy wherein aryl contains from 6 to 10 carbon atoms, for example phenoxycarbonyloxy or 1- or 2-naphthyloxycarbonyloxy, aryl-lower alkoxycarbonyloxy wherein aryl contains from 6 to 12 carbon atoms, for example phenyl-lower alkoxycarbonyloxy, such as benzyloxycarbonyloxy, and also 1- or 2-naphthylmethoxycarbonyloxy or 9-fluorenylmethoxycarbonyloxy, sulfonyloxy, lower alkylsulfonyloxy, for example methyl-, ethyl-, propyl-, isopropyl-, n-butyl-, isobutyl-, sec-butyl-, tert-butyl-, n-pentyl-, isopentyl-, neopentyl-, tert-pentyl-, n-hexyl-, isohexyl- or n-heptyl-sulfonyloxy, phenylsulfonyloxy, 2- or 4-toluenesulfonyloxy and 1- or 2-naphthylsulfonyloxy; halogen, for example chlorine or bromine; carboxy; esterified carboxy selected from lower alkoxycarbonyl, such as methoxycarbonyl, ethoxycarbonyl and tert-butoxycarbonyl, 2-halo-lower alkoxycarbonyl, for example 2-chloro-, 2-bromo-, 2-iodo- or 2,2,2-trichloro-ethoxycarbonyl, phenyl- or naphthyl-lower alkoxycarbonyl, for example benzyloxycarbonyl, and heterocyclyl-lower alkoxycarbonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, it also being possible for the mentioned radicals to be fully or partially saturated, for example in 4-pyridylmethoxycarbonyl; or amidated carboxy selected from carbamoyl, lower alkylcarbamoyl, such as methylcarbamoyl, di-lower alkylcarbamoyl, such as dimethylcarbamoyl, hydroxy-lower alkylcarbamoyl or di(hydroxy-lower alkyl)carbamoyl, such as hydroxymethylcarbamoyl or di(hydroxymethyl)carbamoyl, N-lower alkoxy-lower alkoxy-lower alkylcarbamoyl, such as 2-(2-methoxyethoxy)ethylaminocarbonyl, carboxy-lower alkylcarbamoyl or di(carboxy-lower alkyl)carbamoyl, for example carboxymethyl- or di(carboxymethyl)-carbamoyl, and from carbamoyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, pyrimidin-1-yl-, pyridazin-1-yl-, morpholino-, thiomorpholino- or S,S-dioxothiomorpholino-carbonyl, N-lower alkyl-N-heterocyclyl-lower alkylcarbamoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, for example N-2-pyridylmethyl-N-methylcarbamoyl.

Alkynyl R₁, R₂, R₈ or R₉ contains especially from 2 to 10, preferably, as lower alkynyl, from 2 to 7, especially from 2 to 4, carbon atoms, and is, for example, ethynyl, 1-propynyl or 2-propynyl, it being possible for the mentioned radicals to be unsubstituted or substituted by the radicals mentioned for lower alkenyl R₁, R₂, R₈ or R₉. Heterocyclyl R₁, R₂, R₈ and R₉ is bonded via a carbon atom and contains especially an unsubstituted or substituted heterocyclyl mentioned under heterocyclyl-lower alkanoyl R₁, R₂, R₈ and R₉ and is preferably pyrrolyl that is unsubstituted or substituted by lower alkyl or by phenyl, such as 2- or 3-pyrrolyl, 4- or 5-methylpyrrolyl or 4- or 5-phenylpyrrolyl, thienyl, such as 2-thienyl, furyl, such as 2-furyl, pyridyl, such as 2-, 3- or 4-pyridyl, indolyl that is unsubstituted or substituted by lower alkyl, for example methyl, phenyl-lower alkyl, for example benzyl, lower alkoxy, for example methoxy, phenyl-lower alkoxy, for example benzyloxy, or by halogen, for example chlorine, such as 2-, 3- or 5-indolyl, 1-methyl-, 2-methyl-, 5-methoxy-, 5-benzyloxy-, 5-chloro- or 4,5-dimethylindol-2-yl, 1-benzylindol-2-yl or -3-yl, 4,5,6,7-tetrahydroindol-2-yl, unsubstituted or hydroxy-substituted quinolyl, such as 2-, 3- or 4-quinolyl or 4-hydroxyquinol-2-yl, unsubstituted or hydroxy-substituted isoquinolyl, such as 1-, 3- or 4-isoquinolyl or 1-oxo-1,2-dihydroisoquinol-3-yl, 2-quinoxalinyl, 3,1-benzofuran-2-yl, benz[e]indol-2-yl, β-carbolin-3-yl, cyclohepta[b]pyrrol-5-yl, 3-chromanyl, 3-thiochromanyl, 3-pyrrolidinyl, hydroxypyrrolidinyl, such as 3- or 4-hydroxypyrrolidin-2-yl, oxopyrrolidinyl, such as 5-oxopyrrolidin-2-yl, piperidyl, such as 2-, 3- or 4-piperidyl, morpholinyl, such as 2- or 3-morpholinyl, thiomorpholinyl, such as 2- or 3-thiomorpholinyl, S,S-dioxothiomorpholinyl, such as S,S-dioxothiomorpholin-2- or -3-yl, indolinyl, such as 2- or 3-indolinyl, 1,2,3,4-tetrahydroquinolyl, such as 1,2,3,4-tetrahydroquinol-2-, -3- or -4-yl, 1,2,3,4-tetrahydroisoquinolyl, such as 1,2,3,4-tetrahydroisoquinol-1-, -2- or -3-yl, or 1-oxo-1,2,3,4-tetrahydroisoquinol-3-yl.

Alkyl-substituted sulfonyl R₁, R₂, R₈ and R₉ preferably contains an unsubstituted or substituted alkyl radical mentioned under alkyl R₁, R₂, R₈ and R₉ and is especially lower alkylsulfonyl, such as methylsulfonyl, ethylsulfonyl, n-propylsulfonyl or tert-butylsulfonyl, aryl-lower alkyl-substituted sulfonyl that contains, for example, an unsubstituted or substituted radical mentioned under aryl-lower alkyl R₁, R₂, R₈ and R₉ and is selected especially from the radicals benzyl-, 4-chloro-, 4-methoxy- or 4-nitro-benzyl-, naphthylmethyl-, for example α- or β-naphthylmethyl-, 2-phenylethyl-, 2-α-naphthylethyl-, 2-β-naphthylethyl-, 2-(4-methylphenyl)ethyl-, 2-(4-methoxyphenyl)ethyl-, 3-phenylpropyl-, 3-(p-hydroxyphenyl)-propyl-, 2,2-diphenylethyl- and 2,2-di(4-methoxyphenyl)-ethylsulfonyl, or heterocyclyl-lower alkyl-substituted sulfonyl that contains, for example, an unsubstituted or substituted radical mentioned under heterocyclyl-lower alkyl R₁, R₂, R₈ and R₉ and is selected especially from radicals such as 2- or 3-pyrrolyl-, 2-thienyl-, 2-furyl-, 1-pyrazolyl-, 2-, 3- or 4-pyridyl-, 2-, 3- or 5-indolyl-, (1-methyl-, 2-methyl-, 5-benzyloxy-, 5-chloro- or 4,5-dimethyl-indol-2-yl)-, (1-benzylindol-2-yl or -3-yl)-, 4,5,6,7-tetrahydroindol-2-yl-, (2-, 3- or 4-quinolyl or 4-hydroxyquinol-2-yl)-, (1-, 3- or 4-isoquinolyl or 1-oxo-1,2-dihydroisoquinol-3-yl)-, 3-pyrrolidinyl-, (3- or 4-hydroxypyrrolidin-2-yl)-, 5-oxopyrrolidin-2-yl-, (2- or 3-morpholinyl)-, (2- or 3-thiomorpholinyl)-, (S,S-dioxothiomorpholin-2- or -3-yl)-, (2- or 3-indolinyl)-, (1,2,3,4-tetrahydroquinol-2-, -3- or -4-yl)- and (1,2,3,4-tetrahydroisoquinol-1-, -2- or -3-yl)-methyl-sulfonyl.

Aryl-substituted sulfonyl R₁, R₂, R₈ and R₉ preferably contains an unsubstituted or substituted aryl radical mentioned under aryl-lower alkanoyl R₁, R₂, R₈ and R₉ and is especially phenyl- or 1- or 2-naphthyl-sulfonyl that is unsubstituted or mono- or di-substituted by lower alkyl, such as phenylsulfonyl, 2- or 4-toluenesulfonyl or 1- or 2-naphthylsulfonyl.

Heterocyclyl-substituted sulfonyl R₁, R₂, R₈ and R₉ preferably contains heterocyclyl that is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and from thiomorpholinyl and may be unsubstituted or substituted, especially by lower alkyl, such as methyl, such as morpholinosulfonyl, thiomorpholinosulfonyl, piperidinosulfonyl, 4-methylpiperazinylsulfonyl or piperazinosulfonyl. In especially preferred forms of the invention, heterocyclylsulfonyl as substituent may be absent.

Alkoxy-substituted sulfonyl R₁, R₂, R₈ and R₉ preferably contains an unsubstituted or substituted alkyl radical mentioned under alkyl R₁, R₂, R₈ and R₉ and is selected especially from lower alkoxy-, such as methoxy-, ethoxy- or tert-butoxy-sulfonyl, heterocyclyl-lower alkoxysulfonyl that contains, for example, an unsubstituted or substituted heterocyclyl-lower alkyl radical mentioned under heterocyclyl-lower alkyl R₁, R₂, R₈ and R₉ and is selected especially from 2- or 3-pyrrolyl-, 2-thienyl-, 2-furyl-, 1-pyrazolyl-, 2-, 3- or 4-pyridyl-, 2-, 3- or 5-indolyl-, (1-methyl-, 2-methyl-, 5-benzyloxy-, 5-chloro- or 4,5-dimethyl-indol-2-yl)-, (1-benzylindol-2-yl or -3-yl)-, 4,5,6,7-tetrahydroindol-2-yl-, (2-, 3- or 4-quinolyl or 4-hydroxyquinol-2-yl)-, (1-, 3- or 4-isoquinolyl or 1-oxo-1,2-dihydroisoquinol-3-yl)-, 3-pyrrolidinyl-, (3- or 4-hydroxypyrrolidin-2-yl)-, 5-oxopyrrolidin-2-yl-, (2- or 3-morpholinyl)-, (2- or 3-thiomorpholinyl)-, (S,S-dioxothiomorpholin-2- or -3-yl)-, (2- or 3-indolinyl)-, (1,2,3,4-tetrahydroquinol-2-, -3- or -4-yl)- and (1,2,3,4-tetrahydroisoquinol-1-, -2- or -3-yl)-methoxysulfonyl or -ethoxysulfonyl, aryl-lower alkoxysulfonyl that contains, for example, an unsubstituted or substituted aryl-lower alkyl radical mentioned under aryl-lower alkyl R₁, R₂, R₈ and R₉, such as benzyloxysulfonyl, hydroxy-lower alkoxy-, such as 3-hydroxypropoxy- or 2-hydroxy-3-methylpentyloxysulfonyl, lower alkoxy-lower alkoxy-, for example lower alkoxyethoxy- or lower alkoxypropoxy-, such as methoxyethoxy- or 3-methoxypropoxy-sulfonyl, lower alkoxy-lower alkoxy-lower alkoxy-, such as 2-methoxymethoxy-3-methylpentyloxy-sulfonyl, phenoxy-lower alkoxy- or nitrophenoxy-lower alkoxy-, such as phenoxymethoxy-, phenoxyethoxy- or 4-nitrophenoxymethoxy-sulfonyl, naphthyloxy-lower alkoxy-, such as α- or β-naphthyloxyethoxy-sulfonyl, lower alkanoyloxy-lower alkoxy-, for example lower alkanoyloxyethoxy- or lower alkanoyloxypropoxy-, such as acetoxyethoxy- or 3-acetoxypropoxy-sulfonyl, amino-lower alkoxy-, such as 5-aminopentyloxy-sulfonyl, mono- or di-lower alkylamino-lower alkoxy-, such as dimethylaminoethoxy- or 2-dimethylamino-2-isopropylethoxy-sulfonyl, lower alkanoylamino-lower alkoxy-, such as 4-acetylaminopentyloxy-sulfonyl, lower alkoxycarbonylamino-lower alkoxy-, such as 5-(tert-butoxycarbonylamino)pentyloxy- or 3-ethoxycarbonylamino-2-isobutyl-propoxy-sulfonyl, phenyl-lower alkoxycarbonylamino-lower alkoxy-, such as 5-(benzyloxycarbonylamino)-pentyloxy-sulfonyl, acetoacetoxy-sulfonyl, aminocarbonyl-amino-lower alkoxy-, such as aminocarbonylamino-ethoxy-sulfonyl, N-phenyl-lower alkyl-N-lower alkyl-aminocarbonylamino-lower alkoxy-, for example 2-isobutyl-3-(N-benzyl-N-methylaminocarbonylamino)propoxy-sulfonyl, halo-lower alkoxy-, for example 2-haloethoxy-, such as (2-chloro-, 2-bromo-, 2-iodo- or 2,2,2-trichloro-ethoxy)-, or halopropoxy-, such as (3-chloro- or 3-bromo-propoxy)sulfonyl, carboxy-lower alkoxy-, such as carboxyethoxy- or 3-carboxypropoxy-sulfonyl, lower alkoxycarbonyl-lower alkoxy-, for example lower alkoxycarbonylethoxy- or lower alkoxycarbonylpropoxy-, such as methoxycarbonylethoxy-, 3-methoxy carbonylpropoxy-, ethoxycarbonylethoxy- or 3-ethoxycarbonylpropoxy-sulfonyl, 2-halo-lower alkoxycarbonyl-lower alkoxy-, such as (2-chloro-, 2-bromo-, 2-iodo- or 2,2,2-trichloro-ethoxycarbonyl-2-ethoxy or 3-propoxy)-sulfonyl, lower alkylsulfonyl-lower alkoxy-, such as (2-ethylsulfonyl- or 2-tert-butylsulfonylmethoxy)-sulfonyl, carbamoyl-lower alkoxy-, such as carbamoylethoxy- or 3-carbamoylpropoxy-sulfonyl, and carbamoyl-lower alkoxysulfonyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it also being possible for the radical so formed to be fully or partially unsaturated, for example in the form of (piperidino-, pyrazin-1-yl-, piperazin-1-yl-, pyridazin-1-yl-, pyridazin-1-yl-, morpholino-, thiomorpholino- or S,S-dioxothiomorpholino-carbonyl-lower alkoxy)-, such as in 2-morpholinocarbonyl-ethoxy-, 3-(morpholinocarbonyl)-propoxy- or 3-(morpholinocarbonyl)-2-isobutyl-propoxy-sulfonyl.

Aryloxy-substituted sulfonyl R₁, R₂, R₈ and R₉ preferably contains an unsubstituted or substituted aryl radical mentioned under aryl-lower alkanoyl R₁, R₂, R₈ and R₉ and is selected especially from benzyloxysulfonyl and 1- or 2-naphthyloxysulfonyl.

Sulfamoyl R₁, R₂, R₈ and R₉ substituted at the nitrogen atom may be substituted preferably by the same radicals as may carbamoyl in carbamoyl-lower alkanoyl R₁, R₂, R₈ and R₉ and is selected especially from mono- or di-lower alkylsulfamoyl, such as N,N-dimethylsulfamoyl, N-(phenyl- or naphthyl-lower alkyl)sulfamoyl, such as 3-benzylsulfamoyl, and sulfamoyl substituted at the nitrogen atom by a radical selected from ethylene, trimethylene, tetramethylene or pentamethylene wherein a carbon atom may have been replaced by nitrogen, lower alkyl-substituted, such as methyl-substituted, nitrogen, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, methylpiperazin-1-yl-, pyrimidin-1-yl-, pyridazin-1-yl-, morpholino-, thiomorpholino- or S,S-dioxothiomorpholino-sulfonyl, such as 4-methylpiperazinylsulfonyl or morpholinosulfonyl-2-isopropyl-propyl.

Phosphoryl R₁, R₂, R₈ and R₉ substituted by one or two identical or different radicals selected from alkyl, cycloalkyl, cycloalkyl-lower alkyl, aryl, aryl-lower alkyl, hydroxy, alkoxy, cycloalkoxy, cycloalkyl-lower alkoxy, aryloxy and aryl-lower alkoxy preferably contains as unsubstituted or substituted alkyl one or two of the unsubstituted or substituted radicals mentioned under alkyl, especially lower alkyl R₁, R₂, R₈ and R₉, for example lower alkyl, such as methyl, ethyl, n-propyl, isopropyl, n-butyl and tert-butyl, cycloalkyl-lower alkyl that is a lower alkyl radical substituted especially terminally by one of the cycloalkyl radicals mentioned under cycloalkyl-lower alkanoyl R₁, R₂, R₈ and R₉, such as cyclopropylmethyl, cyclobutylmethyl, cyclopentylmethyl, 2-cyclopentylethyl, 3-cyclopentylpropyl, cyclohexylmethyl, 2-cyclohexylethyl or 3-cyclohexylpropyl, aryl-lower alkyl, as defined under aryl-lower alkyl R₁, R₂, R₈ or R₉, especially benzyl, unsubstituted, mono- or poly-substituted preferably by lower alkyl, for example methyl, phenyl, hydroxy, lower alkoxy, for example methoxy, halogen, for example chlorine, and/or by nitro, such as 4-methoxy-, 4-chloro- or 4-nitro-benzyl, naphthylmethyl, such as α- or β-naphthylmethyl, 2-phenylethyl, 2-α-naphthylethyl, 2-β-naphthylethyl, 2-lower alkylphenylethyl, such as 2-(4-methylphenyl)ethyl, 2-lower alkoxyphenylethyl, such as 2-(4-methoxyphenyl)ethyl, 2,2-diphenylethyl, 2,2-di(4-methoxyphenyl)-ethyl, 2,2,2-triphenylethyl, 2,2-dibenzylethyl, 2,p-diamino-phenylethyl, or phenyl-lower alkyl substituted in the 2- and p-positions by two radicals selected from phenyl-lower alkoxycarbonyl, for example benzyloxycarbonyl, and lower alkanoylamino, for example pivaloylamino, such as 2,p-dibenzyloxycarbonylamino-phenylethyl or 2-pivaloylamino-p-benzyloxycarbonylamino-phenylethyl, 3-phenylpropyl, 3-(p-hydroxyphenyl)-propyl, 3-α- or 3-p-naphthylpropyl, 2-benzyl-3-(1-pyrazolyl)-propyl, 3-phenyl- or 3-α-naphthyl-2-hydroxypropyl, 3-phenyl- or 3-α-naphthyl-2-lower alkoxy-propyl, such as 3-phenyl- or 3-α-naphthyl-2-neopentyloxy-propyl, 3-phenyl- or 3-α-naphthyl-2-lower alkanoyloxy-propyl, such as 3-phenyl- or 3-α-naphthyl-2-pivaloyloxy- or -2-acetoxy-propyl, 2-benzyl- or 1- or 2-naphthyl-3-(N-methoxy-N-methylamino)-propyl, 3-phenyl- or 3-α-naphthyl-2-dimethylaminomethyl-propyl, 3-α-naphthyl-2-acetoacetoxy-propyl, 3-α-naphthyl-2-ethylaminocarbonyloxy-propyl or 3-α-naphthyl-2-(2-amino- or 2-benzyloxycarbonylamino-2-methylpropionyloxy)-propyl, 3-phenyl- or 3-α-naphthyl-2-carboxymethyl-propyl, 3-phenyl- or 3-α-naphthyl-2-lower alkoxycarbonyl-propyl, such as 3-α-naphthyl-2-ethoxycarbonylpropyl, 3-phenyl- or 3-α-naphthyl-2-benzyloxycarbonylmethyl-propyl, 2-(S)-benzyl-3-tert-butylsulfonyl-propyl, 3-phenyl-2-phosphono- or -phosphono methyl-propyl, 3-phenyl-2-dimethoxyphosphoryl- or dimethoxyphosphorylmethyl-propyl, 3-phenyl-2-diethoxyphosphoryl- or -diethoxyphosphorylmethyl-propyl, 3-phenyl-2-ethoxy- or -methoxy-hydroxyphosphorylpropyl, 3-phenyl- or 3-α-naphthyl-2-carbamoyl-propyl, 3-phenyl- or 3-α-naphthyl-2-tert-butylcarbamoyl-propyl, 3-phenyl- or 3-α-naphthyl-2-(2-dimethylaminoethyl)carbamoyl-propyl, 3-α-naphthyl-2-(carboxy- or tert-butoxycarbonyl)methylcarbamoyl-propyl, 3-phenyl- or 3-α-naphthyl-2-(3-hydroxy-2-propyl)-carbamoyl-propyl, 3-phenyl- or 3-α-naphthyl-2-(2,2-dimethoxyethyl)-carbamoyl-propyl, 3-phenyl- or 3-α-naphthyl-2-(5-amino-5-carboxypentyl)-carbamoyl-propyl, 3-phenyl- or 3-α-naphthyl-2-cyano-propyl, 3-phenyl- or 3-α-naphthyl-2-cyanomethyl-propyl, 3-phenyl- or 3-α-naphthyl-2-acetonyl-propyl, 4-hydroxyphenylbutyl, 4-phenyl- or 4-α-naphthyl-3-carboxybutyl, 4-phenyl- or 4-α-naphthyl-3-benzyloxycarbonyl-butyl, 2-benzyl- or 2-α-naphthylmethyl-4-cyano-butyl, 2-benzyl-4-(2-benzofuranyl)-4-oxobutyl, 2-benzyl- or 2-α-naphthylmethyl-5-dimethylamino-pentyl, 2-benzyl- or 2-α-naphthylmethyl-4-oxo-pentyl, 2-benzyl- or 2-α-naphthylmethyl-4,4-dimethyl-3-oxo-pentyl, 2-benzyl- or 2-α-naphthylmethyl-5-dimethylamino-4-oxo-pentyl, or 2-benzyl- or -2-α-naphthylmethyl-5,5-dimethyl-4-oxo-hexyl, as cycloalkyl one of the radicals mentioned under cycloalkyl-lower alkanoyl R₁, R₂, R₈ and R₉, especially cyclopropyl, cyclobutyl, cyclopentyl or cyclohexyl, as aryl one of the unsubstituted or substituted radicals mentioned under aryl-lower alkanoyl R₁, R₂, R₈ and R₉, preferably phenyl, unsubstituted, mono- or poly-substituted by lower alkyl, for example methyl, halo-lower alkyl, for example trifluoromethyl, phenyl, hydroxy, lower alkoxy, for example methoxy, halogen, for example fluorine or chlorine, and/or by nitro, such as 4-methyl-, 3-hydroxy-, 4-methoxy-, 4-chloro- or 4-nitro-phenyl, naphthyl, such as α- or β-naphthyl, or anilinophenyl substituted in the phenyl radical by one or two radicals selected from lower alkyl, such as methyl or ethyl, hydroxy, lower alkoxy, such as methoxy, amino, mono- or di-lower alkylamino, such as ethylamino or dimethylamino, halogen, such as fluorine or chlorine, carboxy, sulfo, carbamoyl, sulfamoyl and cyano, and/or at the amino group by one or two radicals selected from lower alkyl and benzyl, such as 2-(o,o-dichloroanilino)-phenyl or 2-(o,o-dichloro-N-benzylanilino)-phenyl, as alkoxy one of the unsubstituted or substituted radicals mentioned in the case of alkoxy-substituted sulfonyl R₁, R₂, R₈ and R₉, especially lower alkoxy, such as methoxy, ethoxy or tert-butoxy, cycloalkyl-lower alkoxy with one of the radicals mentioned under phosphoryl R₁, R₂, R₈ and R₉ substituted by cycloalkyl-lower alkyl, bonded via oxygen, such as cyclopropylmethoxy, cyclobutylmethoxy, cyclopentylmethoxy, 2-cyclopentylethoxy, 3-cyclopentylpropoxy, cyclohexylmethoxy, 2-cyclohexylethoxy or 3-cyclohexylpropoxy, aryl-lower alkoxy with one of the radicals mentioned under sulfonyl R₁, R₂, R₈ and R₉ substituted by aryl-lower alkoxy, bonded via oxygen, especially benzyloxy, hydroxy-lower alkoxy, such as 3-hydroxypropoxy or 2-hydroxy-3-methylpentyloxy, lower alkoxy-lower alkoxy, for example lower alkoxyethoxy or lower alkoxypropoxy, such as methoxyethoxy or 3-methoxypropoxy, lower alkoxy-lower alkoxy-lower alkoxy, such as 2-methoxymethoxy-3-methylpentyloxy, phenoxy-lower alkoxy or nitrophenoxy-lower alkoxy, such as phenoxymethoxy, phenoxyethoxy or 4-nitrophenoxymethoxy, naphthyloxy-lower alkoxy, such as α- or β-naphthyloxyethoxy, lower alkanoyloxy-lower alkoxy, such as lower alkanoyloxyethoxy or lower alkanoyloxypropoxy, such as acetoxyethoxy or 3-acetoxypropoxy, amino-lower alkoxy, such as 5-aminopentyloxy, mono- or di-lower alkylamino-lower alkoxy, such as dimethylaminoethoxy or 2-dimethylamino-2-isopropylethoxy, lower alkanoylamino-lower alkoxy, such as 4-acetylaminopentyloxy, lower alkoxycarbonylamino-lower alkoxy, such as 5-(tert-butoxycarbonylamino)-pentyloxy or 3-ethoxycarbonylamino-2-isobutyl-propoxy, phenyl-lower alkoxycarbonylamino-lower alkoxy, such as 5-(benzyloxycarbonylamino)-pentyloxy, aminocarbonylamino-lower alkoxy, such as aminocarbonylamino-ethoxy, N-phenyl-lower alkyl-N-lower alkyl-aminocarbonylamino-lower alkoxy, such as 2-isobutyl-3-(N-benzyl-N-methylaminocarbonylamino)propoxy, halo-lower alkoxy, for example 2-haloethoxy, such as 2-chloro-, 2-bromo-, 2-iodo- or 2,2,2-trichloro-ethoxy, or halopropoxy, such as 3-chloro- or 3-bromo-propoxy, carboxy-lower alkoxy, such as carboxyethoxy or 3-carboxypropoxy, lower alkoxycarbonyl-lower alkoxy, for example lower alkoxycarbonylethoxy or lower alkoxycarbonylpropoxy, such as methoxycarbonylethoxy, 3-methoxycarbonylpropoxy, ethoxycarbonylethoxy or 3-ethoxycarbonylpropoxy, 2-halo-lower alkoxycarbonyl-lower alkoxy, such as 2-chloro-, 2-bromo-, 2-iodo- or 2,2,2-trichloro-ethoxycarbonyl-2-ethoxy or -3-propoxy, lower alkylsulfonyl-lower alkoxy, such as 2-ethylsulfonyl- or 2-tert-butylsulfonyl-methoxy, carbamoyl-lower alkoxy, such as carbamoylethoxy or 3-carbamoylpropoxy, or carbamoyl-lower alkoxy substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it also being possible for the ring formed to be fully or partially unsaturated, for example in the form of (piperidino-, pyrazin-1-yl-, piperazin-1-yl-, pyrimidin-1-yl-, pyridazin-1-yl-, morpholino-, thiomorpholino- or S,S-dioxothiomorpholino-carbonyl)-lower alkoxy, such as in 2-morpholinocarbonyl-ethoxy, 3-(morpholinocarbonyl)-propoxy or 3-(morpholinocarbonyl)-2-isobutyl-propoxy, as cycloalkoxy one of the cycloalkyl radicals mentioned under cycloalkyl-lower alkanoyl R₁, R₂, R₈ and R₉, bonded via oxygen, such as cyclobutoxy, cyclopentyloxy or cyclohexyloxy, or as aryloxy one of the radicals mentioned under aryloxy-substituted sulfonyl R₁, R₂, R₈ and R₉, especially benzyloxy or 1- or 2-naphthyloxy, and is especially preferably lower alkylphosphoryl, such as tert-butylphosphoryl, hydroxy-lower alkoxyphosphoryl, such as hydroxy-methoxy-phosphoryl or hydroxyethoxy-phosphoryl, or di-lower alkoxyphosphoryl, such as dimethoxy-phosphoryl or diethoxy-phosphoryl.

The phosphono radicals and the individual definitions of the radicals R₁, R₂, R₈ and R₉ mentioned for substituted phosphoryl may also, independently of one another, be omitted from the definition of the radicals R₁, R₂, R₈ and R₉ in the compounds of formula I.

Preferably not more than one of the radicals R₁ and R₂ and of the radicals R₈ and R₉ is defined by the radicals mentioned under acyl, sulfo, substituted sulfo, phosphono or substituted phosphoryl, while the other radical is selected from the remaining substituents mentioned.

A heterocyclic ring formed by the pairs of substituents RI and R₂, and R₈ and R₉, independently of one another, together with the nitrogen atom to which they are bonded, and comprising the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring, is preferably piperidino, pyrazin-1-yl, piperazin-1-yl, pyrimidin-1-yl, pyridazin-1-yl, morpholino, thiomorpholino, S,S-dioxothiomorpholino, succinimido, malimido, oxalimido, maleimido, phthalimido or naphthalene-1,8-dicarbonylimido, preferably only one of the pairs of substituents R₁ and R₂ or R₈ and R₉ forming one of the said heterocyclic rings.

Unsubstituted or substituted alkyl R₃, R₄ or R₇ is preferably one of the unsubstituted or substituted radicals mentioned under alkyl R₁, R₂, R₈ and R₉ and is selected especially from lower alkyl, for example methyl, ethyl, propyl, isopropyl, n-butyl, isobutyl or tert-butyl, cycloalkyl-lower alkyl that contains, for example, the cycloalkyl radicals mentioned under cycloalkyl-lower alkanoyl R₁, R₂, R₈ or R₉, such as cyclopropyl, cyclobutyl, cyclopentyl, cyclohexyl or cycloheptyl, cycloalkyl being unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and being bonded, preferably terminally, to lower alkyl, especially methyl, ethyl, n-propyl, isopropyl, n-butyl, isobutyl or tert-butyl, such as cyclobutyl-, cyclopentyl-, cyclohexyl- or cycloheptyl-lower alkyl, such as -methyl or -ethyl, especially preferably cyclohexyl-lower alkyl, such as cyclohexylmethyl, bicycloalkyl-lower alkyl wherein bicycloalkyl contains, for example, from 5 to 10, especially from 6 to 9, carbon atoms, for example bicycloalkyl-methyl or -ethyl, preferably having from 8 to 11 carbon atoms, such as decahydronaphthyl-2-methyl, endo- or exo-norbornyl-2-methyl, bicyclo[2.2.2]oct-2-ylmethyl or bicyclo[3.3.1]non-9-ylmethyl, and also bicyclo-hexyl-, -heptyl-, -octyl-, -nonyl- or -decyl-ethyl or -3-propyl, for example bicyclo[3.1.0]hex-1-, -2- or -3-yl-, bicyclo[4.1.0]hept-1- or -7-yl-, bicyclo[2.2.1]hept-2-yl-, for example endo- or exo-norbornyl-, bicyclo[3.2.1]oct-2-yl-, bicyclo[3.3.0]oct-3-yl- or bicyclo[3.3.1]non-9-yl-, and also α- or β-decahydronaphthyl-ethyl or -3-propyl, tricycloalkyl-lower alkyl, wherein tricycloalkyl contains, for example, from 8 to 10 carbon atoms, for example tricycloalkyl-methyl or -ethyl, preferably having from 8 to 11 carbon atoms, such as 1- or 2-adamantylmethyl, and also tricyclo[5.2.1.0.sup.2,6]dec-8-yl- or adamantyl-, such as 1-adamantyl-ethyl, aryl-lower alkyl, for example as defined under aryl-lower alkyl R₁, R₂, R₈ or R₉, that is unsubstituted or substituted as defined there, for example phenyl-lower alkyl, such as benzyl, 2-phenylethyl, 3-phenylpropyl, 4-fluoro-, 4-cyano-, 4-methoxy- or 4-hydroxybenzyl, or 1- or 2-naphthyl-methyl or -2-ethyl, especially phenyl-lower alkyl, as last defined, heterocyclyl-lower alkyl, for example as defined under heterocyclyl-lower alkyl R₁, R₂, R₈ or R₉, that is unsubstituted or substituted as defined there and is selected especially from pyrimidin-1-yl-, piperidino-, pyrazin-1-yl-, piperazin-1-yl-, pyridazin-1-yl-, morpholino-, thiomorpholino- or S,S-dioxothiomorpholino-lower alkyl, such as in 2-morpholinoethyl, 3-morpholino-propyl or 3-morpholino-2-isobutyl-propyl, unsubstituted or lower alkyl- or phenyl-substituted pyrrolyl-lower alkyl, such as 2- or 3-pyrrolyl-methyl, -ethyl or -n-propyl, 4- or 5-methylpyrrolyl-methyl, -ethyl or -n-propyl or 4- or 5-phenylpyrrolylmethyl, -ethyl or -n-propyl, thienyl-lower alkyl, such as 2-thienyl-methyl, -ethyl or -n-propyl, 1-imidazolylmethyl, furyl-lower alkyl, such as 2-furyl-methyl, -ethyl or -n-propyl, pyridyl-lower alkyl, such as 2-, 3- or 4-pyridyl-methyl, -ethyl or -n-propyl, indolyl-lower alkyl that is unsubstituted or substituted by lower alkyl, for example methyl, phenyl-lower alkyl, for example benzyl, lower alkoxy, for example methoxy, phenyl-lower alkoxy, for example benzyloxy, or by halogen, for example chlorine, such as 2-, 3- or 5-indolyl-methyl, -ethyl or -n-propyl, 1-methyl-, 2-methyl-, 5-methoxy-, 5-benzyloxy-, 5-chloro- or 4,5-dimethyl-indol-2-yl-, 1-benzylindol-2-yl- or -3-yl-methyl, -ethyl or -n-propyl, 4,5,6,7-tetrahydroindol-2-yl-methyl, -ethyl or -n-propyl, cyclohepta[b]pyrrol-5-yl-methyl, -ethyl or -n-propyl, unsubstituted or hydroxy-substituted quinolyl-lower alkyl, for example 2-, 3- or 4-quinolyl- or 4-hydroxyquinol-2-yl-methyl, -ethyl or -n-propyl, unsubstituted or hydroxy-substituted isoquinolyl-lower alkyl, such as 1-, 3- or 4-isoquinolyl- or 1-oxo-1,2-dihydroisoquinol-3-yl-methyl, -ethyl or -n-propyl, 2-quinoxalinyl-methyl, -ethyl or -n-propyl, 3,1-benzofuran-2-yl-methyl, -ethyl or -n-propyl, benz[e]-indol-2-yl-methyl, -ethyl or -n-propyl, β-carbolin-3-yl-methyl, -ethyl or -n-propyl, 3-chromanyl-methyl, -ethyl or -n-propyl, 3-thiochromanyl-methyl, -ethyl or -n-propyl, 3- or 4-pyrrolidinyl-methyl, -ethyl or -n-propyl, hydroxypyrrolidinyl-lower alkyl, such as 3- or 4-hydroxypyrrolidin-2-yl-methyl, -ethyl or -n-propyl, oxopyrrolidinyl-lower alkyl, such as 5-oxopyrrolidin-2-yl-methyl, -ethyl or -n-propyl, piperidinyl-lower alkyl, such as 2-3- or 4-piperidinyl-methyl, -ethyl or -n-propyl, morpholinyl-lower alkyl, such as 2- or 3-morpholinyl-methyl, -ethyl or -n-propyl, thiomorpholinyl-lower alkyl, such as 2- or 3-thiomorpholinyl-methyl, -ethyl or -n-propyl, S,S-dioxothiomorpholinyl-lower alkyl, such as S,S-dioxothiomorpholin-2- or -3-yl-methyl, -ethyl or -n-propyl, indolinyl-lower alkyl, such as 2- or 3-indolinyl-methyl, -ethyl or -n-propyl, 1,2,3,4-tetrahydroquinolyl-lower alkyl, such as 1,2,3,4-tetrahydroquinol-2-, -3- or -4-yl-methyl, -ethyl or -n-propyl, 1,2,3,4-tetrahydroisoquinolyl-lower alkyl, such as 1,2,3,4-tetrahydroisoquinol-1-, -2- or -3-yl-methyl, -ethyl or -n-propyl, and 1-oxo-1,2,3,4-tetrahydroisoquinol-3-yl-methyl, -ethyl or -n-propyl, hydroxy-lower alkyl, such as 3-hydroxypropyl or 2-hydroxy-3-methylpentyl, lower alkoxy-lower alkyl, for example lower alkoxyethyl or lower alkoxypropyl, such as 2-methoxyethyl, 2-ethoxyethyl or 3-methoxypropyl, phenoxy-lower alkyl or nitrophenoxy-lower alkyl, such as phenoxymethyl, phenoxyethyl or 4-nitrophenoxymethyl, naphthyloxy-lower alkyl, such as α- or β-naphthyloxyethyl, lower alkanoyloxy-lower alkyl, for example lower alkanoyloxyethyl or lower alkanoyloxypropyl, such as acetoxyethyl or 3-acetoxypropyl, acetoacetoxy-lower alkanoyl, arylmercapto-lower alkyl wherein aryl contains from 6 to 10-carbon atoms, for example phenyl or naphthyl, such as phenylmercaptomethyl, amino-lower alkyl, such as 5-aminopentyl, mono- or di-lower alkylamino-lower alkyl, such as dimethylaminoethyl or 2-dimethylamino-2-isopropylethyl, phenyl- or naphthyl-amino-lower alkyl, such as 3-phenylaminopropyl, lower alkanoylamino-lower alkyl, such as 4-acetylaminopentyl, piperazinylcarbamoyl-lower alkyl substituted at the nitrogen atom by lower alkyl, such as methyl, such as 4-methylpiperazinylcarbonylmethyl, lower alkoxycarbonylamino-lower alkyl, such as 5-(tert-butoxycarbonylamino)-pentyl or 3-ethoxycarbonylamino-2-isobutyl-propyl, phenyl-lower alkoxycarbonylamino-lower alkyl, such as 5-(benzyloxycarbonylamino)pentyl, aminocarbonylamino-lower alkyl, such as 2-aminocarbonylamino-ethyl, N-phenyl-lower alkyl-N-lower alkylaminocarbonylamino-lower alkyl, such as 2-isobutyl-3-(N-benzyl-N-methylaminocarbonylamino)propyl, halo-lower alkyl wherein halogen is selected from fluorine, chlorine, bromine and iodine, for example 2-haloethyl, such as 2-fluoro-, 2-chloro-, 2-bromo-, 2-iodo-, 2,2,2-trifluoro- or 2,2,2-trichloro-ethyl, trifluoro-lower alkyl, such as trifluoromethyl, or halopropyl, such as 3-fluoro-, 3-chloro- or 3-bromo-propyl, carboxy-lower alkyl, such as carboxyethyl or 3-carboxypropyl, lower alkoxycarbonyl-lower alkyl, for example lower alkoxycarbonylethyl or lower alkoxycarbonylpropyl, such as methoxycarbonylethyl, 3-methoxycarbonylpropyl, ethoxycarbonylethyl or 3-ethoxycarbonylpropyl, 2-halo-lower alkoxycarbonyl-lower alkyl, such as 2-chloro-, 2-bromo-, 2-iodo- or 2,2,2-trichloro-ethoxycarbonyl-2-ethyl or -3-propyl, phenyl- or naphthyl-lower alkoxycarbonyl-lower alkyl, for example benzyloxycarbonyl-lower alkyl, such as 3-benzyloxycarbonyl-2,2-dimethylpropyl, heterocyclyl-lower alkoxycarbonyl-lower alkyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, βcarbolinyl and a benzo-fused, cyclopenta-, cyclohexa- or cyclohepta-fused derivative of those radicals, it also being possible for the mentioned radicals to be fully or partially saturated, such as in 4-pyridylmethyloxycarbonyl-2-ethyl or -3-propyl or 2-morpholinocarbonyloxy-4-methylpentyl, lower alkylsulfonyl-lower alkyl, for example 2-ethylsulfonyl- or 2-tert-butylsulfonymethmethyl, arylsulfonyl-lower alkyl wherein aryl preferably contains from 6 to 10 carbon atoms, for example phenyl or naphthyl, such as phenylsulfomethyl, carbamoyl-lower alkyl, such as carbamoylethyl or 3-carbamoylpropyl, lower alkylcarbamoyl-lower alkyl, for example lower alkylcarbamoylethyl or methylcarbamoyl-lower alkyl, such as 2-methylcarbamoylethyl, di-lower alkylcarbamoyl-lower alkyl, for example 2-di-lower alkylcarbamoylethyl or dimethylcarbamoyl-lower alkyl, such as 2-dimethylcarbamoylethyl, hydroxy-lower alkylcarbamoyl- or di(hydroxy-lower alkyl)carbamoyl-lower alkyl, such as 2-hydroxymethylcarbamoyl- or di(hydroxymethyl)carbamoyl-2-ethyl or -3-propyl, N-lower alkoxy-lower alkoxy-lower alkylcarbamoyl-lower alkyl, such as 2-isobutyl-3-(2-(2-methoxyethoxy)ethylaminocarbonyl)-propyl, carboxy-lower alkylcarbamoyl- or di(carboxy-lower alkyl)carbamoyl-lower alkyl, for example carboxymethyl- or di(carboxymethyl)-carbamoyl-2-ethyl or -3-propyl, carbamoyl-lower alkyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, pyrimidin-1-yl-, pyridazin-1-yl-, morpholino-, thiomorpholino- or S,S-dioxothiomorpholino-carbonyl-lower alkyl, such as in 2-morpholinocarbonyl-ethyl, 3-(morpholinocarbonyl)-propyl or 3-(morpholinocarbonyl)-2-isobutyl-propyl, N-lower alkyl-N-heterocyclyl-lower alkylcarbamoyl-lower alkyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, such as 2-(N-2-pyridylmethyl)-N-methylcarbamoyl-ethyl, sulfamoyl-lower alkyl, such as 2-sulfamoylethyl, N-(phenyl- or naphthyl-lower alkyl)sulfamoyl-lower alkyl, such as 3-benzylaminosulfonyl-2-isopropyl-propyl, or sulfamoyl-lower alkyl substituted at the nitrogen atom by a radical selected from ethylene, trimethylene, tetramethylene and pentamethylene wherein a carbon atom may have been replaced by nitrogen, nitrogen substituted by lower alkyl, such as methyl, oxygen, sulfur or by sulfur mono- or di-substituted by oxygen, it also being possible for the radical so formed to be fully or partially unsaturated, for example in the form of piperidino-, pyrazin-1-yl-, piperazin-1-yl-, 4-methylpiperazin-1-yl-, pyrimidin-1-yl-, pyridazin-1-yl-, morpholino-, thiomorpholino- or S,S-dioxothiomorpholino-sulfonyl-lower alkyl, for example 3-(4-methylpiperazinylsulfonyl)-2-isopropyl-propyl or 3-(morpholinosulfonyl)-2-isopropyl-propyl, oxo-lower alkyl (wherein oxo is not present at the carbon atom that is bonded to the nitrogen atom carrying the radical R₇), such as 3-oxo-n-butyl or 3-oxo-n-pentyl, cyano-lower alkyl, such as cyanomethyl, 2-cyanoethyl, 2- or 3-cyano-n-propyl or 2-, 3- or 4-cyano-n-butyl, hydroxy-carboxy-lower alkyl, such as 2-hydroxy-2-carboxyethyl or 2-hydroxy-3-carboxypropyl, α-naphthyloxy-carboxy-lower alkyl, such as 2-α-naphthyloxy-4-carboxy-n-butyl, hydroxy-lower alkoxycarbonyl-lower alkyl, for example 2-hydroxy-2-lower alkoxycarbonyl-ethyl or -propyl or hydroxy-ethoxy- or hydroxy-methoxy-carbonyl-lower alkyl, such as 2-hydroxy-2-ethoxy- or -methoxy-carbonylethyl or 2-hydroxy-3-ethoxy- or -methoxy-carbonyl-propyl, α-naphthyloxy-lower alkoxycarbonyl-lower alkyl, for example α-naphthyloxy-lower alkoxycarbonyl-2-ethyl, -2-propyl or -2-butyryl or α-naphthyloxyethoxycarbonyl-lower alkyl, such as α-naphthyloxyethoxycarbonyl-2-ethyl, 2-α-naphthyloxy-3-ethoxycarbonylpropyl or 2-α-naphthyloxy-4-tert-butoxycarbonylbutyl, lower alkylcarbonylhalo-lower alkyl, such as 3-ethoxycarbonyl-2-difluoromethyl, α-naphthyloxy-benzyloxycarbonyl-lower alkyl, such as 2-α-naphthyloxy-3-benzyloxycarbonyl-propyl, esterified hydroxy-lower alkoxycarbonyl-lower alkyl wherein the hydroxy group is esterified by lower alkanoyl, for example acetyl, propionyl or pivaloyl, cycloalkyl-lower alkanoyl wherein cycloalkyl contains from 3 to 7 carbon atoms and lower alkanoyl is as last defined, for example cyclohexylcarbonyl or 2-cyclohexyl- or 2-cyclopentyl-acetyl, bicycloalkyl-lower alkanoyl wherein bicycloalkyl contains, for example, from 5 to 10, especially from 6 to 9, carbon atoms, such as in bicyclo-hexyl-, -heptyl-, -octyl-, -nonyl- or -decyl-acetyl or -3-propionyl, for example bicyclo[3.1.0]hex-1-, -2- or -3-yl-, bicyclo-[4.1.0]hept-1- or -7-yl-, bicyclo[2.2.1]hept-2-yl-, for example endo- or exo-norbornyl-, bicyclo[3.2.1]oct-2-yl-, bicyclo[3.3.0]oct-3-yl- or bicyclo[3.3.1]non-9-yl-, and also α- or β-decahydronaphthyl-acetyl or -3-propionyl, tricycloalkyl-lower alkanoyl wherein tricycloalkyl contains, for example, from 8 to 10 carbon atoms, for example in tricyclo[5.2.1.0.sup.2,6]dec-8-yl- or adamantyl-such as 1-adamantyl-acetyl, aryl-lower alkanoyl wherein aryl contains from 6 to 14 carbon atoms, for example phenyl, indenyl, indanyl, naphthyl, anthryl, phenanthryl, acenaphthyl or fluorenyl, and may be unsubstituted or mono- to tri-substituted by lower alkyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl, di-lower alkoxyphosphoryl, carbamoyl, sulfamoyl, nitro and/or by cyano, lower alkoxycarbonyl, for example tert-butoxycarbonyl, 2-halo-lower alkoxycarbonyl, as defined above, or by phenyl- or fluorenyl-lower alkoxycarbonyl, for example benzyloxycarbonyl or 9-fluorenylmethoxycarbonyl, such as 2-acetoxy-2-methoxycarbonyl-ethyl, 2-benzoyloxy-, 2-(1- or 2-naphthoyloxy)-, 2-(phenyl-2-acetoxy)-, 2-(1- or 2-naphthyl-2-acetoxy)-, 2-(4-methylphenyl-2-acetoxy)-, 2-(4-methoxyphenyl-2-acetoxy)- or 2-(2-(o,o-dichlorophenyl)-2-acetoxy)-2-methoxycarbonyl-ethyl or -3-propyl, dihydroxy-carboxy-lower alkyl, such as 2,3-dihydroxy-3-carboxy-propyl, dihydroxy-lower alkoxycarbonyl-lower alkyl, such as 2,3-dihydroxy-3-ethoxy- or -methoxy-carbonyl-propyl, dihydroxy-lower alkoxycarbonyl-lower alkyl esterified by lower alkanoyl, such as acetyl, propionyl or butyryl, lower alkoxycarbonyl, for example tert-butoxycarbonyl, phenyl- or fluorenyl-lower alkoxycarbonyl, such as benzyloxycarbonyl or 9-fluorenylmethoxycarbonyl, lower alkylsulfonyl or by toluenesulfonyl, for example di-lower alkanoyloxy-lower alkoxy-propyl, such as 2,3-diacetoxy-3-methoxycarbonyl-propyl, α-naphthyloxy-di-lower alkylamino-lower alkyl, such as 2-α-naphthyloxy-5-dimethylamino-pentyl, α-naphthyloxy-carbamoyl-lower alkyl, such as 2-α-naphthyloxy-4-carbamoyl-butyl, α-naphthyloxy-oxo-lower alkyl (wherein oxo is not present at the carbon atom that is bonded to the nitrogen atom carrying the radical R₇), such as 2-α-naphthyloxy-4-oxo-pentyl, or α-naphthyloxy-cyano-lower alkyl, such as 2-α-naphthyloxy-cyano-ethyl or 2-α-naphthyloxy-4-cyano-butyl.

Lower alkyl that is unsubstituted or mono- or poly-substituted by hydroxy, lower alkoxy, amino, mono- or di-lower alkylamino, fluorine, chlorine or by cyano is preferred, and lower alkyl, such as methyl, ethyl, propyl, isopropyl, n-butyl, isobutyl or tert-butyl, is very especially preferred.

Cycloalkyl R₃, R₄ or R₇ is preferably as defined under cycloalkyl-lower alkanoyl R₁, R₂, R₈ or R₉, for example cyclopropyl, cyclobutyl, cyclopentyl, cyclohexyl or cycloheptyl, cycloalkyl being unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano, such as cyclobutyl, cyclopentyl, cyclohexyl or cycloheptyl, especially cyclohexyl.

Aryl R₃, R₄ or R₇ is preferably one of the unsubstituted or substituted aryl radicals mentioned under aryl-lower alkanoyl R₁, R₂, R₈ or R₉ and is especially phenyl, naphthyl or fluorenyl that is unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano, such as phenyl, 4-methoxyphenyl, 4-fluorophenyl, 4-nitrophenyl, 4-cyanophenyl or 1- or 2-naphthyl.

Heterocyclyl R₃, R₄ or R₇ is preferably as defined under heterocyclyl R₁, R₂, R₈ or R₉, which is unsubstituted or substituted as defined there, and is selected especially from 2- or 3-pyrrolyl, 4- or 5-methylpyrrolyl or 4- or 5-phenylpyrrolyl, 2-thienyl, 2-furyl, 2-, 3- or 4,5-dimethyl-indol-2-yl, 1-benzylindol-2- or -3-yl, 4,5,6,7-tetrahydroindol-2-yl, cyclohepta[b]pyrrol-5-yl, 2-, 3- or 4-quinolyl or 4-hydroxyquinol-2-yl, 1-, 3- or 4-isoquinolyl, 1-oxo-1,2-dihydroisoquinol-3-yl, 2-quinoxalinyl, 3,1-benzofuran-2-yl, benz[e]indol-2-yl, β-carbolin-3-yl, 3-chromanyl, 3-thiochromanyl, 3-pyrrolidinyl, 3- or 4-hydroxypyrrolidin-2-yl, such as 5-oxopyrrolidin-2-yl, 2-, 3- or 4-piperidinyl, 2- or 3-morpholinyl, 2- or 3-thiomorpholinyl, S,S-dioxothiomorpholin-2- or -3-yl, 2- or 3-indolinyl, 1,2,3,4-tetrahydroquinol-2-, -3- or -4-yl, 1,2,3,4-tetrahydroisoquinol-1-, -2- or -3-yl, or 1-oxo-1,2,3,4-tetrahydroisoquinol-3-yl.

Unsubstituted or substituted alkenyl R₃, R₄ or R₇ is preferably as defined under alkenyl R₁, R₂, R₈ or R₉, especially lower alkenyl that is unsubstituted or substituted as defined there, for example lower alkenyl, such as vinyl, allyl or 2- or 3-butenyl, cycloalkyl-lower alkenyl wherein cycloalkyl is as defined in the case of cycloalkyl-lower alkanoyl R₁, R₂, R₈ or R₉ and is unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded especially to the terminal carbon atom of lower alkenyl, such as cyclobutyl-, cyclopentyl-, cyclohexyl- or cycloheptyl-methyl-2-vinyl, -2- or -3-allyl or -2-, -3- or -4-but-2-enyl, aryl-lower alkenyl having an unsubstituted or substituted aryl radical defined under aryl R₃ or R₄ that is bonded, preferably terminally, to lower alkenyl, and having a lower alkenyl radical defined under lower alkenyl R₁, R₂, R₈ or R₉, such as styryl, 3-phenylallyl (cinnamyl), 2-(α-naphthyl)-vinyl or 2-(β-naphthyl)-vinyl, or unsubstituted or substituted heterocyclyl-lower alkenyl that contains, for example, an unsubstituted or substituted lower alkenyl radical mentioned under alkenyl R₁, R₂, R₈ or R₉, for example vinyl, allyl or 2- or 3-butenyl, that is substituted, preferably at the terminal carbon atom, by an unsubstituted or substituted heterocyclyl radical mentioned under heterocyclyl R₁, R₂, R₈ or R₉, for example in the form of pyrimidin-1-yl-, piperidino-, pyrazin-1-yl-, piperazin-1-yl-, pyridazin-1-yl-, morpholino-, thiomorpholino- or S,S-dioxothiomorpholino-lower alkenyl, such as in 2-morpholino-vinyl, 3-morpholinoallyl or 4-morpholino-2- or -3-butenyl, unsubstituted or lower alkyl- or phenyl-substituted pyrrolyl-lower alkenyl, such as 2- or 3-pyrrolyl-vinyl or -allyl, 4- or 5-methylpyrrolylvinyl or -allyl or 4- or 5-phenylpyrrolyl-vinyl or -allyl, thienyl-lower alkenyl, such as 2-thienyl-vinyl or -allyl, furyl-lower alkenyl, such as 2-furyl-vinyl or -allyl, pyridyl-lower alkenyl, such as 2-, 3- or 4-pyridyl-vinyl or -allyl, indolyl-lower alkenyl that is unsubstituted or substituted by lower alkyl, for example methyl, phenyl-lower alkyl, for example benzyl, lower alkoxy, for example methoxy, phenyl-lower alkoxy, for example benzyloxy, or by halogen, for example chlorine, such as 2-, 3- or 5-indolyl-vinyl or -allyl, 1-methyl-, 2-methyl-, 5-methoxy-, 5-benzyloxy-, 5-chloro- or 4,5-dimethyl-indol-2-yl-, 1-benzylindol-2-yl- or -3-yl-vinyl or -allyl, 4,5,6,7-tetrahydroindol-2-yl-methyl, -ethyl or -n-propyl, cyclohepta[b]pyrrol-5-yl-vinyl or -allyl, unsubstituted or hydroxy-substituted quinolyl-lower alkenyl, for example 2-, 3- or 4-quinolyl- or 4-hydroxyquinol-2-yl-vinyl or -allyl, unsubstituted or hydroxy-substituted isoquinolyl-lower alkenyl, such as 1-, 3- or 4-isoquinolyl- or 1-oxo-1,2-dihydroisoquinol-3-yl-vinyl or -allyl, 2-quinoxalinyl-vinyl or -allyl, 3,1-benzofuran-2-yl-vinyl or -allyl, benz[e]indol2-yl-vinyl or -allyl, β-carbolin-3-yl-vinyl or -allyl, 3-chromanyl-vinyl or -allyl, 3-thiochromanyl-vinyl or -allyl, 3-pyrrolidinyl-vinyl or -allyl, hydroxypyrrolidinyl-lower alkenyl, such as 3- or 4-hydroxypyrrolidin-2-yl-vinyl or -allyl, oxopyrrolidinyl-lower alkenyl, such as 5-oxopyrrolidin-2-yl-vinyl or -allyl, piperidinyl-lower alkenyl, such as 2-, 3- or 4-piperidinyl-vinyl or -allyl, morpholinyl-lower alkenyl, such as 2- or 3-morpholinyl-vinyl or -allyl, thiomorpholinyl-lower alkenyl, such as 2- or 3-thiomorpholinyl-vinyl or -allyl, S,S-dioxothiomorpholinyl-lower alkenyl, such as S,S-dioxothiomorpholin-2- or -3-yl-vinyl or -allyl, indolinyl-lower alkenyl, such as 2- or 3-indolinyl-vinyl or -allyl, 1,2,3,4-tetrahydroquinolyl-lower alkenyl, such as 1,2,3,4-tetrahydroquinol-2-, -3- or -4-yl-vinyl or -allyl, 1,2,3,4-tetrahydroisoquinolyl-lower alkenyl, such as 1,2,3,4-tetrahydroisoquinol-1-, -2- or -3-yl-vinyl or -allyl, or 1-oxo-1,2,3,4-tetrahydroisoquinol-3-yl-vinyl or -allyl.

Unsubstituted or substituted alkylene formed by R₃ and R₄ together contains especially an alkylene radical having up to 20 carbon atoms, it also being possible for the mentioned radicals to contain one or more double bonds, preferably lower alkylene, for example ethylene, trimethylene, tetramethylene, pentamethylene, hexamethylene or heptamethylene, that is unsubstituted or substituted, especially by unsubstituted or substituted aryl, as defined under aryl-lower alkanoyl R₁, R₂, R₈ or R₉, preferably by phenyl, indenyl, indanyl, naphthyl, anthryl, phenanthryl, acenaphthyl or fluorenyl, which are unsubstituted or mono- to tri-substituted by radicals such as lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, phenyl-, naphthyl- or fluorenyl-lower alkoxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or cyano, and/or is substituted in the same manner as lower alkanoyl in aryl-lower alkanoyl R₁, R₂, R₈ or R₉, especially by lower alkyl, for example methyl, hydroxy, lower alkoxy, for example methoxy or ethoxy, lower alkanoyloxy, for example acetoxy or propionyloxy, benzoyloxy, phenylacetoxy or 1- or 2-naphthoyloxy, lower alkoxycarbonyloxy, for example isopropoxycarbonyloxy or tert-butoxycarbonyloxy, phenyl-lower alkoxycarbonyloxy, for example benzyloxycarbonyloxy, 9-fluorenylmethoxycarbonyloxy, sulfonyloxy, lower alkylsulfonyloxy, for example methyl- or ethyl-sulfonyloxy, phenylsulfonyloxy, 2- or 4-toluenesulfonyloxy, amino, mono- or di-lower alkylamino, for example mono- or di-methylamino or -ethyl-amino, lower alkanoylamino, for example acetylamino or pivaloylamino, carboxy, lower alkoxycarbonyl, for example isopropoxy- or tert-butoxy-carbonyl, benzyloxycarbonyl, 9-fluorenylmethoxycarbonyl, lower alkanoyl, for example acetyl or propionyl, lower alkylsulfonyl, such as methylsulfonyl or tert-butylsulfonyl, phosphono, carbamoyl, mono- or di-lower alkylcarbamoyl, for example N-methylcarbamoyl or N,N-dimethylcarbamoyl, piperidino, pyrazin-1-yl, pyrimidin-1-yl, pyridazin-1-yl, morpholino, thiomorpholino or S,S-dioxothiomorpholino, sulfamoyl, oxo and/or by cyano, such as ethylene, ethyl-ethylene, trimethylene, propylene or tetramethylene.

Unsubstituted or substituted alkylidene formed by R₃ and R₄ together contains up to 20 carbon atoms and no double bonds or one or more double bonds in addition to the linking double bond, and is preferably lower alkylidene, for example methylene, ethylidene, propylidene, butylidene or pentylidene, that is unsubstituted or substituted especially by cycloalkyl, such as mentioned under cycloalkyl-lower alkanoyl R₁, R₂, R₈ or R₉, for example cyclopropyl, cyclobutyl, cyclopentyl or cyclohexyl, cycloalkenyl, such as mentioned under cycloalkenyl-lower alkanoyl R₁, R₂, R₈ or R₉, for example cyclohexen-1-yl or 1,4-cyclohexadienyl, unsubstituted or substituted aryl, such as defined under aryl-lower alkanoyl R₁, R₂, R₈ or R₉, preferably by phenyl, indenyl, indanyl, naphthyl, anthryl, phenanthryl, acenaphthyl or fluorenyl, which are unsubstituted or mono- to tri-substituted by radicals such as lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, phenyl-, naphthyl- or fluorenyl-lower alkoxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or cyano, and/or is substituted in the same manner as lower alkanoyl in aryl-lower alkanoyl R₁, R₂, R₈ or R₉, especially by lower alkyl, for example methyl, hydroxy, lower alkoxy, for example methoxy or ethoxy, lower alkanoyloxy, for example acetoxy or propionyloxy, benzoyloxy, phenylacetoxy or 1- or 2-naphthoyloxy, lower alkoxycarbonyloxy, for example isopropoxycarbonyloxy or tert-butoxycarbonyloxy, phenyl-lower alkoxycarbonyloxy, for example benzyloxycarbonyloxy, 9-fluorenylmethoxycarbonyloxy, sulfonyloxy, lower alkylsulfonyloxy, for example methyl- or ethyl-sulfonyloxy, phenylsulfonyloxy, 2- or 4-toluenesulfonyloxy, amino, mono- or di-lower alkylamino, for example mono- or di-methyl-amino or -ethyl-amino, lower alkanoylamino, for example acetylamino or pivaloylamino, carboxy, lower alkoxycarbonyl, for example isopropoxy- or tert-butoxy-carbonyl, benzyloxycarbonyl, 9-fluorenylmethoxycarbonyl, lower alkanoyl, for example acetyl or propionyl, lower alkylsulfonyl, such as methylsulfonyl or tert-butylsulfonyl, phosphono, carbamoyl, mono- or di-lower alkylcarbamoyl, for example N-methylcarbamoyl or N,N-dimethylcarbamoyl, piperidino, pyrazin-1-yl, pyrimidin-1-yl, pyridazin-1-yl, morpholino, thiomorpholino or S,S-dioxothiomorpholino, sulfamoyl, oxo and/or by cyano, such as ethylidene, propylidene, butylidene, benzylidene or cinnamylidene.

Unsubstituted or substituted benzo-fused alkylene formed by R₃ and R₄ together contains up to 20 carbon atoms and is preferably lower alkylene, for example ethylene, trimethylene, tetramethylene, pentamethylene, hexamethylene or heptamethylene, onto which a benzene ring has been fused, and is unsubstituted or substituted especially by unsubstituted or substituted aryl, as defined under aryl-lower alkanoyl R₁, R₂, R₈ or R₉, preferably by phenyl, indenyl, indanyl, naphthyl, anthryl, phenanthryl, acenaphthyl or fluorenyl, which are unsubstituted or mono- to tri-substituted by radicals such as lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, phenyl-, naphthyl- or fluorenyl-lower alkoxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or cyano, and may also be substituted by lower alkyl, for example methyl, hydroxy, lower alkoxy, for example methoxy or ethoxy, lower alkanoyloxy, for example acetoxy or propionyloxy, benzoyloxy, phenylacetoxy or 1- or 2-naphthoyloxy, lower alkoxycarbonyloxy, for example isopropoxycarbonyloxy or tert-butoxycarbonyloxy, phenyl-lower alkoxycarbonyloxy, for example benzyloxycarbonyloxy, 9-fluorenylmethoxycarbonyloxy, sulfonyloxy, lower alkylsulfonyloxy, for example methyl- or ethyl-sulfonyloxy, phenylsulfonyloxy, 2- or 4-toluenesulfonyloxy, amino, mono- or di-lower alkylamino, for example mono- or di-methyl-amino or -ethyl-amino, lower alkanoylamino, for example acetylamino or pivaloylamino, carboxy, lower alkoxycarbonyl, for example isopropoxy- or tert-butoxycarbonyl, benzyloxycarbonyl, 9-fluorenylmethoxycarbonyl, lower alkanoyl, for example acetyl or propionyl, lower alkylsulfonyl, such as methylsulfonyl or tert-butylsulfonyl, phosphono, carbamoyl, mono- or di-lower alkylcarbamoyl, for example N-methylcarbamoyl or N,N-dimethylcarbamoyl, piperidino, pyrazin-1-yl, pyrimidin-1-yl, pyridazin-1-yl, morpholino, thiomorpholino or S,S-dioxothiomorpholino, sulfamoyl, oxo and/or by cyano, such as ortho-phenylene.

When, in the compounds of formula I substituted by the mentioned radicals, nitrogen atoms having free hydrogen and/or hydroxy groups are vicinal with respect to double or triple bonds (as in the case of unsubstituted or substituted alkenyl or alkynyl R₁, R₂, R₈ or R₉), the corresponding tautomeric imino and oxo compounds are always also included.

Salts of compounds of formula I are preferably acid addition salts, salts with bases or, where several salt-forming groups are present, can also be mixed salts or internal salts. Salts are especially the pharmaceutically acceptable, non-toxic salts of compounds of formula I.

In the groups of compounds of formula I mentioned below, it may be advantageous, for example in order to replace rather general definitions with more specific definitions, to use definitions of radicals from the above-mentioned general definitions or to insert or omit definitions from the other groups.

Preference is given to a compound of formula I according to formula (I) 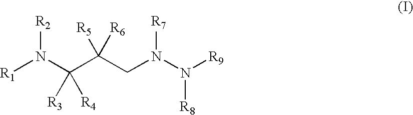
wherein R₁ and R₉ are each independently of the other hydrogen; lower alkanoyl, such as formyl, acetyl, propionyl, butyryl or pivaloyl, especially acetyl; aryl-lower alkanoyl wherein aryl is preferably as defined above under the general definitions of aryl-lower alkanoyl and is unsubstituted or substituted as defined there, i.e. aryl has from 6 to 14 carbon atoms, as in phenyl, indenyl, indanyl, naphthyl, anthryl, phenanthryl, acenaphthyl or fluorenyl, and may be unsubstituted or especially mono- to tri-substituted by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, for example in diphenyl-, dibenzyl- or triphenyl-lower alkanoyl, such as diphenyl-, dibenzyl- or triphenyl-acetyl, and wherein lower alkanoyl is unsubstituted or substituted by carbamoyl or by carbamoyl substituted at the nitrogen atom by one or two radicals selected from lower alkyl, carboxy-lower alkyl, lower alkoxycarbonyl-lower alkyl, di-lower alkylamino-lower alkyl, aminocarboxy-lower alkyl, hydroxy-lower alkyl and di-lower alkoxy-lower alkyl, preferably as described under aryl-lower alkanoyl above in the general definitions, for example 4-chloro-4-methoxy- or 4-nitro-benzoyl, naphthylcarbonyl, such as α- or β-naphthylcarbonyl, or 1,8-naphthalene-dicarbonyl bonded to the amino group via both carbonyl groups, indenylcarbonyl, such as 1-, 2- or 3-indenylcarbonyl, indanylcarbonyl, such as 1- or 2-indanylcarbonyl, phenanthrenylcarbonyl, such as 9-phenanthrenylcarbonyl, phenyl-lower alkanoyl, such as phenylacetyl or 3-phenylpropionyl, α-naphthylacetyl, β-naphthylacetyl, lower alkylphenylacetyl, such as 4-methylphenylacetyl, lower alkoxyphenylacetyl, such as 4-methoxyphenylacetyl, 3-phenylpropionyl, 3-(p-hydroxyphenyl)propionyl, diphenylacetyl, di(4-methoxyphenyl)acetyl, triphenylacetyl, 2,2-dibenzylacetyl, 3-α- or 3-β-naphthylpropionyl, phenyl-lower alkanoyl wherein the lower alkanoyl radical is substituted by carbamoyl, such as 2(R,S)-carbamoyl-3-phenylpropionyl, 3-phenyl- or 3-α-naphthyl-2-carbamoylpropionyl, 3-phenyl- or 3-α-naphthyl-2-tert-butylcarbamoylpropionyl, 3-phenyl- or 3-α-naphthyl-2-(2-dimethylaminoethyl) carbamoylpropionyl, 3-α-naphthyl-2-(carboxy- or tert-butoxycarbonyl)methyl carbamoylpropionyl, 3-phenyl- or 3-α-naphthyl-2-(3-hydroxy-2-propyl) carbamoylpropionyl, 3-phenyl- or 3-α-naphthyl-2-(2,2-dimethoxyethyl)-carbamoylpropionyl, 3-phenyl- or 3-α-naphthyl-2-(5-amino-5-carboxypentyl)-carbamoylpropionyl, especially phenyl-lower alkanoyl, such as phenylacetyl, or phenyl-lower alkanoyl wherein the lower alkanoyl radical is substituted by carbamoyl, such as 2(R,S)-carbamoyl-3-phenylpropionyl; heterocyclyl-lower alkanoyl that is preferably as defined above for heteroaryl-lower alkanoyl R₁, R₂, R₈ and R₉, especially heterocyclyl-lower alkanoyl wherein lower alkanoyl is unsubstituted and wherein heterocyclyl is preferably a single or a double ring system having from 3 to 10 ring atoms, is bonded via a carbon atom or, especially, via a nitrogen atom, contains up to three further hetero atoms selected from oxygen, nitrogen, sulfur, selenium, and sulfur linked to 1 or 2 oxygen atoms, and may be unsaturated or partially or fully saturated, for example thienyl-, furyl-, pyranyl-, pyrrolyl-, imidazolyl-, pyrazolyl-, oxazolyl-, isoxazolyl-, thiazolyl-, furazanyl-, tetrazolyl-, pyridyl-, pyrazinyl-, pyrimidinyl-, pyridazinyl-, azepinyl-, indolyl-, benzimidazolyl-, 1H-indazolyl-, quinolyl-, isoquinolyl-, quinoxalinyl-, quinazolinyl-, cinnolyl-, purinyl-, pteridinyl-, naphthyridinyl-, 4H-quinolizinyl-, 3,1-benzofuranyl-, 4,1-benzoxazinyl-, 4,1-benzothiazinyl-, cyclohexa[b]pyrrolyl-, cyclohepta[b]pyrrolyl-, cyclohexa[d]pyrazolyl-, cyclohexa[b]pyridyl-, cyclohexa[b]pyrazinyl-, cyclohexa[b]pyrimidinyl-, cyclohexa[b]-1,4-oxazinyl-, cyclohexa[b]-1,4-thiazinyl-, pyrrolidinyl-, pyrrolinyl-, imidazolidyl-, 2-imidazolinyl-, 2,3-dihydropyridyl-, piperidyl-, piperazinyl-, 2,3,5,6-tetrahydropyrazinyl-, morpholinyl-, thiomorpholinyl-, S,S-dioxo-thiomorpholinyl-, indolinyl-, isoindolinyl-, 4,5,6,7-tetrahydroindolyl-, 1,2,3,4-tetrahydroquinolyl-, 1,2,3,4-tetrahydroisoquinolyl-, chromanyl-, thiochromanyl-, 1,2,3,4-tetrahydro-3,1-benzodiazinyl-, 3,4-dihydro-3H-4,1-benzoxazinyl- or 3,4-dihydro-3H-4,1-benzothiazinyl-lower alkanoyl, the mentioned heterocyclyl radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthyloxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxyphosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxyethylcarbamoyl, sulfamoyl, nitro, oxo and/or by cyano, with heterocyclyl-lower alkanoyl being selected especially from pyrrolylcarbonyl that is unsubstituted or substituted by lower alkyl or by phenyl, for example 2- or 3-pyrrolylcarbonyl, 4- or 5-methylpyrrolylcarbonyl or 4- or 5-phenylpyrrolyl-2-carbonyl, thienylcarbonyl, such as 2-thienylcarbonyl, furylcarbonyl, such as 2-furylcarbonyl, pyridylcarbonyl, such as 2-, 3- or 4-pyridylcarbonyl, pyrimidin-1-ylcarbonyl, indolylcarbonyl that is unsubstituted or substituted by lower alkyl, such as methyl, phenyl-lower alkyl, such as benzyl, lower alkoxy, such as methoxy, phenyl-lower alkoxy, such as benzyloxy, or by halogen, such as chlorine, such as 2-, 3- or 5-indolylcarbonyl, 1-methyl-5-methyl-, 5-methoxy-, 5-benzyloxy-, 5-chloro- or 4,5-dimethyl-indolyl-2-carbonyl, 1-benzyl-indolyl-2- or -3-carbonyl, 4,5,6,7-tetrahydroindolyl-2-carbonyl, unsubstituted or hydroxy-substituted quinolyl-lower alkanoyl, for example quinolylcarbonyl, such as 2-, 3- or 4-quinolylcarbonyl or 4-hydroxyquinolyl-2-carbonyl, unsubstituted or hydroxy-substituted isoquinolylcarbonyl, such as 1-, 3- or 4-isoquinolylcarbonyl or 1-oxo-1,2-dihydroisoquinolyl-3-carbonyl, 2-quinoxalinylcarbonyl, 2-(3,1-benzofuranyl)carbonyl, cyclohepta[b]pyrrolyl-5-carbonyl, 3-chromanylcarbonyl, 3-thiochromanylcarbonyl, pyrrolidinyl-3-carbonyl, hydroxypyrrolidinylcarbonyl, such as 3- or 4-hydroxypyrrolidinyl-2-carbonyl, oxo-pyrrolidinylcarbonyl, such as 5-oxopyrrolidinyl-2-carbonyl, piperidylcarbonyl, such as piperidinocarbonyl or 2-, 3- or 4-piperidylcarbonyl, pyrazinylcarbonyl, such as pyrazin-1-ylcarbonyl, piperazinylcarbonyl, such as piperazin-1-ylcarbonyl, morpholinyl-lower alkanoyl, for example morpholinylcarbonyl, such as morpholinocarbonyl, thiomorpholinyl-lower alkanoyl, for example thiomorpholinylcarbonyl, such as thiomorpholinocarbonyl, S,S-dioxothiomorpholinylcarbonyl, such as S,S-dioxothiomorpholinocarbonyl, indolinylcarbonyl, such as 2- or 3-indolinylcarbonyl, 1,2,3,4-tetrahydroquinolylcarbonyl, such as 1,2,3,4-tetrahydroquinolyl-2-, -3- or -4-carbonyl, 1,2,3,4-tetrahydroisoquinolylcarbonyl, such as 1,2,3,4-tetrahydroisoquinolyl-1-, -3- or -4-carbonyl or 1-oxo-1,2,3,4-tetrahydroisoquinolyl-3-carbonyl, tetrazolyl-lower alkanoyl, such as 3-(tetrazol-1-yl)propionyl, and pyridyl-lower alkanoyl, for example pyridylacetyl, such as 2-, 3- or 4-pyridylacetyl, heterocyclyl-lower alkanoyl being selected more especially from morpholino-lower alkanoyl, such as morpholinocarbonyl, thiomorpholino-lower alkanoyl, such as thiomorpholinocarbonyl, pyridyl-lower alkanoyl, such as 2-, 3-, or 4-pyridylacetyl, quinolinyl-lower alkanoyl, such as quinoline-2-carbonyl, and tetrazolyl-lower alkanoyl, such as 3-tetrazol-1-ylpropionyl; amino-lower alkanoyl substituted at the amino nitrogen atom by heterocyclyl-lower alkanoyl wherein heterocyclyl-lower alkanoyl is as defined above for heterocyclyl-lower alkanoyl R₁, R₂, R₈ or R₉, preferably as defined for heterocyclyl-lower alkanoyl R₁ and R₉, especially amino-lower alkanoyl substituted at the amino nitrogen atom by N-morpholino- or by N-thiomorpholino-carbonyl, more especially N-morpholino- or N-thiomorpholino-carbonylamino-lower alkanoyl, such as N-morpholino- or N-thiomorpholino-carbonylamino-acetyl; halo-lower alkanoyl containing up to three halogen atoms, especially α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl, especially trifluoroacetyl; (N-heterocyclyl-lower alkylcarbamoyl)lower alkanoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and from thiomorpholinyl, especially 2-(N-morpholino-lower alkylcarbamoyl)-lower alkanoyl, such as 2(R,S)-(N-(2-morpholinoethyl)-carbamoyl)-3-methylbutyryl, or 2-(N-(pyridyl-lower alkyl)-carbamoyl)-lower alkanoyl, such as 2(R,S)-(N-(2-pyridylmethyl)-carbamoyl)-3-methylbutyryl; lower alkoxycarbonyl, especially methoxy-, ethoxy-, isopropoxy-, isobutoxy- or tert-lower alkoxy-carbonyl, for example methoxycarbonyl, tert-butoxycarbonyl or isobutoxycarbonyl; aryl-lower alkoxycarbonyl wherein aryl preferably has from 6 to 14 carbon atoms and is, for example, phenyl, biphenylyl, 1- or 2-naphthyl, fluorenyl, or phenyl that is mono- or poly-substituted by lower alkyl, for example methyl or tert-butyl, hydroxy, lower alkoxy, for example methoxy, ethoxy or tert-butoxy, halogen, for example chlorine or bromine, and/or by nitro, for example phenyl-lower alkoxycarbonyl, such as benzyloxycarbonyl, 4-methoxybenzyloxycarbonyl, 4-nitrobenzyloxycarbonyl or fluorenyl-lower alkoxycarbonyl, such as 9-fluorenylmethoxycarbonyl, especially phenyl-lower alkoxycarbonyl, such as benzyloxycarbonyl; heterocyclyl-lower alkoxycarbonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, and from morpholinyl and from thiomorpholinyl and is unsubstituted or substituted, especially by lower alkyl, such as by methyl, for example 1-methylpyrrolidin-2-ylmethoxycarbonyl, 2-furylmethoxycarbonyl, 2-tetrahydrofuranyl-lower alkoxycarbonyl, such as 2-tetrahydrofurylmethoxycarbonyl, 1-methyl-2-piperidylmethoxycarbonyl or 2-morpholinoethoxycarbonyl, or 2-, 3- or 4-pyridylmethoxycarbonyl, especially tetrahydrofuranyl-lower alkoxycarbonyl, such as 2(R,S)-tetrahydrofuranylmethoxycarbonyl; lower alkylsulfonyl, for example methyl- or ethyl-sulfonyl, such as methylsulfonyl; heterocyclylsulfonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholinyl and from thiomorpholinyl and may be unsubstituted or substituted, especially by lower alkyl, such as methyl, such as morpholinosulfonyl, thiomorpholinosulfonyl, piperidinosulfonyl, 4-methylpiperazinylsulfonyl or piperazinosulfonyl; N-heterocyclyl-lower alkyl-N-lower alkylcarbamoyl that is as defined above under unsubstituted or substituted N-alkyl- or N,N-dialkyl-carbamoyl R₁, R₂, R₈ or R₉, wherein heterocyclyl is preferably selected from thienyl, furyl, pyranyl, pyrrolyl, imidazolyl, pyrazolyl, oxazolyl, isoxazolyl, thiazolyl, furazanyl, pyridyl, pyrazinyl, pyrimidinyl, pyridazinyl, azepinyl, indolyl, benzimidazolyl, 1H-indazolyl, quinolyl, isoquinolyl, quinoxalinyl, quinazolinyl, cinnolyl, purinyl, pteridinyl, naphthyridinyl, 4H-quinolizinyl, 3,1-benzofuranyl, benz[e] indolyl, 4,1-benzoxazinyl, 4,1-benzothiazinyl, carbazolyl, β-carbolinyl, phenazinyl, phenanthridyl, acridyl, phenoxazinyl, phenothiazinyl, 1-azaacenaphthenyl, cyclohexa[b]pyrrolyl, cyclohepta[b]pyrrolyl, cyclohexa[d]pyrazolyl, cyclohexa[b]pyridyl, cyclohexa[b]pyrazinyl, cyclohexa[b)pyrimidinyl, cyclohexa [b]-1,4-oxazinyl, cyclohexa[b]-1,4-thiazinyl, pyrrolidinyl, pyrrolinyl, imidazolidinyl, 2-imidazolinyl, 2,3-dihydropyridyl, piperidyl, piperazinyl, 2,3,5,6-tetrahydropyrazinyl, morpholinyl, thiomorpholinyl, S,S-dioxothiomorpholinyl, indolinyl, isoindolinyl, 4,5,6,7-tetrahydroindolyl, 1,2,3,4-tetrahydroquinolyl, 1,2,3,4-tetrahydroisoquinolyl, chromanyl, thiochromanyl, 1,2,3,4-tetrahydro-3,1-benzodiazinyl, 3,4-dihydro-3H-4,1-benzoxazinyl, 3,4-dihydro-3H-4,1-benzothiazinyl, 2,3,4,5-tetrahydro-1H-5,1-benzazepinyl and 5,6-dihydrophenanthridinyl, the mentioned radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthylmethoxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxyphosphoryl, for example, dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxyethylcarbamoyl, sulfamoyl, nitro, oxo and/or by cyano, and is especially pyridyl, such as 2-, 3- or 4-pyridyl, more especially N-pyridyl-lower alkyl-N-lower alkylcarbamoyl, such as N-(2-,3- or .sup.4-pyridylmethyl)-N-methylcarbamoyl; or an acyl radical of an amino acid the amino function of which is free or acylated by one of the other radicals mentioned hitherto for R₁ and R₉, the amino acid radicals, each independently of the others, preferably being as defined for unsubstituted or substituted amino acids as acyl R₁, R₂, R₈ and R₉, especially the radical of a natural α-amino acid having the L-configuration, such as those normally occurring in proteins, or an epimer of such an amino acid, i.e. having the unnatural D-configuration, or the D,L-isomeric mixture thereof, a homologue of such an amino acid, for example wherein the amino acid side chain has been lengthened or shortened by one or two methylene groups, wherein the amino group is in the β-position and/or wherein a methyl group has been replaced by hydrogen, a substituted aromatic amino acid wherein the aromatic radical has from 6 to 14 carbon atoms, for example a substituted phenylalanine or phenylglycine wherein phenyl may be mono- or poly-substituted by lower alkyl, for example methyl, hydroxy, lower alkoxy, for example methoxy, lower alkanoyloxy, for example acetoxy, amino, lower alkylamino, for example methylamino, di-lower alkylamino, for example dimethylamino, lower alkanoylamino, for example acetylamino or pivaloylamino, lower alkoxycarbonylamino, for example tert-butoxycarbonylamino, arylmethoxycarbonylamino wherein aryl preferably has from 6 to 14 carbon atoms, for example benzyloxycarbonylamino or 9-fluorenylmethoxycarbonylamino, halogen, for example fluorine, chlorine, bromine or iodine, carboxy and/or by nitro, a benzo-fused phenylalanine or phenylglycine, such as α-naphthylalanine, or a hydrogenated phenylalanine or phenylglycine, such as a cyclohexylalanine or cyclohexylglycine, especially the radical, bonded via a carboxy group, of an amino acid selected from glycine (H-Gly-OH), alanine (H-Ala-OH), valine (H-Val-OH), norvaline (α-aminovaleric acid), leucine (H-Leu-OH), isoleucine (H-Ile-OH), norleucine (α-aminohexanoic acid, H-Nle-OH), serine (H-Ser-OH), homoserine (α-amino-y-hydroxybutyric acid), threonine (H-Thr-OH), methionine (H-Met-OH), cysteine (H-Cys-OH), proline (H-Pro-OH), trans-3- and trans-4-hydroxyproline, phenylalanine (H-Phe-OH), tyrosine (H-Tyr-OH), 4-aminophenylalanine, 4-chlorophenylalanine, 4-carboxyphenylalanine, β-phenylserine (β-hydroxyphenylalanine), phenylglycine, α-naphthylalanine. (H-Nal-OH), cyclohexylalanine (H-Cha-OH), cyclohexylglycine, tryptophan (H-Trp-OH), indoline-2-carboxylic acid, 1,2,3,4-tetrahydroisoquinoline-3-carboxylic acid, aspartic acid (H-Asp-OH), asparagine (H-Asn-OH), aminomalonic acid, aminomalonic acid monoamide, glutamic acid (H-Glu-OH), glutamine (H-Gln-OH), histidine (H-His-OH), arginine (H-Arg-OH), lysine (H-Lys-OH), .delta.-hydroxylysine, ornithine (α, .delta.-diaminovaleric acid), 3-aminopropanoic acid, α,γ-diaminobutyric acid and α,β-diaminopropionic acid, more especially the radical of an amino acid selected from valine, alanine, leucine, isoleucine, glycine, glutamic acid and asparagine, wherein each of the mentioned amino acids (with the exception of glycine) may be in the D-, L- or (D,L)-form, preferably (with the exception of Val, which may also be in the (D)- or (D,L)-form) in the L-form, the α-amino group is unsubstituted or N-acylated by one of the radicals mentioned above for R₁ and R₉, especially by lower alkanoyl, phenyl-lower alkanoyl, such as phenylacetyl, phenyl-lower alkanoyl wherein the lower alkanoyl radical is substituted by carbamoyl, such as 2(R,S)-carbamoyl-3-phenylpropionyl, morpholino-lower alkanoyl, such as morpholinocarbonyl, thiomorpholino-lower alkanoyl, such as thiomorpholinocarbonyl, pyridyl-lower alkanoyl, such as 2-, 3- or 4-pyridylacetyl, quinolinyl-lower alkanoyl, such as quinoline-2-carbonyl, tetrazolyl-lower alkanoyl, such as 3-tetrazol-1-ylpropionyl, amino-lower alkanoyl substituted at the amino nitrogen atom by N-morpholino- or N-thiomorpholino-carbonyl, for example N-morpholino- or N-thiomorpholino-carbonylamino-lower alkanoyl, such as N-morpholino- or N-thiomorpholino-carbonylaminoacetyl, halo-lower alkanoyl containing up to three halogen atoms, for example α-haloacetyl, such as α-fluoro-, α-chloro-, α-bromo-α-iodo-, (α,α,α-trifluoro- or α,α,α-trichloro-acetyl, or halopropionyl, such as β-chloro- or β-bromo-propionyl, especially trifluoroacetyl, 2-(N-morpholino-lower alkylcarbamoyl)-lower alkanoyl, such as 2(R,S)-(N-(2-morpholinoethyl)-carbamoyl-3-methylbutyryl, 2-(N-(pyridyl-lower alkyl)-carbamoyl)-lower alkanoyl, such as 2(R,S)-(N-(2-pyridylmethyl)-carbamoyl)-lower alkanoyl, lower alkoxycarbonyl, phenyl-lower alkoxycarbonyl, tetrahydrofuranyl-lower alkoxycarbonyl, such as 2(R,S)-tetrahydrofuranylmethoxycarbonyl, lower alkylsulfonyl, morpholinosulfonyl, thiomorpholinosulfonyl, piperidinosulfonyl, 4-methylpiperazinylsulfonyl or piperazinosulfonyl or N-pyridyl-lower alkyl-N-lower alkylcarbamoyl, such as N-(2-, 3- or 4-pyridylmethyl)-N-methylcarbamoyl, special preference being given to N-morpholinocarbonyl-glycine, N-(N-(2-, 3- or 4-pyridyl)methyl-N-methylaminocarbonyl)-glycine, valine, N-(trifluoroacetyl)-valine, N-phenylacetyl valine, N-(2- or 3-pyridyl)-acetyl-valine, N-acetyl-valine, N-(2-carbamoyl-3-phenylpropionyl)-valine, N-(2(R,S)-carbamoyl-3-phenylpropionyl)-valine, N-(2- or 3-pyridylacetyl)-valine, N-2-tetrahydrofurylmethoxycarbonyl-valine, N-(3-(tetrazol-1-yl)propionyl)-valine, N-(quinoline-2-carbonyl)-valine, N-methoxycarbonyl-valine, N-isobutoxycarbonyl-valine, N-tert-butoxycarbonyl-valine, N-benzyloxycarbonyl-valine, N-(morpholinocarbonyl)-valine, N-(thiomorpholinocarbonyl)-valine, N-(S, S-dioxothiomorpholinocarbonyl)-valine, N-(N-2-pyridylmethyl-N-methylaminocarbonyl)-valine, N-morpholinocarbonylaminoacetyl-valine, N-methylsulfonyl-valine, N-acetyl-isoleucine, N-propionyl-isoleucine, N-(benzyloxycarbonyl)-isoleucine, N-benzyloxycarbonylglutamic acid, asparagine, N-benzyloxycarbonyl-asparagine or quinoline-2-carbonylasparagine, the mentioned amino acid radicals preferably being in the (L)- or (D,L)-form, and in the case of valine also in the (D)-form, with the proviso that not more than one of the two radicals R₁ and R₉ may be hydrogen,
R₂, R₄, R₆ and R₈ are hydrogen,
R₃ is lower alkyl, such as isobutyl or n-butyl; cycloalkyl-lower alkyl as defined above for cycloalkyl-lower alkyl R₃, R₄ and R₇, wherein, preferably, cycloalkyl has from 3 to 7 carbon atoms and is unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded, preferably terminally, to lower alkyl, especially methyl, ethyl, n-propyl, isopropyl, n-butyl, isobutyl or tert-butyl, such as cyclobutyl-, cyclopentyl-, cyclohexyl- or cycloheptyl-lower alkyl, such as -methyl or -ethyl, especially cyclohexyl-lower alkyl, most especially cyclohexylmethyl; or is aryl-lower alkyl that is preferably as defined under aryl-lower alkyl R₃, R₄ and R₇, wherein aryl contains especially from 6 to 14 carbon atoms, such as in phenyl, indenyl, indanyl, naphthyl, anthryl, phenanthryl, acenaphthyl or fluorenyl, and is unsubstituted or substituted, and may be, especially, mono- to tri-substituted by lower alkyl, for example methyl, ethyl or isopropyl, halo-lower alkyl, such as trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine or chlorine, carboxy, lower alkoxycarbonyl, benzyl-, naphthyl- or fluorenyl-lower alkoxycarbonyl, lower alkanoyl, sulfo, lower alkylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylsulfamoyl, nitro and/or by cyano, wherein phenyl may be present up to three times, such as in diphenyl-, dibenzyl- or triphenyl-lower alkyl, for example diphenyl-, dibenzyl- or triphenyl-2-ethyl, especially phenyl-lower alkyl that is unsubstituted or substituted by the mentioned substituents, especially benzyl, 2-phenylethyl, 3-phenylpropyl, 4-fluoro-, 4-cyano-, 4-methoxy- or 4-hydroxy-benzyl,
R₅ is hydroxy, and
R₇ is unsubstituted or substituted lower alkyl, preferably unsubstituted or substituted as described above for unsubstituted or substituted alkyl R₃, R₄ or R₇, especially lower alkyl, more especially isobutyl or n-butyl, cycloalkyl-lower alkyl, as last described for cycloalkyl-lower alkyl R₃, especially cyclohexyl-lower alkyl, such as cyclohexylmethyl, or aryl-lower alkyl, as last described for aryl-lower alkyl R₃, especially phenyl-lower alkyl that is unsubstituted or substituted by the mentioned substituents, such as benzyl, 2-phenylethyl, 3-phenylpropyl, 4-fluoro-, 4-cyano-, 4-methoxy- or 4-hydroxy-benzyl,
or a salt thereof where a salt-forming group is present.

Of the last-mentioned compounds of formula I, special preference is given to those wherein
R₁ and R₉ are each independently of the other hydrogen; lower alkanoyl; aryl-lower alkanoyl wherein the lower alkanoyl radical is unsubstituted or substituted by carbamoyl or by carbamoyl substituted at the nitrogen atom by one or two radicals selected from lower alkyl, carboxy-lower alkyl, lower alkoxycarbonyl-lower alkyl, di-lower alkylamino-lower alkyl, aminocarboxy-lower alkyl, hydroxy-lower alkyl or by di-lower alkoxy-lower alkyl, and wherein aryl contains from 6 to 14 carbon atoms; heterocyclyl-lower alkanoyl wherein heterocyclyl contains from 3 to 10 ring atoms and up to 4 hetero atoms selected from O, N, S, Se, and S linked to 1 or 2 oxygen atoms (S═O, O═S,.═O); halo-lower alkanoyl having from 1 to 3 halogen atoms; N-heterocyclyl-lower alkylcarbamoyl-lower alkanoyl; lower alkoxycarbonyl; aryl-lower alkoxycarbonyl wherein aryl contains from 6 to 14 carbon atoms; heterocyclyl-lower alkoxycarbonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated; lower alkylsulfonyl; N-(heterocyclyl-lower alkyl)-carbamoyl-N-lower alkylcarbamoyl; or the radical, bonded via the carboxy group, of an amino acid selected from glycine, alanine, valine, norvaline, leucine, isoleucine, norleucine, serine, homoserine, threonine, methionine, cysteine, proline, trans-3- and trans-4-hydroxyproline, phenylalanine, tyrosine, 4-aminophenylalanine, 4-chlorophenylalanine, 4-carboxyphenylalanine, p-hydroxyphenylalanine, phenylglycine, α-naphthylalanine, cyclohexylalanine, cyclohexylglycine, tryptophan, aspartic acid, asparagine, aminomalonic acid, aminomalonic acid monoamide, glutamic acid, glutamine, histidine, arginine, lysine, γ-hydroxylysine, ornithine, α,γ-diaminobutyric acid or α,β-diaminopropionic acid, the amino acid radicals being unsubstituted or substituted by one of the mentioned radicals R₁ or R₉ with the exception of the radical of one of the amino acids itself;
R₂, R₄, R₆ and R₉ are hydrogen,
R₃ is cycloalkyl-lower alkyl wherein cycloalkyl has from 3 to 7 carbon atoms, or is aryl-lower alkyl wherein aryl has from 6 to 14 carbon atoms,
R₅ is hydroxy, and
R₇ is lower alkyl, cycloalkyl-lower alkyl wherein cycloalkyl has from 3 to 7 carbon atoms, or is aryl-lower alkyl wherein aryl has from 6 to 14 carbon atoms,
and salts of such compounds having salt-forming groups, the general expressions and definitions preferably having the meanings mentioned as preferred in the previous paragraph.

Preference is likewise given to the compounds mentioned in the penultimate section above in which the substituents have all the meanings mentioned with the exception of morpholinosulfonyl, thiomorpholinosulfonyl, piperidinosulfonyl, 4-methylpiperazinylsulfonyl or piperazinosulfonyl as R₁ and/or R₉.

Preference is given also to the compounds of formula I wherein R₁ and R₉ are each independently of the other: hydrogen, lower alkoxycarbonyl, 2-halo-lower alkoxycarbonyl, aryloxycarbonyl wherein aryl has from 6 to 14 carbon atoms, aryl-lower alkoxycarbonyl wherein aryl has from 6 to 14 carbon atoms, heterocyclyl-lower alkoxycarbonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, 2-tri-lower alkylsilyl-lower alkoxycarbonyl, 2-triarylsilyl-lower alkoxycarbonyl wherein aryl is phenyl or 1- or 2-naphthyl, the radical, bonded via the carboxy group, of an amino acid selected from glycine, alanine, valine, norvaline, leucine, isoleucine, norleucine, serine, homoserine, threonine, methionine, cysteine, proline, trans-3- and trans-4-hydroxyproline, phenylalanine, tyrosine, 4-aminophenylalanine, 4-chlorophenylalanine, 4-carboxyphenylalanine, β-phenylserine, phenylglycine, α-naphthylalanine, cyclohexylalanine, cyclohexylglycine, tryptophan, indoline-2-carboxylic acid, 1,2,3,4-tetrahydroisoquinoline-3-carboxylic acid, aspartic acid, asparagine, aminomalonic acid, aminomalonic acid monoamide, glutamic acid, glutamine, histidine, arginine, lysine, γ-hydroxylysine, ornithine, α,γ-diaminobutyric acid and α,β-diaminopropionic acid, it being possible for each of those amino acids to be in the D-, L- or (D,L)-form, preferably in the L-form, and wherein the 60-amino group may be unsubstituted or mono- or di-N-alkylated by lower alkyl, by amino-lower alkyl, by phenyl- or naphthyl-amino-lower alkyl, or by piperazinylcarbonyl-lower alkyl substituted at the nitrogen atom by lower alkyl, or may be N-acylated by lower alkanoyl; by aryl-lower alkanoyl wherein aryl is selected from phenyl, indenyl, indanyl, naphthyl, anthryl, phenanthryl, acenaphthyl and fluorenyl and may be unsubstituted or mono- to tri-substituted by lower alkyl, halo-lower alkyl, phenyl, 1- or 2-naphthyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, halogen, carboxy, lower alkoxycarbonyl, phenyl-, naphthyl- or fluorenyl-lower alkoxycarbonyl, lower alkanoyl, sulfo, lower alkylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, and wherein lower alkanoyl may be unsubstituted or substituted by lower alkyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-, cyclopenta-, cyclohexa- or cyclohepta-fused derivative of those radicals, which may also be fully or partially saturated, hydroxy, lower alkoxy, lower alkanoyloxy, acetoacetoxy, amino- or benzyloxycarbonylamino-lower alkanoyloxy, aryl-lower alkanoyloxy wherein aryl has from 6 to 10 carbon atoms, lower alkoxycarbonyloxy, mono- or di-lower alkylaminocarbonyloxy, aryloxycarbonyloxy wherein aryl has from 6 to 10 carbon atoms, aryl-lower alkoxycarbonyloxy wherein aryl has from 6 to 14 carbon atoms, sulfonyloxy, lower alkylsulfonyloxy, phenylsulfonyloxy, 2- or 4-toluenesulfonyloxy or 1- or 2-naphthylsulfonyloxy, carboxy, esterified carboxy selected from lower alkoxycarbonyl, aryloxycarbonyl wherein aryl has from 6 to 10 carbon atoms, aryl-lower alkoxycarbonyl wherein aryl has from 6 to 12 carbon atoms, lower alkanoyl, lower alkylsulfonyl, hydroxy-lower alkoxyphosphoryl and di-lower alkoxyphosphoryl, carbamoyl, carbamoyl substituted by one or two radicals selected from lower alkyl, carboxy-lower alkyl, lower alkoxycarbonyl-lower alkyl, di-lower alkylamino-lower alkyl, aminocarboxy-lower alkyl, hydroxy-lower alkyl and di-lower alkoxy-lower alkyl or carbamoyl substituted by one radical selected from ethylene, trimethylene, tetramethylene and pentamethylene, in which a carbon atom may have been replaced by nitrogen, oxygen, sulfur or by sulfur mono- or di-substituted by oxygen and which may be unsaturated, and also sulfamoyl, phosphono, benzofuranyl, oxo and/or by cyano and is branched or unbranched; by heterocyclyl-lower alkanoyl selected from thienyl-, furyl-, pyranyl-, pyrrolyl-, imidazolyl-, pyrazolyl-, oxazolyl-, isoxazolyl-, thiazolyl-, furazanyl, pyridyl-, pyrazinyl-, pyrimidinyl-, pyridazinyl-, azepinyl-, indolyl-, benzimidazolyl-, 1H-indazolyl-, quinolyl-, isoquinolyl-, quinoxalinyl-, quinazolinylcinnolyl-, purinyl-, pteridinyl-, naphthyridinyl-, 4H-quinolizinyl-, 3,1-benzofuranyl-, benz[e]indolyl-, 4,1-benzoxazinyl-, 4,1-benzothiazinyl-, carbazolyl-, D-carbolinyl-, phenazinyl-, phenanthridyl-, acridyl-, phenoxazinyl-, phenothiazinyl-, 1-azaacenaphthenyl-, cyclohexa[b]pyrrolyl-, cyclohepta[b]pyrrolyl-, cyclohexa[d] pyrazolyl-, cyclohexa[b]pyridyl-, cyclohexa[b]pyrazinyl-, cyclohexa[b]pyrimidinyl-, cyclohexa[b]-1,4-oxazinyl-, cyclohexa[b]-1,4-thiazinyl-, pyrolidinyl-, pyrrolinyl-, imidazolidinyl-, 2-imidazolinyl-, 2,3-dihydropyridyl-, piperidyl-, piperazinyl-, 2,3,5,6-tetrahydropyrazinyl-, morpholinyl-, thiomorpholinyl-, S,S-dioxothiomorpholinyl-, indolinyl-, isoindolinyl-, 4,5,6,7-tetrahydroindolyl-, 1,2,3,4-tetrahydroquinolyl-, 1,2,3,4-tetrahydroisoquinolyl-, chroman-, thiochroman-, 1,2,3,4-tetrahydro-3,1-benzodiazinyl-, 3,4-dihydro-3H-4,1-benzoxazinyl-, 3,4-dihydro-3H-4,1-benzothiazinyl-, 2,3,4,5-tetrahydro-1H-5,1-benzazepinyl- and 5,6-dihydrophenanthridinyl-lower alkanoyl, the mentioned heterocyclyl radicals being unsubstituted or substituted by lower alkyl, phenyl, 1- or 2-naphthyl, phenyl-lower alkyl, hydroxy-lower alkyl, lower alkoxy-lower alkyl, phenoxy- or naphthyloxy-lower alkyl, phenyl-lower alkoxy- or naphthyl-lower alkoxy-lower alkyl, lower alkanoyloxy-lower alkyl, phenyl- or naphthyl-lower alkanoyloxy-lower alkyl, lower alkoxycarbonyloxy-lower alkyl, phenyl-, naphthyl- or fluorenyl-lower alkoxycarbonyloxy-lower alkyl, amino-lower alkyl, carboxy-lower alkyl, hydroxy, lower alkoxy, phenyl- or naphthyl-lower alkoxy, amino, lower alkylamino, di-lower alkylamino, carboxy, lower alkoxycarbonyl, phenyl- or naphthyl-lower alkoxycarbonyl, halogen, lower alkanoyl, lower alkylsulfonyl, phosphono, hydroxy-lower alkoxyphosphoryl, dialkoxyphosphoryl, carbamoyl, mono- or di-lower alkylcarbamoyl, hydroxy- or carboxy-lower alkylcarbamoyl, sulfamoyl, nitro, oxo and/or by cyano; by heterocyclyl-lower alkenoyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated; by lower alkoxycarbonyl; by aryl-lower alkoxycarbonyl wherein aryl has from 6 to 14 carbon atoms; by heterocyclyl-lower alkoxycarbonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated and unsubstituted or substituted by lower alkyl; by carboxy-lower alkanoyl; by lower alkoxycarbonyl-lower alkanoyl; by hydroxy-lower alkoxy-lower alkanoyl; by amino-lower alkanoyl; or by benzyloxycarbonylamino-lower alkanoyl wherein the amino group is not bonded in the α- or β-position; by carbamoyl; by phenyl-lower alkylaminocarbonyl; by N-di-lower alkylamino-lower alkyl-N-lower alkylaminocarbonyl; by N-dihydroxy-lower alkyl-N-lower alkylaminocarbonyl; by 2- or 3-pyridyl-lower alkylaminocarbonyl; by N-2-pyridyl-lower alkyl-N-lower alkylaminocarbonyl; by sulfonyl; by lower alkylsulfonyl; by arylsulfonyl wherein aryl has from 6 to 10 carbon atoms and is unsubstituted or substituted by lower alkyl or by lower alkoxy; by heterocyclylsulfonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated; by sulfamoyl; or by sulfamoyl substituted by heterocyclyl-lower alkyl wherein heterocyclyl is as last defined, and/or by lower alkyl;
a carboxy group of the side chain is present in free form or in esterified form as a lower alkyl ester group, as an aryl ester group or as an aryl-lower alkyl ester group, wherein aryl is phenyl, 4-nitrophenyl, naphthyl or biphenylyl, or in amidated form as a carbamoyl, lower alkylcarbamoyl, di-lower alkylaminocarbamoyl, mono- or di-(hydroxy-lower alkyl)-carbamoyl or mono- or di-(carboxy-lower alkyl)-carbamoyl group,
an amino group of the side chain is present in free form or in alkylated form as mono- or di-lower alkylamino or in acylated form as lower alkanoylamino, as amino-lower alkanoylamino, as aryl-lower alkanoylamino wherein aryl has from 6 to 14 carbon atoms and is unsubstituted or substituted by lower alkyl, hydroxy, lower alkoxy, carboxy, carbamoyl or by sulfamoyl, as a lower alkoxycarbonylamino group, an arylmethoxycarbonylamino group wherein aryl has from 6 to 14 carbon atoms, as piperidyl-1-carbonyl, morpholinocarbonyl, thiomorpholinocarbonyl or as S,S-dioxothiomorpholinocarbonyl and/or
a hydroxy group of the side chain is present in free form or in etherified or esterified form as a lower alkoxy, aryl-lower alkoxy, lower alkanoyloxy or lower alkoxycarbonyloxy group, lower alkylsulfonyl, 2- or 3-pyrrolyl-, 2-thienyl-, 2-furyl-, 1-pyrazolyl-, 2-, 3- or 4-pyridyl-, 2-, 3- or 5-indolyl-, (1-methyl-, 2-methyl-, 5-methoxy-, 5-benzyloxy-, 5-chloro- or 4,5-dimethylindol-2-yl)-, (1-benzylindol-2-yl or -3-yl)-, 4,5,6,7-tetrahydroindol-2-yl-, (2-, 3- or 4-quinolyl or 4-hydroxyquinol-2-yl)-, (1-, 3- or 4-isoquinolyl or 1-oxo-1,2-dihydroisoquinol-3-yl)-, 3-pyrrolidinyl-, (3- or 4-hydroxypyrrolidin-2-yl)-, 5-oxopyrrolidin-2-yl-, (2- or 3-morpholinyl)-, (2- or 3-thiomorpholinyl)-, (S,S-dioxothiomorpholin-2- or -3-yl)-, (2- or 3-indolinyl)-, 1,2,3,4-tetrahydroquinol-2-, -3- or -4-yl)- or (1,2,3,4-tetrahydroisoquinol-1-, -2- or -3-yl)-methylsulfonyl, phenyl- or 1- or 2-naphthyl-sulfonyl that is unsubstituted or mono- or di-substituted by lower alkyl, lower alkoxysulfonyl, or benzyloxysulfonyl or 1- or 2-naphthyloxysulfonyl,
with the result that not more than one of the radicals R₁ and R₉ may be hydrogen, and R₂ and R₈ are each independently of the other hydrogen or the same radicals as R₁ and R₉, or the pairs of substituents R₁ and R₉, and R₂ and R₈, each independently of the other, together with the bonding nitrogen atom and a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, form a heterocyclic ring,
R₃ is cycloalkyl-lower alkyl wherein cycloalkyl has from 3 to 7 carbon atoms and is unsubstituted or mono- to tri-substituted by lower alkyl, halo-lower alkyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, carboxy, lower alkoxycarbonyl, phenyl-, naphthyl- or fluorenyl-lower alkoxycarbonyl, lower alkanoyl, lower alkylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded to lower alkyl, or aryl-lower alkyl wherein aryl is selected from phenyl, indenyl, indanyl, naphthyl, anthryl, phenanthryl, acenaphthyl and fluorenyl, which may be unsubstituted or mono- to tri-substituted by lower alkyl, halo-lower alkyl, phenyl, 1- or 2-naphthyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, halogen, carboxy, lower alkoxycarbonyl, phenyl-, naphthyl- or fluorenyl-lower alkoxycarbonyl, lower alkanoyl, sulfo, lower alkylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylsulfamoyl, nitro and/or by cyano, wherein phenyl may be present up to three times, and wherein lower alkyl is unsubstituted or substituted by lower alkyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, hydroxy, lower alkoxy, lower alkanoyloxy, acetoacetoxy, amino- or benzyloxycarbonylamino-lower alkanoyloxy, aryl-lower alkanoyloxy wherein aryl has from 6 to 10 carbon atoms, lower alkoxycarbonyloxy, mono- or di-lower alkylaminocarbonyloxy, aryloxycarbonyloxy wherein aryl has from 6 to 10 carbon atoms, aryl-lower alkoxycarbonyloxy wherein aryl has from 6 to 12 carbon atoms, sulfonyloxy, lower alkylsulfonyloxy, phenylsulfonyloxy, 2- or 4-toluenesulfonyloxy or 1- or 2-naphthylsulfonyloxy, amino, mono- or di-lower alkylamino, N-lower alkoxy-N-lower alkylamino, mono- or di-(phenyl- or naphthyl-lower alkyl)-amino, lower alkanoylamino, carboxy, esterified carboxy selected from lower alkoxycarbonyl, aryloxycarbonyl wherein aryl has from 6 to 10 carbon atoms, aryl-lower alkoxycarbonyl wherein aryl has from 6 to 12 carbon atoms, lower alkanoyl, lower alkylsulfonyl, hydroxy-lower alkoxyphosphoryl and di-lower alkoxyphosphoryl, carbamoyl, carbamoyl substituted by one or two radicals selected from lower alkyl, from carboxy-lower alkyl or lower alkoxycarbonyl-lower alkyl, from di-lower alkylamino-lower alkyl, from aminocarboxy-lower alkyl, from hydroxy-lower alkyl and from di-lower alkoxy-lower alkyl, or carbamoyl substituted by one radical selected from ethylene, trimethylene, tetramethylene and pentamethylene in which a carbon atom may have been replaced by nitrogen, oxygen, sulfur or by sulfur mono- or di-substituted by oxygen and which may be unsaturated, and also sulfamoyl, phosphono, benzofuranyl, oxo and/or by cyano and is unbranched or branched,
R₄ is hydrogen,
R₅ is hydroxy,
R₆ is hydrogen,
   or R₅ and R₆ together are oxo; and
R₇ is lower alkyl, cycloalkyl-lower alkyl wherein cycloalkyl has from 3 to 7 carbon atoms and is unsubstituted or mono- to tri-substituted by lower alkyl, halo-lower alkyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, carboxy, lower alkoxycarbonyl, phenyl-, naphthyl- or fluorenyl-lower alkoxycarbonyl, lower alkanoyl, lower alkylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded to lower alkyl, bicycloalkyl-lower alkyl wherein bicycloalkyl contains from 5 to 10 carbon atoms, tricycloalkyl-lower alkyl wherein tricycloalkyl contains from 8 to 10 carbon atoms, aryl-lower alkyl wherein aryl is selected from phenyl, indenyl, indanyl, naphthyl, anthryl, phenanthryl, acenaphthyl and fluorenyl, which may be unsubstituted or mono- to tri-substituted by lower alkyl, halo-lower alkyl, phenyl, 1- or 2-naphthyl, hydroxy, lower alkoxy, carbamoyl-lower alkoxy, N-lower alkylcarbamoyl-lower alkoxy, N,N-di-lower alkylcarbamoyl-lower alkoxy, amino, mono- or di-lower alkylamino, lower alkanoylamino, halogen, carboxy, lower alkoxycarbonyl, benzyl-, naphthyl- or fluorenyl-lower alkoxycarbonyl, lower alkanoyl, sulfo, lower alkylsulfonyl, phosphono, hydroxy-lower alkoxyphosphoryl, di-lower alkoxyphosphoryl, carbamoyl, mono- or di-lower alkylcarbamoyl, sulfamoyl, mono- or di-lower alkylsulfamoyl, nitro and/or by cyano, wherein phenyl may be present up to three times, and wherein lower alkyl is unsubstituted or substituted by lower alkyl, heterocyclyl selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, hydroxy, lower alkoxy, lower alkanoyloxy, acetoacetoxy, amino- or benzyloxycarbonylamino-lower alkanoyloxy, aryl-lower alkanoyloxy wherein aryl has from 6 to 10 carbon atoms, lower alkoxycarbonyloxy, mono- or di-lower alkylaminocarbonyloxy, aryloxycarbonyloxy wherein aryl has from 6 to 10 carbon atoms, aryl-lower alkoxycarbonyloxy wherein aryl has from 6 to 12 carbon atoms, sulfonyloxy, lower alkylsulfonyloxy, phenylsulfonyloxy, 2- or 4-toluenesulfonyloxy, 1- or 2-naphthylsulfonyloxy, amino, mono- or di-lower alkylamino, N-lower alkoxy-N-lower alkylamino, mono- or di-(phenyl- or naphthyl-lower alkyl)amino, lower alkanoylamino, carboxy, esterified carboxy selected from lower alkoxycarbonyl, aryloxycarbonyl wherein aryl has from 6 to 10 carbon atoms, aryl-lower alkoxycarbonyl wherein aryl has from 6 to 12 carbon atoms, lower alkanoyl, lower alkylsulfonyl, hydroxy-lower alkoxyphosphoryl and di-lower alkoxyphosphoryl, carbamoyl, carbamoyl substituted by one or two radicals selected from lower alkyl, from carboxy-lower alkyl, from lower alkoxycarbonyl-lower alkyl, from di-lower alkylamino-lower alkyl, from aminocarboxy-lower alkyl, from hydroxy-lower alkyl and from di-lower alkoxy-lower alkyl, or carbamoyl substituted by one radical selected from ethylene, trimethylene, tetramethylene and pentamethylene in which a carbon atom may have been replaced by nitrogen, oxygen, sulfur or by sulfur mono- or di-substituted by oxygen and which may be unsaturated, and also sulfamoyl, phosphono, benzofuranyl, oxo (which is not bonded to the carbon atom that is linked to the nitrogen atom bonding the radical R₇) and/or by cyano and is unbranched or branched, heterocyclyl-lower alkyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, for example 4-pyrrolidinylmethyl, 1-imidazolylmethyl, 2-pyridylmethyl, 3-pyridylmethyl, quinolin-2-ylmethyl or indol-2-ylmethyl, hydroxy-lower alkyl, lower alkoxy-lower alkyl, phenoxy-lower alkyl or nitrophenoxy-lower alkyl, naphthyloxy-lower alkyl, lower alkanoyloxy-lower alkyl, acetoacetoxy-lower alkyl, arylmercapto-lower alkyl wherein aryl has from 6 to 10 carbon atoms, amino-lower alkyl, mono- or di-lower alkylamino-lower alkyl, phenyl- or naphthyl-amino-lower alkyl, lower alkanoylamino-lower alkyl, piperazinylcarbonyl-lower alkyl substituted at the nitrogen atom by lower alkyl, lower alkoxycarbonylamino-lower alkyl, phenyl-lower alkoxycarbonylamino-lower alkyl, aminocarbonylamino-lower alkyl, N-phenyl-lower alkyl-N-lower alkylamino carbonylamino-lower alkyl, halo-lower alkyl, carboxy-lower alkyl, lower alkoxycarbonyl-lower alkyl, 2-halo-lower alkoxycarbonyl-lower alkyl, phenyl- or naphthyl-lower alkoxycarbonyl-lower alkyl, heterocyclyl-lower alkoxycarbonyl-lower alkyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, it also being possible for the mentioned radicals to be fully or partially saturated, lower alkylsulfonyl-lower alkyl, arylsulfonyl-lower alkyl wherein aryl has from 6 to 10 carbon atoms, carbamoyl-lower alkyl, lower alkylcarbamoyl-lower alkyl, di-lower alkylcarbamoyl-lower alkyl, hydroxy-lower alkylcarbamoyl- or di(hydroxy-lower alkyl)carbamoyl-lower alkyl, N-lower alkoxy-lower alkoxy-lower alkylcarbamoyl-lower alkyl, carboxy-lower alkylcarbamoyl- or di(carboxy-lower alkyl)carbamoyl-lower alkyl, carbamoyl-lower alkyl substituted at the nitrogen atom by one radical selected from ethylene, trimethylene, tetramethylene and pentamethylene wherein a carbon atom may have been replaced by nitrogen, oxygen, sulfur or by sulfur mono- or di-substituted by oxygen, it also being possible for the radical so formed to be fully or partially unsaturated, N-lower alkyl-N-heterocyclyl-lower alkylcarbamoyl-lower alkyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, sulfamoyl-lower alkyl, N-(phenyl- or naphthyl-lower alkyl)sulfamoyl-lower alkyl, sulfamoyl-lower alkyl substituted at the nitrogen atom by one radical selected from ethylene, trimethylene, tetramethylene and pentamethylene wherein a carbon atom may have been replaced by nitrogen, lower alkyl-substituted nitrogen, oxygen, sulfur or by sulfur mono- or di-substituted by oxygen, it also being possible for the radical so formed to be fully or partially unsaturated, oxo-lower alkyl (wherein oxo is not bonded to the carbon atom that is linked to the nitrogen atom that carries R₇), cyano-lower alkyl, hydroxy-carboxy-lower alkyl, α-naphthyloxy-carboxy-lower alkyl, hydroxy-lower alkoxycarbonyl-lower alkyl, α-naphthyloxy-lower alkoxycarbonyl-lower alkyl, lower alkylcarbonyl-halo-lower alkyl, α-naphthyloxyethoxycarbonyl-lower alkyl, α-naphthyloxy-benzyloxycarbonyl-lower alkyl, esterified hydroxy-lower alkoxycarbonyl-lower alkyl wherein the hydroxy group is esterified by lower alkanoyl, cycloalkyl-lower alkanoyl wherein cycloalkyl has from 3 to 7 carbon atoms, bicycloalkyl-lower alkanoyl wherein bicycloalkyl has from 5 to 10 carbon atoms, tricycloalkyl-lower alkanoyl wherein tricycloalkyl has from 8 to 10 carbon atoms, aryl-lower alkanoyl wherein aryl has from 6 to 14 carbon atoms and may be unsubstituted or mono- to tri-substituted by lower alkyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, carboxy, lower alkoxycarbonyl, phenyl-, naphthyl- or fluorenyl-lower alkoxycarbonyl, lower alkanoyl, lower alkylsulfonyl, phosphono, hydroxy-lower alkoxyphosphoryl, di-lower alkoxyphosphoryl, carbamoyl, sulfamoyl, nitro and/or by cyano, lower alkoxycarbonyl, 2-halo-lower alkoxycarbonyl or by phenyl- or fluorenyl-lower alkoxycarbonyl, dihydroxy-carboxy-lower alkyl, dihydroxy-lower alkoxycarbonyl-lower alkyl, dihydroxy-lower alkoxycarbonyl-lower alkyl esterified by lower alkanoyl, lower alkoxycarbonyl, phenyl- or fluorenyl-lower alkoxycarbonyl, lower alkylsulfonyl or by toluenesulfonyl, α-naphthyloxy-di-lower alkylamino-lower alkyl, α-naphthyloxy-carbamoyl-lower alkyl, α-naphthyloxy-oxo-lower alkyl (wherein oxo is not bonded to the carbon atom that is linked to the nitrogen atom that carries R₇), or α-naphthyloxy-cyano-lower alkyl, and the salts of the mentioned compounds where salt-forming groups are present.

Special preference is given to the compounds of formula I wherein R₁ and R₉ are each independently of the other hydrogen; lower alkanoyl, such as formyl, acetyl, propionyl, butyryl or pivaloyl, especially acetyl; aryl-lower alkanoyl wherein aryl is phenyl, indenyl, indanyl, naphthyl, anthryl, phenanthryl, acenaphthyl or fluorenyl and may be unsubstituted or especially mono- to tri-substituted by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy, N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl, di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, for example in diphenyl-, dibenzyl- or triphenyl-lower alkanoyl, such as diphenyl-, dibenzyl- or triphenyl-acetyl, and wherein lower alkanoyl is unsubstituted or substituted by carbamoyl or by carbamoyl substituted at the nitrogen atom by one or two radicals selected from lower alkyl, carboxy-lower alkyl, lower alkoxycarbonyl-lower alkyl, di-lower alkylamino-lower alkyl, aminocarboxy-lower alkyl, hydroxy-lower alkyl and di-lower alkoxy-lower alkyl, for example by carbamoyl, carbamoyl substituted by one or two radicals selected from methyl, ethyl, propyl, isopropyl, n-butyl, isobutyl, sec-butyl, tert-butyl, n-pentyl, isopentyl, neopentyl, tert-pentyl, n-hexyl, isohexyl and n-heptyl, for example in N-methylcarbamoyl, N-n-butylcarbamoyl or N,N-dimethylcarbamoyl, by carboxymethylcarbamoyl (glycinylcarbonyl), by tert-butoxycarbonylmethylcarbamoyl, by 2-dimethylaminoethyl, by 5-amino-5-carboxypentyl, by hydroxymethyl, by hydroxyethyl or by 2-(2,2-dimethoxyethyl)carbamoyl, for example 4-chloro-, 4-methoxy- or 4-nitro-benzoyl, naphthylcarbonyl, such as α- or β-naphthylcarbonyl, indenylcarbonyl, such as 1-, 2- or 3-indenylcarbonyl, indanylcarbonyl, such as 1- or 2-indanylcarbonyl, phenanthrenylcarbonyl, such as 9-phenanthrenylcarbonyl, phenyl-lower alkanoyl, such as phenylacetyl or 3-phenylpropionyl, phenyl-lower alkanoyl wherein the lower alkanoyl radical is substituted by carbamoyl, such as 2(R,S)-carbamoyl-3-phenylpropionyl, α-naphthylacetyl, β-naphthylacetyl, lower alkylphenylacetyl, such as 4-methylphenylacetyl, lower alkoxyphenylacetyl, such as 4-methoxyphenylacetyl, 3-(p-hydroxyphenyl)-propionyl, diphenylacetyl, di-(4-methoxyphenyl)-acetyl, triphenylacetyl, 2,2-dibenzylacetyl, 3-α- or 3-β-naphthylpropionyl, 3-phenyl- or 3-α-naphthyl-2-carbamoylpropionyl, 3-phenyl- or 3-α-naphthyl-2-tert-butylcarbamoyl-propionyl, 3-phenyl- or 3-α-naphthyl-2-(2-dimethylaminoethyl)carbamoylpropionyl, 3-α-naphthyl-2-(carboxy- or tert-butoxycarbonyl)methylcarbamoyl-propionyl, 3-phenyl- or 3-α-naphthyl-2-(3-hydroxy-2-propyl)carbamoyl-propionyl, 3-phenyl- or 3-α-naphthyl-2-(2,2-dimethoxyethyl)-carbamoylpropionyl, 3-phenyl- or 3-α-naphthyl-2-(5-amino-5-carboxypentyl)-carbamoylpropionyl, especially phenyl-lower alkanoyl, such as phenylacetyl, or phenyl-lower alkanoyl wherein the lower alkanoyl radical is substituted by carbamoyl, such as 2(R,S)-carbamoyl-3-phenyl-propionyl; heterocyclyl-lower alkanoyl wherein lower alkanoyl is unsubstituted and wherein heterocyclyl is selected from thienyl, furyl, pyranyl, pyrrolyl, imidazolyl, pyrazolyl, oxazolyl, isoxazolyl, thiazolyl, furazanyl, tetrazolyl, pyridyl, pyrazinyl, pyrimidinyl, pyridazinyl, azepinyl, indolyl, benzimidazolyl, 1H-indazolyl, quinolyl, isoquinolyl, quinoxalinyl, quinazolinyl, cinnolyl, purinyl, pteridinyl, naphthyridinyl, 4H-quinolizinyl, 3,1-benzofuranyl, 4,1-benzoxazinyl, 4,1-benzothiazinyl, cyclohexa[b]pyrrolyl, cyclohepta[b]pyrrolyl, cyclohexa[d]pyrazolyl, cyclohexa[b]pyridyl, cyclohexa[b]pyrazinyl, cyclohexa[b]pyrimidinyl, cyclohexa[b]-1,4-oxazinyl, cyclohexa[b]-1,4-thiazinyl, pyrrolidinyl, pyrrolinyl, imidazolidyl, 2-imidazolinyl, 2,3-dihydropyridyl, piperidyl, piperazinyl, 2,3,5,6-tetrahydropyrazinyl, morpholinyl, thiomorpholinyl, S,S-dioxo-thiomorpholinyl, indolinyl, isoindolinyl, 4,5,6,7-tetrahydroindolyl, 1,2,3,4-tetrahydroquinolyl, 1,2,3,4-tetrahydroisoquinolyl, chromanyl, thiochromanyl, 1,2,3,4-tetrahydro-3,1-benzodiazinyl, 3,4-dihydro-3H-4,1-benzoxazinyl and 3,4-dihydro-3H-4,1-benzothiazinyl, the mentioned heterocyclyl radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthyloxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, -chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxyphosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, heterocyclyl-lower alkanoyl being selected especially from unsubstituted or lower alkyl- or phenyl-substituted pyrrolylcarbonyl, for example 2- or 3-pyrrolylcarbonyl, 4- or 5-methylpyrrolylcarbonyl or 4- or 5-phenylpyrrolyl-2-carbonyl, thienylcarbonyl, such as 2-thienylcarbonyl, furylcarbonyl, such as 2-furylcarbonyl, pyridylcarbonyl, such as 2-, 3- or 4-pyridylcarbonyl, pyrimidin-1-ylcarbonyl, indolylcarbonyl that is unsubstituted or substituted by lower alkyl, such as methyl, phenyl-lower alkyl, such as benzyl, lower alkoxy, such as methoxy, phenyl-lower alkoxy, such as benzyloxy, or by halogen, such as chlorine, such as 2-, 3- or 5-indolylcarbonyl, 1-methyl-5-methyl-, 5-methoxy-, 5-benzyloxy-, 5-chloro- or 4,5-dimethyl-indolyl-2-carbonyl, 1-benzylindolyl-2- or -3-carbonyl, 4,5,6,7-tetrahydroindolyl-2-carbonyl, unsubstituted or hydroxy-substituted quinolyl-lower alkanoyl, for example quinolylcarbonyl, such as 2-, 3- or 4-quinolylcarbonyl or 4-hydroxyquinolyl-2-carbonyl, unsubstituted or hydroxy-substituted isoquinolylcarbonyl, such as 1-, 3- or 4-isoquinolylcarbonyl or 1-oxo-1,2-dihydroisoquinolyl-3-carbonyl, 2-quinoxalinylcarbonyl, 2-(3,1-benzofuranyl)-carbonyl, cyclohepta[b]pyrrolyl-5-carbonyl, 3-chromanylcarbonyl, 3-thiochromanylcarbonyl, pyrrolidinyl-3-carbonyl, hydroxypyrrolidinylcarbonyl, such as 3- or 4-hydroxypyrrolidinyl-2-carbonyl, oxopyrrolidinylcarbonyl, such as 5-oxopyrrolidinyl-2-carbonyl, piperidylcarbonyl, such as piperidinocarbonyl or 2-, 3- or 4-piperidylcarbonyl, pyrazinylcarbonyl, such as pyrazin-1-ylcarbonyl, piperazinylcarbonyl, such as piperazin-1-ylcarbonyl, morpholinyl-lower alkanoyl, for example morpholinylcarbonyl, such as morpholinocarbonyl, thiomorpholinyl-lower alkanoyl, for example thiomorpholinylcarbonyl, such as thiomorpholinocarbonyl, S,S-dioxothiomorpholinylcarbonyl, such as S,S-dioxothiomorpholinocarbonyl, indolinylcarbonyl, such as 2- or 3-indolinylcarbonyl, 1,2,3,4-tetrahydroquinolylcarbonyl, such as 1,2,3,4-tetrahydroquinolyl-2-, -3- or -4-carbonyl, 1,2,3,4-tetrahydroisoquinolylcarbonyl, such as 1,2,3,4-tetrahydroisoquinolyl-1-, -3- or -4-carbonyl or 1-oxo-1,2,3,4-tetrahydroisoquinolyl-3-carbonyl, tetrazolyl-lower alkanoyl, such as 3-(tetrazol-1-yl)propionyl, and pyridyl-lower alkanoyl, for example pyridylacetyl, such as 2-, 3- or 4-pyridylacetyl, heterocyclyl-lower alkanoyl being selected more especially from morpholino-lower alkanoyl, such as morpholinocarbonyl, thiomorpholino-lower alkanoyl, such as thiomorpholinocarbonyl, pyridyl-lower alkanoyl, such as 2-, 3- or 4-pyridylacetyl, quinolinyl-lower alkanoyl, such as quinoline-2-carbonyl, and tetrazolyl-lower alkanoyl, such as 3-tetrazol-1-ylpropionyl; amino-lower alkanoyl substituted at the amino nitrogen atom by heterocyclyl-lower alkanoyl wherein heterocyclyl-lower alkanoyl is as defined for heterocyclyl-lower alkanoyl R₁ and R₉, especially amino-lower alkanoyl substituted at the amino nitrogen atom by N-morpholino- or N-thiomorpholino-carbonyl, especially N-morpholino- or N-thiomorpholino-carbonylamino-lower alkanoyl, such as N-morpholino- or N-thiomorpholino-carbonylamino-acetyl; halo-lower alkanoyl containing up to three halogen atoms, especially α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl, for example trifluoroacetyl; (N-heterocyclyl-lower alkylcarbamoyl)-lower alkanoyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, from morpholine and from thiomorpholine, especially 2-(N-morpholino-lower alkylcarbamoyl)-lower alkanoyl, such as 2(R,S)-(N-(2-morpholinoethyl)-carbamoyl)-3-methyl-butyryl, or 2-(N-(pyridyl-lower alkyl)-carbamoyl)-lower alkanoyl, such as 2(R,S)-(N-(2-pyridylmethyl)-carbamoyl)-3-methyl-butyryl; lower alkoxycarbonyl, especially methoxy-, ethoxy-, isopropoxy-, isobutoxy- or tert-lower alkoxy-carbonyl, for example methoxycarbonyl, tert-butoxycarbonyl or isobutoxycarbonyl; aryl-lower alkoxycarbonyl wherein aryl is phenyl, biphenylyl, 1- or 2-naphthyl, fluorenyl, or phenyl that is mono- or poly-substituted by lower alkyl, for example methyl or tert-butyl, hydroxy, lower alkoxy, for example methoxy, ethoxy or tert-butoxy, halogen, for example chlorine or bromine, and/or by nitro, for example phenyl-lower alkoxycarbonyl, such as benzyloxycarbonyl, 4-methoxybenzyloxycarbonyl, 4-nitrobenzyloxycarbonyl or fluorenyl-lower alkoxycarbonyl, such as 9-fluorenylmethoxycarbonyl, especially phenyl-lower alkoxycarbonyl, such as benzyloxycarbonyl; heterocyclyl-lower alkoxycarbonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl, isoquinolyl, quinoxalinyl, β-carbolinyl and a benzo-fused, cyclopenta-, cyclohexa- or cyclohepta-fused derivative of those radicals, which may also be fully or partially saturated, and from morpholinyl and thiomorpholinyl and is unsubstituted or substituted by lower alkyl, for example methyl, such as 1-methylpyrrolidin-2-yl-methoxycarbonyl, 2-furylmethoxycarbonyl, 2-tetrahydrofuryl-methoxycarbonyl, 1-methyl-2-piperidyl-methoxycarbonyl or 2-morpholino-ethoxycarbonyl, or 2-, 3- or 4-pyridylmethoxycarbonyl, especially tetrahydrofuranyl-lower alkoxycarbonyl, such as 2(R,S)-tetrahydrofuranylmethoxycarbonyl; lower alkylsulfonyl, for example methyl- or ethyl-sulfonyl, such as methylsulfonyl; morpholinosulfonyl, thiomorpholinosulfonyl, piperidinosulfonyl, 4-methylpiperazinylsulfonyl or piperazinosulfonyl; N-heterocyclyl-lower alkyl-N-lower alkylcarbamoyl wherein heterocyclyl is selected from thienyl, furyl, pyranyl, pyrrolyl, imidazolyl, pyrazolyl, oxazolyl, isoxazolyl, thiazolyl, furazanyl, pyridyl, pyrazinyl, pyrimidinyl, pyridazinyl, azepinyl, indolyl, benzimidazolyl, 1H-indazolyl, quinolyl, isoquinolyl, quinoxalinyl, quinazolinyl, cinnolyl, purinyl, pteridinyl, naphthyridinyl, 4H-quinolizinyl, 3,1-benzofuranyl, benz[e]indolyl, 4,1-benzoxazinyl, 4,1-benzothiazinyl, carbazolyl, β-carbolinyl, phenazinyl, phenanthridyl, acridyl, phenoxazinyl, phenothiazinyl, 1-azaacenaphthenyl, cyclohexa[b]pyrrolyl, cyclohepta[b]pyrrolyl, cyclohexa[d]pyrazolyl, cyclohexa[b]pyridyl, cyclohexa[b]pyrazinyl, cyclohexa[b]pyrimidinyl, cyclohexa [b]-1,4-oxazinyl, cyclohexa[b]-1,4-thiazinyl, pyrrolidinyl, pyrrolinyl, imidazolidinyl, 2-imidazolinyl, 2,3-dihydropyridyl, piperidyl, piperazinyl, 2,3,5,6-tetrahydropyrazinyl, morpholinyl, thiomorpholinyl, S,S-dioxothiomorpholinyl, indolinyl, isoindolinyl, 4,5,6,7-tetrahydroindolyl, 1,2,3,4-tetrahydroquinolyl, 1,2,3,4-tetrahydroisoquinolyl, chromanyl, thiochromanyl, 1,2,3,4-tetrahydro-3,1-benzodiazinyl, 3,4-dihydro-3H-4,1-benzoxazinyl, 3,4-dihydro-3H-4,1-benzothiazinyl, 2,3,4,5-tetrahydro-1H-5,1-benzazepinyl and 5,6-dihydrophenanthridinyl, the mentioned radicals being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, phenoxy- or naphthyloxy-lower alkyl, for example 2-phenoxyethyl, 1- or 2-naphthyloxymethyl, phenyl-lower alkoxy- or naphthyl-lower alkoxy-lower alkyl, for example benzyloxy-lower alkyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxy-lower alkyl, phenyl-, naphthyl- or fluorenyl-lower alkoxycarbonyloxy-lower alkyl, for example 2-benzyloxycarbonyloxyethyl or 9-fluorenylmethoxycarbonyloxyethyl, amino-lower alkyl, for example aminomethyl, 2-aminoethyl or 2-aminopropyl, carboxy-lower alkyl, for example carboxymethyl or 2-carboxyethyl, hydroxy, lower alkoxy, for example methoxy or ethoxy, phenyl- or naphthyl-lower alkoxy, for example benzyloxy or 1- or 2-naphthylmethoxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, lower alkylsulfonyl, for example methyl- or ethyl-sulfonyl, phosphono, hydroxy-lower alkoxyphosphoryl or dialkoxyphosphoryl, for example dimethoxy- or diethoxy-phosphoryl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxy-ethylcarbamoyl, sulfamoyl, nitro, oxo and/or by cyano, and is especially pyridyl, such as 2-, 3- or 4-pyridyl, especially N-pyridyl-lower alkyl-N-lower alkylcarbamoyl, such as N-(2-, 3- or 4-pyridylmethyl)-N-methylcarbamoyl; or an acyl radical of an amino acid the amino function of which is free or acylated by one of the other radicals mentioned hitherto for R, and R₉, the amino acids being selected from glycine (H-Gly-OH), alanine (H-Ala-OH), valine (H-Val-OH), norvaline α-aminovaleric acid), leucine (H-Leu-OH), isoleucine (H-Ile-OH), norleucine α-aminohexanoic acid, H-Nle-OH), serine (H-Ser-OH), homoserine α-amino-y-hydroxybutyric acid), threonine (H-Thr-OH), methionine (H-Met-OH), cysteine (H-Cys-OH), proline (H-Pro-OH), trans-3- and trans-4-hydroxyproline, phenylalanine (H-Phe-OH), tyrosine (H-Tyr-OH), 4-aminophenylalanine, 4-chlorophenylalanine, 4-carboxyphenylalanine, β-phenylserine (β-hydroxyphenylalanine), phenylglycine, α-naphthylalanine (H-Nal-OH), cyclohexylalanine (H-Cha-OH), cyclohexylglycine, tryptophan (H-Trp-OH), indoline-2-carboxylic acid, 1,2,3,4-tetrahydroisoquinoline-3-carboxylic acid, aspartic acid (H-Asp-OH), asparagine (H-Asn-OH), aminomalonic acid, aminomalonic acid monoamide, glutamic acid (H-Glu-OH), glutamine (H-Gln-OH), histidine (H-His-OH), arginine (H-Arg-OH), lysine (H-Lys-OH), delta.-hydroxylysine, ornithine (α,.delta.-diaminovaleric acid), 3-aminopropanoic acid, α,γ-diaminobutyric acid and α,β-diaminopropionic acid, more especially the radical of an amino acid selected from valine, alanine, leucine, isoleucine, glycine, glutamic acid and asparagine, it being possible for each of the mentioned amino acids (with the exception of glycine) to be in the D-, L- or (D,L)-form, preferably (with the exception of Val, which may also be in the (D)- or (D,L)-form) in the L-form, and the α-amino group being unsubstituted or N-acylated by one of the radicals mentioned above for R₁ and R₉, especially by lower alkanoyl, phenyl-lower alkanoyl, such as phenylacetyl, phenyl-lower alkanoyl wherein the lower alkanoyl radical is substituted by carbamoyl, such as 2(R,S)-carbamoyl-3-phenylpropionyl, morpholino-lower alkanoyl, such as morpholinocarbonyl, thiomorpholino-lower alkanoyl, such as thiomorpholinocarbonyl, pyridyl-lower alkanoyl, such as 2-, 3- or 4-pyridylacetyl, quinolinyl-lower alkanoyl, such as quinoline-2-carbonyl, tetrazolyl-lower alkanoyl, such as 3-tetrazol-1-ylpropionyl, amino-lower alkanoyl substituted at the amino nitrogen atom by N-morpholino- or N-thiomorpholino-carbonyl, for example N-morpholino- or N-thiomorpholino-carbonylamino-lower alkanoyl, such as N-morpholino- or N-thiomorpholino-carbonylaminoacetyl, halo-lower alkanoyl containing up to three halogen atoms, for example α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl, especially trifluoroacetyl, 2-(N-morpholino-lower alkylcarbamoyl)-lower alkanoyl, such as 2(R,S)-(N-(2-morpholinoethyl)-carbamoyl)-3-methyl-butyryl, 2-(N-(pyridyl-lower alkyl)-carbamoyl)-lower alkanoyl, such as 2(R,S)-(N-(2-pyridylmethyl)-carbamoyl)-lower alkanoyl, lower alkoxycarbonyl, phenyl-lower alkoxycarbonyl, tetrahydrofuranyl-lower alkoxycarbonyl, such as 2(R,S)-tetrahydrofuranylmethoxycarbonyl, lower alkylsulfonyl or N-pyridyl-lower alkyl-N-lower alkylcarbamoyl, such as N-(2-, 3- or. 4-pyridylmethyl)-N-methylcarbamoyl, greatest preference being given to N-morpholinocarbonyl-glycine, N-(N-(2-, 3- or 4-pyridyl)methyl-N-methylaminocarbonyl)-glycine, valine, N-(trifluoroacetyl)-valine, N-phenylacetyl-valine, N-(2- or 3-pyridyl)-acetyl-valine, N-acetyl-valine, N-(2-carbamoyl-3-phenylpropionyl)-valine, N-(2(R,S)-carbamoyl-3-phenylpropionyl)-valine, N-(2- or 3-pyridylacetyl)-valine, N-2-tetrahydrofurylmethoxycarbonyl-valine, N-(3-(tetrazol-1-yl)-propionyl)-valine, N-(quinoline-2-carbonyl)-valine, N-methoxycarbonyl-valine, N-isobutoxycarbonyl-valine, N-tert-butoxycarbonyl-valine, N-benzyloxycarbonyl-valine, N-(morpholinocarbonyl)-valine, N-(thiomorpholinocarbonyl)valine, N-(S,S-dioxothiomorpholinocarbonyl)-valine, N-(N-2-pyridylmethyl-N-methylaminocarbonyl)-valine, N-morpholinocarbonylaminoacetyl-valine, N-methylsulfonyl-valine, morpholinosulfonyl-valine, N-acetyl-isoleucine, N-propionyl-isoleucine, N-(benzyloxycarbonyl)isoleucine, N-benzyloxycarbonyl-glutamic acid, asparagine, N-benzyloxycarbonylasparagine and quinoline-2-carbonyl-asparagine, wherein each of the amino acid radicals is preferably in the (L)- or (D,L)-form, and in the case of valine also in the (D)-form; with the proviso that not more than one of the radicals R₁ and R₉ is hydrogen,
R₂, R₄, R₆ and R₈ are hydrogen,
R₃ is lower alkyl, such as isobutyl or n-butyl; cycloalkyl-lower alkyl wherein cycloalkyl has from 3 to 7 carbon atoms and is unsubstituted or mono- to tri-substituted by lower alkyl, such as isopropyl, halo-lower alkyl, such as trifluoromethyl, hydroxy, lower alkoxy, carbamoyl-lower alkoxy, N-lower alkylcarbamoyl-lower alkoxy or N,N-di-lower alkylcarbamoyl-lower alkoxy, amino, mono- or di-lower alkylamino, halogen, such as fluorine, chlorine or bromine, carboxy, lower alkoxycarbonyl, phenyl-, naphthyl- or fluorenyl-lower alkoxycarbonyl, lower alkanoyl, lower alkylsulfonyl, for example methylsulfonyl, phosphono, hydroxy-lower alkoxyphosphoryl or di-lower alkoxyphosphoryl, carbamoyl, mono- or di-lower alkylaminocarbamoyl, sulfamoyl, mono- or di-lower alkylsulfamoyl, nitro and/or by cyano and is bonded, preferably terminally, to lower alkyl, especially methyl, ethyl, n-propyl, isopropyl, n-butyl, isobutyl or tert-butyl, such as cyclobutyl-, cyclopentyl-, cyclohexyl- or cycloheptyl-lower alkyl, such as -methyl or -ethyl, especially cyclohexyl-lower alkyl, more especially cyclohexylmethyl; or aryl-lower alkyl wherein aryl is phenyl, indenyl, indanyl, naphthyl, anthryl, phenanthryl, acenaphthyl or fluorenyl and is unsubstituted or substituted by lower alkyl, for example methyl, ethyl or isopropyl, halo-lower alkyl, such as trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine or chlorine, carboxy, lower alkoxycarbonyl, benzyl-, naphthyl- or fluorenyl-lower alkoxycarbonyl, lower alkanoyl, sulfo, lower alkylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylsulfamoyl, nitro and/or by cyano, wherein phenyl may be present up to three times, such as in diphenyl-, dibenzyl- or triphenyl-lower alkyl, for example diphenyl-, dibenzyl- or triphenyl-2-ethyl, especially phenyl-lower alkyl that is unsubstituted or substituted by the mentioned substituents, especially benzyl, 4-fluoro- or 4-cyano-benzyl,
R₅ is hydroxy, and
R₇ is unsubstituted lower alkyl, especially isobutyl or n-butyl; or cycloalkyl-lower alkyl, for example as last described for cycloalkyl-lower alkyl R₃, especially cyclohexyl-lower alkyl, more especially cyclohexylmethyl; or aryl-lower alkyl as last described for aryl-lower alkyl R₃, especially phenyl-lower alkyl that is unsubstituted or substituted by the mentioned substituents, more especially benzyl, 4-fluoro- or 4-cyano-benzyl,
or a salt thereof where at least one salt-forming group is present.

Special preference is given also to the compounds of formula I wherein R₁ is lower alkoxycarbonyl, phenyl-lower alkoxycarbonyl, the monovalent radical, bonded via the carboxy group, of an aliphatic amino acid selected from valine, alanine, leucine and isoleucine, or the radical, bonded via the carboxy group, of an aliphatic amino acid as defined above that is acylated at the amino nitrogen atom by one of the radicals phenyl-lower alkanoyl, morpholinyl-lower alkanoyl, thiomorpholinyl-lower alkanoyl, S,S-dioxothiomorpholinyl-lower alkanoyl, pyridyl-lower alkanoyl, lower alkoxycarbonyl and phenyl-lower alkoxycarbonyl, all the mentioned amino acids being in the D-, D,L- or L-form, preferably in the L-form, R₂ is hydrogen, R₃ is phenyl-lower alkyl, R₄ is hydrogen, R₅ is hydroxy, R₆ is hydrogen, R₇ is lower alkyl, cyclohexyl-lower alkyl or phenyl-lower alkyl, R₈ is hydrogen and R₉ is one of the radicals mentioned for R₁ and the asymmetric carbon atoms carrying the radicals R₃ and R₅ are in the S-configuration, and the pharmacologically acceptable salts of such compounds.

Very special preference is given to the compounds of formula I wherein R₁ is tert-butoxycarbonyl, benzyloxycarbonyl, the monovalent radical, bonded via the carboxy group, of the amino acid valine or the radical, bonded via the carboxy group, of alanine acylated at the amino nitrogen atom by one of the radicals phenylacetyl, 3-pyridylacetyl, morpholinocarbonyl, thiomorpholinocarbonyl, tert-butoxycarbonyl and benzyloxycarbonyl, R₂ is hydrogen, R₃ is benzyl, R₄ is hydrogen, R₅ is hydroxy, R₆ is hydrogen, R₇ is isobutyl, cyclohexylmethyl or benzyl, R₈ is hydrogen and R₉ is one of the radicals mentioned for R₁ and the asymmetric carbon atoms carrying the radicals R₃ and R₅ are in the S-configuration, and the pharmacologically acceptable salts of such compounds.

Great preference is given to the compounds of formula I wherein R₁ and R₉ are each independently of the other hydrogen, lower alkanoyl, such as acetyl, phenyl-lower alkanoyl, such as phenylacetyl, phenyl-lower alkanoyl wherein the lower alkanoyl radical is substituted by carbamoyl, such as 2(R,S)-carbamoyl-3-phenyl-propionyl, morpholino-lower alkanoyl, such as morpholinocarbonyl, thiomorpholino-lower alkanoyl, such as thiomorpholino-carbonyl, pyridyl-lower alkanoyl, such as 2-, 3- or 4-pyridylacetyl, quinolyl-lower alkanoyl, such as quinoline-2-carbonyl, tetrazolyl-lower alkanoyl, such as 3-tetrazol-1-yl-propionyl, amino-lower alkanoyl substituted at the amino nitrogen atom by N-morpholino- or N-thiomorpholino-carbonyl, for example N-morpholino- or N-thiomorpholino-carbonylamino-lower alkanoyl, such as N-morpholino- or N-thiomorpholinocarbonylamino-acetyl, halo-lower alkanoyl containing up to three halogen atoms, such as trifluoroacetyl, 2-(N-morpholino-lower alkylcarbamoyl)-lower alkanoyl, such as 2(R,S)-(N-(2-morpholinoethyl)-carbamoyl-3-methyl-butyryl, 2-(N-pyridyl-lower alkylcarbamoyl)-lower alkanoyl, such as 2(R,S)-(N-(2-pyridylmethyl)-carbamoyl)-3-methylbutyryl, lower alkoxycarbonyl, such as methoxy-, isobutoxy- or tert-lower alkoxycarbonyl, phenyl-lower alkoxycarbonyl, such as benzyloxycarbonyl, tetrahydrofuranyl-lower alkoxycarbonyl, such as 2(R,S)-tetrahydrofuranyl-methoxycarbonyl, lower alkylsulfonyl, for example methyl- or ethyl-sulfonyl, morpholinosulfonyl, thiomorpholinosulfonyl, N-pyridyl-lower alkyl-N-lower alkylcarbamoyl, or an acyl radical of an amino acid selected from glycine, alanine, valine, leucine, isoleucine, glutamic acid and asparagine in the (D)-, (L)- or (D,L)-form, wherein the α-amino group is unsubstituted or acylated by one of the other radicals R₁ or R₂ mentioned hitherto, greatest preference being given to N-morpholinocarbonyl-glycine, N-(N-(2-, 3- or 4-pyridyl)methyl-N-methylaminocarbonyl)-glycine, valine, N-(trifluoroacetyl)-valine, N-phenylacetyl-valine, N-(2- or 3-pyridyl)-acetyl-valine, N-acetyl-valine, N-(2-carbamoyl-3-phenylpropionyl)-valine, N-(2(R,S)-carbamoyl-3-phenyl-propionyl)-valine, N-(2- or 3-pyridylacetyl)-valine, N-2-tetrahydrofurylmethoxycarbonyl-valine, N-(3-(tetrazol-1-yl)propionyl)-valine, N-(quinoline-2-carbonyl)-valine, N-methoxycarbonyl-valine, N-isobutoxycarbonyl-valine, N-tert-butoxycarbonyl-valine, N-benzyloxycarbonyl-valine, N-(morpholinocarbonyl)-valine, N-(thiomorpholinocarbonyl)-valine, N-(S,S-dioxothiomorpholinocarbonyl)-valine, N-(N-2-pyridylmethyl-N-methylaminocarbonyl)-valine, N-morpholinocarbonylaminoacetyl-valine, N-methylsulfonyl-valine, morpholinosulfonyl-valine, N-acetyl-isoleucine, N-propionyl-isoleucine, N-(benzyloxycarbonyl)-isoleucine, N-benzyloxycarbonyl-glutamic acid, asparagine, N-benzyloxycarbonyl-asparagine and quinoline-2-carbonyl-asparagine, wherein the amino acid radicals are each preferably in the (L)- or (D,L)-form, and in the case of valine also in the (D)-form; with the proviso that not more than one of the radicals R₁ and R₉ is hydrogen,
R₂, R₄, R₆ and R₈ are hydrogen,
R₃ is lower alkyl, such as n-butyl or isobutyl, cyclohexyl-lower alkyl, such as cyclohexylmethyl, or phenyl-lower alkyl that is unsubstituted or substituted by halogen, such as fluorine, lower alkoxy, such as methoxy, or by cyano, especially benzyl, 4-fluorobenzyl or 4-cyanobenzyl,
R₅ is hydroxy, and
R₇ is lower alkyl; cyclohexyl-lower alkyl; or phenyl-lower alkyl that is unsubstituted or substituted by halogen, such as fluorine, lower alkoxy, such as methoxy, or by cyano; as last defined for R₃,
   or a salt thereof where salt-forming groups are present, still greater preference being given to those compounds in which R₁ and/or R₉ are not morpholinosulfonyl or thiomorpholinosulfonyl.

Most preferred of all, are the compounds mentioned in the Examples and their salts.

Synthesis of Compounds

The compounds of formula I and salts of such compounds having at least one salt-forming group are obtained by means of processes known per se, for example as follows:
a) a hydrazine derivative of the formula 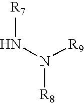
wherein the radicals are as defined above, is added to an epoxide of the formula 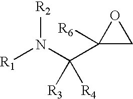
wherein the radicals are as defined above, free functional groups, with the exception of those participating in the reaction, being optionally in protected form, and any protecting groups present are removed, or
b) for the preparation of compounds of formula I wherein R₁ and R₉ are acyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two identical or different radicals selected from substituted or unsubstituted alkyl, unsubstituted or substituted cycloalkyl, aryl, hydroxy, unsubstituted or substituted alkoxy, cycloalkoxy and aryloxy; R₂ and R₈ are hydrogen, unsubstituted or substituted alkyl, alkenyl or alkynyl, or heterocyclyl, and the remaining radicals are as defined, an amino compound of the formula 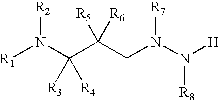
wherein the radicals are as defined immediately above, is condensed with an acid of the formula
   R₉—OH
or with a reactive acid derivative thereof, wherein R₉ is as defined immediately above, free functional groups, with the exception of those participating in the reaction, being optionally in protected form, and any protecting groups present are removed, or
c) for the preparation of compounds of formula I wherein R₁ and R₉ are acyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl that is substituted by one or two identical or different radicals selected from substituted or unsubstituted alkyl, unsubstituted or substituted cycloalkyl, aryl, hydroxy, unsubstituted or substituted alkoxy, cycloalkoxy and aryloxy; R₂ and R₈ are hydrogen, unsubstituted or substituted alkyl, alkenyl or alkynyl, or heterocyclyl, and the remaining radicals are as defined, an amino compound of the formula 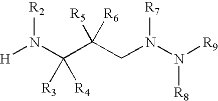
wherein the radicals are as defined immediately above, is condensed with an acid of the formula
   R₁—OH
or with a reactive acid derivative thereof, wherein R₁ is as defined immediately above, free functional groups, with the exception of those participating in the reaction, being optionally in protected form, and any protecting groups present are removed, or
d) for the preparation of compounds of formula I wherein R₁ and R₉ are two identical radicals selected from acyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; and phosphoryl that is substituted by one or two identical or different radicals selected from substituted or unsubstituted alkyl, unsubstituted or substituted cycloalkyl, aryl, hydroxy, unsubstituted or substituted alkoxy, cycloalkoxy and aryloxy; R₂ and R₈ are hydrogen, unsubstituted or substituted alkyl, alkenyl or alkynyl, or heterocyclyl, and the remaining radicals are as defined, a diamino compound of the formula 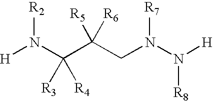
wherein the radicals are as defined immediately above, is condensed with an acid suitable for introducing the identical radicals R₁ and R₉, or with reactive acid derivatives thereof, wherein R₁ and R₉ are as defined immediately above, free functional groups, with the exception of those participating in the reaction, being optionally in protected form, and any protecting groups present are removed, or
e) for the preparation of a compound of formula I wherein in place of the radical R₇ there is a radical R₇″ which is unsubstituted or substituted alkyl or cycloalkyl, in a compound of the formula I′ 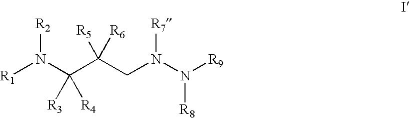
wherein R₇″ is hydrogen and the remaining radicals are as defined above, the radical R₇″ is introduced by substitution with a compound of the formula
   R₇″—X
wherein X is a leaving group and R₇″ is unsubstituted or substituted alkyl or cycloalkyl, free functional groups, with the exception of those participating in the reaction, being optionally in protected form, and any protecting groups present are removed, or
f) in a compound of formula I wherein the substituents are as defined above, with the proviso that in the compound of formula I in question at least one functional group is protected by protecting groups, the protecting groups present are removed and, if desired, a compound of formula I obtainable in accordance with any one of processes a) to f) above having at least one salt-forming group is converted into its salt or an obtainable salt is converted into the free compound or into a different salt and/or any isomeric mixtures that are obtainable are separated and/or a compound of formula I according to the invention is converted into a different compound of formula I according to the invention.

The above processes are described in detail in U.S. Pat. No. 5,753,652, herein incorporated by reference in its entirety.

The acylated compounds according to the invention are compounds of formula 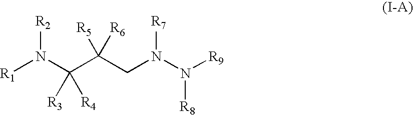
wherein R₁ and R₉ are each independently of the other hydrogen; acyl; unsubstituted or substituted alkyl; sulfo; or sulfonyl substituted by unsubstituted or substituted alkyl, aryl or by heterocyclyl, with the proviso that not more than one of the radicals R₁ and R₉ is hydrogen; and
R₂ and R₈ are each independently of the other hydrogen or unsubstituted or substituted alkyl;
R₃ and R₄ are each independently of the other hydrogen, unsubstituted or substituted alkyl, unsubstituted or substituted cycloalkyl or aryl;
R₅ is acyloxy;
R₆ is hydrogen; and
R₇ is unsubstituted or substituted alkyl, unsubstituted or substituted cycloalkyl or aryl; and salts of the mentioned compounds where salt-forming groups are present, with the exception of the compound wherein R₁ and R₉ are each acetyl, R₂, R₃, R₄, R₆ and R₈ are each hydrogen, R₅ is acetoxy and R₇ is 2,2-[N-ethoxycarbonylmethyl)-N-methyl)hydrazin-1-ylcarbonylmethyl. In another aspect, the invention provides a method of treating or preventing Alzheimer's disease in a patient in need of such treatment comprising administering a therapeutically effective amount of a compound of Formula (I-A) or a pharmaceutically acceptable salt thereof: 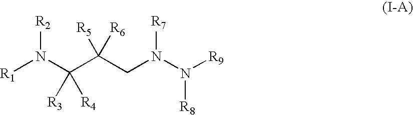
wherein R₁ and R₉ are each independently of the other hydrogen; acyl; unsubstituted or substituted alkyl; sulfo; or sulfonyl substituted by unsubstituted or substituted alkyl, aryl or by heterocyclyl, with the proviso that not more than one of the radicals R₁ and R₉ is hydrogen;
R₂ and R₈ are each independently of the other hydrogen or unsubstituted or substituted alkyl;
R₃ and R₄ are each independently of the other hydrogen, unsubstituted or substituted alkyl, unsubstituted or substituted cycloalkyl or aryl;
R₅ is acyloxy;
R₆ is hydrogen; and
R₇ is unsubstituted or substituted alkyl, unsubstituted or substituted cycloalkyl or aryl; and salts of the mentioned compounds where salt-forming groups are present, with the exception of the compound wherein R₁ and R₉ are each acetyl, R₂, R₃, R₄, R₆ and R₈ are each hydrogen, R₅ is acetoxy and R₇ is 2,2-[N-ethoxycarbonylmethyl)-N-methyl]hydrazin-1-ylcarbonylmethyl.

In another aspect, the invention provides a method of treating a patient who has, or in preventing a patient from getting, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I-A), or a pharmaceutically acceptable salt thereof: 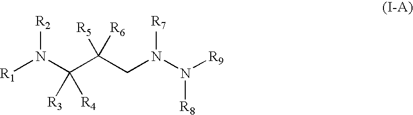
wherein R₁ and R₉ are each independently of the other hydrogen; acyl; unsubstituted or substituted alkyl; sulfo; or sulfonyl substituted by unsubstituted or substituted alkyl, aryl or by heterocyclyl, with the proviso that not more than one of the radicals R₁ and R₉ is hydrogen;
R₂ and R₈ are each independently of the other hydrogen or unsubstituted or substituted alkyl;
R₃ and R₄ are each independently of the other hydrogen, unsubstituted or substituted alkyl, unsubstituted or substituted cycloalkyl or aryl;
R₅ is acyloxy;
R₆ is hydrogen; and
R₇ is unsubstituted or substituted alkyl, unsubstituted or substituted cycloalkyl or aryl; and salts of the mentioned compounds where salt-forming groups are present, with the exception of the compound wherein R₁ and R₉ are each acetyl, R₂, R₃, R₄, R₆ and R₈ are each hydrogen, R₅ is acetoxy and R₇ is 2,2-[N-ethoxycarbonylmethyl)-N-methyl]hydrazin-1-ylcarbonylmethyl.

In another aspect, preferred compounds of the invention are represented by the following:
1-[2(S)-palmitoyloxy-3(S)-(N-(methoxy-carbonyl)-(L)-valyl)amino-4-phenyl-butyl]-1-[cyclohexylmethyl]-2-[N-methoxy-carbonyl-(L)-valyl]hydrazine;
1[-2(S)-(methoxy-acetoxy)-3(S)-(N-(methoxy-carbonyl)-(L)-valyl)amino-4-phenylbutyl]-1-[cyclohexylmethyl]-2-[N-methoxy-carbonyl-(L)-valyl]hydrazine;
1-[2(S)-(2-pyridyl-carbonyl)oxy-3(S)-(tert-butoxy-carbonyl)amino-4-phenyl-butyl]-1-[cyclohexylmethyl]-2-[tert-butoxy-carbonyl]hydrazine;
1-[2(S)-(2-pyridylcarbonyl)oxy-3(S)-(N-quinoline-2-carbonyl)-(L)-asparaginyl)amino4-phenylbutyl-1-[phenylmethyl]-2-[N-(methoxycarbonyl)-(L)-valyl]hydrazine;
1-[2(S)-butyryloxy-3(S)-(N-quinoline-2-carbonyl)-(L)-asparaginyl)amino-4-phenylbutyl-1-[phenylmethyl]-2-[N-(methoxycarbonyl)-(L)-valyl]hydrazine;
1-[2(S)-(2-pyridylcarbonyl)oxy-3(S)-(N-quinoline-2-carbonyl)-(L)-asparaginyl)amino4-phenylbutyl-1-[4-methoxyphenylmethyl]-2-[N-(benzyloxycarbonyl)-(L) valyl]hydrazine;
1-[2(S)-(methoxy-acetyl)oxy-3(S)-(N-quinoline-2-carbonyl)-(L)-asparaginyl)amino-4-phenylbutyl-1-[4-methoxyphenylmethyl]-2-[N-(benzyloxycarbonyl)-(L)-valyl)hydrazine;
1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-octanoyloxy-3(S)-(N-(methoxycarbonyl)-(L)-valyl)amino-4-phenyl-butyl]-1-[cyclohexylmethyl]-2-[N-15, (methoxycarbonyl)-(L)-valyl]hydrazine;
1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-furylcarbonyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-imidazolylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-pyridylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]hydrazine;
1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-benzyloxycarbonyl-N-methyl-aminoacetyl)oxy-3(S)-(N-(methoxycarbonyl)(L)-valyl)amino-4-phenyl-butyl]-1-[cyclohexylmethyl]-2-[N-(methoxycarbonyl)(L)-valyl]hydrazine;
1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-morpholinomethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl)hydrazine; and
1-[2(S)-(3-carboxypropionyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
   or a pharmaceutically acceptable salt thereof.

In the description of compounds of formula I-A the term “lower” used in the definition of groups or radicals, for example lower alkyl, lower alkoxy, lower alkanoyl etc., means that, unless expressly otherwise defined, the groups or radicals so defined contain up to and including a maximum of 7, and preferably up to and including 4, carbon atoms. In the case of lower alkenyl or lower alkynyl, from 2 to 7, preferably from 3 to 7, and especially 3 or 4, carbon atoms are present.

Unless indicated to the contrary, where substituted the radicals R₁, R₂, R₃, R₄, R₇, R₈ and/or R₉ are mono- or poly-substituted, especially mono- to tri-substituted, for example mono-substituted, by identical or different substituents.

If a radical that is defined by referring back to another substituent is defined “independently” of the radical used for the definition, it means that if both radicals are present in a compound they need not be identical, although they can, however, be identical.

The carbon atoms in compounds of formula I-A substituted by R₃ and R₄ and by R₅ and R₆ may, if they are asymmetric, be in the (R)-, (S)- or (R,S)-configuration, as may also any other asymmetric carbon atoms present. Accordingly, the present compounds may be in the form of isomeric mixtures or in the form of pure isomers, especially in the form of diastereoisomeric mixtures, pairs of enantiomers or pure enantiomers. Preferred compounds of formula I-A are those wherein the carbon atoms substituted by R₃ and by R₅ have the (S)-configuration and any other asymmetric carbon atoms that may be present are, independently of one another, in the (R)-, (S)- or (R,S)-configuration.

Unless otherwise indicated, the general terms and names used in the description of the present invention preferably have the following meanings:

Acyl R₁ or R₉ has, for example, up to 25, preferably up to 19, carbon atoms and is especially the acyl group of a carboxylic acid, of a semiester of carbonic acid, of an unsubstituted or N-substituted carbamic acid or of an unsubstituted or substituted amino acid.

Preferred acyl groups R₁ or R₉ of a carboxylic acid are unsubstituted or substituted alkanoyl, alkenoyl or alkynoyl having up to 19 carbon atoms, for example n-decanoyl, or preferably lower alkanoyl, such as formyl, acetyl, propionyl, butyryl or pivaloyl; or also or especially 3,3-dimethylbutyryl; or substituted lower alkanoyl wherein preferably up to four, especially (except in the case of halogen which may be present up to three times as a substituent) up to two, substituents may be present, especially one substituent (except in the case of halogen which may be present up to three times as a substituent), the substituents being selected especially from cycloalkyl-lower alkanoyl wherein cycloalkyl has, for example, from 3 to 7 carbon atoms and lower alkanoyl is as defined above, for example cycloalkylcarbonyl, for example having a total of from 4 to 8 carbon atoms, such as cyclopropyl-, cyclobutyl-, cyclopentyl- or cyclohexyl-carbonyl, or 2-cyclohexyl- or 2-cyclopentyl-acetyl, cycloalkenyl-lower alkanoyl wherein cycloalkenyl has, for example, from 3 to 7 carbon atoms, such as cycloalkenylcarbonyl, for example having from 4 to 8 carbon atoms, such as 1-cyclohexenylcarbonyl, 1,4-cyclohexadienylcarbonyl or 1-cyclohexenylacetyl or 1,4-cyclohexadienylacetyl, bicycloalkyl-lower alkanoyl wherein bicycloalkyl contains, for example, from 5 to 10 carbon atoms, for example bicycloalkylcarbonyl, preferably having from 8 to 11 carbon atoms, such as decahydronaphthyl-2-carbonyl, bicyclohexyl-, bicycloheptyl-, bicyclooctyl-, bicyclononyl- or bicyclodecyl-acetyl or -3-propionyl, bicycloalkenylcarbonyl, preferably having from 8 to 12 carbon atoms, especially in bicycloalkenylcarbonyl, such as 5-norbornen-2-ylcarbonyl or bicyclo[2.2.2]octen-2-yl-carbonyl, tricycloalkyl-lower alkanoyl wherein tricycloalkyl contains, for example, from 8 to 10 carbon atoms, for example tricycloalkylcarbonyl, preferably having from 8 to 11 carbon atoms, such as 1- or 2-adamantylcarbonyl, aryl-lower alkanoyl wherein aryl has from 6 to 14 ring carbon atoms, such as in phenyl, indenyl, indanyl, naphthyl, anthryl, phenanthryl, acenaphthyl or fluorenyl, and may be unsubstituted or mono- to tri-substituted especially by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, wherein phenyl may be present up to three times, for example in diphenyl-, dibenzyl- or triphenyl-lower alkanoyl, such as diphenyl-, dibenzyl- or tri-phenyl-acetyl, and wherein lower alkanoyl may be unsubstituted or substituted, for example by carboxy; lower alkoxycarbonyl, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyl; aryl-lower alkoxycarbonyl wherein aryl has from 6 to 12 carbon atoms, for example benzyloxycarbonyl; carbamoyl, carbamoyl substituted at the nitrogen atom by one or two radicals selected from lower alkyl, such as methyl, ethyl, propyl, isopropyl, n-butyl, isobutyl, sec-butyl and tert-butyl, for example in N-methyl-carbamoyl, N-n-butyl-carbamoyl or N,N-dimethylcarbamoyl, from carboxy-lower alkyl or lower alkoxycarbonyl-lower alkyl, for example in the form of carboxymethylcarbamoyl (glycinylcarbonyl) or in the form of tert-butoxycarbonylmethylcarbamoyl, from di-lower alkylamino-lower alkyl, for example 2-dimethylaminoethyl, from hydroxy-lower alkyl, for example hydroxymethyl or hydroxyethyl, and from di-lower alkoxy-lower alkyl, for example 2-(2,2-dimethoxyethyl); and/or by cyano and is unbranched or branched, selected especially from phenyl-lower alkanoyl, such as benzoyl, phenylacetyl or 3-phenylpropionyl, which may be unsubstituted or mono- or poly-substituted at the phenyl ring, for example by lower alkyl, for example methyl, phenyl, halogen, for example fluorine or chlorine, hydroxy, lower alkoxy, for example methoxy, and/or by nitro, such as 4-chloro-, 4-methoxy- or 4-nitro-benzoyl, naphthylcarbonyl, such as α- or β-naphthylcarbonyl or 1,8-naphthalenedicarbonyl bonded to the amino group via both carbonyl groups, indenylcarbonyl, such as 1-, 2- or 3-indenylcarbonyl, indanylcarbonyl, such as 1- or 2-indanylcarbonyl, phenanthrenylcarbonyl, such as 9-phenanthrenylcarbonyl, α-naphthylacetyl, β-naphthylacetyl, lower alkylphenylacetyl, such as 4-methylphenylacetyl, lower alkoxyphenylacetyl, such as 4-methoxyphenylacetyl, 3-(p-hydroxyphenyl)-propionyl, diphenylacetyl, di(4-methoxyphenyl)acetyl, triphenylacetyl, 2,2-dibenzylacetyl, 3-α- or 3-β-naphthylpropionyl, phenyl-lower alkanoyl wherein the lower alkanoyl radical is substituted by carbamoyl, for example 2-carbamoyl-3-phenylpropionyl, such as 2-(R,S)carbamoyl-3-phenylpropionyl, 3-α-naphthyl-2-carbamoylpropionyl, 3-phenyl- or 3-α-naphthyl-2-tert-butylcarbamoyl-propionyl, 3-phenyl- or 3-α-naphthyl-2-(2-dimethylaminoethyl)carbamoylpropionyl, 3-α-naphthyl-2-(carboxy- or tert-butoxy-carbonyl-)methylcarbamoyl-propionyl, 3-phenyl- or 3-α-naphthyl-2-(3-hydroxy-2-propyl)carbamoyl-propionyl, 3-phenyl- or 3-α-naphthyl-2-(2,2-dimethoxyethyl)carbamoylpropionyl and 3-phenyl- or 3-α-naphthyl-2-(5-amino-5-carboxypentyl)-carbamoylpropionyl, especially phenyl-lower alkanoyl, such as phenylacetyl, or phenyl-lower alkanoyl wherein the lower alkanoyl radical is substituted by carbamoyl, such as 2-(R,S)carbamoyl-3-phenylpropionyl, heterocyclyl-lower alkanoyl wherein lower alkanoyl is unsubstituted or substituted as defined above under aryl-lower alkanoyl R₁ or R₉ and wherein heterocyclyl is preferably a single or double ring system having from 3 to 10 ring atoms, is bonded via a carbon atom or, especially, via a nitrogen atom and contains up to 3 further hetero atoms selected from oxygen, nitrogen, sulfur, and sulfur linked to 1 or 2 oxygen atoms; the mentioned ring system may also be fused with 1 or 2 phenyl or naphthyl radicals, it being possible for naphthyl also to be fused-on by two sides, or with 1 or 2 cycloalkyl radicals, cycloalkyl preferably having from 5 to 7 ring atoms; and which may be unsaturated or partially or fully saturated, for example thienyl, furyl, pyrrolyl, imidazolyl, pyrazolyl, oxazolyl, thiazolyl, tetrazolyl, pyridyl, pyrazinyl, pyrimidinyl, pyridazinyl, indolyl, benzimidazolyl, quinolyl, isoquinolyl, 3,1-benzofuranyl, cyclohexa[b]pyrrolyl, cyclohexa[b]pyridyl, cyclohexa[b]pyrazinyl, cyclohexa[b]pyrimidinyl, pyrrolidinyl, pyrrolinyl, imidazolidyl, piperidyl, piperazinyl, morpholinyl, thiomorpholinyl, S,S-dioxo-thiomorpholinyl, indolinyl, isoindolinyl, 4,5,6,7-tetrahydroindolyl, 1,2,3,4-tetrahydroquinolyl or 1,2,3,4-tetrahydroisoquinolyl, with heterocyclyl, for example one of the last-mentioned radicals, being unsubstituted or substituted by lower alkyl, for example methyl, phenyl, 1- or 2-naphthyl, phenyl-lower alkyl, for example benzyl, hydroxy-lower alkyl, for example hydroxymethyl or 2-hydroxyethyl, lower alkoxy-lower alkyl, for example methoxymethyl or 2-methoxyethyl, lower alkanoyloxy-lower alkyl, for example acetoxymethyl, phenyl- or naphthyl-lower alkanoyloxy-lower alkyl, for example benzoyloxy-, phenylacetoxy- or 1- or 2-naphthoyloxy-methyl, -2-ethyl or -2-(2,2-dimethylethyl), lower alkoxycarbonyloxy-lower alkyl, for example tert-butoxycarbonyloxy-lower alkyl, phenyl-lower alkoxycarbonyloxy-lower alkyl, for example 2-benzyloxycarbonyloxyethyl, amino-lower alkyl, for example aminomethyl, hydroxy, lower alkoxy, for example methoxy or ethoxy, amino, lower alkylamino, for example methyl-, ethyl- or tert-butyl-amino, di-lower alkylamino, for example dimethyl- or diethyl-amino, carboxy, lower alkoxycarbonyl, for example methoxy-, isopropoxy-, sec-butoxy- or tert-butoxy-carbonyl, phenyl- or naphthyl-lower alkoxycarbonyl, for example benzyloxycarbonyl, halogen, for example fluorine, chlorine, bromine or iodine, especially chlorine or bromine, lower alkanoyl, for example acetyl or pivaloyl, carbamoyl, mono- or di-lower alkylcarbamoyl, for example N-methylcarbamoyl, N-n-butylcarbamoyl or N,N-dimethylcarbamoyl, hydroxy- or carboxy-lower alkylcarbamoyl, for example hydroxy- or carboxy-methylcarbamoyl or hydroxy- or carboxyethylcarbamoyl, nitro, oxo and/or by cyano; especially in heterocyclyl-lower alkanoyl wherein lower alkanoyl is unsubstituted or substituted independently by one of the substituents defined above under aryl-lower alkanoyl R₁ or R₉; with heterocyclyl-lower alkanoyl being selected especially from pyrrolylcarbonyl, for example 2- or 3-pyrrolylcarbonyl, thienylcarbonyl, such as 2-thienylcarbonyl, furylcarbonyl, such as 2-furylcarbonyl, indolylcarbonyl, such as 2-, 3- or 5-indolylcarbonyl, 4,5,6,7-tetrahydroindolyl-2-carbonyl, quinolyl-lower alkanoyl, for example quinolylcarbonyl, such as 2-, 3- or 4-quinolylcarbonyl, isoquinolylcarbonyl, such as 1-, 3- or 4-isoquinolylcarbonyl, piperidylcarbonyl, such as piperidinocarbonyl or 2-, 3- or 4-piperidylcarbonyl, piperazinylcarbonyl, such as piperazin-1-ylcarbonyl, morpholinyl-lower alkanoyl, for example morpholino-lower alkanoyl, such as morpholinocarbonyl, thiomorpholinyl-lower alkanoyl, for example thiomorpholino-lower alkanoyl, such as thiomorpholinocarbonyl, S,S-dioxothiomorpholinylcarbonyl, such as S,S-dioxothiomorpholinocarbonyl, 1,2,3,4-tetrahydroquinolylcarbonyl, such as 1,2,3,4-tetrahydroquinolyl-2-, -3- or -4-carbonyl, 1,2,3,4-tetrahydroisoquinolylcarbonyl, such as 1,2,3,4-tetrahydroisoquinolyl-1-, -3- or -4-carbonyl, tetrazolyl-lower alkanoyl, such as 3-(tetrazol-1-yl)-propionyl, and pyridyl-lower alkanoyl, for example pyridylcarbonyl, such as 2-, 3- or 4-pyridylcarbonyl, or pyridylacetyl, such as 2-, 3- or 4-pyridylacetyl; and heterocyclyl-lower alkanoyl being selected most especially from morpholinocarbonyl, thiomorpholinocarbonyl, quinolin-2-ylcarbonyl, 3-(tetrazol-1-yl)-propionyl, 2-pyridylcarbonyl and 2- or 3-pyridylacetyl, hydroxy-lower alkanoyl, such as 3-hydroxypropionyl or 2-hydroxy-3-methylpentanoyl, hydroxy-lower alkoxy-lower alkanoyl, such as 3-hydroxy-n-propoxycarbonyl, lower alkoxy-lower alkanoyl, for example lower alkoxyacetyl or lower alkoxypropionyl, such as methoxyacetyl, ethoxyacetyl or 3-methoxypropionyl, lower alkoxy-lower alkoxy-lower alkanoyl, such as 2-methoxymethoxy-3-methylpentanoyl or also or especially 2-(methoxy)ethoxyacetyl, lower alkanoyloxy-lower alkanoyl wherein lower alkanoyloxy is, for example, acetoxy, propionyloxy, butyryloxy, isobutyryloxy or pivaloyloxy, such as acetoxyacetyl or 3-acetoxypropionyl, amino-lower alkanoyl wherein the amino group is not in the α- or β-position, such as 5-aminopentanoyl, lower alkanoylamino-lower alkanoyl wherein the amino group is not in the α- or β-position of the lower alkanoyl radical, such as 5-pivaloylamino-pentanoyl, lower alkoxycarbonylamino-lower alkanoyl wherein the amino group is not in the α- or β-position of the lower alkanoyl radical, such as 5-(tert-butoxycarbonylamino)-pentanoyl, phenyl-lower alkoxycarbonylamino that is not the in α- or β-position relative to the bonding carboxy group of the acyl radical, especially in phenyl-lower alkoxycarbonylamino-lower alkanoyl wherein the amino group is not in the α- or β-position of the lower alkanoyl radical, such as 5-benzyloxycarbonylaminopentanoyl or 6-benzyloxycarbonylaminohexanoyl, amino-lower alkanoyl substituted at the amino nitrogen atom by heterocyclyl-lower alkanoyl as defined above for heterocylyl-lower alkanoyl R₁ or R₉, especially by N-morpholino- or N-thiomorpholino-carbonyl, especially by N-morpholino- or N-thiomorpholino-carbonylamino-lower alkanoyl, such as N-morpholino- or N-thiomorpholinocarbonylamino-acetyl, halo-lower alkanoyl containing up to 3 halogen atoms, for example α-haloacetyl, such as α-fluoro-, α-chloro-, α-bromo-, α-iodo-, α,α,α-trifluoro- or α,α,α-trichloro-acetyl, or halopropionyl, such as β-chloro- or β-bromo-propionyl, carboxy-lower alkanoyl, for example carboxyacetyl or β-carboxypropionyl, lower alkoxycarbonyl-lower alkanoyl, for example lower alkoxycarbonylacetyl or lower alkoxycarbonylpropionyl, such as methoxycarbonylacetyl, 3-methoxycarbonylpropionyl, ethoxycarbonylacetyl, 3-ethoxycarbonylpropionyl or 3-tert-butoxycarbonylpropionyl, sulfonyl-lower alkanoyl, such as 3-sulfonylpropionyl, carbamoyl-lower alkanoyl, such as carbamoylacetyl or 3-carbamoylpropionyl, alkylcarbamoyl, especially in lower alkylcarbamoyl-lower alkanoyl, for example lower alkylcarbamoylacetyl or methylcarbamoyl-lower alkanoyl, such as methylcarbamoylacetyl, di-lower alkylcarbamoyl-lower alkanoyl, for example di-lower alkylcarbamoylacetyl or dimethylcarbamoyl-lower alkanoyl, such as dimethylcarbamoylacetyl, carbamoyl-lower alkanoyl substituted at the nitrogen atom by a radical selected from ethylene, trimethylene, tetramethylene and pentamethylene wherein a carbon atom may have been replaced by nitrogen, oxygen, sulfur or by sulfur mono- or di-substituted by oxygen, especially in correspondingly N-substituted carbamoyl-lower alkanoyl, it also being possible for the radical so formed to be fully or partially unsaturated, for example in the form of piperidino-, pyrazin-1-yl-, piperazin-1-yl-, pyrimidin-1-yl-, pyridazin-1-yl-, morpholino-, thiomorpholino- or S,S-dioxothiomorpholino-carbonyl-lower alkanoyl, such as in morpholinocarbonyl-acetyl, 3-(morpholinocarbonyl)-propionyl or 3-(morpholinocarbonyl)-2-isobutyl-propionyl, N-heterocyclyl-lower alkylcarbamoyl-lower alkanoyl or N-lower alkyl-N-heterocyclyl-lower alkylcarbamoyl-lower alkanoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and thiomorpholinyl, such as N-methyl-2-(N-2-pyridylmethyl)-carbamoylacetyl, 2-(N-morpholino-lower alkylcarbamoyl)-lower alkanoyl, such as 2(R,S)-(N-(2-morpholinoethyl)-carbamoyl)-3-methyl-butyryl, or 2-(N-(pyridyl-lower alkyl)-carbamoyl)-lower alkanoyl, such as (2(R,S)-(N-(2-pyridylmethyl)-carbamoyl)-3-methyl)-butyryl, oxo-lower alkanoyl, such as acetoacetyl or propionylacetyl, and cyano-lower alkanoyl, such as cyanoacetyl, 2- or 3-cyanopropionyl or 2-, 3- or 4-cyanobutyryl; lower alkenoyl having from 3 to 7 carbon atoms, preferably having 3 or 4 carbon atoms, such as acryloyl, vinylacetyl, crotonoyl or 3- or 4-pentenoyl, or lower alkynoyl having from 3 to 7, preferably 3 or 4, carbon atoms, for example propioloyl or 2- or 3-butynoyl.

Preferred acyl groups R₁ or R₉ of a semiester of carbonic acid are lower alkoxycarbonyl, for example methoxy-, ethoxy-, n-propoxy-, isopropoxy-, isobutoxy- or tert-lower alkoxy-carbonyl, or also or especially n-propoxycarbonyl, such as tert-butoxycarbonyl or isobutoxycarbonyl, 2-halo-lower alkoxycarbonyl, such as 2-chloro-, 2-bromo-, 2-iodo- or 2,2,2-trichloroethoxycarbonyl, aryl-lower alkoxycarbonyl, for example arylmethoxy-carbonyl, wherein aryl has from 6 to 14 carbon atoms and is, for example, phenyl, biphenylyl, 1- or 2-naphthyl, fluorenyl, or phenyl mono- or poly-substituted by lower alkyl, for example methyl or tert-butyl, hydroxy, lower alkoxy, for example methoxy, ethoxy or tert-butoxy, halogen, for example chlorine or bromine, and/or by nitro, for example phenyl-lower alkoxycarbonyl, such as benzyloxycarbonyl or 4-methoxybenzyloxycarbonyl, heterocyclyl-lower alkoxycarbonyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and from thiomorpholinyl and may be unsubstituted or substituted, especially by lower alkyl, such as methyl, such as furylmethoxycarbonyl, tetrahydrofuryl-lower alkoxycarbonyl, such as 2-tetrahydrofuryl-methoxycarbonyl, 2-morpholino-ethoxycarbonyl, or 2-, 3- or 4-pyridylmethoxycarbonyl, lower alkenyloxycarbonyl wherein preferably the lower alkenyl radical is bonded to the bonding oxygen atom via a saturated carbon atom, such as allyloxycarbonyl, lower alkoxy-lower alkoxycarbonyl, such as 2-methoxyethoxycarbonyl, or (lower alkoxy-lower alkoxy)-lower alkoxycarbonyl, such as 2-(2-methoxyethoxy)ethoxycarbonyl.

Preferred acyl groups R₁ or R₉ of an unsubstituted or substituted carbamic acid, are carbamoyl or unsubstituted or substituted N-alkyl- or N,N-dialkyl-carbamoyl wherein the alkyl radical has up to 12 carbon atoms, preferably unsubstituted or substituted lower alkyl- or di-lower alkyl-carbamoyl, such as methyl-, ethyl-, propyl-, tert-butyl-, dimethyl-, diethyl- or di-n-propyl-carbamoyl, the substituents being selected from phenyl, for example in benzylcarbamoyl, N-phenyl-lower alkyl-N-lower alkylcarbamoyl, such as N-benzyl-N-methylcarbamoyl, or dibenzylcarbamoyl, heterocyclyl that is independently as defined as a substituent of lower alkanoyl R₁ and R₉, preferably pyridyl, such as 2-, 3- or 4-pyridyl, more especially in N-heterocyclyl-lower alkyl-N-lower alkylcarbamoyl, for example N-pyridyl-lower alkyl-N-lower alkylcarbamoyl, such as N-(2-, 3- or 4-pyridylmethyl)-N-methyl-carbamoyl, or in N-heterocyclyl-lower alkylcarbamoyl, for example 2- or 3-pyridyl-lower alkylaminocarbonyl, such as 2- or 3-pyridylmethylaminocarbonyl, hydroxy, for example in, hydroxymethyl, 2-hydroxyethyl, 3-hydroxypropyl, 2-hydroxypropyl, and lower alkoxy, preferably in lower alkoxy-lower alkyl, for example methoxymethyl or 2-methoxyethyl; or also or especially N-lower alkyl-N-morpholino-lower alkyl-aminocarbonyl, such as N-methyl-N-(2-morpholinoethyl)aminocarbonyl, or N-morpholino-lower alkylaminocarbonyl, such as N-(2-morpholinoethyl)aminocarbonyl.

Preferred acyl groups R₁ or R₉ of an unsubstituted or substituted amino acid are formed by the amino acid residues, bonded via the carbonyl of their carboxy group, of an α- or β-amino acid, especially a natural α-amino acid having the L-configuration, such as those normally occurring in proteins, or an epimer of such an amino acid, that is to say having the unnatural D-configuration, or a D,L-isomeric mixture thereof, a homologue of such an amino acid, for example wherein the amino acid side chain has been lengthened or shortened by one or two methylene groups, wherein the amino group is in the β-position and/or wherein a methyl group has been replaced by hydrogen, a substituted aromatic amino acid wherein the aromatic radical has from 6 to 14 carbon atoms, for example a substituted phenylalanine or phenylglycine wherein the phenyl may be mono- or poly-substituted by lower alkyl, for example methyl, hydroxy, lower alkoxy, for example methoxy, lower alkanoyloxy, for example acetoxy, amino, lower alkylamino, for example methylamino, di-lower alkylamino, for example dimethylamino, lower alkanoylamino, for example acetylamino or pivaloylamino, lower alkoxycarbonylamino, for example tert-butoxycarbonylamino, arylmethoxycarbonylamino wherein aryl preferably has from 6 to 14 carbon atoms, for example benzyloxycarbonylamino or 9-fluorenylmethoxycarbonylamino, halogen, for example fluorine, chlorine, bromine or iodine, carboxy and/or by nitro, a benzo-fused phenylalanine or phenylglycine, such as α-naphthylalanine, or a hydrogenated phenylalanine or phenylglycine, such as cyclohexylalanine or cyclohexylglycine.

Those amino acids can be substituted at free amino or hydroxy functions, preferably at a free amino function, by one of the radicals mentioned above under acyl R₁ or R₉ as the acyl group of a carboxylic acid, a semiester of carbonic acid or an unsubstituted or N-substituted carbamic acid or by one of the radicals mentioned below under unsubstituted or substituted alkyl R₁, R₂, R₈ or R₉.

Especially preferred is the radical, bonded via its carbonyl group, of an amino acid selected from glycine (H-Gly-OH), alanine (H-Ala-OH), valine (H-Val-OH), norvaline (α-aminovaleric acid), leucine (H-Leu OH), isoleucine (H-Ile-OH), norleucine (α-aminohexanoic acid, H-Nle-OH), serine (H-Ser-OH), homoserine (α-amino-γ-hydroxybutyric acid), threonine (H-Thr-OH), methionine (H-Met-OH), cysteine (H-Cys-OH), proline (H-Pro-OH), trans-3- and trans-4-hydroxyproline, phenylalanine (H-Phe-OH), tyrosine (H-Tyr-OH), 4-aminophenylalanine, 4-chlorophenylalanine, 4-carboxyphenylalanine, β-phenylserine (β-hydroxyphenylalanine), phenylglycine, α-naphthylalanine (H-Nal-OH), cyclohexylalanine (H-Cha-OH), cyclohexylglycine, tryptophan (H-Trp-OH), indoline-2-carboxylic acid, 1,2,3,4-tetrahydroisoquinoline-3-carboxylic acid, aspartic acid (H-Asp-OH), asparagine (H-Asn-OH), aminomalonic acid, aminomalonic acid monoamide, glutamic acid (H-Glu-OH), glutamine (H-Gln-OH), histidine (H-His-OH), arginine (H-Arg-OH), lysine (H-Lys-OH), .delta.-hydroxylysine, ornithine (α, .delta.-diaminovaleric acid), 3-aminopropanoic acid, α,γ-diaminobutyric acid and α,β-diaminopropionic acid, especially preferably the radical of an aliphatic amino acid selected from valine, alanine, leucine and isoleucine, or an amino acid selected from glycine, glutamic acid and asparagine, it being possible for each of the mentioned amino acids (with the exception of glycine) to be in the D-, L- or (D,L)-form, preferably in the L-form (with the exception of Val which may also be in the (D)- or (D,L)-form), wherein the α-amino group may be unsubstituted or mono- or di-N-alkylated, for example by lower alkyl, such as methyl or n-propyl, or by amino-lower alkyl, such as 3-aminopropyl, or may be N-acylated by one of the acyl radicals mentioned above under acyl R₁ as a radical of a carboxylic acid, of a semiester of carbonic acid or of an unsubstituted or N-substituted carbamic acid, preferably by lower alkanoyl, such as acetyl; by aryl-lower alkanoyl wherein aryl is selected from phenyl, indenyl, indanyl, naphthyl, anthryl, phenanthryl, acenaphthyl and fluorenyl and may be unsubstituted or mono- to tri-substituted especially by lower alkyl, for example methyl, ethyl or propyl, halo-lower alkyl, for example trifluoromethyl, phenyl, 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, and wherein lower alkanoyl may be unsubstituted or substituted especially by carboxy, lower alkoxycarbonyl, for example methoxy-, ethoxy-, n-propoxy-, isopropoxy-, n-butoxy-, isobutoxy-, sec-butoxy-, tert-butoxy-, n-pentyloxy-, isopentyloxy-, neopentyloxy-, tert-pentyloxy-, n-hexyloxy-, isohexyloxy- or n-heptyloxy-carbonyl, aryl-lower alkoxycarbonyl wherein aryl has from 6 to 12 carbon atoms, for example benzyloxycarbonyl, carbamoyl, carbamoyl substituted by one or two radicals selected from lower alkyl, such as methyl, ethyl, propyl, isopropyl, n-butyl, isobutyl, sec-butyl, tert-butyl, n-pentyl, isopentyl, neopentyl, tert-pentyl, n-hexyl, isohexyl or n-heptyl, for example in N-methylcarbamoyl, N-n-butylcarbamoyl or N,N-dimethylcarbamoyl, from carboxy-lower alkyl or lower alkoxycarbonyl-lower alkyl, for example in the form of carboxymethylcarbamoyl (glycinylcarbonyl) or tert-butoxycarbonylmethylcarbamoyl, from di-lower alkylamino-lower alkyl, for example 2-dimethylaminoethyl, from hydroxy-lower alkyl, for example hydroxymethyl or hydroxyethyl, and from di-lower alkoxy-lower alkyl, for example 2-(2,2-dimethoxyethyl), and/or by cyano and is unbranched or branched, with phenyl-lower alkanoyl, such as phenylacetyl, or phenyl-lower alkanoyl wherein the lower alkanoyl radical is substituted by carbamoyl, such as 2-carbamoyl-3-phenyl-propionyl, being especially preferred; by heterocyclyl-lower alkanoyl wherein heterocyclyl is independently as defined as a substituent of lower alkanoyl R₁ and is especially morpholino, thiomorpholino, pyridyl, quinolyl or tetrazolyl, more especially pyridylcarbonyl, such as 2-, 3- or 4-pyridylcarbonyl, morpholinocarbonyl, thiomorpholinocarbonyl, S,S-dioxothiomorpholinocarbonyl, indol-2-ylcarbonyl, quinolin-2-ylcarbonyl, pyridylacetyl, -such as 2- or 3-pyridylacetyl, imidazolylacetyl, such as imidazol-1-ylacetyl, morpholinylacetyl, such as morpholinoacetyl, pyridylpropionyl, such as 3-(2- or 3-pyridyl)propionyl, pyrrolidinylpropionyl, such as 3-(4-pyrrolidinyl)propionyl, morpholinylpropionyl, such as 3-morpholinopropionyl, or tetrazolylpropionyl, such as 3-(tetrazol-1-yl)-propionyl; by halo-lower alkanoyl containing up to 3 halogen atoms, for example α-haloacetyl, such as α-fluoro-, α-chloro-, α-bromo-, α-iodo-, ααα-trifluoro- or α,α,α-trichloro-acetyl, or halopropionyl, such as β-chloro- or β-bromo-propionyl; by lower alkoxy-lower alkoxy-lower alkanoyl; by lower alkoxycarbonyl, such as tert-butoxycarbonyl; by aryl-lower alkoxycarbonyl wherein aryl has from 6 to 14 carbon atoms and is selected, for example, from phenyl, naphthyl and fluorenyl, for example phenyl-lower alkoxycarbonyl, such as benzyloxycarbonyl, or 9-fluorenylmethoxycarbonyl; by heterocyclyl-lower alkoxycarbonyl wherein heterocyclyl is selected especially from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated and unsubstituted or substituted especially by lower alkyl, such as methyl, for example 2-furylmethoxycarbonyl, tetrahydrofuryl-lower alkoxycarbonyl, such as 2-tetrahydrofuryl-methoxycarbonyl, or 2-morpholino-ethoxycarbonyl; by lower alkenyloxycarbonyl (preferably with a saturated carbon atom at the bonding oxygen atom), such as allyloxycarbonyl; by lower alkoxy-lower alkoxycarbonyl, such as 2-methoxyethoxycarbonyl; by (lower alkoxy-lower alkoxy)-lower alkoxycarbonyl, such as 2-(2-methoxyethoxy)ethoxycarbonyl; by carboxy-lower alkanoyl, such as 3-carboxypropionyl; by lower alkoxycarbonyl-lower alkanoyl; by amino-lower alkanoyl substituted at the amino nitrogen atom by heterocyclyl-lower alkanoyl wherein heterocyclyl is preferably independently as defined above as a substituent of lower alkanoyl R₁ or R₉, especially by N-morpholino- or N-thiomorpholino-carbonyl, for example N-morpholino- or N-thiomorpholino-carbonylamino-lower alkanoyl, such as N-morpholino- or N-thiomorpholino-carbonylamino-acetyl; by carbamoyl; by phenyl-lower alkylaminocarbonyl, such as benzylaminocarbonyl; by N-heterocyclyl-lower alkyl-N-lower alkylcarbamoyl or N-heterocyclyl-lower alkylcarbamoyl wherein heterocyclyl is independently as defined above as a substituent of lower alkanoyl R₁ or R₉, especially as pyridyl, such as 2-, 3- or 4-pyridyl, especially 2- or 3-pyridyl-lower alkylaminocarbonyl, such as 2- or 3-pyridylmethylaminocarbonyl; or by N-2-, N-3- or N-4-pyridyl-lower alkyl-N-lower alkylaminocarbonyl, such as N-2-, N-3- or N-4-pyridylmethyl-N-methylaminocarbonyl; by heterocyclyl-lower alkylcarbamoyl-lower alkanoyl wherein heterocyclyl is independently as defined in the definition thereof as a substituent of lower alkyl R₁, R₂, R₈ or R₉, for example 2-(N-morpholino-lower alkylcarbamoyl)-lower alkanoyl, such as 2(R,S)-(N-(2-morpholinoethyl)-carbamoyl)-3-methyl-butyryl, or 2-(N-(pyridyl-lower alkyl)carbamoyl)-lower alkanoyl, such as (2(R,S)-(N-(2-pyridylmethyl)-carbamoyl)-3-methyl)butyryl; by sulfonyl; by lower alkanesulfonyl, such as methane- or ethane-sulfonyl; by arylsulfonyl (aryl-SO₂) wherein aryl has from 6 to 10 carbon atoms and, for example, is selected from phenyl and naphthyl and is unsubstituted or especially substituted by lower alkyl, such as methyl, or by lower alkoxy, such as methoxy, such as p-toluenesulfonyl; or by heterocyclylsulfonyl (heterocyclyl-SO₂—) wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and from thiomorpholinyl and may be unsubstituted or substituted especially by lower alkyl, such as methyl, such as morpholinosulfonyl, thiomorpholinosulfonyl, piperidinosulfonyl or piperazinosulfonyl (heterocyclylsulfonyl not being a substituent in preferred forms); or is acylated also or especially by lower alkylaminocarbonyl, such as tert-butylaminocarbonyl, N,N-di-lower alkylaminocarbonyl, such as N,N-dimethylaminocarbonyl, lower alkoxy-lower alkylaminocarbonyl, such as N-(2-methoxyethyl)aminocarbonyl, N-lower alkyl-N-morpholino-lower alkylaminocarbonyl, such as N-methyl-N-(2-morpholinoethyl)aminocarbonyl, or N-morpholino-lower alkylaminocarbonyl; and/or a hydroxy group of the side chain is present in etherified or esterified form, for example in the form of lower alkoxy, such as methoxy or tert-butoxy, aryl-lower alkoxy, such as benzyloxy, lower alkanoyloxy, such as acetoxy, or lower alkoxycarbonyloxy, for example tert-butoxycarbonyloxy.

Special preference is given to acyl groups R₁ or R₉, bonded via the carbonyl group of their carboxy function, of an unsubstituted or substituted amino acid selected from phenylalanine, N-(benzyloxycarbonyl)-phenylalanine, tyrosine, tyrosine-O-methyl ether, N-morpholinocarbonyl-glycine, N-(N-(2-, 3- or 4-pyridyl)methyl-N-methylaminocarbonyl)glycine, valine, N-(trifluoroacetyl)-valine, N-phenylacetyl-valine, N-acetyl-vale, N-(3-phenylpropionyl)-valine, N-(2-carbamoyl-3-phenyl-propionyl)-valine, such as N-(2(R,S)carbamoyl-3-phenyl-propionyl)-valine, N-(2- or 3-pyridylacetyl)-valine, N-tetrahydrofurylmethoxycarbonyl-valine, N-(2-methoxy)ethoxycarbonylvaline, N-3-(tetrazol-1-yl)propionyl-valine, N-(indol-2-ylcarbonyl)-valine, N-(quinolin-2-ylcarbonyl)-valine, N-methoxycarbonyl-valine, N-ethoxycarbonyl-valine, N-isobutoxycarbonyl-valine, N-tert-butoxycarbonyl-valine, N-benzyloxycarbonyl-valine, N-(2-furylmethoxycarbonyl)-valine, N-allyloxycarbonyl-valine, N-(morpholinocarbonyl)-valine, N-(thiomorpholinocarbonyl)-valine, N—(S,S-dioxothiomorpholinocarbonyl)-valine, N-(N-2-pyridylmethyl-N-methylaminocarbonyl)-valine, N-(N-3-pyridylmethyl-aminocarbonyl)-valine, N-(N-2-pyridylmethyl-aminocarbonyl)-valine, N-morpholino-carbonylaminoacetyl-valine, N-methanesulfonyl-valine, N-morpholinosulfonyl-valine, N-acetyl-leucine, N-(4-thiomorpholinocarbonyl)-leucine, N-(4-(S,S-dioxothiomorpholino)carbonyl)-leucine, N-(benzyloxycarbonyl)-leucine, N-acetyl-isoleucine, N-propionyl-isoleucine, N-(benzyloxycarbonyl)-isoleucine, N-(tert-butoxycarbonyl)-norleucine, N-benzyloxycarbonyl-glutamic acid, asparagine, glutamine, N-benzyloxycarbonyl-asparagine, quinolin-2-ylcarbonyl-asparagine and N-(morpholinocarbonyl)-asparagine; or also or especially the corresponding radicals of N-(3,3-dimethylbutyryl)-valine, N-(n-propoxycarbonyl)-valine, N-(2-(2-methoxyethoxy)ethoxycarbonyl)-valine, N-(2-methoxyethoxyacetyl)-valine, N-(N,N-dimethylaminocarbonyl)-valine, N-(N-(2-methoxyethyl)amino)-valine, N-(benzylaminocarbonyl)-valine, N-(2-morpholinoethylaminocarbonyl)-valine or N-(N-methyl-N-(2-morpholinoethyl)aminocarbonyl)-valine; the amino acid residues preferably being in the (L)- or (D,L)-form, and in the case of valine also in the (D)-form.

It should be mentioned that on all levels of definitions those compounds of formula I-A are to be regarded as especially preferred wherein R₉ corresponds to the acyl moiety of an α-amino acid that is bonded via the carbonyl group of its carboxy function and that is acylated on its α-amino nitrogen by an acyl group of a carboxylic acid, a semiester of carbonic acid or an unsubstituted or N-substituted carbamic acid, as defined above for R₁ or R₉.

Unsubstituted or substituted alkyl R₁, R₂, R₈ or R₉ is an alkyl radical having from 1 to 20, preferably up to 10, carbon atoms, is branched or unbranched, and is, for example, methyl, ethyl, n-propyl, isopropyl, n-butyl, isobutyl, sec-butyl, tert-butyl, n-pentyl, isopentyl, neopentyl, tert-pentyl, n-hexyl, isohexyl, n-heptyl, n-octyl, n-nonyl or n-decyl. Preference is given to lower alkyl, for example methyl, ethyl, n-propyl, isopropyl, n-butyl, isobutyl, sec-butyl, tert-butyl, n-pentyl, isopentyl, neopentyl, tert-pentyl, n-heptyl, isohexyl or n-heptyl, which is unsubstituted or substituted.

Radicals suitable as substituents in substituted alkyl R₁, R₂, R₈ or R₉, preferably substituted lower alkyl, are the radicals mentioned for lower alkanoyl R₁ and R₉.

Preference is given especially to unsubstituted lower alkyl, such as methyl, ethyl, propyl, isopropyl, n-butyl, isobutyl or tert-butyl.

Alkyl-substituted sulfonyl R₁ or R₉ (alkyl-SO₂—) preferably contains an unsubstituted or substituted alkyl radical mentioned under alkyl R₁, R₂, R₈ and R₉ and is especially lower alkanesulfonyl, such as methanesulfonyl, ethanesulfonyl, n-propanesulfonyl or S-tert-butylsulfonyl, or aryl-lower alkyl-substituted sulfonyl (aryl-lower alkyl-SO₂—) that contains, for example, an unsubstituted or substituted aryl radical as defined for aryl-substituted lower alkyl R₁, R₂, R₈ and R₉ and is selected especially from phenylmethane-, 4-chloro-phenylmethane-4-methoxy-phenylmethane- or 4-nitro-phenylmethane-, naphthylmethane-, for example α- or β-naphthylmethane-, 2-phenylethane-, 2-α-naphthylethane-, 2-β-naphthylethane-, 2-(4-methylphenyl)ethane-, 2-(4-methoxyphenyl)ethane-, 3-phenylpropane-, 3-(p-hydroxyphenyl)-propane-, 2,2-diphenylethane- and 2,2-di(4-methoxyphenyl)ethanesulfonyl.

Aryl-substituted sulfonyl R₁ or R₉ (aryl-SO₂—) preferably contains an unsubstituted or substituted aryl radical mentioned in the definition of aryl as a substituent of lower alkanoyl R₁ or R₉ and is especially benzene- or 1- or 2-naphthalene-sulfonyl that is unsubstituted or mono- or di-substituted by lower alkyl, such as benzenesulfonyl, 2- or 4-toluenesulfonyl or 1- or 2-naphthalenesulfonyl.

Heterocyclyl-substituted sulfonyl R₁ or R₉ (heterocyclyl-SO₂—) preferably contains heterocyclyl that is selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and from thiomorpholinyl and may be unsubstituted or substituted, especially by lower alkyl, such as methyl, such as morpholinosulfonyl, thiomorpholinosulfonyl, piperidinosulfonyl or piperazinosulfonyl. In especially preferred forms of the invention, heterocyclylsulfonyl as substituent may be absent.

Acyloxy R₅ has, for example, up to 25, preferably up to 19, carbon atoms and is especially the acyloxy group, bonded via its carbonyl to the bonding oxygen atom, of a carboxylic acid or of an unsubstituted or substituted amino acid, also an aminocarbonyloxy group, an N-substituted aminocarbonyloxy group or an acyl radical of a semiester of carbonic acid linked via its carbonyl group to the bonding oxygen atom.

A preferred acyloxy group R₅ of a carboxylic acid is, for example, unsubstituted C₁-C₂O alkanoyloxy, for example n-decanoyloxy or palmitoyloxy, C₃-C₂O alkenoyloxy or C₃-C₂O alkynoyloxy, or substituted C₁-C₂O alkanoyloxy, C₃-C₂O alkenoyloxy or C₃-C₂O alkynoyloxy, especially lower alkanoyloxy, octanoyloxy, nonanoyloxy, decanoyloxy, undecanoyloxy, dodecanoyloxy or also or especially palmitoyloxy; C₃-C₇ alkenoyloxy; or C₃-C₇ alkynoyloxy; or substituted lower alkanoyloxy, wherein the substituents are selected, for example, from one or more radicals, preferably from up to three radicals, especially from one radical selected from the group consisting of hydroxy, lower alkoxy, phenoxy, naphthyloxy, lower alkanoyloxy, phenyl-lower alkanoyloxy, such as benzoyloxy or phenylacetoxy, halogen, such as fluorine, chlorine, bromine or iodine, especially fluorine or chlorine, carboxy, lower alkoxycarbonyl, phenyl-lower alkoxycarbonyl, such as benzyloxycarbonyl, carbamoyl, lower alkylcarbamoyl, hydroxy-lower alkylcarbamoyl, di-lower alkylcarbamoyl, bis(hydroxy-lower alkyl)carbamoyl, cyano, oxo, C₃-C₈ cycloalkyl, such as cyclopropyl, cyclobutyl, cyclopentyl or cyclohexyl, C₆-C₁₂ bicycloalkyl, such as decahydronaphth-2-yl, C₉-C₁₄ tricycloalkyl, such as 1- or 2-adamantyl, C₄-C₈ cycloalkenyl, such as 1-cyclohexenyl or 1,4-cyclohexadienyl, heterocyclyl which is preferably a saturated, partially saturated or unsaturated single ring containing from 3 to 7, preferably from 5 to 7, ring atoms and up to four hetero atoms selected from nitrogen, sulfur and oxygen, preferably 1 or 2 of the mentioned hetero atoms, the ring either being present as such or being once or twice, preferably once, benzofused, cyclopenta-, cyclohexa- or cyclohepta-fused, and which may be unsubstituted or substituted especially by lower alkyl, lower alkanoyl, hydroxy, lower alkoxy, phenyl-lower alkoxy, such as benzyloxy, hydroxy-lower alkyl, such as hydroxymethyl, halogen, cyano and/or by trifluoromethyl, for example pyrrolyl, 2,5-dihydropyrrolyl, furyl, thienyl, tetrahydrofuryl, cyclohepta[b]pyrrolyl, pyrrolidinyl, imidazolyl, imidazolidinyl, pyrazolinyl, pyrazolidinyl, triazolyl, such as 1,2,3-, 1,2,4- or 1,3,4-triazolyl, tetrazolyl, such as 1- or 2-tetrazolyl, tetrahydro-oxazolyl, tetrahydroisoxazolyl, tetrahydro-thiazolyl, tetrahydro-isothiazolyl, indolyl, isoindolyl, quinolyl, isoquinolyl, benzimidazolyl, benzofuranyl, pyridyl, pyrimidinyl, piperidinyl, piperazin-1-yl, morpholino, thiomorpholino, S,S-dioxothiomorpholino, 1,2-dihydro- or 1,2,3,4-tetrahydroquinolyl, or 1,2-dihydro- or 1,2,3,4-tetrahydro-isoquinolyl, the mentioned radicals being unsubstituted or substituted as above, especially by lower alkyl, for example in 4-lower alkyl-piperazin-1-yl, such as 4-methyl- or 4-ethyl-piperazin-1-yl, by lower alkanoyl, for example in 4-lower alkanoyl-piperazin-1-yl, such as 4-acetylpiperazin-1-yl, or by hydroxy-lower alkyl, for example in 5-hydroxymethylfuran-2-yl-carbonyl; and aryl, preferably C₆-C₁4 aryl, for example phenyl, naphthyl, such as 1- or 2-naphthyl, or fluorenyl, such as fluoren-9-yl, aryl being unsubstituted or mono- or poly-substituted, preferably mono-substituted, for example, by lower alkyl, for example methyl, halo-lower alkyl, such as trifluoromethyl or chloro- or bromo-methyl, halogen, for example fluorine or chlorine, hydroxy, lower alkoxy, such as methoxy, lower alkanoyloxy, carboxy, lower alkoxycarbonyl, phenyl-lower alkoxycarbonyl, carbamoyl, mono- or di-lower alkylcarbamoyl, mono- or di-hydroxy-lower alkylcarbamoyl, such as heterocyclyl-lower alkyl wherein heterocyclyl is as defined above as a substituent of lower alkanoyloxy R₅, especially heterocyclylmethyl wherein heterocyclyl is bonded via a ring nitrogen atom, for example piperidinomethyl, piperazin-1-ylmethyl, 4-lower alkyl-piperazin-1-ylmethyl, such as 4-methyl- or 4-ethyl-piperazin-1-ylmethyl, 4-lower alkanoyl-piperazin-1-ylmethyl, such as 4-acetyl-piperazin-1-ylmethyl, morpholinomethyl or thiomorpholinomethyl, cyano and/or by nitro, and especially phenyl substituted in the p-position by one of the mentioned radicals.

For example lower alkanoyloxy, such as formyloxy, acetoxy, propionyloxy, pivaloyloxy or heptanoyloxy, such as n-heptanoyloxy, hydroxy-lower alkanoyloxy, for example β-hydroxypropionyloxy, lower alkoxy-lower alkanoyloxy, for example lower alkoxyacetoxy or lower alkoxypropionyloxy, such as methoxyacetoxy or β-methoxypropionyloxy, lower alkanoyloxy-lower alkanoyloxy, for example lower alkanoyloxyacetoxy or lower alkanoyloxypropionyloxy, such as acetoxyacetoxy or β-acetoxypropionyloxy, halo-lower alkanoyloxy, for example α-haloacetoxy, such as α-chloro-, α-bromo-, α-iodo-, α,α,α-trifluoro- or α,α,α-trichloro-acetoxy, or halopropionyloxy, such as β-chloro- or β-bromo-propionyloxy, carboxy-lower alkanoyloxy, for example carboxyacetoxy or 3-carboxypropionyloxy, lower alkoxycarbonyl-lower alkanoyloxy, for example lower alkoxycarbonylacetoxy or lower alkoxycarbonylpropionyloxy, such as methoxycarbonylacetoxy, β-methoxycarbonylpropionyloxy, ethoxycarbonylacetoxy, β-ethoxycarbonylpropionyloxy, tert-butoxycarbonylacetoxy or β-tert-butoxycarbonylpropionyloxy, carbamoyl-lower alkanoyloxy, for example carbamoylacetoxy or β-carbamoylpropionyloxy, lower alkylcarbamoyl-lower alkanoyloxy, di-lower alkylcarbamoyl-lower alkanoyloxy, hydroxy-carboxy-lower alkanoyloxy, hydroxy-lower alkoxycarbonyl-lower alkanoyloxy, dihydroxy-carboxy-lower alkanoyloxy, dihydroxy-lower alkoxycarbonyl-lower alkanoyloxy, heterocyclyl-lower alkanoyloxy, for example pyrrolylcarbonyloxy, such as 2- or 3-pyrrolylcarbonyloxy, furylcarbonyloxy, for example 2-furylcarbonyloxy, 5-hydroxymethyl-furan-2-ylcarbonyloxy, thienylcarbonyloxy, for example 2-thienylcarbonyloxy, imidazolylcarbonyloxy, such as 4-imidazolylcarbonyloxy, imidazolylacetoxy, such as 4-imidazolylacetoxy, imidazolylpropionyloxy, such as 3-(4-imidazolyl)propionyloxy, pyridylcarbonyloxy, for example 2-, 3- or 4-pyridylcarbonyloxy, indolylcarbonyloxy, for example 2-, 3- or 5-indolylcarbonyloxy, 1-methyl-, 5-methyl-, 5-methoxy-, 5-benzyloxy-, 5-chloro- or 4,5-dimethyl-indolyl-2-carbonyloxy, quinolylcarbonyloxy, such as quinolin-2-ylcarbonyloxy, pyrrolidinylcarboxy, such as pyrrolidinyl-3-carbonyloxy, piperidinylcarbonyloxy, for example 2-, 3- or 4-piperidinylcarbonyloxy, morpholinocarbonyloxy, thiomorpholino-carbonyloxy, morpholinoacetoxy, thiomorpholinoacetoxy, or 4-lower alkyl-1-piperazinoacetoxy, such as 4-methylpiperazino-acetoxy, lower alkenoyloxy, for example acryloyloxy, vinylacetoxy, crotonoyloxy or 3- or 4-pentenoyloxy, lower alkynoyloxy, for example propioloyloxy or 2- or 3-butynoyloxy, C₃-C₈ cycloalkylcarbonyloxy or C₃-C₈ cycloalkylacetoxy, for example cyclopropyl-, cyclo-butyl-, cyclopentyl- or cyclohexyl-carbonyloxy, cyclopropylacetoxy, cyclopentylacetoxy or cyclohexylacetoxy, phenyl-lower alkanoyloxy, for example benzoyloxy, phenylacetoxy or 3-phenylpropionyloxy, wherein phenyl is unsubstituted or mono- or poly-substituted by lower alkyl, for example methyl, halo-lower alkyl, such as chloro- or bromo-methyl, halogen, for example fluorine or chlorine, hydroxy, lower alkoxy, for example methoxy, piperidinomethyl, piperazin-1-ylmethyl, 4-lower alkyl-piperazin-1-ylmethyl, such as 4-methyl- or 4-ethyl-piperazin-1-ylmethyl, 4-lower alkanoyl-piperazin-1-ylmethyl, such as 4-acetyl-piperazin-1-ylmethyl, morpholino-lower alkyl, such as morpholinomethyl, thiomorpholinomethyl, cyano and/or by nitro, for example 4-chloromethyl-, 4-bromo-methyl-, 4-fluoro-, 4-chloro-, 4-methoxy-, 4-morpholinomethyl-, 4-thiomorpholino-methyl-, 4-cyano- or 4-nitro-benzoyloxy, or lower alkylphenylacetoxy, such as 4-methylphenylacetoxy.

A preferred acyloxy R₅ of an acyl radical, linked via its carbonyl group to the bonding oxygen atom, of a semiester of carbonic acid is, for example, unsubstituted or substituted alkoxycarbonyloxy, especially unsubstituted or substituted lower alkoxycarbonyloxy, for example methoxy-, ethoxy- or tert-lower alkoxy-carbonyloxy, such as tert-butoxycarbonyloxy, 2-halo-lower alkoxycarbonyloxy, for example 2-chloro-, 2-bromo-, 2-iodo- or 2,2,2-trichloro-ethoxycarbonyloxy; aryl-lower alkoxycarbonyloxy, for example arylmethoxycarbonyloxy, wherein aryl preferably has from 6 to 14 carbon atoms, is unsubstituted or mono- or poly-substituted, preferably mono-substituted, for example, by lower alkyl, for example methyl, halo-lower alkyl, such as trifluoromethyl or chloro- or bromo-methyl, halogen, for example fluorine or chlorine, hydroxy, lower alkoxy, such as methoxy, lower alkanoyloxy, carboxy, lower alkoxycarbonyl, phenyl-lower alkoxycarbonyl, carbamoyl, mono- or di-lower alkylcarbamoyl, mono- or di-hydroxy-lower alkylcarbamoyl, heterocyclyl-lower alkyl wherein heterocyclyl is as defined above as a substituent of lower alkanoyloxy R₅, especially heterocyclylmethyl wherein heterocyclyl is bonded via a ring nitrogen atom, for example piperidinomethyl, piperazin-1-ylmethyl, 4-lower alkyl-piperazin-1-ylmethyl, such as 4-methyl- or 4-ethyl-piperazin-1-ylmethyl, 4-lower alkanoyl-piperazin-1-ylmethyl, such as 4-acetyl-piperazin-1-ylmethyl, morpholinomethyl or thiomorpholinomethyl, cyano and/or by nitro, and is especially phenyl, 1- or 2-naphthyl, fluorenyl, or phenyl mono- or poly-substituted by lower alkyl, for example methyl or tert-butyl, lower alkoxy, for example methoxy, ethoxy or tert-butoxy, hydroxy, halogen, for example fluorine, chlorine or bromine, and/or by nitro, for example phenyl-lower alkoxycarbonyloxy, such as benzyloxycarbonyloxy, 4-methoxybenzyloxycarbonyloxy, 4-nitrobenzyloxycarbonyloxy, diphenyl-lower alkoxycarbonyloxy, such as diphenylmethoxycarbonyloxy, di(4-methoxyphenyl)methoxycarbonyloxy, trityloxycarbonyloxy or fluorenyl-lower alkoxycarbonyloxy, such as 9-fluorenylmethoxycarbonyloxy; or heterocyclyl-lower alkoxycarbonyloxy wherein heterocyclyl is as defined above as a substituent of lower alkanoyloxy R₅, for example furan-2-ylmethoxycarbonyloxy or pyridin-2-, -3- or -4-ylmethoxycarbonyl. The definitions falling under the definition of acyloxy groups R₅ of a semiester of carbonic acid may in preferred forms be omitted from all the definitions of compounds of formula I-A mentioned hereinbefore and hereinafter.

A preferred N-substituted aminocarbonyloxy group as acyloxy R₅ carries at the nitrogen atom one or two substituents selected independently of one another from unsubstituted or substituted lower alkyl (the substituents being selected from those mentioned above for substituted lower alkanoyloxy R₅ and being present in the number there-defined, preferably substituents selected from hydroxy, lower alkoxy, lower alkanoyloxy, phenyl-lower alkanoyloxy, such as benzoyloxy or phenylacetoxy, halogen, such as fluorine, chlorine, bromine or iodine, especially fluorine or chlorine, carboxy, lower alkoxycarbonyl, phenyl-lower alkoxycarbonyl, such as benzyloxycarbonyl, cyano, oxo and phenyl or naphthyl, which are unsubstituted or mono- or poly-substituted, preferably mono-substituted, for example, by lower alkyl, for example methyl, halo-lower alkyl, such as trifluoromethyl or chloro- or bromo-methyl, halogen, for example fluorine or chlorine, hydroxy, lower alkoxy, such as methoxy, lower alkanoyloxy, carboxy, lower alkoxycarbonyl, phenyl-lower alkoxycarbonyl, cyano and/or by nitro, especially phenyl substituted in the p-position by one of the mentioned radicals), especially unsubstituted lower alkyl, such as methyl or ethyl, and aryl which preferably has from 6 to 14 carbon atoms and is unsubstituted or mono- or poly-substituted, preferably mono-substituted, for example, by lower alkyl, for example methyl, halo-lower alkyl, such as chloro- or bromo-methyl, halogen, for example fluorine or chlorine, hydroxy, lower alkoxy, such as methoxy, lower alkanoyloxy, carboxy, lower alkoxycarbonyl, phenyl-lower alkoxycarbonyl, halo-lower alkyl, such as trifluoromethyl, cyano and/or by nitro, the nitrogen atom of the carbamoyl group carrying not more than one aryl radical.

An acyloxy group R₅ of an N-substituted carbamic acid is especially mono- or di-lower alkylaminocarbonyloxy, such as N-methyl-, N-ethyl-, N,N-dimethyl- or N,N-diethylaminocarbonyloxy, or phenyl-lower alkylaminocarbonyloxy wherein phenyl is unsubstituted or substituted by lower alkyl, for example methyl, halo-lower alkyl, such as chloro- or bromo-methyl or trifluoromethyl, halogen, for example fluorine or chlorine, hydroxy, lower alkoxy, such as methoxy, carboxy and/or by cyano, preferably by up to three of those substituents selected independently of one another, especially by one of those substituents, for example in the p-position, such as in N-benzyl-, N-(4-fluorobenzyl)-, N-(4-chlorobenzyl)-, N-(4-trifluoromethylbenzyl)- or N-(4-cyanobenzyl)-aminocarbonyloxy; especially preferred is aminocarbonyloxy substituted by only one radical at the nitrogen atom, for example N-lower alkylaminocarbonyloxy, such as N-methyl- or N-ethyl-aminocarbonyloxy, or phenyl-lower alkylamino-carbonyloxy wherein phenyl is unsubstituted or substituted by lower alkyl, such as methyl, halo-lower alkyl, such as chloro- or bromo-methyl or trifluoromethyl, halogen, such as fluorine or chlorine, hydroxy, lower alkoxy, such as methoxy, carboxy and/or by cyano, preferably by up to three of those substituents selected independently of one another, especially by one of those substituents, for example in the p-position, such as in N-benzyl-, N-(4-fluorobenzyl)-, N-(4-chlorobenzyl)-, N-(4-trifluoromethylbenzyl)- or N-(4-cyanobenzyl)-aminocarbonyloxy. The definitions falling under the definition of acyloxy groups R₅ of an N-substituted carbamic acid, and the radical aminocarbonyloxy R₅ may in preferred forms be omitted from all the definitions of compounds of formula I-A mentioned hereinbefore and hereinafter.

An unsubstituted or substituted amino acid in acyloxy R₅ bonded via its carbonyl to the bonding oxygen atom is preferably formed by the amino acid residues (aminoacyloxy), bonded via the carbonyl of their carboxy group and an oxygen atom, of an α-, β-, γ- or δ-amino acid, especially of a natural α-amino acid having the L-configuration, such as those normally occurring in proteins, or an epimer of such an amino acid, that is to say having the unnatural D-configuration, or a D,L-isomeric mixture thereof, a homologue of such an amino acid, for example wherein the amino acid side chain has been lengthened or shortened by one or two methylene groups, wherein the amino group is in the β-, γ- or δ-position and/or wherein a methyl group has been replaced by hydrogen, a substituted aromatic amino acid wherein the aromatic radical has from 6 to 14 carbon atoms, for example a substituted phenylalanine or phenylglycine wherein the phenyl may be mono- or poly-substituted by lower alkyl, for example methyl, hydroxy, lower alkoxy, for example methoxy, lower alkanoyloxy, for example acetoxy, amino, lower alkylamino, for example methylamino, di-lower alkylamino, for example dimethylamino, lower alkanoylamino, for example acetylamino or pivaloylamino, lower alkoxycarbonylamino, for example tert-butoxycarbonylamino, arylmethoxycarbonylamino wherein aryl preferably has from 6 to 14 carbon atoms, for example benzyloxycarbonylamino or 9-fluorenylmethoxycarbonylamino, halogen, for example fluorine, chlorine, bromine or iodine, carboxy and/or by nitro, a benzo-fused phenylalanine or phenylglycine, such as α-naphthylalanine, or a hydrogenated phenylalanine or phenylglycine, such as cyclohexylalanine or cyclohexylglycine.

Those amino acid residues may be substituted at free amino or hydroxy functions, as described above for amino acid residues R₁ or R₉.

Especially preferred is the residue, bonded via the carbonyl of its carboxy group and an oxygen atom, of an amino acid selected from glycine (H-Gly-OH), alanine (H-Ala-OH), 2-aminobutyric acid, 3-aminobutyric acid, 4-aminobutyric acid, 3-aminopentanoic acid, 4-aminopentanoic acid, 5-aminopentanoic acid, 3-aminohexanoic acid, 4-aminohexanoic acid or 5-aminohexanoic acid, valine (H-Val-OH), norvaline (α-aminovaleric acid), leucine (H-Leu-OH), isoleucine (H-Ile-OH), norleucine α-aminohexanoic acid, H-Nle-OH), serine (H-Ser-OH), homoserine α-amino-γ-hydroxybutyric acid), threonine (H-Thr-OH), methionine (H-Met-OH), cysteine (H-Cys-OH), proline (H-Pro-OH), trans-3- and trans-4-hydroxyproline, phenylalanine (H-Phe-OH), tyrosine (H-Tyr-OH), 4-aminophenylalanine, 4-chlorophenylalanine, 4-carboxyphenylalanine, β-phenylserine (β-hydroxyphenylalanine), phenylglycine, α-naphthylalanine (H-Nal-OH), cyclohexylalanine (H-Cha-OH), cyclohexylglycine, tryptophan (H-Trp-OH), indoline-2-carboxylic acid, 1,2,3,4-tetrahydroisoquinoline-3-carboxylic acid, aspartic acid (H-Asp-OH), asparagine (H-Asn-OH), aminomalonic acid, aminomalonic acid monoamide, glutamic acid (H-Glu-OH), glutamine (H-Gln-OH), histidine (H-His-OH), arginine (H-Arg-OH), lysine (H-Lys-OH), δ-hydroxylysine, ornithine (α,δ-diaminovaleric acid), 3-aminopropanoic acid, α,γ-diaminobutyric acid and α,δ-diaminopropionic acid, especially the residue of an aliphatic amino acid selected from alanine, valine, norvaline, leucine, 3-aminopropionic acid, 2-aminobutyric acid, 3-aminobutyric acid, 4-aminobutyric acid, 3-aminopentanoic acid, 4-aminopentanoic acid, 5-aminopentanoic acid, 3-aminohexanoic acid, 4-aminohexanoic acid or 5-aminohexanoic acid and isoleucine or an amino acid selected from glycine, asparagine, glutamine, methionine, lysine, histidine, proline and phenylalanine, it being possible for each of the mentioned amino acids to be in the D-, L- or (D,L)-form, preferably in the L-form (except in cases where there is no asymmetric carbon atom, for example in the case of glycine), and an amino group is unsubstituted or is mono- or di-N-alkylated, for example by lower alkyl, such as methyl, n-propyl or n-butyl, by pyridyl-lower alkyl, such as 2-, 3- or 4-pyridylmethyl, and/or by phenyl-lower alkyl, such as benzyl, and/or is N-acylated, for example by unsubstituted or substituted lower alkanoyl, as defined above for lower alkanoyloxy R₅, especially by acetyl, propionyl or pivaloyl, by aryl-lower alkanoyl, for example phenyl-lower alkanoyl, such as benzoyl or phenylacetyl, by lower alkoxycarbonyl, such as tert-butoxycarbonyl, or by aryl-lower alkoxycarbonyl, for example phenyl-lower alkoxycarbonyl, such as benzyloxycarbonyl.

Of the last-mentioned residues, preference is given to acyloxy groups R₅ of an unsubstituted or substituted amino acid selected from aminoacetoxy (glycyloxy), N-lower alkylaminoacetoxy, N,N-di-lower alkylaminoacetoxy, N-lower alkyl-N-phenyl-lower alkylaminoacetoxy, N-lower alkyl-N-lower alkoxycarbonylaminoacetoxy and N-phenyl-lower alkoxycarbonyl-N-lower alkylaminoacetoxy, for example N-methylaminoacetoxy, N,N-dimethylaminoacetoxy, N-methyl-N-(n-butyl)aminoacetoxy, N-methyl-N-benzylaminoacetoxy, N-methyl-N-[(2-, 3- or 4-)pyridylmethyl]-aminoacetoxy, such as N-methyl-N-3-pyridylmethylaminoacetoxy, N-methyl-N-tert-butoxycarbonylaminoacetoxy, N-benzyloxycarbonyl-N-lower alkylaminoacetoxy, prolyloxy, histidyloxy, glutamyloxy and asparagyloxy, the amino acid residues preferably being in the (L)- or the (D)- or (D,L)-form (except in cases where there is no asymmetric carbon atom, for example in the case of Gly).

Unsubstituted or substituted alkyl R₃, R₄ or R₇ is preferably one of the radicals mentioned under alkyl R₁, R₂, R₈ and R₉ and is unsubstituted or substituted, especially by the substituents mentioned for lower alkanoyl R₁ or R₉, especially one of those substituents, and is selected especially from lower alkyl, for example methyl, ethyl, propyl, isopropyl, n-butyl, isobutyl or tert-butyl, cycloalkyl-lower alkyl wherein cycloalkyl has, for example, from 3 to 7 carbon atoms, such as cyclopropyl, cyclobutyl, cyclopentyl, cyclohexyl or cycloheptyl, cycloalkyl being unsubstituted or mono- to tri-substituted by lower alkyl, such as isopropyl, halo-lower alkyl, such as trifluoromethyl, hydroxy, lower alkoxy, amino, mono- or di-lower alkylamino, halogen, such as fluorine, chlorine or bromine, nitro and/or by cyano and being bonded, preferably terminally, to lower alkyl, especially methyl, ethyl, n-propyl, isopropyl, n-butyl, isobutyl or tert-butyl, for example cyclobutyl-, cyclopentyl-, cyclohexyl- or cycloheptyl-lower alkyl, such as -methyl or -ethyl, preferably cyclohexyl-lower alkyl, such as cyclohexylmethyl, and aryl-lower alkyl wherein aryl is, for example, independently as defined for aryl as a substituent of lower alkanoyl R₁ or R₉, which is unsubstituted or substituted as there-defined, especially phenyl-lower alkyl, such as benzyl, 2-phenylethyl, 3-phenylpropyl, 4-fluorobenzyl, 4-cyanobenzyl, 4-trifluorobenzyl, 4-hydroxybenzyl, or 4-methoxybenzyl, or: 4-lower alkoxybenzyl (especially having more total alkoxy carbon atoms than in 4-methoxybenzyl), such as 4-isobutoxybenzyl, 3,4-di-lower alkoxybenzyl, such as 3,4-dimethoxybenzyl, phenyl-lower alkoxybenzyl, such as 4-benzyloxybenzyl, 4-(3,4-di-lower alkoxybenzyloxy)benzyl, lower alkoxy-lower alkoxybenzyl, such as 4-(2-methoxyethoxy)benzyl, lower alkylenedioxyphenylmethyl, such as 3,4-methylenedioxyphenylmethyl, or biphenylylmethyl, such as 4-biphenylylmethyl, especially phenyl-lower alkyl, most especially as last defined; or (also or especially) thienylmethyl or tetrahydropyranylmethyl, such as 2-thienylmethyl or 4-(2,3,5,6-tetra-hydro)pyranylmethyl.

Cycloalkyl R₃, R₄ or R₇ is preferably as defined in the definition thereof as a substituent of lower alkanoyl R₁ or R₉, and is, for example, cyclopropyl, cyclobutyl, cyclopentyl, cyclohexyl or cycloheptyl, cycloalkyl being unsubstituted or mono- to tri-substituted by lower alkyl, such as isopropyl, halo-lower alkyl, such as trifluoromethyl, hydroxy, lower alkoxy, amino, mono- or di-lower alkylamino, halogen, such as fluorine, chlorine or bromine, nitro and/or by cyano, such as cyclobutyl, cyclopentyl, cyclohexyl or cycloheptyl, especially cyclohexyl.

Aryl R₃, R₄ or R₇ is preferably independently as defined in the definition thereof as a substituent of lower alkanoyl R₁ or R₉ and, as in that definition, is unsubstituted or substituted, and is especially phenyl that is unsubstituted or mono- to tri-substituted by lower alkyl, such as isopropyl, halo-lower alkyl, such as trifluoromethyl, hydroxy, lower alkoxy, halogen, such as fluorine, chlorine or bromine, nitro and/or by cyano, such as phenyl, 4-methoxyphenyl, 4-fluorophenyl, 4-trifluoromethylphenyl or 4-cyanophenyl.

When, in the compounds of formula I-A, nitrogen atoms having free hydrogen and/or hydroxy groups are vicinal with respect to double or triple bonds, the corresponding tautomeric imino and oxo compounds are always also included.

Salts of compounds of formula I-A are especially acid addition salts, salts with bases or, where several salt-forming groups are present, can also be mixed salts or internal salts.

Salts are especially the pharmaceutically acceptable, non-toxic salts of compounds of formula I-A, the salts being selected from the salts, as defined above, of compounds of formula I (formula I-A being substituted for formula I).

For isolation or purification purposes, it is also possible to use pharmaceutically unacceptable salts.

In the groups of compounds of formula I-A mentioned below, it may be advantageous, for example in order to replace rather general definitions with more specific definitions, to use definitions of radicals from the above-mentioned general definitions.

Preference is given to a compound of formula I-A wherein:
R₁ and R₉ are each independently of the other hydrogen; lower alkanoyl, such as formyl, acetyl, propionyl, butyryl or pivaloyl, or also or especially 3,3-dimethylbutyryl; especially acetyl; aryl-lower alkanoyl wherein aryl has from 6 to 14 carbon atoms, preferably as in phenyl, indenyl, indanyl, naphthyl, anthryl, phenanthryl, acenaphthyl or fluorenyl, and may be unsubstituted or mono- to tri-substituted especially by lower alkyl, for example methyl, ethyl or propyl, halo-lower alkyl, for example trifluoromethyl, phenyl; 1- or 2-naphthyl, hydroxy, lower alkoxy, for example methoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine, chlorine or bromine, carboxy, lower alkoxycarbonyl, such as tert-butoxycarbonyl, phenyl-, naphthyl- or fluorenyl-lower alkoxycarbonyl, such as benzyloxycarbonyl, lower alkanoyl, sulfo, lower alkylsulfonyl, for example methylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylaminosulfonyl, nitro and/or by cyano, and wherein lower alkanoyl is unsubstituted or substituted by carbamoyl or by carbamoyl substituted at the nitrogen atom by one or two radicals selected from lower alkyl, carboxy-lower alkyl, lower alkoxycarbonyl-lower alkyl, di-lower alkylamino-lower alkyl, hydroxy-lower alkyl and di-lower alkoxy-lower alkyl, preferably as described under aryl-lower alkanoyl above in the general definitions, for example 4-chloro-, 4-methoxy- or 4-nitro-benzoyl, naphthylcarbonyl, such as α- or β-naphthylcarbonyl, or 1,8-naphthalenedicarbonyl bonded to the amino group via both carbonyl groups, phenyl-lower alkanoyl, such as phenylacetyl or 3-phenylpropionyl, lower alkylphenylacetyl, such as 4-methylphenylacetyl, lower alkoxyphenylacetyl, such as 4-methoxyphenylacetyl, 3-(p-hydroxyphenyl)-propionyl, diphenylacetyl, di(4-methoxyphenyl)acetyl, triphenylacetyl, 2,2-dibenzylacetyl, 3-α- or 3-β-naphthylpropionyl, phenyl-lower alkanoyl wherein the lower alkanoyl radical is substituted by carbamoyl, such as 2-carbamoyl-3-phenylpropionyl, for example 2(R,S)-carbamoyl-3-phenylpropionyl, 3-phenyl- or 3-α-naphthyl-2-carbamoylpropionyl, 3-phenyl- or 3-α-naphthyl2-tert-butylcarbamoylpropionyl, 3-phenyl- or 3-α-naphthyl-2-(2-dimethylaminoethyl)carbamoylpropionyl, especially phenyl-lower alkanoyl, such as phenylacetyl, or phenyl-lower alkanoyl wherein the lower alkanoyl radical is substituted by carbamoyl, such as 2-carbamoyl-3-phenylpropionyl, for example 2(R,S)-carbamoyl-3-phenylpropionyl; heterocyclyl-lower alkanoyl wherein heterocyclyl is thienyl, furyl, pyrrolyl, imidazolyl, pyrazolyl, oxazolyl, thiazolyl, tetrazolyl, pyridyl, pyrazinyl, pyrimidinyl, pyridazinyl, indolyl, benzimidazolyl, quinolyl, isoquinolyl, 3,1-benzofuranyl, cyclohexa[b]pyrrolyl, cyclohexa[b]pyridyl, cyclohexa[b]pyrazinyl, cyclohexa[b]pyrimidinyl, pyrrolidinyl, pyrrolinyl, imidazolidyl, piperidyl, piperazinyl, morpholinyl, thiomorpholinyl, S,S-dioxothiomorpholinyl, indolinyl, isoindolinyl, 4,5,6,7-tetrahydroindolyl, 1,2,3,4-tetrahydroquinolyl or 1,2,3,4-tetrahydroisoquinolyl, which is bonded via a ring carbon atom or a ring nitrogen atom, in the case of saturated heterocyclic compounds preferably via a ring nitrogen atom, especially indolylcarbonyl, such as 2-, 3- or 5-indolylcarbonyl, quinolyl-lower alkanoyl, for example quinolylcarbonyl, such as 2-, 3- or 4-quinolylcarbonyl, piperidylcarbonyl, such as piperidinocarbonyl or 2-, 3- or 4-piperidylcarbonyl, piperazinylcarbonyl, such as piperazin-1-ylcarbonyl, morpholinyl-lower alkanoyl, for example morpholino-lower alkanoyl, for example morpholinocarbonyl, such as morpholinocarbonyl, thiomorpholinyl-lower alkanoyl, for example thiomorpholino-lower alkanoyl, such as thiomorpholinocarbonyl, such as thiomorpholinocarbonyl, S,S-dioxothiomorpholinyl-carbonyl, such as S,S-dioxothiomorpholinocarbonyl, tetrazolyl-lower alkanoyl, such as 3-(tetrazol-1-yl)-propionyl, and pyridyl-lower alkanoyl, for example pyridylcarbonyl, such as 2-, 3- or 4-pyridylcarbonyl, or pyridylacetyl, such as 2-, 3- or 4-pyridylacetyl, with heterocyclyl-lower alkanoyl being selected especially from morpholino-lower alkanoyl, such as morpholinocarbonyl, thiomorpholino-lower alkanoyl, such as thiomorpholinocarbonyl, pyridyl-lower alkanoyl, such as 2-, 3- or 4-pyridylacetyl, and tetrazolyl-lower alkanoyl, such as 3-tetrazol-1-ylpropionyl; (lower alkoxy-lower alkoxy)-lower alkanoyl; amino-lower alkanoyl substituted at the amino nitrogen atom by heterocyclyl-lower alkanoyl wherein heterocyclyl-lower alkanoyl is independently as defined above for heterocyclyl-lower alkanoyl R₁ or R₉, especially amino-lower alkanoyl substituted at the amino nitrogen atom by N-morpholino-carbonyl or by N-thiomorpholinocarbonyl, more especially N-morpholino- or N-thiomorpholino-carbonylamino-lower alkanoyl, such as N-morpholino- or N-thiomorpholino-carbonylamino-acetyl; halo-lower alkanoyl containing up to three halogen atoms, especially α-haloacetyl, such as α-fluoro-, α-chloro-, α-bromo-, α-iodo-, α,α,α-trifluoro- or α,α,αtrichloro-acetyl, or halopropionyl, such as β-chloro- or β-bromo-propionyl, especially trifluoroacetyl; (N-heterocyclyl-lower alkylcarbamoyl)-lower alkanoyl wherein heterocyclyl is preferably selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and from thiomorpholinyl, especially 2-(N-morpholino-lower alkylcarbamoyl)-lower alkanoyl, such as 2(R,S-(N-(2-morpholinoethyl)-carbamoyl)-3-methylbutyryl, or 2-(N-(pyridyl-lower alkyl)-carbamoyl)-lower alkanoyl, such as 2(R,S)(N-(2-pyridylmethyl)-carbamoyl)-3-methylbutyryl; lower alkoxycarbonyl, especially methoxy-, ethoxy-, isopropoxy-, isobutoxy- or tert-lower alkoxy-carbonyl, for example methoxycarbonyl, tert-butoxycarbonyl or isobutoxycarbonyl; aryl-lower alkoxycarbonyl wherein aryl is phenyl, biphenylyl, 1- or 2-naphthyl, fluorenyl, or phenyl that is mono- or poly-substituted, preferably up to tri-substituted, especially mono-substituted, by lower alkyl, for example methyl or tert-butyl, hydroxy, lower alkoxy, for example methoxy, ethoxy or tert-butoxy, halogen, for example chlorine or bromine, and/or by nitro, for example phenyl-lower alkoxycarbonyl, such as benzyloxycarbonyl or 4-methoxybenzyloxycarbonyl, especially phenyl-lower alkoxycarbonyl, such as benzyloxycarbonyl; heterocyclyl-lower alkoxycarbonyl wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, and from morpholinyl and from thiomorpholinyl and is unsubstituted or substituted by lower alkyl, such as by methyl, for example 2-furylmethoxycarbonyl, or tetrahydrofuryl-lower alkoxycarbonyl, such as 2-tetrahydrofurylmethoxycarbonyl, especially tetrahydrofuryl-lower alkoxycarbonyl, such as 2(R,S)-tetrahydrofurylmethoxycarbonyl; lower alkenyloxycarbonyl wherein the lower alkenyl radical is bonded to the oxygen atom via a saturated carbon atom, for example allyloxycarbonyl; lower alkoxy-lower alkoxycarbonyl, such as 2-methoxyethoxycarbonyl; (lower alkoxy-lower alkoxy)-lower alkoxycarbonyl, such as 2-(2-methoxyethoxy)ethoxycarbonyl; lower alkanesulfonyl, for example methane- or ethane-sulfonyl, especially methanesulfonyl; heterocyclylsulfonyl (heterocyclyl-SO₂—) wherein heterocyclyl is selected from pyrrolyl, furyl, thienyl, imidazolyl, pyrazolyl, oxazolyl, thiazolyl, pyridyl, pyrazinyl, pyrimidinyl, indolyl, quinolyl and isoquinolyl, which may also be fully or partially saturated, from morpholinyl and from thiomorpholinyl and may be unsubstituted or substituted by lower alkyl, such as methyl, such as morpholinosulfonyl, thiomorpholinosulfonyl, piperidinosulfonyl or piperazinosulfonyl; carbamoyl; N-heterocyclyl-lower alkyl-N-lower alkylcarbamoyl wherein heterocyclyl is independently one of the radicals mentioned above in the definition of heterocyclyl-lower alkanoyl R₁ or R₉, especially pyridyl, such as 2-, 3- or 4-pyridyl, preferably N-pyridyl-lower alkyl-N-lower alkylcarbamoyl, such as N-(2-, 3- or 4-pyridylmethyl)-N-methylcarbamoyl; or also or especially N-lower alkyl-N-(morpholino-lower alkyl)-aminocarbonyl, such as N-methyl-N-(2-morpholinoethyl)-aminocarbonyl; or an acyl radical of an amino acid the amino function of which is free or acylated by one of the other radicals mentioned hitherto for R₁ and R₉ with the exception of one of the mentioned aminoacyl radicals, the amino acid residues being selected from the residues, bonded via the carbonyl of their 1-carboxy group, of the amino acids glycine, alanine, valine, norvaline, leucine, isoleucine, norleucine, serine, homoserine, threonine, methionine, cysteine, proline, trans-3- and trans-4-hydroxyproline, phenylalanine, tyrosine, 4-aminophenylalanine, 4-chlorophenylalanine, 4-carboxyphenylalanine, β-phenylserine, phenylglycine, α-naphthylalanine, cyclohexylalanine, cyclohexylglycine, tryptophan, indoline-2-carboxylic acid, 1,2,3,4-tetrahydroisoquinoline-3-carboxylic acid, aspartic acid, asparagine, aminomalonic acid, aminomalonic acid monoamide, glutamic acid, glutamine, histidine, arginine, lysine, δ-hydroxylysine, ornithine, 3-aminopropanoic acid, α,γ-diaminobutyric acid and α,β-diaminopropionic acid, more especially the residues of an amino acid selected from valine, alanine, leucine, isoleucine, glycine, glutamic acid and asparagine, it being possible for each of the mentioned amino acids to be in the D-, L- or (D,L)-form, preferably in the L-form (with the exception of Val, which may also be in the (D)- or (D,L)-form); and the α-amino group being unsubstituted or N-acylated by one of the radicals mentioned above for R₁ and R₉, especially by lower alkanoyl, phenyl-lower alkanoyl, such as phenylacetyl, phenyl-lower alkanoyl wherein the lower alkanoyl radical is substituted by carbamoyl, such as 2(R,S)-carbamoyl-3-phenyl-propionyl, morpholino-lower alkanoyl, such as morpholinocarbonyl, thiomorpholino-lower alkanoyl, such as thiomorpholino-carbonyl, pyridyl-lower alkanoyl, such as 2-, 3- or 4-pyridylacetyl, quinolinyl-lower alkanoyl, such as quinolin-2-ylcarbonyl, tetrazolyl-lower alkanoyl, such as 3-tetrazol-1-ylpropionyl, lower alkoxy-lower alkoxy-lower alkanoyl, amino-lower alkanoyl substituted at the amino nitrogen atom by N-morpholino- or N-thiomorpholinocarbonyl, for example N-morpholino- or N-thiomorpholino-carbonylamino-lower alkanoyl, such as N-morpholino- or N-thiomorpholino-carbonylaminoacetyl, halo-lower alkanoyl containing up to three halogen atoms, for example α-haloacetyl, such as α-fluoro-, α-chloro-, α-bromo-, α-iodo-, α, α,α-trifluoro- or α,α,α-trichloro-acetyl, or halopropionyl, such as β-chloro- or β-bromo-propionyl, especially trifluoroacetyl, 2-(N-morpholino-lower alkylcarbamoyl)-lower alkanoyl, such as 2(R,S)-(N-(2-morpholinoethyl)-carbamoyl-3-methyl-butyryl, 2-(N-(pyridyl-lower alkyl)-carbamoyl)-lower alkanoyl, such as 2(R,S)-(N-(2-pyridylmethyl)-carbamoyl)-lower alkanoyl, lower alkoxycarbonyl, phenyl-lower alkoxycarbonyl, tetrahydrofuryl-lower alkoxycarbonyl, such as 2 (R, S)-tetrahydrofurylmethoxycarbonyl, lower alkenyloxycarbonyl wherein the lower alkenyl radical is bonded via a saturated carbon atom to the bonding oxygen atom, lower alkoxy-lower alkoxycarbonyl, (lower alkoxy-lower alkoxy)-lower alkoxycarbonyl, lower alkanesulfonyl, morpholinosulfonyl, thiomorpholinosulfonyl, piperidinosulfonyl, 4-methylpiperazinylsulfonyl or piperazinosulfonyl or N-pyridyl-lower alkyl-N-lower alkyl-carbamoyl, such as N-(2-, 3- or 4-pyridylmethyl)-N-methyl-carbamoyl; or also or especially N-(phenyl-lower alkyl)-aminocarbonyl, such as N-benzylaminocarbonyl, N-lower alkylaminocarbonyl, such as tert-butylaminocarbonyl, N,N-di-lower alkylaminocarbonyl, such as N,N-dimethylaminocarbonyl, N-(lower alkoxy-lower alkyl)-aminocarbonyl, such as N-(2-methoxyethoxy)-aminocarbonyl or N-(morpholino-lower alkyl)aminocarbonyl, such as N-(2-morpholinoethyl)-aminocarbonyl, or an acyl radical of an amino acid, as defined above, wherein the α-amino group is acylated by one of those radicals,
with the proviso that not more than one of the two radicals R₁ and R₉ may be hydrogen,
R₂, R₄, R₆ and R₈ are hydrogen,
R₃ is lower alkyl, such as isobutyl or n-butyl; C₃-C₇ cycloalkyl-lower alkyl wherein C₃-C₇ cycloalkyl is unsubstituted or mono- to tri-substituted by lower alkyl, such as isopropyl, halo-lower alkyl, such as trifluoromethyl, hydroxy, lower alkoxy, amino, mono- or di-lower alkylamino, halogen, such as fluorine, chlorine or bromine, nitro and/or by cyano and is bonded, preferably terminally, to lower alkyl, especially methyl, ethyl, n-propyl, isopropyl, n-butyl, isobutyl or tert-butyl, such as cyclobutyl-, cyclopentyl-, cyclohexyl- or cycloheptyl-lower alkyl, such as -methyl or -ethyl, especially cyclohexyl-lower alkyl, most especially cyclohexylmethyl; or is aryl-lower alkyl wherein aryl is independently as defined in aryl-lower alkanoyl R₁ or R₉, or is also or especially phenyl-lower alkoxybenzyl, 4-(3,4-di-lower alkoxybenzyloxy)benzyl, lower alkoxy-lower alkoxybenzyl or lower alkylenedioxyphenylmethyl, and is especially phenyl that may be unsubstituted or mono- to tri-substituted by lower alkyl, for example methyl, ethyl or isopropyl, halo-lower alkyl, such as trifluoromethyl, phenyl, 1- or 2-naphthyl, hydroxy, lower alkoxy, for example methoxy, or also or especially isobutoxy, carbamoyl-lower alkoxy, N-lower alkylcarbamoyl-lower alkoxy or N,N-di-lower alkylcarbamoyl-lower alkoxy, amino, mono- or di-lower alkylamino, lower alkanoylamino, for example pivaloylamino, halogen, for example fluorine or chlorine, carboxy, lower alkoxycarbonyl, benzyl-, naphthyl- or fluorenyl-lower alkoxycarbonyl, lower alkanoyl, sulfo, lower alkylsulfonyl, phosphono, hydroxy-lower alkoxyphosphoryl or di-lower alkoxyphosphoryl, carbamoyl, mono- or di-lower alkylcarbamoyl, sulfamoyl, mono- or di-lower alkylsulfamoyl, nitro and/or by cyano, especially phenyl-lower alkyl that is unsubstituted or substituted by the mentioned substituents, especially benzyl, 2-phenylethyl, 3-phenylpropyl, 4-fluoro-, 4-trifluoromethyl-, 4-cyano-, 4-methoxy- or 4-hydroxy-benzyl, or also or especially: 4-lower alkoxybenzyl (especially having more total alkoxy carbon atoms than in 4-methoxybenzyl), such as 4-isobutoxybenzyl, 3,4-di-lower alkoxybenzyl, such as 3,4-dimethoxybenzyl, phenyl-lower alkoxybenzyl, such as 4-benzyloxybenzyl, 4-(3,4-di-lower alkoxybenzyloxy)benzyl, lower alkoxy-lower alkoxybenzyl, such as 4-(2-methoxyethoxy)benzyl, lower alkylenedioxyphenylmethyl, such as 3,4-methylenedioxyphenylmethyl, or biphenylylmethyl, such as 4-biphenylylmethyl, more especially phenyl-lower alkyl, especially as last defined, or also or especially thienylmethyl or tetrahydropyranylmethyl, such as 2-thienylmethyl or 4-(2,3,5,6-tetrahydro)pyranylmethyl,
R₅ is lower alkanoyloxy, octanoyloxy, nonanoyloxy, decanoyloxy, undecanoyloxy, dodecanoyloxy, hydroxy-lower alkanoyloxy, lower alkoxy-lower alkanoyloxy, lower alkanoyloxy-lower alkanoyloxy, halo-lower alkanoyloxy, for example α-haloacetoxy, such as α-chloro-, α-bromo-, α-iodo-, α,α,α-trifluoro- or α,α,α-trichloro-acetoxy, carboxy-lower alkanoyloxy, lower alkoxycarbonyl-lower alkanoyloxy, carbamoyl-lower alkanoyloxy, lower alkylcarbamoyl-lower alkanoyloxy, di-lower alkylcarbamoyl-lower alkanoyloxy, hydroxy-carboxy-lower alkanoyloxy, hydroxy-lower alkoxycarbonyl-lower alkanoyloxy, dihydroxy-carboxy-lower alkanoyloxy, dihydroxy-lower alkoxycarbonyl-lower alkanoyloxy, pyrrolylcarbonyloxy, such as 2- or 3-pyrrolylcarbonyloxy, furyl-lower alkanoyloxy, for example furylcarbonyloxy, such as 2-furylcarbonyloxy, thienylcarbonyloxy, for example 2-thienylcarbonyloxy, imidazolyl-lower alkanoyloxy, for example imidazolylcarbonyloxy, such as 4-imidazolylcarbonyloxy, imidazolylacetoxy, such as 4-imidazolylacetoxy, imidazolylpropionyloxy, such as 3-(4-imidazolylpropionyloxy, pyridyl-lower alkanoyloxy, such as pyridylcarbonyloxy, for example 2-, 3- or 4-pyridylcarbonyloxy, indolylcarbonyloxy, for example 2-, 3- or 5-indolylcarbonyloxy, quinolyl-lower alkanoyloxy, such as quinolinylcarbonyloxy, such as quinolin-2-ylcarbonyloxy, pyrrolidinylcarboxy, such as pyrrolidinyl-3-carbonyloxy, piperidinylcarbonyloxy, for example 2-, 3- or 4-piperidinylcarbonyloxy, morpholinocarbonyloxy, thiomorpholinocarbonyloxy, morpholinoacetoxy, thiomorpholinoacetoxy or 4-lower alkyl-1-piperazinoacetoxy, such as 4-methyl-piperazinoacetoxy, lower alkenoyloxy (wherein the lower alkenoyl radical is preferably bonded to the bonding oxygen atom via a saturated carbon atom), for example acryloyloxy, vinylacetoxy, crotonoyloxy or 3- or 4-pentenoyloxy, lower alkynoyloxy, for example propioloyloxy or 2- or 3-butynoyloxy, C₃-C₈ cycloalkylcarbonyloxy, C₃-C₈ cycloalkylacetoxy, phenyl-lower alkanoyloxy, for example benzoyloxy, phenylacetoxy or 3-phenylpropionyloxy, which may be unsubstituted, mono- or poly-substituted in the phenyl radical by lower alkyl, for example methyl, halo-lower alkyl, such as chloro- or bromo-methyl, halogen, for example fluorine or chlorine, hydroxy, lower alkoxy, for example methoxy, piperidinomethyl, piperazin-1-ylmethyl, 4-lower alkyl-piperazin-1-ylmethyl, such as 4-methyl- or 4-ethyl-piperazin-1-ylmethyl, 4-lower alkanoyl-piperazin-1-ylmethyl, such as 4-acetyl-piperazin-1-ylmethyl, morpholino-lower alkyl, such as morpholinomethyl, thiomorpholinomethyl, cyano and/or by nitro, especially mono-substituted by one of the mentioned substituents, or is the residue, bonded via a carbonyloxy group containing the carbonyl from the carboxy group of the amino acid in question, of an amino acid selected from glycine, alanine, 2-aminobutyric acid, 3-aminobutyric acid, 4-aminobutyric acid, 3-aminopentanoic acid, 4-aminopentanoic acid, 5-aminopentanoic acid, 3-aminohexanoic acid, 4-aminohexanoic acid, 5-aminohexanoic acid, valine, norvaline, leucine, isoleucine, norleucine, serine, homoserine, threonine, methionine, cysteine, proline, phenylalanine, tyrosine, cyclohexylalanine, tryptophan, aspartic acid, asparagine, glutamic acid, glutamine, histidine, arginine, lysine, ornithine, 3-aminopropanoic acid, α,γ-diaminobutyric acid and α,β-diaminopropionic acid; especially the radical, bonded via carbonyloxy, of an aliphatic amino acid selected from alanine, valine, norvaline, leucine, 3-aminopropionic acid, 2-aminobutyric acid, 3-aminobutyric acid, 4-aminobutyric acid, 3-aminopentanoic acid, 4-aminopentanoic acid, 5-aminopentanoic acid, 3-aminohexanoic acid, 4-aminohexanoic acid, 5-aminohexanoic acid and isoleucine or an amino acid selected from glycine, asparagine, glutamine, methionine, lysine, histidine, proline and phenylalanine, it being possible for each of the mentioned amino acids to be in the D-, L- or (D,L)-form, preferably in the L-form (except in cases where there is no asymmetric carbon atom, for example in the case of glycine); and wherein an amino group is unsubstituted or is mono- or di-N-alkylated by lower alkyl, such as methyl, n-propyl or n-butyl, by pyridyl-lower alkyl, such as 2-, 3- or 4-pyridylmethyl, and/or by phenyl-lower alkyl, such as benzyl, and/or is N-acylated by lower alkanoyl, especially acetyl, propionyl or pivaloyl, by phenyl-lower alkanoyl, such as benzoyl or phenylacetyl, by lower alkoxycarbonyl, such as tert-butoxycarbonyl, or by phenyl-lower alkoxycarbonyl, such as benzyloxycarbonyl; or is also or especially palmitoyloxy; and
R₇ is independently of R₃ one of the radicals there-defined, especially lower alkyl, more especially isobutyl or n-butyl, C₃-C₇ cycloalkyl-lower alkyl, especially cyclohexyl-lower alkyl, such as cyclohexylmethyl, or aryl-lower alkyl, as described for aryl-lower alkyl R₃, especially phenyl-lower alkyl that is unsubstituted or substituted by the mentioned substituents, such as benzyl, 2-phenylethyl, 3-phenylpropyl, 4-fluoro-, 4-trifluoromethyl-, 4-cyano-, 4-methoxy- or 4-hydroxy-benzyl, or also or especially 4-lower alkoxybenzyl (especially having more total alkoxy carbon atoms than 4-methoxybenzyl), such as 4-isobutoxybenzyl, 3,4-di-lower alkoxybenzyl, such as 3,4-dimethoxybenzyl, phenyl-lower alkoxybenzyl, such as 4-benzyloxybenzyl, 4-(3,4-di-lower alkoxybenzyloxy)benzyl, lower alkoxy-lower alkoxybenzyl, such as 4-(2-methoxyethoxy)benzyl, lower alkylenedioxyphenylmethyl, such as 3,4-methylenedioxyphenylmethyl, or biphenylylmethyl, such as 4-biphenylylmethyl, especially phenyl-lower alkyl, especially as last defined, or also or especially thienylmethyl or tetrahydropyranylmethyl, such as 2-thienylmethyl or 4-(2,3,5,6-tetrahydro)pyranylmethyl,
or a salt thereof where at least one salt-forming group is present.

Great preference is given to a compound of formula I-A wherein R₁ and R₉ are each independently of the other hydrogen, lower alkanoyl, such as acetyl or propionyl, phenyl-lower alkanoyl, such as phenylacetyl, phenyl-lower alkanoyl wherein the lower alkanoyl radical is substituted by carbamoyl, such as 2-carbamoyl-3-phenyl-propionyl, morpholino-lower alkanoyl, such as morpholinocarbonyl, thiomorpholino-lower alkanoyl, such as thiomorpholino-carbonyl, pyridyl-lower alkanoyl, such as 2-, 3- or 4-pyridylacetyl, quinolyl-lower alkanoyl, such as quinolin-2-ylcarbonyl, tetrazolyl-lower alkanoyl, such as 3-tetrazol-1-yl-propionyl, amino-lower alkanoyl substituted at the amino nitrogen atom by N-morpholino- or N-thiomorpholino-carbonyl, for example N-morpholino- or N-thiomorpholino-carbonylamino-lower alkanoyl, such as N-morpholino- or N-thiomorpholinocarbonylamino-acetyl, halo-lower alkanoyl containing up to three halogen atoms, such as trifluoroacetyl, 2-(N-morpholino-lower alkylcarbamoyl)-lower alkanoyl, such as 2(R,S)(N-(2-morpholinoethyl)-carbamoyl)-3-methyl-butyryl, 2-(N-pyridyl-lower alkylcarbamoyl)-lower alkanoyl, such as 2(R,S)-(N-(2-pyridylmethyl)-carbamoyl)-3-methylbutyryl, lower alkoxycarbonyl, such as methoxy-, ethoxy-, isobutoxy- or tert-lower alkoxy-carbonyl, phenyl-lower alkoxycarbonyl, such as benzyloxycarbonyl, tetrahydrofuryl-lower alkoxycarbonyl, such as 2-tetrahydrofuryl-methoxycarbonyl, lower alkenyloxycarbonyl (preferably having lower alkenyl bonded via a saturated carbon atom to the bonding oxygen atom), such as allyloxycarbonyl, lower alkoxy-lower alkoxycarbonyl, such as 2-methoxyethoxycarbonyl, (lower alkoxy-lower alkoxy)-lower alkoxycarbonyl, such as 2-(2-methoxyethoxy)-ethoxycarbonyl, lower alkanesulfonyl, for example methane- or ethane-sulfonyl, morpholinosulfonyl, thiomorpholinosulfonyl, N-pyridyl-lower alkyl-N-lower alkylcarbamoyl, such as N-(2-pyridylmethyl)-N-methylaminocarbonyl, or an acyl radical, bonded via the carbonyl of its carboxy group, of an amino acid selected from glycine, alanine, valine, leucine, isoleucine, glutamic acid and asparagine in the (D)-, (L)- or (D,L)-form (with the exception of glycine), wherein the α-amino group is unsubstituted or acylated by one of the other radicals R₁ or R₉ mentioned hitherto, with the exception of an acyl radical of an amino acid, greatest preference being given to the acyl radicals of N-morpholinocarbonyl-glycine, N-(N-(2-, 3- or 4-pyridyl)methyl-N-methylaminocarbonyl)-glycine, valine, N-(trifluoroacetyl)-valine, N-phenylacetyl-valine, N-acetyl-valine, N-(2-carbamoyl-3-phenyl-propionyl)-valine, N-(2-, 3- or 4-pyridylacetyl)-valine, N-2-tetrahydrofuryl-[2H]-methoxycarbonyl-valine, N-(2-methoxy)ethoxycarbonyl-valine, N-(2-methoxyethoxy)ethoxycarbonyl-valine, N-(3-(tetrazol-1-yl)-propionyl)-valine, N-(quinolin-2-ylcarbonyl)-valine, N-methoxycarbonyl-valine, N-isobutoxycarbonyl-valine, N-tert-butoxycarbonyl-valine, N-benzyloxycarbonyl-valine, N-(morpholinocarbonyl)-valine, N-(N-(morpholinocarbonyl)aminoacetyl)-valine, N-(thiomorpholinocarbonyl)-valine, N-(N-2-pyridylmethyl-N-methylaminocarbonyl)-valine, N-morpholinocarbonylaminoacetyl-valine, N-methylsulfonylvaline, morpholinosulfonyl-valine, N-acetyl-isoleucine, N-propionyl-isoleucine, N-(benzyloxycarbonyl)-isoleucine, glutamic acid, N-benzyloxycarbonyl-glutamic acid, asparagine, N-benzyloxycarbonyl-asparagine and/or quinolin-2-ylcarbonyl-asparagine, wherein the amino acid residues are each preferably in the (L)- or (D,L)-form, and in the case of valine also in the (D)-form; with the proviso that not more than one of the radicals R₁ and R₉ is hydrogen,
R₂, R₄, R₆ and R₈ are hydrogen,
R₃ is lower alkyl, such as n-butyl or isobutyl, cyclohexyl-lower alkyl, such as cyclohexylmethyl, or phenyl-lower alkyl that is unsubstituted or substituted by halogen, such as fluorine, lower alkoxy, such as methoxy, or by cyano, especially benzyl, 4-fluorobenzyl or 4-cyanobenzyl,
R₅ is lower alkanoyloxy, such as acetoxy, propionyloxy, butyryloxy, pentanoyloxy or pivaloyloxy, octanoyloxy, decanoyloxy, dodecanoyloxy, carboxy-lower alkanoyloxy, such as 3-carboxypropionyloxy, furyl-lower alkanoyloxy, such as 2-furylcarbonyloxy, imidazolyl-lower alkanoyloxy, such as 4-imidazolylcarbonyloxy, 4-imidazolylacetoxy or 3-(4-imidazolyl)-propionyloxy, pyridyl-lower alkanoyloxy, such as 2-, 3- or 4-pyridylcarboxy, 2-pyridylacetoxy or 3-(2-pyridyl)propionyloxy, quinolyl-lower alkanoyloxy, such as quinolin-2-ylcarbonyloxy, aminoacetoxy (glycyloxy), N-lower alkylaminoacetoxy, such as N-methylaminoacetoxy, N,N-di-lower alkylaminoacetoxy, such as N,N-dimethylaminoacetoxy, N-lower alkyl-N-phenyl-lower alkoxycarbonylaminoacetoxy, such as N-benzyloxycarbonyl-N-methyl-aminoacetoxy, phenyl-lower alkanoyloxy, such as benzoyloxy, 4-morpholino-lower alkylbenzoyloxy, such as 4-morpholinomethylbenzoyloxy, 4-halomethylbenzoyloxy, histidyloxy or prolyloxy and
R₇ is as last defined for R₃, especially lower alkyl, such as isobutyl or n-butyl; cyclohexyl-lower alkyl; or phenyl-lower alkyl that is unsubstituted or substituted by halogen, such as fluorine, lower alkoxy, such as methoxy, or by cyano; as last defined for R₃, or a salt thereof where at least one salt-forming group is present, still greater preference being given to those compounds in which R₁ and/or R₉ are not morpholinosulfonyl or thiomorpholinosulfonyl.

Especially preferred is a compound of formula I-A wherein R₁ is lower alkoxycarbonyl, halo-lower alkoxycarbonyl, phenyl-lower alkoxycarbonyl, the monovalent residue, bonded via carbonyl, of an aliphatic amino acid selected from valine, alanine, leucine and isoleucine or the residue, bonded via carbonyl, of an aliphatic amino acid as defined above acylated at the amino nitrogen atom by one of the radicals phenyl-lower alkanoyl, morpholinyl-lower alkanoyl, thiomorpholinyl-lower alkanoyl, pyridyl-lower alkanoyl, lower alkoxycarbonyl and phenyl-lower alkoxycarbonyl, all the mentioned amino acids being in the D-, D,L- or L-form, preferably in the L-form,
R₂ is hydrogen,
R₃ is phenyl-lower alkyl, 4-fluorophenyl-lower alkyl or cyclohexyl-lower alkyl,
R₄ is hydrogen,
R₅ is lower alkanoyloxy, octanoyloxy, decanoyloxy, dodecanoyloxy, carboxy-lower alkanoyloxy, furyl-lower alkanoyloxy, imidazolyl-lower alkanoyloxy, pyridyl-lower alkanoyloxy, quinolyl-lower alkanoyloxy, aminoacetoxy (glycyloxy), N-lower alkylaminoacetoxy, N,N-di-lower alkylaminoacetoxy, N-lower alkyl-N-phenyl-lower alkoxycarbonylaminoacetoxy, phenyl-lower alkanoyloxy, 4-morpholinomethylbenzoyloxy, 4-halomethylbenzoyloxy, histidyloxy or prolyloxy (=pyrrolidin-2-ylcarbonyloxy),
R₆ is hydrogen,
R₇ is lower alkyl, cyclohexyl-lower alkyl, phenyl-lower alkyl, 4-cyanophenyl-lower alkyl or 4-fluorophenyl-lower alkyl,
R₈ is hydrogen; and
R₉ is one of the radicals mentioned for R₁, and the asymmetric carbon atoms carrying the radicals R₃ and R₅ are in the S-configuration, and pharmaceutically acceptable salts thereof.

Great preference is given to a compound of formula I-A wherein R₁ and R₉ are N-methoxycarbonylvalyl, R₂, R₄, R₆ and R₈ are hydrogen, R₃ is benzyl or cyclohexylmethyl, R₅ is lower alkanoyloxy, especially acetoxy, or pyridylcarbonyloxy, especially 2-pyridylcarbonyloxy, and R₇ is cyclohexylmethyl or benzyl, and pharmaceutically acceptable salts thereof, especially an isomer of that compound wherein the carbon atom carrying R₃ and the carbon atom carrying R₅ are in the (S)-configuration.

Great preference is given also to a compound of formula I-A wherein R₁ and R₉ are each independently of the other N-lower alkoxycarbonyl-valyl, R₂ is hydrogen, R₃ is phenylmethyl or cyclohexylmethyl, R₄ is hydrogen, R₅ is palmitoyloxy, lower alkoxy-lower alkanoyloxy or pyridylcarbonyloxy, R₆ is hydrogen, R₇ is phenylmethyl or cyclohexylmethyl and R₈ is hydrogen, or a pharmaceutically acceptable salt thereof.

Of those compounds, special preference is given to the following compounds:
1-[2 (S)-palmitoyloxy-3 (S)-(N-(methoxy-carbonyl)-(L)-valyl)amino-4-phenyl-butyl]-1-[cyclohexylmethyl]-2-[N-methoxy-carbonyl-(L)-valyl]hydrazine of formula I-A, or a pharmaceutically acceptable salt thereof; or
1[-2(S)-(methoxy-acetoxy)-3(S)-(N-(methoxy-carbonyl)-(L)-valyl)amino-4-phenylbutyl]-1-[cyclohexylmethyl]-2-[N-methoxy-carbonyl-(L)-valyl]hydrazine of formula I-A, or a pharmaceutically acceptable salt thereof; or
1-[2(S)-(2-pyridyl-carbonyl)oxy-3(S)-(tert-butoxy-carbonyl)amino-4-phenyl-butyl]-1-[cyclohexylmethyl]-2-[tert-butoxy-carbonyl]hydrazine of formula I-A, or a pharmaceutically acceptable salt thereof.

Especially preferred are, finally, also compounds of formula I-A wherein
R₁ is quinolin2-yl-carbonyl-(L)-asparaginyl,
R₂ is hydrogen,
R₃ is phenylmethyl, 4-lower alkoxyphenylmethyl or 4-benzyloxyphenylmethyl,
R₄ is hydrogen,
R₅ is lower alkanoyloxy, such as butyryloxy, octanoyloxy, decanoyloxy, dodecanoyloxy, palmitoyloxy, lower alkoxy-lower alkanoyloxy, such as methoxyacetoxy, carboxy-lower alkanoyloxy, furyl-lower alkanoyloxy, imidazolyl-lower alkanoyloxy, pyridyl-lower alkanoyloxy, such as especially pyridinylcarbonyloxy, for example 2- or 3-pyridinylcarbonyloxy, quinolyl-lower alkanoyloxy, aminoacetoxy (glycyloxy), N-lower alkylaminoacetoxy, N,N-di-lower alkylaminoacetoxy, N-lower alkyl-N-phenyl-lower alkoxycarbonylaminoacetoxy, phenyl-lower alkanoyloxy, 4-morpholinomethylbenzoyloxy, 4-halomethylbenzoyloxy, histidyloxy or prolyloxy (=pyrrolidin-2-ylcarbonyloxy),
R₆ is hydrogen,
R₇ is phenylmethyl, 4-lower alkoxyphenylmethyl or cyclohexylmethyl,
R₈ is hydrogen; and
R₉ is lower alkoxycarbonyl-(L)-valyl, lower alkoxy-lower alkoxy-lower alkoxycarbonyl-(L)-valyl, phenyl-lower alkoxycarbonyl-(L)-valyl, lower alkanoyl-(L)-valyl, benzylaminocarbonyl or C₃-C₇ alkenyloxycarbonyl, or (less preferably) lower alkoxycarbonyl, or pharmaceutically acceptable salts thereof. Especially preferred are compounds selected from:
1-[2 (S)-(2-pyridylcarbonyl)oxy-3 (S)-(N-quinoline-2-carbonyl)-(L)-asparaginyl)amino4-phenylbutyl-1-[phenylmethyl]-2-[N-(methoxycarbonyl)-(L)-valyl]hydrazine,
1-[2(S)-butyryloxy-3(S)-(N-quinoline-2-carbonyl)-(L)-asparaginyl)amino-4-phenylbutyl-1-[phenylmethyl]-2-[N-(methoxycarbonyl)-(L)-valyl]hydrazine,
1-[2(S)-(2-pyridylcarbonyl)oxy-3(S)-(N-quinoline-2-carbonyl)-(L)-asparaginyl)amino4-phenylbutyl-1-[4-methoxyphenylmethyl]-2-[N-(benzyloxycarbonyl)-(L)-valyl]hydrazine, and
1-[2(S)-(methoxy-acetyl)oxy-3(S)-(N-quinoline-2-carbonyl)-(L)-asparaginyl)amino-4-phenylbutyl-1-[4-methoxyphenylmethyl]-2-[N-(benzyloxycarbonyl)-(L)-valyl]hydrazine; or a pharmaceutically acceptable salt thereof.

Even more preferred compounds of formula 1-A are those selected from:
1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2 (S)-octanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-furylcarbonyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-imidazolylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-pyridylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]hydrazine;
1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-benzyloxycarbonyl-N-methyl-aminoacetyl)oxy-3(S)—(N-(methoxycarbonyl)(L)-valyl)amino-4-phenyl-butyl]-1-[cyclohexylmethyl]-2-[N-(methoxycarbonyl)(L)-valyl]hydrazine;
1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-morpholinomethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine; and
1-[2(S)-(3-carboxypropionyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine, or pharmaceutically acceptable salts thereof.

Most preferred of all, are the compounds mentioned in the Examples and their salts.

The compounds of formula I-A and salts of such compounds having at least one salt-forming group are obtained by means of processes known per se, for example as follows:
i) a hydroxy compound of formula I 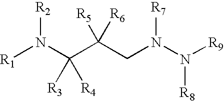
wherein the radicals are as defined for compounds of formula I-A, is acylated with a carboxylic acid of the formula:
   R₅—H
or with a reactive acid derivative thereof, wherein R₅ is as defined for compounds of formula I-A, free functional groups in the starting materials of formulae I and XXII that are not to participate in the reaction being if necessary in protected form, and any protecting groups present are removed, or
ii) for the preparation of compounds of formula I-A wherein (in correspondence with the meaning of definitions for compounds of formula I-A) R₉ is acyl, sulfo, or sulfonyl substituted by unsubstituted or substituted alkyl, aryl or heterocyclyl, and the remaining radicals are as defined, an amino compound of formula 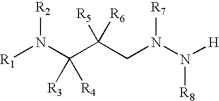
wherein the radicals are as defined immediately above, is condensed with an acid of the formula:
   R₉′—OH (VI-A),
or with a reactive acid derivative thereof, wherein R₉′ is as defined for R₉ (in correspondence with the meaning of definitions for compounds of formula I-A) with the exception of hydrogen and unsubstituted or substituted alkyl, free functional groups, with the exception of those participating in the reaction, being if necessary in protected form, and any protecting groups present are removed, or
iii) for the preparation of compounds of formula I-A wherein (in correspondence with the meaning of definitions for compounds of formula I-A) R₁ is acyl, sulfo, or sulfonyl substituted by unsubstituted or substituted alkyl, aryl or heterocyclyl, and the remaining radicals are as defined, an amino compound of formula 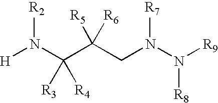
wherein the radicals are as defined immediately above, is condensed with an acid of the formula:
   R₁′—OH(VIII),
or with a reactive acid derivative thereof, wherein (in correspondence with the meaning of definitions for compounds of formula I-A) R₁′ is as defined for R₁ with the exception of hydrogen and unsubstituted or substituted alkyl, free functional groups, with the exception of those participating in the reaction, being if necessary in protected form, and any protecting groups present are removed, or
iv) for the preparation of compounds of formula I-A wherein (in correspondence with the meaning of definitions for compounds of formula I-A) R₁ and R₉ are two identical radicals selected from acyl, sulfo, and sulfonyl substituted by unsubstituted or substituted alkyl, aryl or heterocyclyl, and the remaining radicals are as defined, a diamino compound of formula 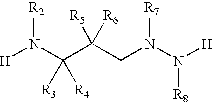
wherein the radicals are as defined immediately above, is condensed with an acid suitable for introducing the identical radicals R₁ and R₉, or with a reactive acid derivative thereof, wherein R₁ and R₉ are as defined immediately above, free functional groups, with the exception of those participating in the reaction, being if necessary in protected form, and any protecting groups present are removed, or
v) for the preparation of a compound of formula I-A wherein in place of R₇ there is a radical R₇″ which (in correspondence with the meaning of definitions for compounds of formula I-A) is unsubstituted or substituted alkyl or cycloalkyl, in a compound of formula I-A′ 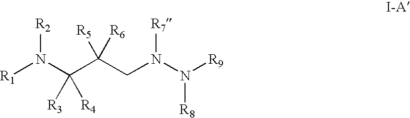
wherein R₇″ is hydrogen and the remaining radicals are as defined (in correspondence with the meaning of definitions for compounds of formula I-A), the radical R₇″ is introduced by substitution with a compound of the formula:
   R₇″—X (XII),
wherein X is a leaving group and R₇″ (in correspondence with the meaning of definitions for compounds of formula I-A) is unsubstituted or substituted alkyl or cycloalkyl, free functional groups, with the exception of those participating in the reaction, being optionally in protected form, and any protecting groups present are removed, or
vi) in a compound of formula I-A wherein the substituents are as defined above with the proviso that in the compound of formula I-A in question at least one functional group is protected by protecting groups, the protecting groups present are removed,
and, if desired, a compound of formula I-A obtainable in accordance with any one of processes i) to vi) above having at least one salt-forming group is converted into its salt or an obtainable salt is converted into the free compound or into a different salt and/or any isomeric mixtures that are obtainable are separated and/or a compound of formula I-A according to the invention is converted into a different compound of formula I-A according to the invention.

The said processes are described in detail in U.S. Pat. No. 5,753,652 and are herein incorporated by reference in its entirety.

Compounds of formula I according to the invention which have advantageous pharmacological properties (especially those described for compounds of formula I and/or, more preferentially, of formula I-A) and to which the present application therefore preferably relates are given below:

Preference is given to compounds of formula I wherein
R₁ is quinolin-2-yl-carbonyl-(L)-asparaginyl,
R₂ is hydrogen,
R₃ is phenylmethyl, 4-lower alkoxyphenylmethyl or 4-benzyloxyphenylmethyl,
R₄ is hydrogen, R₆ is hydrogen,
R₇ is phenylmethyl, 4-lower alkoxyphenylmethyl or cyclohexylmethyl,
R₈ is hydrogen; and
R₉ is lower alkoxycarbonyl-(L)-valyl, lower alkoxy-lower alkoxy-lower alkoxycarbonyl(L)-valyl, phenyl-lower alkoxycarbonyl-(L)-valyl, lower alkanoyl-(L)-valyl, benzylaminocarbonyl or C₃-C₇ alkenyloxycarbonyl, or also lower alkoxycarbonyl, or pharmaceutically acceptable salts thereof,.

More preferably the invention encompasses use of one of those compounds of formula I selected from the compounds having the names:
1-[2(S)-hydroxy-3(S)-(N-(quinoline-2-carbonyl)-(L)-asparaginyl)amino-4-phenyl-butyl]-1-[cyclohexylmethyl]-2-[N-(benzyloxycarbonyl)-(L)-valyl]hydrazine,
1-[2(S)-hydroxy-3(S)-(N-(quinoline-2-carbonyl)-(L)-asparaginyl)amino-4-phenyl-butyl]-1-[cyclohexylmethyl]-2-[tert-butoxy-carbonyl]hydrazine,
1-[2 (S)-hydroxy-3 (S)-(N-(quinoline-2-carbonyl)-(L)-asparaginyl)amino-4-phenyl-butyl]-1-[cyclohexylmethyl]-2-[N-methoxy-carbonyl-(L)-valyl]hydrazine,
1-[2(S)-hydroxy-3(S)-(N-(quinoline-2-carbonyl)-(L)-asparaginyl)amino-4-phenyl-butyl]-1-[cyclohexylmethyl]-2-[3,3-dimethylbutyryl]hydrazine,
1-[2(S)-hydroxy-3(S)-(N-(quinoline-2-carbonyl)-(L)-asparaginyl)amino-4-phenyl-butyl]-1-[cyclohexylmethyl]-2-[tert-butylamino-carbonyl]hydrazine,
1-[2 (S)-hydroxy-3(S)-(N-(quinoline-2-carbonyl)-(L)-asparaginyl)amino-4-phenyl-butyl]-1-[cyclohexylmethyl]-2-[benzylamino-carbonyl]hydrazine,
1-[2(S)-hydroxy-3(S)-(N-(quinoline-2-carbonyl)-(L)-asparaginyl)amino-4-phenyl-butyl]-1-[benzyl]-2-[N-methoxy-carbonyl)-(L)-valyl]hydrazine,
1-2(S)-hydroxy-3(S)-(N-(quinoline-2-carbonyl)-(L)-asparaginyl)amino-4-phenyl-butyl]-1-[benzyl]-2-[N-ethoxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[benzyl]-2-[N-benzyloxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[benzyl]-2-[N-allyloxy-carbonyl)-(L)-valyl]hydrazine,
1-[2(S)-hydroxy-3(S)-(N-(quinoline-.sup.2-carbonyl)-(L)-asparaginyl)amino-4-phenyl-butyl]-1-[4-methoxyphenylmethyl]-2-[N-methoxy-carbonyl)-(L)valyl]hydrazine,
1-[2(S)-hydroxy-3(S)-(N-(quinoline-2-carbonyl)-(L)-asparaginyl)amino-4-phenyl-butyl]-1-[4-methoxyphenylmethyl]-2-[N-benzyloxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine,
1-[2(S)-hydroxy-3(S)-(N-(quinoline-2-carbonyl)-(L)-asparaginyl)amino-4-(4-methoxyphenyl)-butyl]-1-[benzyl]-2-[N-methoxy-carbonyl)-(L)-valyl]hydrazine,
1-[2(S)-hydroxy-3(S)-(N-(quinoline-2-carbonyl)-(L)-asparaginyl)amino-4-(4-methoxyphenyl)-butyl]-1-[benzyl]-2-[N-benzyloxy-carbonyl)-(L)-valyl]hydrazine,
1-[2(S)-hydroxy-3(S)-(N-(quinoline-2-carbonyl)-(L)-asparaginyl)amino-4-(4-benzyloxyphenyl)-butyl]-1-[benzyl]-2-[N-methoxy-carbonyl)-(L)-valyl]hydrazine,
1-[2(S)-hydroxy-3(S)-(N-(quinoline-2-carbonyl)-(L)-asparaginyl)amino-4-(4-benzyloxyphenyl)-butyl]-1-[benzyl]-2-[N-allyloxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[4-methoxyphenylmethyl]-2-[N-methoxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[4-benzyloxyphenylmethyl]-2-[N-methoxy-carbonyl)-(L)-valyl]hydrazine,
1-[2(S)-hydroxy-3(S)-(N-(quinoline-2-carbonyl)-(L)-asparaginyl)amino-4-phenyl-butyl]-1-[2(S)-hydroxy-3(S)-(N-quinoline-2-carbonyl)-(L)-asparaginyl)amino-4-phenylbutyl-1-[phenylmethyl]-2-[N-(2-(2-methoxyethoxy)ethoxycarbonyl)-(L)-valyl]hydrazine,
1-[2(S)-hydroxy-3(S)-(N-quinoline-2-carbonyl)-(L)-asparaginyl)amino-4-phenylbutyl-1-[4-methoxyphenylmethyl]-2-[tert-butoxy-carbonyl)hydrazine, and
1-[2(S)-hydroxy-3(S)-(N-quinoline-2-carbonyl)-(L)-asparaginyl)amino-4-phenylbutyl-1-[4-methoxyphenylmethyl]-2-[N-(ethoxycarbonyl)-(L)-valyl]hydrazine,
   or a pharmaceutically acceptable salt of each of those compounds.

Preference is given also to compounds of formula I wherein R₁ is lower alkenyloxycarbonyl-(L)-valyl, the radicals R₂, R₄, R₆ and R₈ are each hydrogen, R₃ is phenylmethyl, R₇ is cyclohexylmethyl and R₉ is N-lower alkenyloxycarbonyl-(L)-valyl, or pharmaceutically acceptable salts thereof, especially a compound having the name 1-[2 (S)-hydroxy-3 (S)-(N-allyloxycarbonyl-(L)-valyl)amino-4-phenylbutyl]-1-[cyclohexylmethyl]-2-[N-allyloxycarbonyl-(L)-valyl]hydrazine, or a pharmaceutically acceptable salt thereof.

Preference is given also to compounds of formula I wherein R₁ is lower alkoxy-lower alkoxy-lower alkoxycarbonyl-(L)-valyl, the radicals R₂, R₄, R₆ and R₈ are each hydrogen, R₃ is phenylmethyl, R₇ is cyclohexylmethyl and R₉ is lower alkoxy-lower alkoxy-lower alkoxycarbonyl-(L)-valyl, or pharmaceutically acceptable salts thereof, especially a compound having the name
1-[2(S)-hydroxy-3(S)-(N-(2-(2-methoxyethoxy)ethoxy)carbonyl-(L)-valyl)amino-4-phenylbutyl]-1-[cyclohexylmethyl]-2-[N-(2-(2-methoxyethoxy)ethoxy)carbonyl-(L)-valyl]hydrazine, or a pharmaceutically acceptable salt thereof.

Preference is given also to compounds of formula I wherein R₁ is lower alkoxy-lower alkoxy-lower alkanoyl-(L)-valyl, the radicals R₂, R₄, R₆ and R₈ are each hydrogen, R₃ is phenylmethyl, R₇ is cyclohexylmethyl and R₉ is lower alkoxy-lower alkoxy-lower alkanoyl-(L)-valyl, or pharmaceutically acceptable salts thereof, especially a compound having the name
1-[2(S)-hydroxy-3(S)-(N-(2-methoxyethoxy)acetyl-(L)-valyl)amino-4-phenylbutyl]-1-[cyclohexylmethyl]-2-[N-(2-methoxyethoxy) acetyl-(L)-valyl]hydrazine, or a pharmaceutically acceptable salt thereof.

Preference is given also to compounds of formula I wherein R₁ and R₉ are each independently of the other acetyl-(L)-valyl, methoxycarbonyl-(L)-valyl, ethoxycarbonyl-(L)-valyl, dimethylaminocarbonyl-(L)-valyl, (2-methoxyethyl)aminocarbonyl-(L)-valyl, N-(2-morpholin-4-yl-ethyl)aminocarbonyl-(L)-valyl or N-(2-morpholin-4-yl-ethyl)-N-methyl-aminocarbonyl-(L)-valyl, the radicals R₂, R₄, R₆ and R₈ are each hydrogen, R₃ is phenylmethyl, and R₇ is thien-2-ylmethyl or 2,3,5,6-tetrahydropyran-4-ylmethyl, or pharmaceutically acceptable salts thereof, especially a single compound or several compounds selected from:
1-[2(S)-hydroxy-3(S)-(N-acetyl-(L)-valyl)amino-4-phenylbutyl]-1-[thien-2-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[thien-2-yl-methyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[thien-2-yl-methyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[thien-2-ylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenyl-butyl]-1-[thien-2-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[thien-2-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[thien-2-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[2,3,5,6-tetrahydropyran4-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[2,3,5,6-tetrahydropyran-4-ylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[2, 3,5,6-tetrahydropyran-4-ylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[2,3,5,6-tetrahydropyran-4-ylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[2,3,5,6-tetrahydropyran-4-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[2,3,5,6-tetrahydropyran-4-ylmethyl]-2-[N-(N-(2-(morpholin-4-yl)ethyl)aminocarbonyl)-(L)-valyl]hydrazine; and
1-[2(S)-hydroxy-3(S)-(N-(N-(2-(morpholin-4-yl)ethyl)-N-methyl-aminocarbonyl)-(L)-valyl)amino-4-phenylbutyl]-1-[2,3,5,6-tetrahydropyran-4-ylmethyl]-2-[N-(N-(2-(morpholin-4-yl)ethyl)-N-methylaminocarbonyl)-(L)-valyl]hydrazine,
   or a pharmaceutically acceptable salt thereof.

Preference is given also to compounds of formula I wherein R₁ and R₉ are each independently of the other acetyl-(L)-valyl, methoxycarbonyl-(L)-valyl, ethoxycarbonyl-(L)-valyl, dimethylaminocarbonyl-(L)-valyl, (2-methoxyethyl)aminocarbonyl-(L)-valyl, N-(2-morpholin-4-yl-ethyl)aminocarbonyl-(L)-valyl or N-(2-morpholin-4-yl-ethyl)-N-methyl-aminocarbonyl-(L)-valyl, the radicals R₂, R₄, R₆ and R₈ are each hydrogen, R₃ is phenylmethyl, and R₇ is 4-hydroxyphenylmethyl, 4-methoxyphenylmethyl, 4-isobutoxyphenylmethyl, 4-benzyloxyphenylmethyl, 3,4-dimethoxyphenylmethyl, methylene-4,5-dioxyphenylmethyl, 4-(2-methoxyethoxy)phenylmethyl or 4-biphenylylmethyl, or pharmaceutically acceptable salts thereof, especially a single compound or several compounds selected from the compounds having the names:
1-[2(S)-hydroxy-3(S)-(N-acetyl-(L)-valyl)amino-4-phenylbutyl]-1-[4-hydroxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-hydroxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-hydroxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-hydroxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-hydroxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-hydroxyphenylmethyl]-2-[N-(N-(2-(morpholin-4-yl) ethyl) aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-hydroxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-methoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-methoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-methoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-methoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-methoxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-methoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-methoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-benzyloxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-benzyloxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-benzyloxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-benzyloxyphenylmethyl)-2-[N-(N,N-dimethylaminocarbonyl)-(L)-valyl]hydrazine;
1-[2 (S)-hydroxy-3 (S)-(N-(N-(2-methoxyethyl)aminocarbonyl)-(L)-valyl)amino-4-phenylbutyl]-1-[4-benzyloxyphenylmethyl]-2-[N-(N-(2-methoxyethyl)-aminocarbonyl)(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-benzyloxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-benzyloxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(3,4-dimethoxybenzyl)oxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(3,4-dimethoxybenzyl)oxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(3,4-dimethoxybenzyl)oxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(3,4-dimethoxybenzyl)oxyphenylmethyl]-2-[N-(N,N-dimethylaminocarbonyl)(L)-valyl]hydrazine;
1-[2(S)-hydroxy-3(S)-(N-(N-(2-methoxyethyl)aminocarbonyl)-(L)-valyl)amino-4-phenylbutyl]-1-[4-(3,4-dimethoxybenzyl)oxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)aminocarbonyl)-(L)-valyl]hydrazine;
1-[([2(S)-hydroxy-3 (S)-(N-(N-(2-(morpholin-4-yl)ethyl)-N-methyl-aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-isobutoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-isobutoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-isobutoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-isobutoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-isobutoxyphenylmethyl]-2-[N-(N-(2-methoxyethyl)aminocarbonyl)-(L)-valyl]-hydrazine;
1-[2(S)-hydroxy-3(S)-(N-(N-(2-(morpholin-4-yl)ethyl)aminocarbonyl)-(L)-valyl)amino-4-phenylbutyl]-1-[4-isobutoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-isobutoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(2-methoxyethoxy)phenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(2-methoxyethoxy)phenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(2-methoxyethoxy)phenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(2-methoxyethoxy)phenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-(2-methoxyethoxy)phenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(2-methoxyethoxy)phenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(2-methoxyethoxy)phenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[methylene-3,4-dioxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[methylene-3,4-dioxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[methylene-3,4-dioxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[methylene-3,4-dioxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[methylene-3,4-dioxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[methylene-3,4-dioxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[methylene-3,4-dioxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[3,4-dimethoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[3,4-dimethoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[3,4-dimethoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[3,4-dimethoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[3,4-dimethoxyphenylmethyl]-2-[N-(N-(2-methoxyethyl)aminocarbonyl)-(L)-valyl]hydrazine:
1-[2(S)-hydroxy-3(S)-(N-(N-(2-(morpholin-4-yl)ethyl)aminocarbonyl)-(L)-valyl)amino-4-phenylbutyl]-1-[3,4-dimethoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl)-1-[3,4-dimethoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-biphenylylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-biphenylmethyl]-2-N-methoxycarbonyl-(L)-valyl]hydrazine;
1-[2(S)-hydroxy-3(S)-(N-ethoxycarbonyl-(L)-valyl)amino-4-phenylbutyl]-1-[4-biphenylylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-biphenylylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-biphenylylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-biphenylylmethyl]-2-[N-(N-(2-(morpholin-4-yl)ethyl)aminocarbonyl)-(L)-valyl]hydrazine and
1-[2(S)-hydroxy-3(S)-(N-(N-(2-(morpholin-4-yl)ethyl)-N-methyl-aminocarbonyl)-(L)-valyl)amino-4-phenylbutyl]-1-[4-biphenylylmethyl]-2-[N-(N-(2-(morpholin-4-yl)ethyl)N-methylaminocarbonyl)-(L)-valyl]hydrazine,
   or pharmaceutically acceptable salts thereof.

Preference is given also to compounds of formula I wherein R₁ and R₉ are each methoxycarbonyl-(L)-valyl, the radicals R₂, R₄, R₆ and R₈ are each hydrogen, R₃ is phenylmethyl, and R₇ is 4-lower alkoxyphenylmethyl or 4-benzyloxyphenylmethyl, or pharmaceutically acceptable salts thereof.

Preference is given finally to the following compounds of formula I:
1-[2(S)-hydroxy-3(S)-(N-ethoxycarbonyl-(L)-valyl)amino-4-phenylbutyl]-1-[cyclohexylmethyl]-2-[N-ethoxycarbonyl-(L)-valyl]hydrazine, 1-[2(S)-hydroxy-3(S)-(N-trifluoroacetyl-(L)-valyl)amino-4-phenylbutyl]-1-[cyclohexylmethyl]-2-[N-trifluoroacetyl-(L)-valyl]hydrazine,
1-[2(S)-hydroxy-3(S)-(N-(methoxy-carbonyl)-(L)-valyl)amino-4-cyclohexyl-butyl]-1-[cyclohexylmethyl]-2-[N-methoxy-carbonyl-(L)-valyl]hydrazine,
1-[2(S)-hydroxy-3 (S)-(N-(n-propoxy-carbonyl)-(L)-valyl)amino-4-phenyl-butyl]-1-[cyclohexylmethyl]-2-[N-(n-propyl)oxy-carbonyl-(L)-valyl]hydrazine,
1-[2(R)-hydroxy-3(S)-(N-(methoxy-carbonyl)-(L)-valyl)amino-4-phenyl-butyl]-1-[cyclohexylmethyl]-2-[N-methoxy-carbonyl-(L)-valyl]hydrazine,
1-[2(R)-hydroxy-3(R)-(N-(methoxy-carbonyl)-(L)-valyl)amino-4-phenyl-butyl]-1-[cyclohexylmethyl]-2-[N-methoxy-carbonyl-(L)-valyl]hydrazine,
1-[2 (S)-hydroxy-3 (S)-(benzyloxy-carbonyl-amino)-4-phenyl-butyl]-1-[phenylmethyl]-2-[N-methoxy-carbonyl-(L)-valyl]hydrazine,
1-[2(S)-hydroxy-3(S)-(N-(methoxy-carbonyl)-(L)-valyl)amino-4-cyclohexyl-butyl]-1-[phenylmethyl]-2-[N-methoxy-carbonyl)-(L)-valyl]hydrazine,
1-[2(S)-hydroxy-3(S)-(N-methoxycarbonyl-(L)-valyl)amino-4-cyclohexyl-butyl]-1-[4-methoxyphenylmethyl]-2-[N-methoxy-carbonyl)-(L)-valyl]hydrazine,
1-[2(S)-hydroxy-3(S)-(N-methoxycarbonyl-(L)-valyl)amino-4-cyclohexyl-butyl]-1-[4-isobutoxyphenylmethyl]-2-[N-methoxy-carbonyl)-(L)-valyl]hydrazine,
1-[2 (S)-hydroxy-3 (S)-(N-methoxycarbonyl-(L)-valyl)amino-4-cyclohexyl-butyl]-1-[4-ethoxyphenylmethyl]-2-[N-methoxy-carbonyl)-(L)-valyl]hydrazine,
1-[2(S)-hydroxy-3(S)-(N-methoxycarbonyl-(L)-valyl)amino-4-cyclohexyl-butyl]-1-[4-benzyloxyphenylmethyl]-2-[N-methoxy-carbonyl)-(L)-valyl]hydrazine, and/or 1-[2(S)-hydroxy-3(S)-(N-methoxycarbonyl-(L)-valyl)amino-4-phenyl-butyl]-1-[cyclohexylmethyl]-2-[2-pyridylcarbonyl]hydrazine,
   or a pharmaceutically acceptable salt thereof in each case.

The preparation of compounds that can be used as starting materials for the synthesis of the compounds of the invention is described in detail in U.S. Pat. No. 5,753,652, incorporated herein by reference.

Generally, as a result of the close relationship between the compounds of formula I and I-A and their salts and starting materials (starting compounds and intermediates) in free form and in the form of their salts, hereinbefore and hereinafter any reference to the free compounds and their salts should be understood as including the corresponding salts and free compounds, respectively, where appropriate and expedient.

All the process steps can be carried out under reaction conditions known per se, in the absence or, customarily, the presence of solvents or diluents, preferably those that are inert towards the reagents used and are solvents therefor, in the absence or presence of catalysts, condensation agents or neutralising agents, for example ion exchangers, such as cation exchangers, for example in the H⁺ form, and, depending on the nature of the reaction and/or of the reactants, at reduced, normal or elevated temperature, for example in a temperature range from approximately −100° C. to approximately 190° C., preferably from approximately −80° C. to approximately 150° C., for example from −80.° C. to −60.° C., at room temperature, from −20° C. to 40° C. or at the reflux temperature, under atmospheric pressure or in a closed vessel, if necessary under pressure, and/or in an inert atmosphere, for example under an argon or nitrogen atmosphere.

Isomeric mixtures occurring at any stage of the reaction may be separated into the individual isomers, for example diastereoisomers or enantiomers, or into any desired mixtures of isomers, for example racemates or diastereoisomeric mixtures, by any method commonly known in the art.

In certain cases, for example in the case of hydrogenation, it is possible to achieve stereo-selective reactions, which, for example, enable individual isomers to be obtained more easily.

The solvents from which those suitable for a particular reaction can be selected include, for example, water, esters, such as lower alkyl-lower alkanoates, for example ethyl acetate, ethers, such as aliphatic ethers, for example diethyl ether, or cyclic ethers, for example tetrahydrofuran, liquid aromatic hydrocarbons, such as benzene or toluene, alcohols, such as methanol, ethanol or 1- or 2-propanol, nitrites, such as acetonitrile, halogenated hydrocarbons, such as methylene chloride, acid amides, such as dimethylformamide, bases, such as heterocyclic nitrogen bases, for example pyridine, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless the description of the process indicates otherwise. Such solvent mixtures can also be used in the working-up, for example by chromatography or partitioning.

The compounds, including their salts, can also be obtained in the form of hydrates, or their crystals may, for example, include the solvent used for crystallization.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and further processed in situ. In the process of the present invention it is preferable to use those starting materials that lead to the compounds (of formula I or Ib) described in the introduction as being especially valuable. Special preference is given to reaction conditions analogous to those mentioned in the Examples.

Where necessary, protected starting compounds can be used at any stage of the process and the protecting groups removed at suitable stages of the reaction.

Protecting groups, their introduction and their removal are as described (for the preparation of compounds of formula I) under Processes a) and f) above, and in detail in U.S. Pat. No. 5,753,652.

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

A therapeutically effective amount is defined as an amount effective to reduce or lessen at least one symptom of the disease being treated or to reduce or delay onset of one or more clinical markers or symptoms of the disease.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Unless defined otherwise, all scientific and technical terms used herein have the same meaning as commonly understood by one of skill in the art to which this invention belongs.

All patents and publications referred to herein are hereby incorporated by reference for all purposes.

The present invention may be better understood with reference to the following examples. These examples are intended to be representative of specific embodiments of the invention, and are not intended as limiting the scope of the invention.

### EXAMPLES

### Example A

Enzyme Inhibition Assay

The compounds of the invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of maltose binding protein (MBP) and the carboxy terminal 125 amino acids of APP-SW, the Swedish mutation. The beta-secretase enzyme is derived from human brain tissue as described in Sinha et al, 1999, Nature 40:537-540) or recombinantly produced as the full-length enzyme (amino acids 1-501), and can be prepared, for example, from 293 cells expressing the recombinant cDNA, as described in WO00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu 596 at the substrate's APP-SW 751 mutation site.

Specific Assay Procedure:

Compounds are diluted in a 1:1 dilution series to a six-point concentration curve (two wells per concentration) in one 96-plate row per compound tested. Each of the test compounds is prepared in DMSO to make up a 10 millimolar stock solution. The stock solution is serially diluted in DMSO to obtain a final compound concentration of 200 micromolar at the high point of a 6-point dilution curve. Ten (10) microliters of each dilution is added to each of two wells on row C of a corresponding V-bottom plate to which 190 microliters of 52 millimolar NaOAc, 7.9% DMSO, pH 4.5 are pre-added. The NaOAc diluted compound plate is spun down to pellet precipitant and 20 microliters/well is transferred to a corresponding flat-bottom plate to which 30 microliters of ice-cold enzyme-substrate mixture (2.5 microliters MBP-C125SW substrate, 0.03 microliters enzyme and 24.5 microliters ice cold 0.09% TX100 per 30 microliters) is added. The final reaction mixture of 200 micromolar compound at the highest curve point is in 5% DMSO, 20 millimolar NaOAc, 0.06% TX100, at pH 4.5.

Warming the plates to 37 degrees C. starts the enzyme reaction. After 90 minutes at 37 degrees C., 200 microliters/well cold specimen diluent is added to stop the reaction and 20 microliters/well was transferred to a corresponding anti-MBP antibody coated ELISA plate for capture, containing 80 microliters/well specimen diluent. This reaction is incubated overnight at 4 degrees C. and the ELISA is developed the next day after a 2 hour incubation with anti-192SW antibody, followed by Streptavidin-AP conjugate and fluorescent substrate. The signal is read on a fluorescent plate reader.

Relative compound inhibition potency is determined by calculating the concentration of compound that showed a fifty percent reduction in detected signal (IC₅₀) compared to the enzyme reaction signal in the control wells with no added compound.

### Example B

Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds of the invention. Useful substrates include the following:

| | |
|---|---|
| Biotin-SEVNLDAEFRC[Oregon green]KK | [SEQ ID NO: 1] |
| Biotin-SEVKMDAEFRC[Oregon green]KK | [SEQ ID NO: 2] |
| Biotin-GLNIKTEEISEISYEVEFRC[Oregon green]KK | [SEQ ID NO: 3] |
| Biotin-ADRGLTTRPGSGLTNIKTEEISEVNLDAEFC[Oregon green]KK | [SEQ ID NO: 4] |
| Biotin-FVNQHLCₒₓGSHLVEALY-LVCₒₓGERGFFYTPKAC[Oregon green]KK | [SEQ ID NO: 5] |

The enzyme (0.1 nanomolar) and test compounds (0.001-100 micromolar) are incubated in pre-blocked, low affinity, black plates (384 well) at 37 degrees for 30 minutes. The reaction is initiated by addition of 150 millimolar substrate to a final volume of 30 microliter per well. The final assay conditions are: 0.001-100 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/Em530 nm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate.

### Example C

Beta-Secretase Inhibition: P26-P4′SW Assay

Synthetic substrates containing the beta-secretase cleavage site of APP are used to assay beta-secretase activity, using the methods described, for example, in published PCT application WO00/47618.

| The P26-P4′SW substrate is a peptide of the sequence: | |
|---|---|
| (biotin)CGGADRGLTTRPGSGLTNIKTEEISEVNLDAEF | [SEQ ID NO: 6] |
| The P26-P1 standard has the sequence: | |
| (biotin)CGGADRGLTTRPGSGLTNIKTEEISEVNL. | [SEQ ID NO: 7] |

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X100, and is incubated at 37 degrees C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4 degrees C. After washing in TTBS buffer (150 millimolar sodium chloride, 25 millimolar Tris, 0.05% Tween 20, pH 7.5), the samples are incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example D

Assays using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No. 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence (biotin)-SEVNLDAEF [SEQ ID NO: 8], and the cleavage site is between residues 5 and 6. Another preferred substrate has the sequence ADRGLTTRPGSGLTNIKTEEISEVNLDAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example E

Inhibition of Beta-Secretase Activity—Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example F

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with non-treated controls.

### Example G

Inhibition of A Beta Production in Human Patients

Patients suffering from Alzheimer's Disease (AD) demonstrate an increased amount of A beta in the brain. AD patients are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated patients.

### Example H

Prevention of A Beta Production in Patients at Risk for AD

Patients predisposed or at risk for developing AD are identified either by recognition of a familial inheritance pattern, for example, presence of the Swedish Mutation, and/or by monitoring diagnostic parameters. Patients identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Patients administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated patients.

All temperatures are in degrees Celsius.

HPLC refers to high pressure liquid chromatography.

Saline refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

The following Examples serve to illustrate the invention but do not limit the scope or spirit thereof in any way.

### Examples of Compounds

Temperatures are given in degrees Celsius (° C.). Where no temperature is specified, the reaction takes place at room temperature. The R_{f} values, which indicate the ratio of the distance of propagation of the substance in question to the distance of propagation of the eluant front, are determined on thin layer silica gel plates by thin layer chromatography (TLC) in the following solvent systems:

| | | |
|---|---|---|
| A. | chloroform/methanol/water/acetic acid | 75:27:5:0.5 |
| B. | chloroform/methanol/water/acetic acid | 90:10:1:0.5 |
| C. | chloroform/methanol/water/acetic acid | 85:13:1.5:0.5 |
| D. | chloroform/methanol | 95:5 |
| E. | chloroform/methanol | 95:5 |
| F. | hexane/ethyl acetate | 2:1 |
| G. | methylene chloride/ether/methanol | 20:20:1 |
| H. | methylene chloride/ether | 1:1 |
| I. | toluene/ethyl acetate | 2:1 |
| K. | chloroform/methanol | 5:1 |
| J. | methylene chloride/ether | 5:1 |
| L. | hexane/ethyl acetate | 4:1 |
| M. | hexane/ethyl acetate | 5:1 |
| N. | hexane/ethyl acetate | 1:1 |
| O. | ethyl acetate | — |
| P. | methylene chloride/ethanol/NH_{3(aq)} | 90:10:1 |
| Q. | methylene chloride/ether | 10:1 |
| R. | hexane/ethyl acetate | 3:1 |
| S. | methylene chloride/ether | 20:1 |
| T. | chloroform/methanol | 30:1 |
| U. | chloroform/methanol | 15:1 |
| V. | methylene chloride/ether/hexane | 1:1:3 |
| W. | methylene chloride/ether | 20:1 |
| X. | methylene chloride/methanol | 40:1 |
| Y. | toluene/ethyl acetate | 4:1 |
| Z. | methylene chloride/methanol | 30:1 |
| A′. | methylene chloride/methanol | 15:1 |
| B′. | methylene chloride/methanol | 10:1 |
| C′. | hexane/ethyl acetate | 1:3 |
| D′. | ethyl acetate/ethanol | 100:3 |
| E′. | ethyl acetate/ethanol | 20:1 |
| F′. | ethyl acetate/ethanol | 10:1 |
| G′. | methylene chloride/methanol | 9:1 |
| H′. | ethyl acetate/hexane | 3:2 |
| I′. | methylene chloride/methanol | 12:1 |
| J′. | methylene chloride/methanol | 19:1 |
| K′. | methylene chloride/ether/methanol | 10:10:1 |

The abbreviation “R_{f (A)}”, for example, indicates that the R_{f} value was determined in solvent system A. The ratio of solvents to one another is always given in parts by volume.
HPLC gradients
I. 20%→100% a) in b) for 35 min
II. 0%→40% a) in b) for 30 min
III. 20%→60% a) in b) for 60 min
IV. 10%→50% a) in b) for 60 min
V. 20%→100% a) in b) for 20 min

Eluant a): acetonitrile+0.05% TFA; eluant b): water+0.05% TFA. Column (250×4.6 mm) filled with “Reversed-Phase” material C₁₈-Nucleosil (5 μm average particle size, silica gel covalently derivatised with octadecylsilanes, Macherey & Nagel, Duren, FRG). Detection by UV-absorption at 215 nm. The retention times (t_{Ret}) are given in minutes. Flow rate 1 mL/min.

Abbreviations:

The following abbreviations are used throughout the following examples. The same abbreviations are used to identify the eluant systems in flash chromatography and medium-pressure chromatography. The other short forms and abbreviations used have the following meanings:
abs. absolute (indicates that the solvent is anhydrous)
atm physical atmospheres (unit of pressure)-1 atm corresponds to 1.013 bar
Boc tert-butoxycarbonyl
BOP benzotriazol-1-yloxy-tris(dimethylamino)phosphonium hexafluorophosphate brine saturated sodium chloride solution
DCC dicyclohexylcarbodiimide
DIPE diisopropyl ether
DMAP dimethylaminopyridine
DMF dimethylformamide
DMSO dimethyl sulfoxide
EDC N-ethyl-N′-(3-dimethylamino propyl)-carbodiimide hydrochloride
HBTU O-benzotriazol-1-yl-N,N,N′N′-tetramethyluronium hexafluorophosphate
HOBt 1-hydroxybenzotriazole
HV high vacuum
NMM N-methylmorpholine
RE rotary evaporator
RT room temperature
TFA trifluoroacetic acid
THF tetrahydrofuran
Z benzyloxycarbonyl

Mass spectroscopic data are obtained either by conventional MS or according to the “Fast-Atom-Bombardment” (FAB-MS) method. The mass data refer in the former case to the unprotonated molecule ion (M)⁺ or to the protonated molecule ion (M+H)⁺.

The values for proton nuclear resonance spectroscopy (¹H-NMR) are given in ppm (parts per million) based on tetramethylsilane as the internal standard. s=singlet, d=doublet, t=triplet, q=quartet, m=multiplet, dd=double doublet, br=broad.

The values for IR spectra are given in cm⁻¹, and the solvent in question is in round brackets. Where given, s indicates a strong, m a medium and w a weak intensity of the band in question.

The residue referred to as -[Phe^{NN}Phe] is the divalent residue of 3(S)-amino-4-phenyl-1-(N-benzylhydrazino)-butan-2(S)-ol and has the following formula:

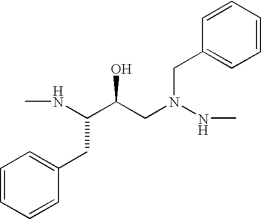

The residue referred to as -[Phe^{NN}Cha] is the divalent residue of 3(S)-amino-4-phenyl-1-(N-cyclohexylmethylhydrazino)-butan-2(S)-ol and has the following formula:

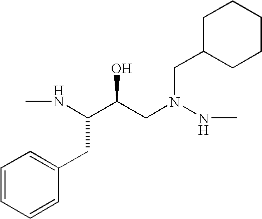

The residue referred to as -[Phe^{NN}Leu] is the divalent residue of 3(S)-amino-4-phenyl-1-(N-isobutylhydrazino)-butan-2(S)-ol and has the following formula:

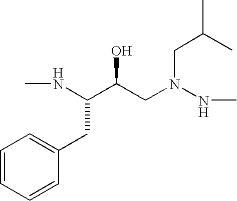

The residue referred to as -[Phe^{NN}Nle] is the residue of 3(S)-amino-4-phenyl-1-(N-n-butylhydrazino)-butan-2(S)-ol and has the following formula:

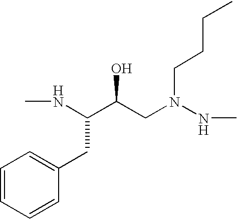

The residue referred to as -[Phe^{NN}(p-F)Phe] is the divalent residue of 3(S)-amino-4-phenyl-1-(N-(p-fluorophenylmethyl)-hydrazino)-butan-2(S)-ol and has the following formula:

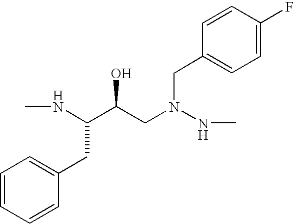

The residue referred to as -[(p-F)Phe^{NN}(p-F)Phe] is the divalent residue of 3(S)-amino-4-(p-fluorophenyl)-1-(N-(p-fluorophenylmethyl)-hydrazino)-butan-2(S)-ol and has the following formula:

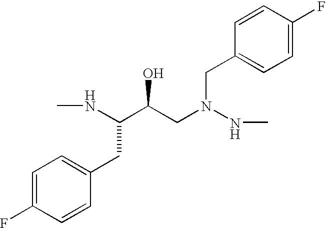

The residue referred to as -[Phe^{NN}(p-CN)Phe) is the divalent residue of 3(S)-amino-4-phenyl-1-(N-(p-cyanophenylmethyl)-hydrazino)-butan-2(S)-ol and has the following formula:

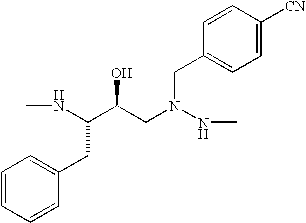

The residue referred to as -[Cha^{NN}Leu] is the divalent residue of 3(S)-amino-4-cyclohexyl-1-(N-isobutyl-hydrazino)-butan-2(S)-ol and has the following formula:

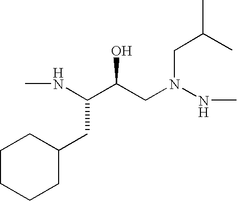

The divalent radical of 1-[2(S)-acetoxy-3(S)-amino-4-phenylbutyl]-[1-cyclohexylmethyl)hydrazine has the following formula:

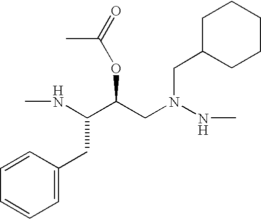

The abbreviations customarily used in peptide chemistry are used to name divalent residues of natural α-amino acids. However, contrary to customary peptide nomenclature in which the amino terminus is on the left-hand side and the carboxy terminus is on the right-hand side, amino acids that are on the right-hand side of the residues -[Phe^{NN}Phe], -[Phe^{NN}Cha], -[Phe^{NN} Leu], -[Phe^{NN}Nle], -[Phe^{NN}(p-F)Phe], -[(p-F)Phe^{NN}(p-F)Phe], -[Phe^{NN}(p-CN)Phe], or -[Cha^{NN} Leu] in the compound names, have the linking carboxy group on the left, which is indicated by an arrow (←) symbolising the reversal of the direction of linkage. The configuration at the α-carbon atom, if it is known, is indicated by the prefix (L)- or (D)-. Tyrosine residues etherified at the phenylic hydroxy group by the radical R are designated by Tyr(OR). Nle denotes the residue of norleucine.

### Example 1

### Boc-[Phe^{NN}Phe]-Boc

A solution of 300 mg (1.14 mmol) of (2R)-[1′(S)-Boc-amino-2′-phenylethyl]oxirane (J. Org. Chem. 50, 4615 (1985)) and 253 mg (1.14 mmol) of tert-butyl-3-benzyl-carbazate (J. Chem. Soc., Perkin I, 1712 (1975)) in 4 ml of methanol is heated under reflux for 12 hours. After cooling the reaction mixture to 0° C., a large portion of the title compound precipitates. The mother liquor is concentrated by evaporation and the residue is dissolved in a small amount of methylene chloride. After the dropwise addition of hexane a further amount of the title compound is obtained in the form of a white precipitate. FAB-MS: (M+H)⁺=486, t_{Ret} (I)=26.8 min, R_{f} (E)=0.70.

### Example 2

### Z-(L)-Val-[Phe^{NN}Phe←((L)-Val-Z)

191 mg (0.76 mmol) of Z-(L)-valine, 336 mg (0.76 mmol) of BOP and 103 mg (0.76 mmol) of HOBt are dissolved in 5 ml of a 0.3M solution of NMM in DMF, and after 10 minutes 100 mg (0.25 mmol) of H-[Phe^{NN}Phe]-H.3HCl are added and the mixture is stirred for 2 hours at RT under a nitrogen atmosphere. The reaction mixture is concentrated by evaporation, and the residue is dissolved in methylene chloride and washed twice with saturated sodium hydrogen carbonate solution. The organic phases are filtered through cotton wadding and concentrated by evaporation and the residue is purified by means of chromatography on silica gel with methylene chloride/ether (1:1). Lyophilisation of the product-containing fractions from dioxane yields the title compound in the form of a white solid. FAB-MS: (M+H)⁺=752, t_{Ret} (I)=27.8 min, R_{f} (E)=0.45.

The starting material is prepared as follows:

### a) H-[Phe^{NN}Phe]-H.3HCl

A solution of 280 mg (0.58 mmol) of Boc-[Phe^{NN}Phe]-Boc from Example 1 in 10 ml of 4N hydrogen chloride in dioxane is stirred for 2 hours at RT under a nitrogen atmosphere and then lyophilised. Lyophilisation once more from dioxane/tert-butanol yields the title compound in the form of a flocculent solid. FAB-MS: (M+H)⁺=286, t_{Ret} (II)=23.1 min, R_{f} (C)=0.17.

### Example 3

### Boc-(L)-Val-[Phe^{NN}Phe]←((L)-Val-Boc)

The title compound is obtained in a manner analogous to that described in Example 2 from 50 mg (0.13 mmol) of H-[Phe^{NN}Phe]-H.3HCl, 83 mg (0.83 mmol) of Boc-(L)-valine, 168 mg (0.38 mmol) of BOP, 51 mg (0.38 mmol) of HOBt and 2.5 ml of 0.3M NMM in DMF after chromatographic purification on silica gel with chloroform/methanol (95:5) and lyophilisation from dioxane. FAB-MS: (M+H)⁺=684, t_{Ret} (I)=27.4 min, R_{f} (E)=0.38.

### Example 4

### Boc-[Phe^{NN}Cha]-Boc

The title compound is obtained analogously to Example 1, from 231 mg (0.88 mmol) of (2R,3S)-1-[3-Boc-amino-2-phenylethyl]oxirane and 200 mg (0.88 mmol) of tert-butyl-3-cyclohexylmethyl-carbazate, in the form of a white precipitate from hexane. FAB-MS: (M+H)⁺=492, t_{Ret} (I)=30.4 min, R_{f} (E)=0.78.

The starting material is prepared as follows:

### a) tert-Butyl-3-cyclohexylmethyl-carbazate

10.2 g (45.1 mmol) of cyclohexylcarbaldehyde-tert-butoxycarbonylhydrazone, dissolved in 400 ml of methanol, are hydrogenated in the presence of 5.1 g of 5% platinum on carbon at RT and 4 atm hydrogen pressure. When the reaction is complete, the catalyst is removed by filtration and the filtrate is concentrated by evaporation. The residue is dissolved in methylene chloride and washed with water. Concentration by evaporation of the organic phase yields the title compound in the form of a colourless resin. ¹H-NMR (200 MHz, CDCl₃): 6.1 (s, br, 1H), 3.9 (s, br, 1H), 2.65 (d, 2H), 1.8-0.75 (m, 11H), 1.45 (s, 9H), t_{Ret} (I)=32.0 min, R_{f} (E)=0.75.

### b) Cyclohexylcarbaldehyde-tert-butoxycarbonylhydrazone

A solution of 10.8 g (81.2 mmol) of tert-butylcarbazate and 10.1 g (90 mmol) of cyclohexylcarbaldehyde in 400 ml of ethanol is heated under reflux for 2 hours. Half of the solvent is then removed by distillation and the title compound is precipitated by the addition of water. It is directly further used in a).

### Example 5

### H-(L)-Val-[Phe^{NN}Phe]←((L)-Val)-H.3HCl

A solution of 40 mg (0.06 mmol) of Boc-(L)-Val-[Phe^{NN}Phe]←((L)-Val)-Boc from Example 3 in 4 ml of 4N hydrogen chloride in dioxane is stirred at RT for 1 h. The mixture is then diluted with dioxane and, after lyophilisation, the title compound is obtained in the form of the hydrochloride. FAB-MS: (M+H)⁺=484, t_{Ret} (II)=25.8 min, R_{f} (A)=0.45.

### Example 6

### N-Thiomorpholinocarbonyl-(L)-Val-[Phe^{NN}Phe]←(N-thiomorpholinocarbonyl-(L)-Val)

35 μl (0.25 mmol) of triethylamine and 16 mg (0.1 mmol) of (4-thiomorpholinylcarbonyl)chloride are added in succession at RT to a solution of 20 mg (0.03 mmol) of H-(L)-Val-[Phe^{NN}Phe]←((L)-Val)-H.3HCl from Example 5 in 0.5 ml of DMF, and the mixture is stirred for 1 h at RT. The reaction mixture is diluted with chloroform and washed with saturated sodium hydrogen carbonate solution. The organic phase is filtered through cotton wadding and concentrated by evaporation, and the residue is chromatographed on silica gel with a gradient of chloroform/methanol (15:1→8:1). The product fractions are concentrated by evaporation and precipitated with methylene chloride/DIPE. Lyophilisation from dioxane yields the title compound in the form of a flocculent solid. FAB-MS: (M+H)⁺=742, t_{Ret} (I)=21.6 min, R_{f} (D)=0.54.

The starting material is prepared as follows:

### a) (4-Thiomorpholinylcarbonyl)chloride

A solution of 10 g (97 mmol) of thiomorpholine in 200 ml of toluene is added dropwise at 0° C. to a solution of 85 ml (165 mmol) of 20% phosgene in toluene and the white suspension is stirred for 1 h at RT. Excess phosgene is driven off by introducing nitrogen, the suspension is filtered, and the filtrate is concentrated by evaporation. The title compound is obtained in the form of a yellow oil. IR (CH₂Cl₂, cm⁻¹): 1735, 1450, 1440, 1405, 1370, 1290, 1180.

### Example 7

### N-Morpholinocarbonyl-(L)-Val-[Phe^{NN}Phe]←(N-morpholinocarbonyl-(L)-Val)

210 μl (1.52 mmol) of triethylamine are added to a solution of 100 mg (0.25 mmol) of H-[Phe^{NN}Phe]-H.3HCl from Example 2a), 163 mg (0.76 mmol) of N-morpholino-carbonyl-(L)-valine and 288 mg (0.76 mmol) of HBTU in 2 ml of DMF and the mixture is stirred for 16 h at RT under a nitrogen atmosphere. The reaction mixture is fully concentrated by evaporation, and the residue is dissolved in methylene chloride and washed with saturated sodium hydrogen carbonate solution. The organic phase is filtered through cotton wadding, concentrated by evaporation and chromatographed on silica gel with methylene chloride/methanol (15:1). The title compound is precipitated from methylene chloride/hexane and, after lyophilisation from dioxane/tert-butanol, is obtained in the form of a flocculent solid. FAB-MS: (M+H)⁺=710, t_{Ret} (I)=16.3 min, R_{f} (E)=0.16.

The starting material is prepared as follows:

### a) N-Morpholinocarbonyl-(L)-valine

2.7 g (8.4 mmol) of N-morpholinocarbonyl-(L)-valine-benzyl ester are dissolved in 75 ml of ethyl acetate and the solution is hydrogenated for 3 h in the presence of 500 mg of 10% palladium on carbon at 1 atm hydrogen pressure and RT. The catalyst is filtered off and, after concentrating the solvent by evaporation, the title compound is obtained in the form of a colorless oil. ¹H-NMR (300 MHz, CD₃OD): 4.15 (m, 1H), 3.65 (m, 4H), 3.40 (m, 4H), 2.12 (m, 1H), 0.95 (2d, 6H).

### b) N-Morpholinocarbonyl-(L)-valine-benzyl Ester

0.8 ml (8.1 mmol) of (morpholinocarbonyl)chloride (preparation: J. Med. Chem. 31, 2277 (1988)) and 4.1 mL (24.1 mmol) of N-ethyldiisopropylamine are added to a solution of 4 g (10.5 mmol) of (L)-valine-benzyl ester 4-toluenesulfonate in 56 mL of methylene chloride and the mixture is stirred at RT for 24 h. The reaction mixture is diluted with ethyl acetate and washed in succession with 1N hydrochloric acid, water, saturated sodium hydrogen carbonate solution and brine. The organic phase is dried over sodium sulfate and concentrated by evaporation. Chromatography on silica gel with ethyl acetate yields N-morpholinocarbonyl-(L)-valine-benzyl ester in the form of a colourless oil. The ester is directly further used in a).

### Example 8

### Phenylacetyl-(L)-Val-[Phe^{NN}Phe]←(N-phenylacetyl-(L)-Val)

The title compound is obtained analogously to Example 7 from 100 mg (0.25 mmol) of H-[Phe^{NN}Phe]-H.3HCl from Example 2a), 143 mg (0.61 mmol) of phenylacetyl-(L)-valine (preparation: Mem. Tokyo Univ. Agric. 20, 51 (1978)), 230 mg (0.61 mmol) of HBTU and 200 μL (1.42 mmol) of triethylamine after chromatographic purification with methylene chloride/ether/methanol (20:20:1) and lyophilisation from dioxane/tert-butanol. FAB-MS: (M+H)⁺=720, t_{Ret} (I)=23.7 min, R_{f} (G)=0.21.

### Example 9

### N-(3-Pyridylacetyl)(L)-Val-[Phe^{NN}Phe]←(N-(3pridylacetyl)-(L)-Val)

The title compound is obtained analogously to Example 7 in the form of a white solid from 100 mg (0.25 mmol) of H-[Phe^{NN} Phe]-H.3HCl from Example 2a, 576 mg (1.52 mmol) of HBTU, 358 mg (1.52 mmol) of N-(3-pyridylacetyl)-(L)-valine and 316 μL (2.3 mmol) of triethylamine after chromatographic purification with chloroform/methanol (5:1) and lyophilisation from dioxane/tert-butanol. FAB-MS: (M+H)⁺=722, t_{Ret} (II)=27.9 min, R_{f} (A)=0.71.

The starting material is prepared as follows:

### a) N-(3-Pyridylacetyl)-(L)-valine

3.4 g of N-(3-pyridylacetyl)-(L)-valine-tert-butyl ester are dissolved in 20 mL of trifluoroacetic acid/methylene chloride (1:1) and the solution is stirred at RT for 16 h. The reaction solution is fully concentrated by evaporation and the residue is digested with DIPE. The title compound is obtained in the form of a white amorphous solid. ¹H-NMR (200 MHz, CD₃OD): 8.9-8.6 (m, broad, 1H), 8.5 (m, 1H), 7.95 (m, 1H), 4.33 (m, 1H), 3.93 (s, 2H), 2.2 (m, 1H), 0.98 (2d, 6H).

### b) N-(3-Pyridylacetyl)-(L)-valine-tert-butyl Ester

4.2 mL of triethylamine are added dropwise at 0° C. to a solution of 3.36 g (16 mmol) of (L)-valine-tert-butyl ester.HCl, 2 g (14.5 mmol) of 3-pyridylacetic acid and 2.17 mL (14.3 mmol) of cyanophosphonic acid diethyl ester in 20 mL of DMF. The reaction mixture is stirred for 48 h at RT, and then diluted with methylene chloride and washed with 10% citric acid as well as saturated sodium hydrogen carbonate solution. The organic phase is filtered through cotton wadding and, after removal of the solvent by evaporation, yields N-(3-pyridylacetyl)-(L)-valine-tert-butyl ester, which is directly further used in a).

### Example 10

### Boc-(L)-Val-[Phe^{NN}Cha]←((L)-Val)-Boc

The title compound is obtained analogously to Example 7 in the form of a flocculent solid starting from 500 mg (1.25 mmol) of H-[Phe^{NN}Cha]-H.3HCl, 1.08 g (4.98 mmol) of Boc-(L)-valine, 1.89 g (4.98 mmol) of HBTU and 1.39 mL (9.96 mmol) of triethylamine after chromatographic purification on silica gel with methylene chloride/ether (1:1) and lyophilisation from dioxane. FAB-MS: (M+H)⁺=690, t_{Ret} (I)=29.3 min, R_{f} (H)=0.48.

The starting material is prepared as follows:

### a) H-[Phe^{NN}Cha]-H.3HCl

1.10 g (2.2 mmol) of Boc-[Phe^{NN}Cha]-Boc from Example 4 is dissolved in 20 mL of 4N hydrogen chloride in dioxane and the solution is stirred at RT for 3 h. Lyophilisation of the reaction solution yields the title compound in the form of the hydrochloride. FAB-MS: (M+H)⁺=292, t_{Ret} (II)=27.3 min.

### Example 11

### Z-(L)-Val-[Phe^{NN}Cha]←((L)-Val)-Z

The title compound is obtained analogously to Example 2 from 50 mg (0.12 mmol) of H-[Phe^{NN}Cha]-H.3HCl from Example 10a, 94 mg (0.37 mmol) of Z-(L)-valine, 165 mg (0.37 mmol) of BOP, 51 mg (0.37 mmol) of HOBt and 2.5 mL of 0.3M NMM in DMF after chromatographic purification on silica gel with methylene chloride/ether (1:1) and lyophilisation from dioxane. FAB-MS: (M+H)⁺=758, t_{Ret} (I)=29.1 min, R_{f} (H)=0.55.

### Example 12

### Boc-[Phe^{NN}Leu]-Boc

The title compound is obtained analogously to Example 1, starting from 1.0 g (3.8 mmol) of (2R)-[1′(S)-Boc-amino-2′-phenylethyl]oxirane and 715 mg (3.8 mmol) of tert-butyl-3-isobutyl-carbazate (preparation: J. Chem. Soc., Perkin I, 1712 (1975)), in the form of a precipitate from hexane. FAB-MS: (M+H)⁺=452, t_{Ret} (I)=27.2 min, R_{f} (I)=0.55.

### Example 13

### Z-(L)-Val-[Phe^{NN}Leu]←((L)-Val)-Z

The title compound is obtained analogously to Example 2 starting from 60 mg (0.17 mmol) of H-[Phe^{NN}Leu]-H.3HCl, 125 mg (0.50 mmol) of Z-(L)-valine, 221 mg (0.50 mmol) of BOP, 67 mg (0.50 mmol) of HOBt and 3.3 mL of 0.3M NMM in DMF after chromatographic purification on silica gel with methylene chloride/ether (1:1) and lyophilisation from dioxane. FAB-MS: (M+H)⁺=718, t_{Ret} (I)=26.8 min, R_{f} (H)=0.38.

The starting material is prepared as follows:

### a) H-[Phe^{NN}Leu]-H.3HCl

The title compound is obtained analogously to Example 10a), in the form of a lyophilisate, from 1.21 g (2.48 mmol) of Boc-[Phe^{NN}Leu]-Boc from Example 12. FAB-MS: (M+H)⁺=252, t_{Ret} (II)=20.9 min, R_{f} (K)=0.23.

### Example 14

### H-(L)-Val-[Phe^{NN} Cha]←((L)-Val)-H.3HCl

The title compound is obtained analogously to Example 10a), from 632 mg (0.91 mmol) of Boc-(L)-Val-[Phe^{NN}Cha]←((L)-Val)-Boc from Example 10, in the form of the hydrochloride after lyophilisation. FAB-MS: (M+H)⁺=490, t_{Ret} (II)=29.4 min, R_{f} (K)=0.23.

### Example 15

### N-(3-Pyridylacetyl)-(L)-Val-[Phe^{NN}Leu]←(N-(3-pyridylacetyl)-(L)-Val)

The title compound is obtained analogously to Example 9 from 90 mg (0.25 mmol) of H-[Phe^{NN}Leu]-H.3HCl from Example 13a), 358 mg (1.52 mmol) of N-(3-pyridylacetyl)-(L)-valine, 576 mg (1.52 mmol) of HBTU and 316 μL (2.5 mmol) of triethylamine after chromatographic purification with methylene chloride/methanol (15:1) and lyophilisation from dioxane/tert-butanol/water. FAB-MS: (M+H)⁺+688, t_{Ret} (IV)=15.5 min, R_{f} (D)=0.37.

### Example 16

### N-Trifluoroacetyl-[Phe^{NN}(p-F)Phe]-Boc

A solution of 4.0 g (15.4 mmol) of 2(R)-[1′(S)-(trifluoroacetylamino)-2′-phenylethyl]oxirane and 3.89 g (16.2 mmol) of tert-butyl-3-(p-fluorophenyl-methyl)-carbazate in 35 mL of methanol are heated at 80° C. for approximately 20 h in a bomb tube. The reaction mixture is concentrated by evaporation, the residue is dissolved in a small amount of dichloromethane, and the title compound is precipitated therefrom using hexane (refrigerator). Column chromatography (SiO₂, methylene chloride/ether 95:7) yields further product. TLC R_{f} (J)=0.57; t_{Ret} (I)=24.3 min; FAB-MS (M+H)⁺=500.

The starting materials are prepared as follows:

### a) N-3(S)-(Boc-amino)-2(R,S)-hydroxy-4-phenyl-1-trimethylsilyl-butane

Under a nitrogen atmosphere, 24.7 g (1.02 mol) of magnesium is placed in 100 mL of abs. ether and over a period of 35 minutes a small amount of iodine and, at the same time, 132.5 mL (0.95 mol) of chloromethyltrimethylsilane and 300 mL of ether are added, the temperature being maintained at 38° C. by means of an ice bath. The reaction mixture obtained is then stirred for 1.5 h at RT. After the mixture has been cooled to −60° C., a suspension of 48.6 g (0.195 mol) of N-Boc-phenylalaninal (preparation: D. J. Kempf, J. Org. Chem. 51, 3921 (1986)) in 1.1 L of ether is added over a period of 40 min. Over a period of 90 min the reaction mixture is warmed to RT and stirred for a further 90 min at that temperature. The mixture is then poured onto 2 L of ice-water and 1.5 L of 10% aqueous citric acid. The separated aqueous phase is extracted twice with 500 mL of ether. All ether extracts are washed with 500 mL of 10% citric acid and twice with brine. After drying over sodium sulfate the residue is concentrated in vacuo and the resulting title compound is further used without additional purification. TLC R_{f} (L)=0.6; FAB-MS (M+H)⁺=338.

### b) 1-Phenyl-3-buten-2(S)-amine

35.6 mL (0.28 mol) of an approximately 48% solution of boron trifluoride in ether are added at 5° C., over a period of 10 min, to a solution of 18.8 g (0.055 mol) of 3(S)-(Boc-amino)-2(R,S)-hydroxy-4-phenyl-1-trimethylsilyl-butane in 420 mL of methylene chloride. The reaction mixture is then stirred at RT for 16 h, cooled to 10° C. and, over a period of 20 min, 276 mL of a 4N sodium hydroxide solution is added. The aqueous phase is removed and extracted twice with 400 mL of methylene chloride each time. The combined organic extracts are washed with brine and dried over sodium sulfate. The title product is further used without additional purification. TLC R_{f} (C)=0.15; IR (methylene chloride) (cm⁻¹): 3370, 3020, 2920, 1640, 1605.

### c) N-Trifluoroacetyl-1-phenyl-3-buten-2(S)-amine

17.0 mL (121 mmol) of trifluoroacetic acid anhydride are added dropwise, at 0° C., to 11.9 g (81 mmol) of 1-phenyl-3-buten-2(S)-amine dissolved in 210 mL of methylene chloride and 70 mL of pyridine. The mixture is stirred for 0.5 h at 0° C. and then extracted twice with dilute HCl, water and brine. The aqueous phases are washed a further twice with methylene chloride, dried with sodium sulfate and concentrated by evaporation: TLC R_{f} (M)=0.4.

### d) 2(R)-[1′(S)-(Trifluoroacetylamino)-2′-phenylethyl]-oxirane

54.28 g (314 mmol) of m-chloroperbenzoic acid are added to a solution of 14.5 g (60 mmol) of N-trifluoroacetyl-1-phenyl-3-buten-2(S)-amine in 600 mL of chloroform and the mixture is stirred for 24 h at RT to complete the reaction. The reaction mixture is washed twice with 10% sodium sulfite solution, twice with saturated sodium carbonate solution, water and brine. The aqueous phases are extracted a further twice with methylene chloride and the combined organic phases are dried with sodium sulfate and concentrated by evaporation to yield the title compound, which is used in the next step without being further purified: TLC R_{f} (N)=0.6.

### e) p-Fluorophenylcarbaldehyde-tert-butoxycarbonylhydrazone

32 g (242 mmol) of tert-butylcarbazate and 30 g (242 mmol) of p-fluorobenzaldehyde in 300 mL of ethanol are reacted analogously to Example 4b) for 3 h at 80.° C. to form the title compound, which crystallized on cooling and diluting with water: TLC R_{f} (N)=0.48; t_{Ret} (I)=19.4 min.

### f) tert-Butyl-3-(p-fluorophenyl-methyl)-carbazate

55 g (231 mmol) of p-fluorophenylcarbaldehyde-tert-butoxycarbonylhydrazone in 500 mL of THF are hydrogenated with 5.5 g of palladium (5%) on carbon analogously to Example 4a) to yield the title compound: ¹H-NMR (200 MHz, CD₃OD): 7.35 (dd, 8 and 6 Hz, 2H), 7.05 (t, 8 Hz, 2H), 3.9 (s, 2H), 1.45 (s, 9H).

### Example 17

### N-Morpholinocarbonyl-(L)-Val-[Phe^{NN}(p-F)Phe]-Boc

A mixture of 185 mg (0.80 mmol) of N-morpholinocarbonyl-(L)-valine (for preparation see Example 7a)), 270 mg (0.67 mmol) of H-[Phe^{NN}(p-F)Phe]-Boc, 311 mg (0.70 mmol) of BOP and 95 mg (0.70 mmol) of HOBT is dissolved at RT in 6.8 mL of 0.3M NMM/DMF and stirred for 5 h at RT. The reaction mixture is concentrated by evaporation under HV and the residue is partitioned between 4 portions of methylene chloride and 2 portions of 1M sodium carbonate solution, water and brine. The combined organic phases are dried over sodium sulfate, concentrated by evaporation and purified by column chromatography (SiO₂, ethyl acetate): TLC R_{f} (O)=0.38; t_{Ret} (I)=21.8 min; FAB-MS (M+H)⁺=616.

The starting material is prepared as follows:

### a) H-[Phe^{NN}(p-F)Phe]-Boc

At 70° C., 15 mL of a 1M aqueous solution of potassium carbonate are added dropwise to a solution of 0.3 g (0.6 mmol) of N-trifluoroacetyl-[Phe^{NN}(p-F)Phe]-Boc (for preparation see Example 16) in 50 mL of methanol under a nitrogen atmosphere and the mixture is stirred for 25 h at that temperature. The reaction mixture is concentrated by evaporation under HV, methylene chloride is added to the residue and the mixture is washed twice with water and brine. The aqueous phases are extracted twice with methylene chloride and the organic phases are dried with sodium sulfate and concentrated by evaporation. The crude product is used in the next step without being further purified: t_{Ret} (I)=16.2 min.

### Example 18

### N-Morpholinocarbonyl-(L)-Val-[Phe^{NN}(p-F)Phe]←-((L)-Val)-Z

129 mg (0.34 mmol) of HBTU are added to a solution of 86 mg (0.34 mmol) of Z-(L)-Val and 160 mg (0.31 mmol) of N-morpholinocarbonyl-(L)-Val-[Phe^{NN}(p-F)Phe]-H in 2.7 mL of 0.25M NMM/CH₃CN (0.25M NMM in CH₃CN). After 4 h at RT the mixture is concentrated by evaporation and the residue is partitioned between 3 portions of methylene chloride and 2 portions of saturated sodium hydrogen carbonate solution and brine. The organic phases are dried with sodium sulfate and concentrated by evaporation to yield the title compound which is obtained in pure form after digestion from methylene chloride/ether 1:1: TLC R_{f} (P)=0.4; t_{Ret} (I)=22.4 min; FAB-MS (M+H)⁺=749.

The starting material is prepared as follows:

### a) N-Morpholinocarbonyl-(L)-Val-[Phe^{NN}(p-F)Phe]-H

210 mg (0.34 mmol) of N-morpholinocarbonyl-(L)-Val-[Phe^{NN}(p-F)Phe]-Boc (Example 17) are dissolved in 105 mL of formic acid and the solution is stirred for 4 h at RT and then concentrated by evaporation. The residue is taken up in methylene chloride and the solution is washed with saturated sodium hydrogen carbonate solution and brine. Extraction of the aqueous phases with 2 portions of methylene chloride, drying the organic phases with sodium sulfate and concentrating by evaporation yields the title compound, which is used in the next step without being further purified: t_{Ret} (I)=12.9.

### Example 19

### N-Morpholinocarbonyl-(L)-Val-[Phe^{NN}(p-F)Phe]←((L)-Val)-H

160 mg (0.21 mmol) of N-morpholinocarbonyl-(L)-Val-[Phe^{NN}(p-F)Phe]←((L)-Val)-Z (Example 18) in 6 mL of ethanol are hydrogenated with 40 mg of palladium (10%) on carbon at normal pressure. Filtration through Celite (siliceous earth, filter aid from Fluka, Buchs, Switzerland), concentration by evaporation and lyophilisation from dioxane yield the title compound: t_{Ret} (hydrochloride, I)=13.4 min; FAB-MS (M+H)⁺=615.

### Example 20

### N-Morpholinocarbonyl-(L)-Val-[Phe^{NN}(p-F)Phe]←((L)-Val)-(N-morpholinocarbonyl-Gly)

54 mg (0.143 mmol) of HBTU are added to a solution of 26.9 mg (0.143 mmol) of N-morpholinocarbonyl-glycine and 80 mg (0.130 mmol) of N-morpholinocarbonyl-(L)-Val-[Phe^{NN}(p-F)Phe]←-((L)-Val)-H in 1.1 ml of 0.25M NMM/CH₃CN and the mixture is stirred for 16 h at RT. The mixture is concentrated by evaporation and the residue is partitioned between 3 portions of ethyl acetate and water and 2 portions of saturated sodium hydrogen carbonate solution, water and brine. The organic phases are dried with sodium sulfate and concentrated by evaporation to yield the title compound which, after dissolving in a small amount of DMF and precipitating with DIPE is obtained in pure form: t_{Ret} (I)=15.1 min; FAB-MS (M+H)⁺=785.

The starting material is prepared as follows:

### a) N-Morpholinocarbonyl-glycine-benzyl Ester

Analogously to Example 7b), 7.69 g (22.8 mmol) of glycine-benzyl ester 4-toluenesulfonate and 2.8 g (19 mmol) of (morpholinocarbonyl)chloride in 118 mL of methylene chloride and 9 mL (53 mmol) of N-ethyldiisopropylamine are reacted for 18 h. The title compound is obtained in pure form after extraction with methylene chloride and digestion with hexane: t_{Ret} (I)=11.6 min.

### b) N-Morpholinocarbonyl-glycine

Analogously to Example 7a), 4.8 g (18.3 mmol) of N-morpholinocarbonyl-glycine-benzyl ester in 100 mL of ethyl acetate are hydrogenated with 1 g of palladium (10%) on carbon to yield the title compound: ¹H-NMR (300 MHz, CDCl₃): 3.88 (s, 2H), 3.64 (s, 4H), 3.50 (s, 2H), 3.35 (s, 4H).

### Example 21

### Z-(L)-Val-[Phe^{NN}(p-F)Phe]-Boc

463 mg (1.22 mmol) of HBTU are added to a solution of 335 mg (1.33 mmol) of Z-(L)-Val and 448 mg (1.11 mmol) of H-[Phe^{NN}(p-F)Phe]-Boc (for preparation see Example 17a)) in 9.4 mL of 0.25M NMM/CH₃CN (0.25M NMM in CH₃CN). After stirring for 16 h at RT, the mixture is concentrated by evaporation and the residue is partitioned between 3 portions of methylene chloride and 2 portions of saturated sodium hydrogen carbonate solution and brine. The organic phases are dried with sodium sulfate and concentrated by evaporation to yield the title compound, which is purified by column chromatography (SiO₂, hexane/ethyl acetate 4:1→1:1): t_{Ret} (I)=26.6 min; FAB-MS (M+H)⁺=637.

### Example 22

### Z-(L)-Val-[Phe^{NN}(p-F)Phe]←-((L)-Val)-Boc

Analogously to Example 18, 165 mg (0.76 mmol) of Boc-(L)-Val and 371 mg (0.69 mmol) of Z-(L)-Val-[Phe^{NN}(p-F)Phe]-H in 6 mL of 0.25M NMM/CH₃CN are reacted with 289 mg (0.76 mmol) of HBTU to yield the title compound, which can be crystallized directly from the reaction solution and filtered off: t_{Ret} (I)=27.2 min; FAB-MS (M+H)⁺=736.

The starting material is prepared as follows:

### a) Z-(L)-Val-[Phe^{NN}(p-F)Phe]-H

Analogously to Example 18a), 440 mg (0.69 mmol) of Z-(L)-Val-[Phe^{NN}(p-F)Phe]-Boc are deprotected with 212 mL of formic acid to yield the title compound: t_{Ret} (I)=17.8 min.

### Example 23

### Z-(L)-Val-[Phe^{NN}(p-F) Phe]←((L)-Val)-H

Analogously to Example 18a), 250 mg (0.34 mmol) of Z-(L)-Val-[Phe^{NN}(p-F)Phe]←((L)-Val)-Boc (Example 22) are deprotected with 50 ml of formic acid to yield the title compound: t_{Ret} (I)=18.0 min; FAB-MS (M+H)⁺=636.

### Example 24

### Z-(L)-Val-[Phe^{NN}(p-F)Phe]←-((L)-Val)←(N-morpholinocarbonyl-Gly)

Analogously to Example 20, 32 mg (0.17 mmol) of N-morpholinocarbonyl-glycine (Example 20b)) and 99 mg (0.16 mmol) of Z-(L)-Val-[Phe^{NN}(p-F)Phe]←-((L)-Val)-H in 1.3 mL of 0.25M NMM/CH₃CN are reacted with 65 mg (0.17 mmol) of HBTU to yield the title compound, which crystallizes directly from the reaction solution: t_{Ret} (I)=21.1 min; FAB-MS (M+H)⁺=806.

### Example 25

### Z-(L)-Asn-[Phe^{NN}(p-F) Phe]-Boc

3.0 g (7.8 mmol) of Z-(L)-asparagine-p-nitrophenyl ester (Bachem, Bubendorf/Switzerland) are added to a solution of 2.09 g (5.2 mmol) of H-[Phe^{NN}(p-F)Phe]-Boc (for preparation see Example 17a)) in 68 mL of DMF and 2.7 mL (16 mmol) of N-ethyl-diisopropylamine. The mixture is stirred for 16 h at RT and concentrated by evaporation under HV, and the residue is taken up in a large amount of methylene chloride (poorly soluble) and washed with 2 portions of 5% potassium carbonate solution. The aqueous phases are extracted twice more with a large amount of methylene chloride, and the combined organic phases are dried with sodium sulfate and concentrated by evaporation. The title compound is obtained by dissolving the crude product in a small amount of methanol and precipitating by the addition of toluene at −20° C.: t_{Ret} (I)=21.2 min.

### Example 26

### H-(L)-Asn-[Phe^{NN}(p-F)Phe]-Boc

Analogously to Example 19, 0.40 g (0.61 mmol) of Z-(L)-Asn-[Phe^{NN}(p-F)Phe]-Boc are hydrogenated in 20 mL of methanol to yield the title compound: t_{Ret} (I)=14.9 min.

### Example 27

### Quinoline-2-carbonyl-(L)-Asn-[Phe^{NN}(p-F)Phe]-Boc

Analogously to Example 17, 134 mg (0.78 mmol) of quinoline-2-carboxylic acid (Fluka, Buchs/Switzerland) in 4 mL of 0.3M NMM/DMF are reacted with 344 mg (0.78 mmol) of BOP, 105 mg (0.78 mmol) of HOBT and 268 mg (0.52 mmol) of H-(L)-Asn-[Phe^{NN}(p-F)Phe]-Boc. Since, according to HPLC, there is still some H-(L)-Asn-[Phe^{NN}(p-F)Phe]-Boc present after stirring for 16 h at RT, a further 299 mg of BOP, 70 mg of HOBT, 89 mg of quinaldic acid and 113 μL of NMM are added. After a further 16 h the mixture is concentrated by evaporation and the residue is partitioned between 3 portions of methylene chloride and 2 portions of saturated sodium hydrogen carbonate solution and brine. The combined organic phases are dried with sodium sulfate and concentrated by evaporation. The crude product is dissolved in a small amount of DMF, precipitated with DIPE and cooled to −20° C. to yield the title compound: t_{Ret} (I)=22.8 min; FAB-MS (M+H)⁺=673.

### Example 28

### Z-(L)-Asn-[Phe^{NN}(p-F)Phe]←((L)-Val)-Z

88 mg (0.35 mmol) of Z-(L)-Val in 3.8 mL of 0.3M NMM/DMF are activated with 153 mg (0.35 mmol) of BOP and 47 mg (0.35 mmol) of HOBT and, after 15 min, 144 mg (0.23 mmol) of Z-(L)-Asn-[Phe^{NN} (p-F)Phe]-H.2HCl are added. The reaction mixture is stirred for 14 h at RT and then concentrated by evaporation, the residue is dissolved in 2 mL of methanol and partitioned between 3 portions of methylene chloride and 2 portions of 1M sodium carbonate solution, and the organic phases are dried with sodium sulfate and concentrated by evaporation. Repeated dissolution of the crude product in a small amount of DMF and precipitation with DIPE yield the title compound: t_{Ret} (I)=22.2 min; FAB-MS (M+H)⁺=785.

The starting material is prepared as follows:

### a) Z-(L)-Asn-[Phe^{NN}(p-F)Phe]-H.2HCl

Under a nitrogen atmosphere, 2 mL of 4N HCl/dioxane (Fluka, Buchs/Switzerland) are added to a solution of 150 mg (0.23 mmol) of Z-(L)-Asn-[Phe^{NN}(p-F)Phe]-Boc (Example 25) in 1 mL of dioxane. The reaction mixture is stirred for 1.5 h at RT and then lyophilised, and the lyophilisate is directly further reacted.

### Example 29

### Trifluoroacetyl-[Phe^{NN}(p-F)Phe]←((L)-Val)-Z

Analogously to Example 17, 239 mg (0.95 mmol) of Z-(L)-Val in 10.5 mL of 0.3M NMM/DMF are reacted for 15 h with 421 mg (0.95 mmol) of BOP, 129 mg (0.95 mmol) of HOBT and 0.3 g (0.63 mmol) of N-trifluoroacetyl-[Phe^{NN}(p-F)Phe]-H. Column chromatography (SiO₂, methylene chloride/ether 10:1) and precipitation from DMF solution with DIPE yield the title compound: TLC R_{f} (O)0.15; t_{Ret} (I)=25.9 min; FAB-MS (M+H)⁺=633.

The starting material is prepared as follows:

### a) N-Trifluoroacetyl-[Phe^{NN}(p-F)Phe]-H

At 0° C., 5 mL of trifluoroacetic acid are added to 0.20 g (0.40 mmol) of N-trifluoroacetyl-[Phe^{NN}(p-F)Phe]-Boc (for preparation see Example 16) in 5 mL of methylene chloride. The reaction mixture is stirred for 4 h at 0° C. and for 2 h at RT and then concentrated by evaporation. Lyophilisation of the residue from dioxane yields the title compound, which is further reacted without being purified: t_{Ret} (I)=14.7 min.

### Example 30

### Z-(L)-Asn-[Phe^{NN}Phe]-Boc

Analogously to Example 25, 167 mg (0.34 mmol) of H-[Phe^{NN}Phe]-Boc in 3.6 ml of DMF and 0.18 mL (1 mmol) of N-ethyl-diisopropylamine are reacted with 0.20 g (0.52 mmol) of Z-(L)-asparagine-p-nitrophenyl ester to yield the title compound, which is obtained in pure form by column chromatography (SiO₂, ethyl acetate): TLC R_{f} (O)=0.19; t_{Ret} (I)=20.9 min.

The starting material is prepared as follows:

### a) N-Trifluoroacetyl-[Phe^{NN}Phe]-Boc

Analogously to Example 16, 1.82 g (7.0 mmol) of 2(R)-[1′(S)-(trifluoroacetylamino)-2′-phenylethyl]-oxirane (Example 16d)) and 1.58 g (7.1 mmol) of tert-butyl-3-benzylcarbazate (J. Chem., Perkin I, 1712 (1975)) in 15 mL of methanol are reacted in a bomb tube to yield the title compound, which is isolated by column chromatography (SiO₂, methylene chloride/ether 50:1): TLC R_{f} (J)=0.38; t_{Ret} (I)=24.5 min.

### b) H-[Phe^{NN}Phe]-Boc

Analogously to Example 17a), 258 mg (0.53 mmol) of N-trifluoroacetyl-[Phe^{NN}Phe]-Boc in 60 mL of methanol are reacted with 10.7 mL of 1M potassium carbonate solution to yield the title compound.

### Example 31

### Z-(L)-Val-[(p-F) Phe^{NN}(p-F) Phe]-Boc

Analogously to Example 21, 18 mg (0.070 mmol) of Z-(L)-Val and 27 mg (0.064 mmol) of H-[(p-F)Phe^{NN}(p-F)Phe]-Boc in 0.6 mL of 0.25M NMM/CH₃CN are reacted with 26.6 mg (0.070 mmol) of HBTU to yield the title compound, which is purified by dissolving in a small amount of methylene chloride and precipitating with DIPE: FAB-MS (M+H)⁺=655.

The starting material is prepared as follows:

### a) N-Boc-(p-fluorophenylalanine)

In 0.4 1 of dioxane/water 1:1 20 g (109 mmol) of p-fluorophenylalanine (Fluka, Buchs, Switzerland) are reacted with 35.5 g (163 mmol) of Boc-anhydride and 150 g (1.09 mol) of potassium carbonate. After 4 h, the reaction mixture is acidified with citric acid solution and extracted with 3 portions of ethyl acetate. The organic phases are washed with 10% citric acid, water and brine, dried with sodium sulfate and concentrated by evaporation. Dissolution of the residue in a small amount of methylene chloride and crystallisation by the addition of hexane yields the title compound: t_{Ret} (I)=16.9 min.

### b) N-Boc-(p-fluorophenylalaminol)

At from −5° C. to −10° C. 9.66 mL (69 mmol) of triethylamine are added to a solution of 17.9 g (63 mmol) of N-Boc-(p-fluorophenylalanine) in 73 mL of abs. THF, and a solution of 9.05 mL (69 mmol) of chloroformic acid isobutyl ester in 44 mL of abs. THF is added dropwise thereto. After stirring for 0.5 h at RT, the resulting precipitate is filtered off with suction. The filtrate is added dropwise, with cooling, to 4.77 g (126 mmol) of sodium borohydride in 28 mL of water. The mixture is stirred for 4 h at RT and acidified with 10% citric acid, the THF is partially removed by evaporation using a RE and the residue is partitioned between 3 portions of ethyl acetate and 2 portions of 2N sodium hydroxide solution, water, saturated sodium hydrogen carbonate solution and brine. The organic phases are dried with sodium sulfate, concentrated by evaporation, dissolved in a small amount of methylene chloride and crystallized by the addition of hexane to yield the title compound: TLC R_{f} (N)=0.36; t_{Ret} (I)=16.8 min; ¹H-NMR (200 MHz, CD₃OD): 7.24 (dd, 8 and 5 Hz, 2H), 6.98 (t, 8 Hz, 2H), 3.73 (m, 1H), 3.47 (d, 5 Hz, 2H), 2.88 (dd, 13 and 6 Hz, 1H), 2.62 (dd, 13 and 8 Hz, 1H), 1.36 (s, 9H).

### c) N-Boc-(p-fluorophenylalaninal)

Under a nitrogen atmosphere, 4.44 mL (62.4 mmol) of DMSO dissolved in 76 mL of methylene chloride are added dropwise to a solution, cooled to −60° C., of 4.0 mL (46.8 mmol) of oxalyl chloride in 44 mL of methylene chloride. After stirring for 15 minutes, resulting in a clear reaction solution, 8.4 g (31.2 mmol) of N-Boc-(p-fluorophenylalaminol) in the form of a solution in 185 mL of methylene chloride/THF 1:1 are added (→precipitation) and the mixture is then stirred for 25 min. 17.3 mL (124.8 mmol) of triethylamine dissolved in 38 mL of methylene chloride are then added. After the mixture has been stirred for 30 min, 278 mL of a 20% potassium hydrogen sulfate solution are added dropwise, followed by 220 mL of hexane. The mixture is left to warm to RT, and the aqueous phase is removed and extracted with 2 portions of ether. The organic phases are washed with saturated sodium hydrogen carbonate solution, water and brine, dried with sodium sulfate and concentrated by evaporation to yield the title compound, which is used in the next step without being further purified: ¹H-NMR (200 MHz, CDCl₃): 9.63 (s, 1H), 6.9-7.2 (2 m, 4H), 5.04 (m, 1H), 4.42 (m, 1H), 3.10 (m, 2H), 1.43 (s, 9H).

### d) N-3(S)-(Boc-amino)-2(R,S)-hydroxy-4-(p-fluorophenyl)-1-trimethylsilyl-butane

Analogously to Example 16a), 1.63 g (67 mmol) of magnesium in 33 mL of abs. ether are reacted with 8.3 mL (60 mmol) of chloromethyltrimethylsilane to form the Grignard compound which, after reaction with 13 mmol of N-Boc-(p-fluorophenylalaninal), extraction and column chromatography (SiO₂, hexane/ethyl acetate 5:1→4:1), yields the title compound in the form of a diastereoisomeric mixture: TLC R_{f} (L)=0.32; t_{Ret} (I)=24.9 min (22%)/25.5 min (78%); FAB-MS (M+H)⁺=356.

### e) 1-(p-Fluorophenyl)-3-buten-2(S)-amine

Analogously to Example 16b), 1.1 g (3.1 mmol) of N-3(S)-(Boc-amino)-2(R,S)-hydroxy-4-(p-fluorophenyl)-1-trimethylsilyl-butane in 22 mL of methylene chloride are reacted with 1.9 mL (15.5 mmol) of an approximately 48% solution of boron trifluoride in ether to yield the title compound: ¹H-NMR (300 MHz, CDCl₃): 7.2-7.10 and 7.05-6.9 (2 m, each 2H), 5.9-5.8 (m, 1H), 5.2-5.0 (m, 2H), 3.57 (m, 1H), 2.79 (dd, 12 and 6H), 2.62 (dd, 12 and 8 Hz, 1H), 1.7 (sb, 2H).

### f) N-Trifluoroacetyl-1-(p-fluorophenyl)-3-buten-2(S)-amine

Analogously to Example 16c), 364 mg (2.2 mmol) of 1-(p-fluorophenyl)-3-buten-2(S)amine in 1.8 mL of methylene chloride and 5.4 mL of pyridine are reacted with 460 μL (3.3 mmol) of trifluoroacetic acid anhydride to yield the title compound, which after digestion with hexane is obtained in pure form: TLC R_{f} (F)=0.58; MS (M)⁺=261.

### g) 2(R)-[1′(S)-(Trifluoroacetylamino)-2′-(p-fluorophenyl)ethyl]-oxirane

Analogously to Example 16d), 359 mg (1.37 mmol) of N-trifluoroacetyl-1-(p-fluorophenyl)-3-buten-2(S)-amine in 9 mL of chloroform are oxidized with 1.18 g (6.87 mmol) of m-chloroperbenzoic acid to yield the title compound: TLC R_{f} (R)=0.45.

### h) N-Trifluoroacetyl-[(p-F)Phe^{NN}(p-F)Phe)-Boc

Analogously to Example 16, 415 mg (1.49 mmol) of 2(R)-[1′(S)-(trifluoroacetylamino)-2′-(p-fluorophenyl)ethyl]-oxirane and 377 mg (1.57 mmol) of tert-butyl-3-(p-fluorophenyl-methyl)-carbazate in 9 mL of methanol are reacted to yield the title compound: TLC R_{f} (S)=0.53; FAB-MS (M+H)⁺=518; ¹H-NMR (300 MHz, CD₃OD): 7.4-7.3 and 7.3-7.2 (2 m, each 2H), 7.05-6.9 (m, 4H), 4.23 (m, 1H), 3.90-3.65 (m, 3H), 3.03-2.78 and 2.74-2.60 (2 m, each 2H), 1.30 (s, 9H).

### i) H-[(p-F)Phe^{NN}(p-F)Phe]-Boc

Analogously to Example 17a), 285 mg (0.55 mmol) of N-trifluoroacetyl-[(p-F)Phe^{NN}(p-F)Phe]-Boc in 45 mL of methanol are reacted with 14 mL of 1M potassium carbonate solution to yield the title compound: t_{Ret} (I)=16.4 min.

### Example 32

### Z-(L)-Val-[(p-F) Phe^{NN}(p-F)Phe]-H

Analogously to Example 18a), 215 mg (0.33 mmol) of Z-(L)-Val-[(p-F)Phe^{NN}(p-F)Phe]-Boc are deprotected with 100 mL of formic acid to yield the title compound: FAB-MS (M+H)⁺=555.

### Example 33

### Z-(L)-Val-(p-F)Phe^{NN}(p-F)Phe]←(N-(N-(2-pyridylmethyl)-N-methylaminocarbonyl)-(L)-Val)

Analogously to Example 18, 23.6 mg (0.089 mmol) of N-(N-(2-pyridylmethyl)-N-methylaminocarbonyl)-(L)-valine (for preparation see EP 402646 A1, 19 Dec. 1990) and 45 mg (0.081 mmol) of Z-(L)-Val-[(p-F)Phe^{NN}(p-F)Phe]-H are reacted with 33.8 mg (0.089 mmol) of HBTU in 0.76 mL of 0.25M NMM/CH₃CN to yield the title compound which is recrystallized with DMF/DIPE: TLC R_{f} (O)=0.39; FAB-MS (M+H)⁺=802.

### Example 34

### Z-(L)-Val-[(p-F) Phe^{NN}(p-F)Phe]←(N-(2(R,S)-carbamoyl-3-phenyl-propionyl)(L)-Val)

Analogously to Example 18, 26.0 mg (0.089 mmol) of N-(2(R,S)-carbamoyl-3-phenyl-propionyl)-(L)-valine (preparation: Synth., Struct., Funct., Proc. Am. Pept. Symp., 7^{th}, 85, (1981)) and 45 mg (0.081 mmol) of Z-(L)-Val-[(p-F)Phe^{NN}(p-F)Phe]-H (Example 32) are reacted with 33.8 mg (0.089 mmol) of HBTU in 0.76 mL of 0.25M NMM/CH₃CN to yield the title compound which is recrystallized with DMF/DIPE: R_{f} (P)=0.64; FAB-MS (M+H)⁺=829.

### Example 35

### Acetyl-Val-[Phe^{NN}Phe]←(N-acetyl-Val)

Analogously to Example 7, the title compound is obtained from 100 mg (0.25 mmol) of H-[Phe^{NN}Phe]-H.3HCl from Example 2a), 121 mg (0.76 mmol) of N-acetyl-(L)-valine, 288 mg (0.76 mmol) of HBTU and 0.211 mL (1.52 mmol) of triethylamine in DMF after lyophilisation from dioxane. FAB-MS: (M+H)⁺=568, t_{Ret} (I)=15.0 min., R_{f} (B)=0.46.

### Example 36

### Z-(D)-Val-[Phe^{NN}Phe]←((D)-Val)-Z

Analogously to Example 2, the title compound is obtained from 50 mg (0.123 mmol) of H-[Phe^{NN}Phe]-H.3HCl from Example 2a), 95 mg (0.38 mmol) of Z-(D)-valine, 168 mg (0.38 mmol) of BOP, 51 mg (0.38 mmol) of HOBt and 2.53 mL of 0.3M NMM in DMF after lyophilisation from dioxane. FAB-MS: (M+H)⁺=752, t_{Ret} (I)=26.4 min, R_{f} (H)=0.21.

### Example 37

### Quinoline-2-carbonyl-Val-Phe^{NN}Phe]→(N-quinoline-2-carbonyl-Val)

145 mg (0.53 mmol) of N-(quinoline-2-carbonyl)-(L)-valine, 235 mg (0.53 mmol) of BOP and 72 mg (0.53 mmol) of HOBt are dissolved in 3.5 mL of a 0.3M solution of NMM in DMF, after 10 min 70 mg (0.18 mmol) of H-[Phe^{NN}Phe]-H.HCl (Example 2a)) are added, and the mixture is stirred for 5 h at RT under a nitrogen atmosphere. The reaction mixture is concentrated by evaporation and the residue is dissolved in methylene chloride and washed twice with saturated sodium hydrogen carbonate solution, once with 10% citric acid and once again with saturated sodium hydrogen carbonate solution. The organic phases are filtered through cotton wadding and concentrated by evaporation, and the residue is precipitated twice from methylene chloride/methanol by the addition of DIPE. Lyophilisation from dioxane yields the title compound in the form of a white solid (mixture of two diastereoisomers differentiable by HPLC). FAB-MS: (M+H)⁺=794, t_{Ret} (A)=29.1 and 29.3 min, R_{f} (B)=0.81.

### a) N-(Quinoline-2-carbonyl)-(L)-valine

3.28 g (15.9 mmol) of N,N-dicyclohexylcarbodiimide and 2.0 mL (14.5 mmol) of triethylamine are added to a solution of 2.5 g (14.5 mmol) of (L)-valyl-tert-butyl ester and 2.5 g (14.5 mmol) of quinoline-2-carboxylic acid in 100 mL of methylene chlorides (10:1) and the mixture is stirred for 18 h at RT. The reaction mixture is cooled to −18° C. and filtered off from the urea. The filtrate is concentrated by evaporation, and the residue is dissolved in methylene chloride and washed once with saturated sodium hydrogen carbonate solution and once with water. The organic phases are filtered through cotton wadding, concentrated by evaporation and, after chromatographic purification on silica gel with hexane/ethyl acetate (2:1), yield N-(quinoline-2-carbonyl)-(L)-valyl-tert-butyl ester. 2.59 g (12.2 mmol) thereof are left at RT in methylene chloride/TFA (1:1) for 4.5 h. After concentration by evaporation the residue is purified by chromatography on silica gel with hexane/ethyl acetate (2:1). The product-containing fractions are concentrated by evaporation, dissolved in methylene chloride again, and converted into the hydrochloride of the title compound by washing with 1N sodium hydroxide solution and 1N hydrochloric acid. ¹H-NMR (200 MHz, CD₃OD): 1.05 and 1.07 (2 d, J=6 Hz, 6H), 2.40 (m, 1H), 4.65 (m, 1H), 7.70 (m, 1H), 7.85 (m, 1H), 8.00 (dxd, 1H), 8.20 (m, 2H), 8.48 (d, 1H).

### Example 38

### Acetyl-(L)-Val-[Phe^{NN}Cha]←-(N-acetyl-(L)-Val)

Analogously to Example 37, the title compound is obtained from 160 mg (0.40 mmol) of H-[Phe^{NN}Cha]-H.3HCl from Example 10a), 190 mg (1.19 mmol) of N-acetyl-(L)-valine, 525 mg (1.19 mmol) of BOP, 160 mg (1.19 mmol) of HOBt and 7.9 ml of 0.3M NMM in DMF after precipitation from chloroform/methanol with DIPE and lyophilisation from dioxane. FAB-MS: (M+H)⁺=574, t_{Ret} (I)=18.1 min, R_{f} (B)=0.30.

### Example 39

### N-(3-Pyridylacetyl)-(L)-Val-[Phe^{NN}Cha]←(N-(3-pyridylacetyl)-(L)-Val).3HCl

Analogously to Example 7, the title compound is obtained from 100 mg (0.25 mmol) of H-[Phe^{NN}Cha]-H.3HCl from Example 10a), 358 mg (1.52 mmol) of N-(3-pyridylacetyl)-(L)-valine from Example 9a), 576 mg (1.52 mmol) of HBTU and 0.316 ml (2.28 mmol) of triethylamine in DMF after chromatographic purification on silica gel with methylene chloride/methanol (15:1) and lyophilisation of the product-containing fractions from dioxane. FAB-MS: (M+H)⁺=728, t_{Ret} (I)=11.3 min, R_{f} (U)=0.21.

### Example 40

### Acetyl-Ile-[Phe^{NN}Cha]←(N-acetyl-Ile)

Analogously to Example 37, the title compound is obtained from 160 mg (0.40 mmol) of H-[Phe^{NN}Cha]-H.3HCl from Example 10a), 206 mg (1.19 mmol) of N-acetyl-(L)-isoleucine, 525 mg (1.19 mmol) of BOP, 160 mg (1.19 mmol) of HOBt and 7.9 ml of 0.3M NMM in DMF after precipitation from methylene chloride/methanol by the addition of DIPE and lyophilisation from dioxane/tert-butanol (mixture of 2 diastereoisomers differentiable by HPLC). FAB-MS: (M+H)⁺=602, t_{Ret} (I)=20.4 and 20.7 min, R_{f} (D)=0.33.

### Example 41

### Thiomorpholinocarbonyl-(L)-Val-[Phe^{NN}Cha]←(N-thiomorpholinocarbonyl-(L)-Val)

Analogously to Example 6, the title compound is obtained starting from 70 mg (0.12 mmol) of H-(L-Val)-[Phe^{NN}Cha]←(N-(L)-Val)-H.3HCl from Example 14, 58 mg (0.35 mmol) of (4-thiomorpholinylcarbonyl)chloride from Example 6a) and 0.127 mL of triethylamine in 2 mL of DMF after chromatographic purification on silica gel with methylene chloride/methanol (95:5) and lyophilisation of the product-containing fractions from dioxane. FAB-MS: (M+H)⁺=748, t_{Ret} (I)=24.0 min, R_{f} (B)=0.70.

### Example 42

### Z-(L)-Glu-[Phe^{NN}(p-F)Phe]←((L)-Glu)-Z

A solution of 130 mg (0.14 mmol) of Z-(L)-Glu(O-tert-butyl)-[Phe^{NN}(p-F)Phe]←((L)-Glu(O-tert-butyl))-Z [(Glu(O-tert-butyl) here denotes the radical of glutamic acid esterified at the γ-carboxy group by a tert-butyl radical] in 8 mL of methylene chloride/TFA (1:1) is stirred for 3 h at RT. The solvent is evaporated off under reduced pressure and the residue is precipitated from methylene chloride by the addition of DIPE. The title compound is obtained after lyophilisation from dioxane/tert-butanol. FAB-MS: (M+H)⁺=830, t_{Ret} (I)=19.6 min, R_{f} (B)=0.32.

### a) Z-(L)-Glu(O-tert-butyl)-[Phe^{NN}(p-F)Phe]←((L)-Glu(O-tert-butyl))-Z

Analogously to Example 37, the title compound is obtained from 100 mg (0.24 mmol) of H-[Phe^{NN}(p-F)Phe]-H.3HCl, 245 mg (0.73 mmol) of Z-(L)-glutamic acid tert-butyl ester, 321 mg (0.73 mmol) of BOP, 98 mg (0.73 mmol) of HOBt and 4.8 mL of 0.3M NMM in DMF after chromatographic purification on silica gel with methylene chloride/ether (1:1). t_{Ret} (I)=30.2 min, R_{f} (H)=0.17.

### b) H-[Phe^{NN}(p-F)Phe]-H.3HCl

Analogously to Example 2a), the title compound is obtained from 1.77 g (3.51 mmol) of Boc-[Phe^{NN}(p-F)Phe]-Boc after lyophilisation. FAB-MS: (M+H)⁺=304, R_{f} (K)=0.19.

### c) Boc-[Phe^{NN}(p-F)Phe]-Boc

Analogously to Example 1, the title compound is obtained starting from 2.0 g (7.60 mmol) of (2R)-[1′(S)-Boc-amino-2′-phenylethyl]oxirane and 2.17 g (9.04 mmol) of tert-butyl-3-(4-fluorophenyl-methyl)-carbazate from Example 16f) after chromatographic purification on silica gel with hexane/ethyl acetate (2:1). FAB-MS: (M+H)⁺=504, t_{Ret} (I)=26.2 min, R_{f} (F)=0.26.

### Example 43

### N-(2-Pyridylmethyl)-N-methylaminocarbonyl-(L)-Val-[Phe^{NN}(p-F)-Phe]←(N-(N-(2-pyridylmethyl)-N-methylaminocarbonyl)-(L)-Val)

Analogously to Example 37, the title compound is obtained from 70 mg (0.17 mmol) of H-[Phe^{NN}(p-F)Phe]-H.3HCl from Example 42b), 135 mg (0.51 mmol) of N-(N-(2-pyridyl-methyl)-N-methylaminocarbonyl)-(L)-valine (preparation as described in EP 0 402 646 A1 of 19 Dec. 1990), 225 mg (0.51 mmol) of BOP, 69 mg (0.51 mmol) of HOBt and 3.4 mL of 0.3M NMM in DMF after chromatography on silica gel with methylene chloride/methanol (15:1) and lyophilisation of the product-containing fractions from dioxane. FAB-MS: (M+H)⁺=798, t_{Ret} (IV)=35 min, R_{f} (U)=0.21.

### Example 44

### N-(3-(Tetrazol-1-yl)-propionyl)-Val-[Phe^{NN}(p-F)Phe]←(N-(3-(tetrazol-1-yl)-propionyl)-Val)

Analogously to Example 37, the title compound is obtained from 100 mg (0.24 mmol) of H-[Phe^{NN})p-F)Phe]-H.3HCl (from Example 42b), 146 mg (0.61 mmol) of N-(3-(tetrazol-1-yl)-propionyl)-(L)-valine, 268 mg (0.61 mmol) of BOP, 82 mg (0.61 mmol) of HOBt and 4 mL of 0.3M NMM in DMF after precipitation from methylene chloride by the addition of DIPE and lyophilisation from dioxane (4 diastereoisomers differentiable by HPLC). FAB-MS: (M+H)³⁰=750, t_{Ret} (III)=30.8; 31.4; 32.4 and 32.8 min, R_{f} (K)=0.5.

### Example 44a

### N-(3-(Tetrazol-1-yl)-propionyl)-(L)-valine

Analogously to Example 9b, starting from 4 g (16.4 mmol) of (L)-valine-benzyl ester-HCl, 2.1 g (14.9 mmol) of 3-(tetrazol-1-yl)-propionic acid (preparation: U.S. Pat. No. 4,794,109 A of 27 Dec. 1988), 2.4 mL of cyanophosphonic acid diethyl ester and 4.4 ml of triethylamine in DMF, N-(3-(tetrazol-1-yl)-propionyl)-(L)-valine-benzyl ester is obtained after chromatographic purification on silica gel with methylene chloride/methanol (30:1). 2.66 g (8.03 mmol) thereof are hydrogenated in methanol/water (9:1) in the presence of 530 mg of 10% palladium on carbon, at 1 atm hydrogen pressure, to yield the title compound after precipitation from methanol/DIPE. ¹H-NMR (200 MHz, CD₃OD): 0.9 (d, J=7 Hz, 6H), 2.1 (m, 1H), 2.95 (m, 2H), 4.29 (d, J=6 Hz, 1H), 4.78 (m, 2H), 9.15 (s, 1H).

### Example 45

### Z-(L)-Val-[Phe^{NN}(p-F)Phe]←((L)-Val)-Z

Analogously to Example 37, the title compound is obtained from 100 mg (0.24 mmol) of H-[Phe^{NN}(p-F)Phe]-H.3HCl (from Example 42b), 182 mg (0.38 mmol) of Z-(L)-valine, 321 mg (0.73 mmol) of BOP, 98 mg (0.73 mmol) of HOBt and 4.8 mL of 0.3M NMM in DMF after precipitation from methylene chloride by the addition of DIPE and lyophilisation from dioxane. FAB-MS: (M+H)⁺=770, t_{Ret} (I)=26.3 min, R_{f}(H)=0.25.

### Example 46

### Acetyl-Val-[Phe^{NN}(p-F)Phe]←(N-acetyl-Val)

Analogously to Example 37, the title compound is obtained from 80 mg (0.19 mmol) of H-[Phe^{NN}(p-F)Phe]-H.3HCl from Example 42b), 124 mg (0.78 mmol) of N-acetyl-(L)-valine, 344 mg (0.78 mmol) of BOP, 105 mg (0.76 mmol) of HOBt and 4.5 mL of 0.3M NMM in DMF after re-precipitation twice from methylene chloride/methanol by the addition of DIPE and lyophilisation from dioxane/tert-butanol. FAB-MS: (M+H)⁺=586, tmt (I)=15.8 min, R_{f} (E)=0.32.

### Example 47

### Acetyl-Val-[Phe^{NN}(p-CN)Phe]←(N-acetyl-Val)

Analogously to Example 37, the title compound is obtained in the form of a mixture of 2 diastereoisomers differentiable by HPLC from 80 mg (0.19 mmol) of H-[Phe^{NN}(p-CN)Phe]-H.3HCl, 124 mg (0.78 mmol) of N-acetyl-(L)-valine, 344 mg (0.78 mmol) of BOP, 105 mg (0.78 mmol) of HOBt and 4.5 mL of 0.3M NMM in DMF after precipitation from methylene chloride/methanol by the addition of DIPE and lyophilisation from dioxane. FAB-MS: (M+H)⁺=593, t_{Ret} (I)=14.4 and 14.6 min, R_{f} (D)=0.39.

### a) H-[Phe^{NN}(p-CN)Phe]-H.3HCl

Analogously to Example 2a), the title compound is obtained from 2.69 g (5.27 mmol) of Boc-[Phe^{NN}(p-CN)Phe]-Boc after lyophilisation. FAB-MS: (M+H)⁺=311, R_{f} (K)=0.16.

### b) Boc-[Phe^{NN}(p-CN)Phe]-Boc

Analogously to Example 1, the title compound is obtained from 2.0 g (7.60 mmol) of (2R)-[1′(S)-Boc-amino-2′-phenylethyl]oxirane and 1.87 g (7.6 mmol) of tert-butyl-3-(4-cyanophenyl-methyl)-carbazate after crystallization from methanol/DIPE. FAB-MS: (M+H)⁺=511, t_{Ret} (I)=25 min, R_{f} (Y)=0.19.

### c) tert-Butyl-3-(4-cyanophenyl-methyl)-carbazate

Analogously to Example 4b), 10 g (76.3 mmol) of 4-cyanobenzaldehyde and 10 g (76.3 mmol) of tert-butylcarbazate in ethanol are reacted to yield 4-cyanophenylcarbaldehyde-tert-butoxycarbonylhydrazone. 11.1 g thereof are hydrogenated in 150 mL of THF in the presence of 2 g of 10% palladium on carbon at 2 atm hydrogen pressure to yield the title compound. ¹H-NMR (200 MHz, CDCl₃): 7.65 (d, J=8 Hz, 2H), 7.45 (d, J=8 Hz, 2H), 6.08 (s, br, 1H), 4.3 (s, br, 1H), 4.02 (s, 2H), 1.45 (s, 9H).

### Example 48

### Z-(L)-Val-Phe^{NN}(p-CN)Phe]←((L)-Val)-Z

Analogously to Example 37, the title compound is obtained from 70 mg (0.17 mmol) of H-[Phe^{NN}(p-CN)Phe]-H.3HCl (from Example 47a)), 125 mg (0.5 mmol) of Z-(L)-valine, 221 mg (0.5 mmol) of BOP, 68 mg (0.5 mmol) of HOBt and 3.33 mL of 0.3M NMM in DMF after precipitation from methylene chloride by the addition of hexane and lyophilisation from dioxane. FAB-MS: (M+H)⁺=777, t_{Ret} (1)=25.3 min, R_{f} (D)=0.69.

### Example 49

### Z-(L)-Ile-[Phe^{NN}Leu]←((L)-Ile)-Z

Analogously to Example 37, the title compound is obtained from 70 mg (0.19 mmol) of H-[Phe^{NN}Leu]-H.3HCl (from Example 13a)), 154 mg (0.58 mmol) of Z-(L)-isoleucine, 257 mg (0.58 mmol) of BOP, 79 mg (0.58 mmol) of HOBt and 3.88 mL of 0.3M NMM in DMF after chromatography on silica gel with methylene chloride/ether (3:1) and precipitation of the product-containing fractions from methylene chloride/DIPE and lyophilisation from dioxane. FAB-MS: (M+H)⁺=746, t_{Ret} (1)=28.2 min, R_{f} (H)=0.39.

### Example 50

### Isobutoxycarbonyl-(L)-Val-[Phe^{NN}Leu]←(N-isobutoxycarbonyl-(L)-Val)

Analogously to Example 37, the title compound is obtained from 70 mg (0.19 mmol) of H-[Phe^{NN}Leu]-H.3HCl (from Example 13a)), 130 mg (0.58 mmol) of N-(isobutoxycarbonyl)-(L)-valine, 256 mg (0.58 mmol) of BOP, 78 mg (0.58 mmol) of HOBt and 3.9 mL of 0.3M NMM in DMF after chromatography on silica gel with methylene chloride/ether (1:1) and lyophilisation of the product-containing fractions from dioxane. FAB-MS: (M+H)⁺=650, t_{Ret} (I)=26.4 min, R_{f} (H)=0.38.

### a) N-(Isobutoxycarbonyl)-(L)-valine

11.2 mL (85.3 mmol) of isobutyl chloroformate are added to a solution of 10 g (85.3 mmol) of (L)-valine in 100 mL of 2N sodium hydroxide solution and the solution is stirred at RT for 18 h. The reaction solution is washed with methylene chloride, acidified with 4N hydrochloric acid and extracted with methylene chloride. The organic extracts are washed with brine and filtered through cotton wadding to yield the title compound in the form of a colourless resin after concentration by evaporation. ¹H-NMR (200 MHz, CD₃OD): 0.95 (m, 12H), 1.9 (m, 1H), 2.15 (m, 1H), 3.85 (d, J=7 Hz, 2H), 4.05 (d broad, 1H).

### Example 51

### N-(3-(Tetrazol-1-yl)-propionyl)-(L)-Val-[Phe^{NN}Leu]←(N-3-(tetrazol-1-yl)-propionyl-(L)-Val)

Analogously to Example 37, the title compound is obtained from 150 mg (0.42 mmol) of H-[Phe^{NN}Leu]-H.3HCl (from Example 13a)), 251 mg (1.04 mmol) of N-(3-(tetrazol-1-yl-propionyl)-(L)-valine from Example 44a, 460 mg (1.04 mmol) of BOP, 140 mg (1.04 mmol) of HOBt and 6.9 mL of 0.3M N-methylmorpholine in DMF after precipitation from methylene chloride/DIPE and lyophilisation from dioxane/tert-butanol/water. FAB-MS: (M+H)⁺=689, t_{Ret} (I)=14.7 min, R_{f} (K)=0.36.

### Example 52

### Acetyl-Val-[Phe^{NN}Leu]←-(N-acetyl-Val)

Analogously to Example 37, the title compound is obtained from 70 mg (0.19 mmol) of H-[Phe^{NN}Leu]-H.3HCl (from Example 13a)), 184 mg (1.16 mmol) of N-acetyl-(L)-valine, 512 mg (1.16 mmol) of BOP, 156 mg (1.16 mmol) of HOBt and 7.8 mL of 0.3M NMM in DMF after precipitation from methylene chloride/methanol by the addition of DIPE and lyophilisation from dioxane/tert-butanol/water (2 diastereoisomers differentiable according to HPLC). FAB-MS: (M+H)⁺=534, t_{Ret} (I)=14.7 and 15.1 min, R_{f} (D)=0.35.

### Example 53

### Boc-(L)-Val-[Phe^{NN}Leu]←((L)-Val)-Boc

Analogously to Example 7, the title compound is obtained from 300 mg (0.83 mmol) of H-[Phe^{NN}Leu]-H.3HCl (from Example 13a)), 722 mg (3.33 mmol) of Boc-(L)-valine, 1.262 g (3.33 mmol) of HBTU and 0.927 mL (6.66 mmol) of triethylamine in DMF after chromatographic purification on silica gel with methylene chloride/ether (1:1), precipitation of the product-containing fractions and lyophilisation from dioxane. FAB-MS: (M+H)⁺=650, t_{Ret} (I)=26.3 min. R_{f} (H)=064.

### Example 54

### H-(L)-Val-[Phe^{NN}Leu]←((L)-Val)-H.3HCl

Analogously to Example 5, the title compound is obtained from 396 mg (0.61 mmol) of Boc-(L)-Val-[Phe^{NN}Leu]←((L)-Val)-Boc from Example 53 and 10 mL 4N hydrogen chloride in dioxane after lyophilisation of the reaction solution. FAB-MS: (M+H)⁺=450, t_{Ret} (II)=24.1 min, R_{f} (K)=0.25.

### Example 55

### N-Thiomorpholinocarbonyl-(L)-Val-[Phe^{NN}Leu]←(N-thiomorpholinocarbonyl(L)-Val)

Analogously to Example 6, the title compound is obtained in the form of an amorphous solid starting from 100 mg (0.16 mmol) of H-(L)-Val-[Phe^{NN}Leu]←(L)-Val-H-3HCl, 78.5 mg (0.47 mmol) of (4-thiomorpholinylcarbonyl)chloride from Example 6a and 0.172 mL of triethylamine in DMF after chromatographic purification on silica gel with methylene chloride/methanol (95:5), precipitation of the product-containing fractions from methylene chloride/hexane and lyophilisation from dioxane. FAB-MS: (M+H)⁺=708, t_{Ret} (I)=21.4 min, R_{f} (E)=0.45.

### Example 56

### 2(R,S)-Tetrahydrofuryl-methoxycarbonyl-(L)-Val-[Cha^{NN}Leu]←(N-2(R,S)-tetrahydrofuryl-methoxycarbonyl-(L)-Val)

Analogously to Example 37, the title compound is obtained from 80 mg (0.22 mmol) of H-[Cha^{NN}Leu]-H.3HCl, 160 mg (0.65 mmol) of N-(2(R,S)-tetrahydrofuryl-methoxycarbonyl)-(L)-valine, 289 mg (0.65 mmol) of BOP, 88 mg (0.65 mmol) of HOBt and 4.35 mL of 0.3M NMM in DMF after chromatographic purification on silica gel with ethyl acetate and lyophilisation of the product-containing fractions from dioxane. FAB-MS: (M+H)⁺=712, t_{Ret} (I)=22.4 min, R_{f} (E)=0.21.

### a) H-[Cha^{NN}Leu]-H.3HCl

Analogously to Example 5, 100 mg (83%) of the title compound are obtained from 150 mg (0.33 mmol) of Boc-[Cha^{NN}Leu]-Boc and 10 mL of 4N hydrogen chloride in dioxane after lyophilisation of the reaction solution. R_{f} (K)=0.26.

### b) Boc-[Cha^{NN}Leu]-Boc

A solution of 200 mg (0.24 mmol) of Boc-[Phe^{NN}Leu]-Boc (Example 12) in 15 mL of methanol is hydrogenated for 4 h at 1 atm hydrogen pressure in the presence of 10 mg of Nishimura-catalyst (Rh(III)- and Pt(IV)-oxide monohydrate, Degussa). The catalyst is removed by filtration, the solvent is fully concentrated by evaporation and the compound is obtained after crystallization from methylene chloride/hexane. t_{Ret} (I)=26.7 min, R_{f} (V)=0.21.

### c) N-(2(R,S)-Tetrahydrofuryl-methoxycarbonyl)-(L)-valine

Analogously to Example 50a, the title compound is obtained in the form of a mixture of 2 diastereoisomers from 7 g (60 mmol) of (L)-valine and 9.8 g (60 mmol) of 2(R,S)-tetrahydrofurylmethyl-chloroformate (Heterocycles 27, 1155 (1988)) in 100 mL of 2N sodium hydroxide solution and 30 mL of dioxane. t_{Ret} (II)=23.5 and 23.8 min.

### Example 57

### Z-Val-[Phe^{NN}Leu]←(N-(3-(tetrazol-1-yl)-propionyl)-Val)

Analogously to Example 37, the title compound is obtained (in the form of 2 diastereoisomers differentiable by HPLC) from 100 mg (0.21 mmol) of Z-(L)-Val-[Phe^{NN}Leu]-H, 75 mg (0.31 mmol) of N-(3-(tetrazol-1-yl)-propionyl)-(L)-valine from Example 44a, 137 mg (0.31 mmol) of BOP, 42 mg (0.31 mmol) of HOBt and 2 mL of 0.3M NMM in DMF after precipitation from methylene chloride/hexane and lyophilisation from dioxane/tert-butanol. FAB-MS: (M+H)⁺=708, t_{Ret} (I)=21.1 and 21.1 min, R_{f} (D)=0.45.

### a) Z-(L)-Val-[Phe^{NN}Leu]-H

A solution of 250 mg (0.43 mmol) of Z-(L)-Val-[Phe^{NN}Leu]-Boc in 5 mL of formic acid is stirred for 7.5 h at RT. After that time no more starting material can be detected by HPLC analysis (t_{Ret} (I)=27.5 min), and the reaction solution is concentrated by evaporation. The residue is dissolved in chloroform and washed with saturated sodium hydrogen carbonate solution. The chloroform phase is filtered through cotton wadding and yields the crude title compound after removal of the solvent by evaporation. t_{Ret} (I)=16.7 min, R_{f} (K)=0.21.

### b) Z-(L)-Val-[Phe^{NN}Leu]-Boc

Analogously to Example 37, the title compound is obtained from 230 mg (0.653 mmol) of H-[Phe^{NN}Leu]-Boc, 247 mg (0.98 mmol) of Z-(L)-valine, 434 mg (0.98 mmol) of BOP, 133 mg (0.98 mmol) of HOBt and 6.5 mL of 0.3M NMM in DMF after precipitation from methylene chloride/methanol by the addition of DIPE. FAB-MS: (M+H)⁺=585, t_{Ret} (I)=27.5 min, R_{f} (C)=0.71.

### c) H-[Phe^{NN}Leu]-Boc

Analogously to Example 17a), the title compound is obtained starting from 1.27 g (2.84 mmol) of N-trifluoroacetyl-[Phe^{NN}Leu]-Boc and 24 mL of 1N aqueous sodium carbonate solution in 90 mL of methanol by precipitation from methylene chloride by the addition of DIPE. t_{Ret} (I)=14.9 min, R_{f} (K)=0.38.

### d) N-Trifluoroacetyl-[Phe^{NN}Leu]]-Boc

Analogously to Example 16, the title compound is obtained starting from 3 g (11.57 mmol) of 2(R)-[1′(S)-(trifluoroacetylamino)-2′-phenylethyl]-oxirane from Example 16d) and 2.3 g (12.15 mmol) of tert-butyl-3-isobutyl-carbazate (preparation: J. Chem. Soc. Perkin I, 1712 (1975)) after chromatographic purification on silica gel with methylene chloride/ether (20:1). t_{Ret} (I)=24.7 min, R_{f} (W)=0.36.

### Example 58

### Acetyl-Val-[Phe^{NN}Leu]←(N-(2(R,S)-carbamoyl-3-phenyl-propionyl)-Val)

Analogously to Example 37, the title compound is obtained (in the form of 2 diastereoisomers differentiable by HPLC) from 140 mg (0.3 mmol) of acetyl-(L)-Val-[Phe^{NN}Leu]-H.2HCl, 132 mg (0.45 mmol) of N-(2(R,S)-carbamoyl-3-phenyl-propionyl)-(L)-valine (preparation: Synth., Struct., Funct., Proc. Am. Pept. Symp., 7^{th}, 85, (1981)), 199 mg (0.45 mmol) of BOP, 61 mg (0.45 mmol) of HOBt and 3.5 mL of 0.3M NMM in DMF after precipitation from methylene chloride/DIPE and lyophilisation from dioxane. FAB-MS: (M+H)⁺=667, t_{Ret} (I)=17.9 and 18.4 min, R_{f} (D)=0.33.

### a) Acetyl-Val-[Phe^{NN}Leu]H.2HCl

Analogously to Example 2a), the title compound is obtained starting from 230 mg (0.46 mmol) of acetyl-(L)-Val-[Phe^{NN}Leu]-Boc after lyophilisation. t_{Ret} (I)=10.5 min, R_{f} (D)=0.38.

### b) Acetyl-Val-[Phe^{NN}Leu]-Boc

Analogously to Example 37, the title compound is obtained from 250 mg (0.71 mmol) of H-[Phe^{NN}Leu]-Boc from Example 57c), 170 mg (1.07 mmol) of N-acetyl-(L)-valine, 471 mg (1.07 mmol) of BOP, 144 mg (1.07 mmol) of HOBt and 7.1 mL of 0.3M NMM in DMF after precipitation from methylene chloride by the addition of DIPE and lyophilisation from dioxane. FAB-MS: (M+H)⁺=493, t_{Ret} (I)=20.5 min, R_{f} (D)=0.59.

### Example 59

### N-Morpholinocarbonyl-(L)-Val-[Phe^{NN}Leu]←(N-(3-(tetrazol-1-yl)-propionyl)-Val)

Analogously to Example 37, the title compound is obtained (in the form of 2 diastereoisomers differentiable by HPLC) from 100 mg (0.19 mmol) of N-morpholinocarbonyl-(L)-Val-[Phe^{NN}Leu]-H.2HCl, 67 mg (0.38 mmol) of N-(3-(tetrazol-1-yl)-propionyl)-(L)-valine from Example 44a, 124 mg (0.28 mmol) of BOP, 38 mg (0.28 mmol) of HOBt and 2.1 mL of 0.3M NMM in DMF after precipitation from methylene chloride by the addition of DIPE and lyophilisation from dioxane. FAB-MS: (M+H)⁺=687, t_{Ret} (I)=15.2 and 15.4 min, R_{f} (D)=0.25.

### a) N-Morpholinocarbonyl-(L)-Val-[Phe^{NN}Leu]-H.2HCl

Analogously to Example 2a), the title compound is obtained starting from 279 mg (0.49 mmol) of N-morpholinocarbonyl-(L)-Val-[Phe^{NN}Leu]-Boc after lyophilisation. FAB-MS: (M+H)⁺=464, t_{Ret} (II)=30.3 min, R_{f} (D)=0.46.

### b) N-Morpholinocarbonyl-(L)-Val-[Phe^{NN}Leu]-Boc

Analogously to Example 37, the title compound is obtained from 250 mg (0.71 mmol) of H-[Phe^{NN}Leu]-Boc (from Example 57c)), 265 mg (1.07 mmol) of N-morpholinocarbonyl-(L)-valine from Example 7a), 471 mg (1.07 mmol) of BOP, 144 mg (1.07 mmol) of HOBt and 7.1 mL of 0.3M NMM in DMF after precipitation from methylene chloride/hexane and lyophilisation from dioxane. FAB-MS: (M+H)⁺=564, t_{Ret} (I)=21.5 min, R_{f} (K)=0.69.

### Example 60

### N-Trifluoroacetyl-[Phe^{NN}Leu]←(N-(2(R,S)-carbamoyl-3-phenyl-propionyl)-(L)-Val)

Analogously to Example 37, the title compound is obtained (in the form of 2 diastereoisomers differentiable by HPLC) from 136 mg (0.32 mmol) of N-trifluoroacetyl-[Phe^{NN}Leu]-H.2HCl, 142 mg (0.49 mmol) of N-(2(R,S)-carbamoyl-3-phenyl-propionyl)-(L)-valine (preparation: Synth., Struct., Funct., Proc. Am. Pept. Symp., 7^{th}, 85, (1981)), 215 mg (0.49 mmol) of BOP, 66 mg (0.49 mmol) of HOBt and 3.5 mL of 0.3M NMM in DMF after chromatographic purification on silica gel with chloroform/methanol (15:1), precipitation of the product-containing fractions from methylene chloride/DIPE and lyophilisation from dioxane/tert-butanol. FAB-MS: (M+H)⁺=622, t_{Ret} (I)=21.6 and 22.0 min, R_{f} (K)=0.26.

### a) N-Trifluoroacetyl-[Phe^{NN}Leu]-H.2HCl

Analogously to Example 2a), the title compound is obtained starting from 300 mg (0.67 mmol) of N-trifluoroacetyl-[Phe^{NN}Leu]-Boc from Example 57d) after lyophilisation. R_{f} (W)<0.1.

### Example 61

### Z-(L)-Val-[Phe^{NN}Nle]←(N-(2(R,S)-(N-(2-morpholinoethyl)-carbamoyl)-3-methyl)-butyryl)

Analogously to Example 37, the title compound is obtained (in the form of 2 diastereoisomers differentiable by HPLC) from 100 mg (0.17 mmol) of Z-(L)-Val-[Phe^{NN}Nle]-H.2HCl, 69 mg (0.27 mmol) of 2(R,S)-(N-(2-morpholinoethyl)-carbamoyl)-3-methylbutyric acid (isopropylmalonic acid N-(2-morpholinoethyl)monoamide), 119 mg (0.27 mmol) of BOP, 36 mg (0.27 mmol) of HOBt and 2.1 mL of 0.3M NMM in DMF after precipitation from methylene chloride/DIPE and lyophilisation from dioxane. FAB-MS: (M+H)⁺=725, t_{Ret} (I)=17.2 and 17.6 min, R_{f} (D)=0.56.

### a) Z-(L)-Val-[Phe^{NN}Nle]-H.2HCl

Analogously to Example 2a), the title compound is obtained starting from 310 mg (0.53 mmol) of Z-(L)-Val-[Phe^{NN}Nle]-Boc after lyophilisation. t_{Ret} (I)=16.4 min, R_{f} (U)=0.25.

### b) Z-(L)-Val-[Phe^{NN}Nle]-Boc

Analogously to Example 37, the title compound is obtained from 250 mg (0.71 mmol) of H-[Phe^{NN}Nle]-Boc, 268 mg (1.07 mmol) of Z-(L)-valine, 472 mg (1.07 mmol) of BOP, 144 mg (1.07 mmol) of HOBt and 7.1 mL of 0.3M NMM in DMF after chromatographic purification on silica gel with methylene chloride/methanol (40:1) and precipitation of the product-containing fractions from methylene chloride/DIPE. t_{Ret} (I)=25.6 min, R_{f} (X)=0.17.

### c) H-[Phe^{NN}Nle]-Boc

Analogously to Example 17a), the title compound is obtained starting from 830 mg (1.85 mmol) of N-trifluoroacetyl-[Phe^{NN}Nle]-Boc after precipitation from methylene chloride/DIPE. t_{Ret} (I)=15.4 min, R_{f} (K)=0.54.

### d) N-Trifluoroacetyl-[Phe^{NN}Nle]-Boc

Analogously to Example 16, the title compound is obtained starting from 1 g (3.86 mmol) of 2(R)-[1′(S)-(trifluoroacetylamino)-2′-phenylethyl]-oxirane from Example 16d) and 720 mg (3.86 mmol) of tert-butyl-3-butyl-carbazate after chromatographic purification on silica gel with methylene chloride/ether (20:1). t_{Ret} (I)=25.3 min, R_{f} (Q)=0.43.

### e) tert-Butyl-3-butyl-carbazate

Analogously to Example 4b), the corresponding tert-butoxycarbonyl-hydrazone (25 g, 99%) is obtained from 18.0 g (136.2 mmol) of tert-butyl-carbazate and 12.3 mL (136.2 mmol) of n-butanal in the form of a crude product, which is hydrogenated as described in Example 4a) in the presence of 10 g of 5% platinum on carbon at 4 atm hydrogen pressure. Chromatographic purification of the crude product on silica gel with hexane/ethyl acetate (1:1) yields the title compound. R_{f} (N)=0.44, ¹H-NMR (200 MHz, CD₃OD). 0.92 (t, J=7 Hz, 3H), 1.43 (s, 9H), 1.30 to 1.50 (m, 4H), 2.75 (t, J=7 Hz, 2H).

### f) 2(R,S)-(N-(2-Morpholinoethyl)-carbamoyl)-3-methylbutyric Acid

Analogously to Example 9b) there is obtained from 7 g (43.7 mmol) of racemic isopropylmalonic acid monomethyl ester (Chem. Ber. 119, 1196 (1986)), 6.3 mL (48.1 mmol) of aminoethyl-morpholine, 6.6 mL (43.7 mmol) of cyanophosphonic acid diethyl ester and 12.8 mL (91.8 mmol) of triethylamine in DMF, 2(R,S)-(N-(2-morpholinoethyl)-carbamoyl)-3-methyl-butyric acid methyl ester (isopropylmalonic acid N-morpholinoethylamide methyl ester). This is stirred for 5 h in a mixture of 28 mL of 2N sodium hydroxide solution and 28 mL of dioxane at RT, acidified with 2N hydrochloric acid and fully concentrated by evaporation. The residue is digested with ethanol, filtered off, and concentration by evaporation of the filtrate yields the title compound. ¹H-NMR (200 MHz, CD₃OD): 0.95 and 1.00 (2 d, J=7H, 6H), 2.25 (m, 4H), 2.70 (m, 6H), 2.75 (d, J=8 Hz, 1H), 3.45 (m, 2H), 3.75 (m, 4H).

### Example 62

### Z-(L)-Val-[Phe^{NN}Nle]←(N-(3-(tetrazol-1-yl)-propionyl)-Val)

Analogously to Example 37, the title compound is obtained (in the form of 2 diastereoisomers differentiable by HPLC) from 100 mg (0.18 mmol) of Z-(L)-Val-[Phe^{NN}Nle]-H.2HCl (from Example 61a)), 65 mg (0.27 mmol) of N-(3-(tetrazol-1-yl)-propionyl)-(L)-valine from Example 44a, 119 mg (0.27 mmol) of BOP, 36 mg (0.27 mmol) of HOBt and 2.1 mL of 0.3M N-methylmorpholine in DMF after precipitation from methylene chloride/DIPE and lyophilisation from dioxane/tert-butanol. FAB-MS: (M+H)⁺=708, t_{Ret} (I)=20.3 and 20.6 min, R_{f} (D)=0.43.

### Example 63

### Z-(L)-Val-[Phe^{NN}Nle]←(N-(2(R,S)-(N-(2-pyridylmethyl)-carbamoyl)-3-methyl)-butyryl) (dibenzenesulfonate)

Analogously to Example 37, the title compound is obtained in the form of the free amine from 95 mg (0.17 mmol) of Z-(L)-Val-[Phe^{NN}Nle]-H.2HCl from Example 61a), 60 mg (0.26 mmol) of (R,S)-isopropylmalonic acid N-(2-picolyl)-monoamide, 113 mg (0.26 mmol) of BOP, 35 mg (0.26 mmol) of HOBt and 2.0 mL of 0.3M NMM in DMF after chromatographic purification on silica gel with methylene chloride/methanol (15:1). The free amine is dissolved in methylene chloride, 2 equivalents of benzenesulfonic acid are added, and precipitation is effected by the addition of DIPE. Lyophilisation from tert-butanol yields the dibenzenesulfonate salt (in the form of 2 diastereoisomers differentiable by HPLC). FAB-MS: (M+H)⁺=703, t_{Ret} (,)=17.7 and 18.0 min, R_{f} (D)=0.54.

### a) Isopropylmalonic acid N-(2-picolyl)monoamide

10.6 mL (103 mmol) of N-methylmorpholine are added to a solution of 15 g (93.6 mmol) of isopropylmalonic acid monomethyl ester (preparation: Chem. Ber. 119, 1196 (1986)) in 150 mL of THF and subsequently 13.5 mL (103 mmol) of isobutyl chloroformate are added dropwise thereto. After 30 min 15.3 mL (150 mmol) of 2-picolylamine are added and the resulting suspension is stirred for 2 h. The reaction mixture is diluted with 1N sodium hydroxide solution and water and washed with methylene chloride, and the organic phase is filtered through cotton wadding and concentrated by evaporation. Crystallization of the residue yields isopropylmalonic acid N-(2-picolylamide) methyl ester, which is hydrolyzed in 2N sodium hydroxide solution and dioxane as described in Example 61f) to yield the title compound. t_{Ret} (II)=16.0 min.

### Example 64

### Z-(L)-Val-[Phe^{NN}(p-F) Phe]←(N-(3-(tetrazol-1-yl)-propionyl)-(L)-Val) (benzenesulfonate)

Analogously to Example 37, the title compound is obtained in the form of the free amine from 100 mg (0.16 mmol) of Z-(L)-Val-[Phe^{NN}(p-F)Phe]-H from Example 22a), 59 mg (0.25 mmol) of N-(3-(tetrazol-1-yl)-propionyl)-(L)-valine from Example 44a, 109 mg (0.25 mmol) of BOP, 33 mg (0.25 mmol) of HOBt and 1.19 mL of 0.3M N-methylmorpholine in DMF after precipitation from methylene chloride/DIPE. The free amine is dissolved in methylene chloride/methanol, 1 equivalent of benzenesulfonic acid is added, and precipitation is effected by adding hexane. Lyophilisation from tert-butanol yields the title compound in the form of the benzenesulfonate salt. FAB-MS: (M+H)⁺=760, t_{Ret} (I)=21.6 min, R_{f} (B)=0.49.

### Example 65

### Methylsulfonyl-[Phe^{NN}Phe]←(N-phenylacetyl-(L)-Val)

132 mg (0.28 mmol) of methylsulfonyl-[Phe^{NN}Phe]-H.2HCl is reacted analogously to Example 7 with 197 mg (0.84 mmol) of N-phenylacetyl-(L)-valine (preparation: Mem. Tokyo Univ. Agric. 20, 51 (1978)), 317 mg (0.84 mmol) of HBTU and 0.23 mL (1.67 mmol) of triethylamine in DMF to yield the title compound after precipitation from methanol by the addition of ether. FAB-MS: (M+H)⁺=581, t_{Ret} (I)=20.2 min, R_{f} (B)=0.64.

### a) Methylsulfonyl-[Phe^{NN}Phe]-H.2HCl

Analogously to Example 2a), the title compound is obtained starting from 130 mg (0.28 mmol) of methylsulfonyl-[Phe^{NN}Phe]-Boc after lyophilisation. FAB-MS: (M+H)⁺=364, t_{Ret} (II)=28.5 min, R_{f} (K)=0.56.

### b) Methylsulfonyl-[Phe^{NN}Phe]-Boc

Analogously to Example 16a), the title compound is obtained as a diastereoisomeric mixture in a ratio of 4:1 starting from 1.1 g (4.56 mmol) of 2(R)-[1′(S)-(methylsulfonylamino)-2′-phenylethyl]oxirane and 1.11 g (5.02 mmol) of tert-butyl-3-benzyl-carbazate (preparation: J. Chem. Soc. Perkin I, 1712 (1975)). By crystallization from methylene chloride/hexane the ratio in favour of the 2S-diastereoisomer is improved to 10:1. FAB-MS: (M+H)⁺=464, t_{Ret} (I)=21.3 min, R_{f} (N)=0.26.

### c) 2(R)-[1′(S)-(Methylsulfonylamino)-2′-phenylethyl]oxirane

2.36 g (13.6 mmol) of methansulfonic acid anhydride and 1.88 mL (13.6 mmol) of triethylamine are added at 0° C. to a solution of 1 g (6.8 mmol) of 1-phenyl-3-buten-2(S)-amine from Example 16b) in 10 mL methylene chloride and the mixture is stirred for 1 h. The reaction mixture is washed with water and saturated sodium hydrogen carbonate solution and the organic phase is filtered through cotton wadding and concentrated by evaporation to yield 2(S)-methylsulfonylamino-1-phenyl-3-butene. 1 g (4.4 mmol) of that crude product is dissolved in 30 mL of methylene chloride, 3.05 g (17.7 mmol) of 4-chloroperbenzoic acid are added at RT and stirring is carried out for 18 h. The reaction solution is washed 5 times with 10% aqueous sodium sulfite solution, filtered through cotton wadding and fully concentrated by evaporation. According to ¹H-NMR the crude product contains both the (2R)- and the (2S)-epimer in a ratio of 4:1. ¹H-NMR (200 MHz, CD₃ OD): 2.30 and 2.52 (2 s, together 3H), 2.6 to 3.2 (m, 5H), 3.55 (m, 1H) 7.32 (m, 5H).

### Example 66

### Methoxycarbonyl-(L)-Val-[Phe^{NN}Leu]←(N-methoxycarbonyl-(L)-Val)

Analogously to Example 37, the title compound is obtained from 200 mg (0.55 mmol) of H-[Phe^{NN}Leu]-H.3HCl (from Example 13a)), 291 mg (1.66 mmol) of N-methoxycarbonyl-(L)-valine (preparation: Chem. Lett. 705, (1980)), 735 mg (1.66 mmol) of BOP, 225 mg (1.66 mmol) of HOBt and 11 mL of 0.3M NMM in DMF after precipitation from methylene chloride/DIPE and lyophilisation from dioxane. FAB-MS: (M+H)⁺=566, t_{Ret} (I)=18.6 min, R_{f} (U)=0.33.

### Example 67

### Methoxycarbonyl-(L)-Val-[Phe^{NN}(p-F)Phe]←(N-methoxycarbonyl-(L)-Val)

Analogously to Example 37, the title compound is obtained from 200 mg (0.48 mmol) of H-[Phe^{NN}(p-F)Phe]-H.3HCl (from Example 42b)), 255 mg (1.45 mmol) of N-methoxycarbonyl-(L)-valine (preparation: Chem. Lett. 705, (1980)), 643 mg (1.45 mmol) of BOP, 196 mg (1.45 mmol) of HOBt and 9.7 mL of 0.3M NMM in DMF after precipitation from methylene chloride/DIPE and lyophilisation from dioxane. FAB-MS: (M+H)⁺=618, t_{Ret} (I)=19.5 min, R_{f} (U)=0.22.

### Example 68

### Methoxycarbonyl-(L)-Val-[Phe^{NN}(p-CN)Phe]←(N-methoxycarbonyl-(L)-Val)

Analogously to Example 37, the title compound is obtained from 200 mg (0.48 mmol) of H-[Phe^{NN}(p-CN)Phe]-H.3HCl (from Example 47a)), 250 mg (1.43 mmol) of N-methoxycarbonyl-(L)-valine (preparation: Chem. Lett. 705, (1980)), 631 mg (1.43 mmol) of BOP, 193 mg (1.43 mmol) of HOBt and 9.5 mL of 0.3M NMM in DMF after chromatographic purification on silica gel with methylene chloride/methanol (15:1) and lyophilisation of the product-containing fractions from dioxane. FAB-MS: (M+H)⁺=625, t_{Ret} (I)=18 min, R_{f} (U)=0.31.

### Example 69

### Z-(L)-Val-[(p-F)Phe^{NN}(p-F)Phe]←(N-(2(R,S)-(N-(2-morpholinoethyl)-carbamoyl)-3-methyl)-butyryl)

Analogously to Example 18, 23.0 mg (0.089 mmol) of 2(R,S)—(N-(2-morpholinoethyl)-carbamoyl)-3-methylbutyric acid (Example 61f)) and 45 mg (0.081 mmol) of Z-(L)-Val-[(p-F)Phe^{NN}(p-F)Phe]-H (Example 32) are reacted with 33.8 mg (0.089 mmol) of HBTU in 0.76 ml of 0.25M NMM/CH₃CN to yield the title compound which is re-precipitated with DMF/DIPE: TLC R_{f} (P)=0.42; FAB-MS (M+H)⁺=795.

### Example 70

### Z-(L)-Val-[(p-F)Phe^{NN}(p-F)Phe]←(N-(2(R,S)-(N-(2-pyridylmethyl)-carbamoyl)-3-methyl)-butyryl)

Analogously to Example 18, 21.0 mg (0.089 mmol) of rac. isopropylmalonic acid N-(2-picolyl)amide (Example 63a)) and 45 mg (0.081 mmol) of Z-(L)-Val-[(p-F)Phe^{NN}(p-F)Phe]-H (Example 32) are reacted with 33.8 mg (0.089 mmol) of HBTU in 0.76 mL of 0.25M NMM/CH₃CN to yield the title compound which is re-precipitated with DMF/DIPE: TLC R_{f} (P)=0.52; FAB-MS (M+H)⁺=773.

### Example 71

The following compounds can be prepared analogously to one of the aforementioned processes:
A) Z-(L)-Val-[(p-F) Phe^{NN}(p-F)Phe]←((L)-Val).rarw.(N-morpholinocarbonyl-Gly);
B) N-Morpholinocarbonyl-(L)-Val-[(p-F)Phe^{NN}(p-F)Phe]←((L)-Val)←(N-morpholinocarbonyl-Gly)
C) N-(Quinoline-2-carbonyl)-(L)-Asn-[Phe^{NN}(p-F)Phe]←((L)-Val)-Z
D) N-(Morpholinosulphonyl)-(L)-Val-[Phe^{NN}Leu]←(N(morpholino-sulphonyl)-(L)-Val)
E) N-(Quinoline-2-carbonyl)-(L)-Asn-[Phe^{NN}Cha]←((L)-Val)-Z(=1-[2(S)-hydroxy-3(S)-(N-(quinoline-2-carbonyl)-(L)-asparagyl)amino-4-phenyl-butyl]-1-[cyclohexylmethyl]-2-[N-(benzyloxycarbonyl)-(L)-valyl]-hydrazine).

Under a nitrogen atmosphere, 27 mg (0.107 mmol) of Z-valine in 0.59 mL of a 0.3M solution of NMM in DMF are activated with 47 mg (0.107 mmol) of BOP and 14 mg (0.107 mmol) of HOBT and, after 15 min, 50 mg (0.089 mmol) of 1-[2(S)-hydroxy-3(S)-(N-(quinoline-2-carbonyl)-(L)-asparagyl)amino-4-phenyl-butyl]-1-[cyclohexylmethyl]-hydrazine are added. The mixture is stirred for 18 h at RT and concentrated by evaporation under HV. The residue is dissolved in methylene chloride and washed with saturated NaHCO₃ solution, water and brine, the aqueous phases are extracted twice with methylene chloride, and the organic phases are dried with Na₂SO₄ and concentrated by evaporation. Column chromatography (SiO₂, ethyl acetate/ethanol 100:3) yields the pure title compound: TLC R_{f} (D′)=0.21; t_{Ret} (V)=16.7 min; FAB-MS (M+H)⁺=794.

The starting material is prepared as follows:

### a) 1-[2(S)-Hydroxy-3(S)-(N-(quinoline-2-carbonyl)-(L)-asparagyl)amino-4-phenyl-butyl]-1-[cyclohexylmethyl]-hydrazine

Analogously to Example 98 (see below), 921 mg (1.39 mmol) of 1-[2(S)-hydroxy-3(S)-(N-(quinoline-2-carbonyl)-(L)-asparagyl) amino-4-phenyl-butyl]-1-[cyclohexylmethyl]-2-[tert-butoxy-carbonyl]-hydrazine (Example 100 A), see below) in 37 mL of formic acid are reacted to form the title compound and used directly in the next step.
F) N-(Quinoline-2-carbonyl)-(L)-Asn-[Phe^{NN}(p-F)Phe]←(N-(methoxycarbonyl)-(L)-Val)

### Example 72

### 1-[2(S)-Acetoxy-3(S)-(N-(2-methoxyethoxycarbonyl)-( )-valyl)amino-4-phenyl-butyl]-1-[cyclohexylmethyl]-2-[N-(2-methoxyethoxycarbonyl)-(L)-valyl]-hydrazine

Under a nitrogen atmosphere, 200 mg (0.29 mmol) of N-(2-methoxy-ethoxycarbonyl)-(L)-Val-[Phe^{NN}Cha]←-(N-(2-methoxy-ethoxycarbonyl)-(L)-Val) in 4 mL of THF and 60 μL (0.43 mmol) of triethylamine are acetylated with 40 μL (0.43 mmol) of acetic anhydride for 3 h at RT in the presence of 0.5 mg (0.003 mmol) of DMAP. The reaction mixture is partitioned between 3 portions of methylene chloride, water, saturated NaHCO₃ solution and brine. The title compound is obtained from the organic phases after drying with Na₂SO₄, concentrating by evaporation and column chromatography (SiO₂, methylene chloride/methanol 30:1): TLC R_{f} (Z)0.17; t_{Ret} (1)=22.5 min; FAB-MS (M+H)⁺=736.

The starting materials are prepared as follows:

### a) N-(2-Methoxy-ethoxycarbonyl)-(L)-Val-[Phe^{NN}Cha]←(N-(2-methoxy-ethoxycarbonyl)-(L)-Val)

Analogously to Example 2, 820 mg (3.74 mmol) of N-(2-methoxy-ethoxycarbonyl)-(L)-valine are activated with 1.65 g (3.74 mmol) of BOP and 505 mg (3.74 mmol) of HOBT in 25 mL of a 0.3M solution of NMM in DMF and, after 10 min, reacted with 500 mg (1.25 mmol) of H-[Phe^{NN}Cha]-H (hydrochloride salt) (see Example 10a) for 18 h. The reaction mixture is concentrated by evaporation under HV, and the residue is dissolved in CHCl₃ and washed with 10% citric acid solution, saturated NaHCO₃ solution and brine. The aqueous phases are extracted with 2 portions of CHCl₃, and the organic phases are dried with Na₂SO₄ and concentrated by evaporation. Column chromatography (SiO₂, CHCl₃/MeOH 30:1) and precipitation with hexane from a CH₂Cl₂ solution yields the title compound: TLC R_{f} (T)=0.37; t_{Ret} (I)=21.5 min; FAB-MS (M+H)⁺=694.

### b) Chloroformic Acid (2-methoxy-ethyl) Ester

Under a nitrogen atmosphere, 13.3 mL (168 mmol) of 2-methoxy-ethanol are added dropwise at from 0 to 5° C. to 100 mL (202 mmol) of a 20% solution of phosgene in toluene, and the mixture is stirred for 90 min at 0° C. and for 18 h at RT to complete the reaction. The reaction mixture is extracted with water, and the organic phase is filtered through cotton wadding and concentrated by evaporation: IR (CH₂Cl₂): inter alia 3055 w, 2995 w, 2935 w, 2895 w, 2825 w, 1775 s, 1167 s, 1127 s; ¹H-NMR (200 MHz, CDCl₃): 3.38 (s, 3H), 3.64 and 4.44 (2t, J=5 Hz, each 2H).

### c) N-(2-Methoxy-ethoxycarbonyl)-(L)-valine

A solution of 3.06 g (22.1 mmol) of chloroformic acid (2-methoxy-ethyl) ester in 18 mL of dioxane is added to 2.59 g (22.1 mmol) of L-valine in 26.4 mL of 2N NaOH and the mixture is then stirred for 18 h at RT. The reaction mixture is extracted with chloroform, and the inorganic phase is acidified with 4N HCl and extracted again with chloroform. The chloroform phase last obtained is dried and concentrated by evaporation to yield the title compound: ¹H-NMR (200 MHz, CDCl₃): 0.92 and 0.99 (2 d, J=7 Hz, 6H), 2.2 (m, 1H), 3.38 (s, 3H), 3.59 and 4.24 (2m, each 2H), 4.3 (m, 1H), 5.4 (d, J=9 Hz, HN), 8.5 (sb, 1H).

### Example 73

### 1-[2(S)-Acetoxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine

Analogously to Example 72, 200 mg (0.33 mmol) of N-(methoxycarbonyl)-(L)-Val-[Phe^{NN}Cha]←-(N-(methoxycarbonyl)-(L)-Val) in 4 mL of THF and 68 μL (0.50 mmol) of triethylamine are reacted with 46 μL (0.50 mmol) of acetic anhydride in the presence of 1.2 mg (0.01 mmol) of DMAP. Precipitation with DIPE from a concentrated solution of the crude product in methanol yields the pure title compound: TLC R_{f} (A′)=0.42; t_{Ret} (I)=22.6 min; FAB-MS (M+H)⁺=648.

The starting material is prepared as follows:

### a) N-(Methoxycarbonyl)-(L)-Val-[Phe^{NN}Cha]←(N-(methoxycarbonyl)-(L)-Val) (=1-2(S)-hydroxy-3(S)-(N-(methoxycarbonyl)-(L)-valyl)-amino-4-phenyl-butyl]-1-[cyclohexylmethyl]-2-[N-(methoxycarbonyl)-(L)-valyl]-hydrazine)

Analogously to Example 2, 1.47 g (8.4 mmol) of N-(methoxycarbonyl)-(L)-valine are activated with 3.71 g (8.4 mmol) of BOP and 1.13 g (8.4 mmol) of HOBT in 54 ml of a 0.3M solution of NMM in DMF and, after 15 min, reacted with 1.12 g (2.8 mmol) of H-[Phe^{NN}Cha]-H (hydrochloride salt) (see Example 10a) for 18 h. The reaction mixture is concentrated by evaporation using a RE at 50° C. (←brown residue), and the residue is dissolved in methylene chloride and washed twice with saturated NaHCO₃ solution and brine. The aqueous phases are extracted with 2 portions of methylene chloride and the organic phases are dried with Na₂SO₄ and concentrated by evaporation. Filtration through silica gel (methylene chloride/methanol 15:1) and precipitation twice with DIPE from a concentrated methylene chloride solution yields the title compound: TLC R_{f} (U)=0.33; t_{Ret} (I)=21.5 min; FAB-MS (M+H)⁺=606.

The starting material is prepared as follows:

### b) N-(Methoxycarbonyl)-(L)-valine

5.67 g (60 mmol) of chloroformic acid methyl ester are added to 7.0 g (60 mmol) of L-valine in 100 mL of 2N NaOH and 30 mL of dioxane (→exothermic reaction) and the mixture is then stirred for 18 h at RT. The reaction mixture is extracted with methylene chloride and the aqueous phase is acidified with 27 mL of 4N HCl and extracted again with methylene chloride. Drying and concentration by evaporation of the latter methylene chloride phase yields the title compound: t_{Ret} (I)=7.2 min; .¹H-NMR (200 MHz, CD₃OD): 0.96 (t, J=7 Hz, 6H), 2.16 (m, 1H), 3.67 (s, 3H), 4.06 (m, 1H), 7.07 (d, J=8 Hz, HN_{partially exchanged}).

### Example 74

### 1-[2 (S)-(2-Pyridylcarbonyl) oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine

Under a nitrogen atmosphere, 56 μL (0.4 mmol) of 1-chloro-N,N,2-trimethyl-1-propenamine (B. Haveaux, A., Dekoker, M. Rens, A. R. Sidani, J. Toye, L. Ghosez, M. Murakami, M. Yoshioka, and W. Nagata, Organic Syntheses 59, 26 (1980)) are added at 0° C. to 81 mg (0.66 mmol) of 2-picolinic acid in 4 mL of methylene chloride. After 45 min at RT, 1.3 mL of pyridine, 100 mg (0.165 mmol) of N-(methoxycarbonyl)-(L)-Val-[Phe^{NN}Cha]←(N-(methoxycarbonyl)-(L)-Val) (Example 73a) and a spatula tip of DMAP are added and the mixture is stirred for 18 h at RT. The dark reaction mixture is partitioned between 3 portions of methylene chloride, 2 portions of saturated NaHCO₃ solution, water and brine. Column chromatography (SiO₂, ethyl acetate) of the concentration residue of the methylene chloride phase dried with Na₂SO₄ yields the pure title compound: TLC: R_{f} (O)=0.23; t_{Ret} (I)=22.5 min; FAB-MS (M+H)⁺=711.

### Example 75

The following are prepared in accordance with one of the aforementioned processes:
a) 1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine
b) 1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
c) 1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
d) 1-[2(S)-octanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
e) 1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
f) 1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
g) 1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
h) 1-[2(S)-(2-furylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
i) 1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
j) 1-[2(S)-(4-imidazolylacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
k) 1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
l) 1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
m) 1-[2(S)-(2-pyridylacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
n) 1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]-hydrazine;
o) 1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
p) 1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
q) 1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
r) 1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
s) 1-[2(S)-(N-benzyloxycarbonyl-N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
t) 1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
u) 1-[2(S)-(4-morpholinomethylbenzoyl)oxy-3(S)-(N-(methoxycarbonyl)-(L)-valyl)-amino-4-phenyl-butyl]-1-[cyclohexylmethyl]-2-[N-(methoxycarbonyl)-(L)-valyl]-hydrazine;
v) 1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine;
w) 1-[2(S)-(3-carboxypropionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]-hydrazine.

### Example 76

From the title compounds of Examples 1 to 70, there are prepared in accordance with one of the above-mentioned processes the monoacetylated derivatives that contain a 2(S)-acetoxy group instead of the free 2(S)-hydroxy group in the relevant central divalent radical derivatized from butan-2(S)-ol and designated -[Phe^{NN}Phe], -[Phe^{NN}Cha], -[Phe^{NN}Leu], -[Phe^{NN}Nle], -[Phe M (p-F)Phe], -[(p-F)Phe^{NN}(p-F)Phe], -[Phe^{NN}(p-CN)Phe] or -[Cha^{NN}Leu].

### Example 77

The following compounds are prepared in accordance with one of the above-mentioned processes (the starting materials are indicated in square brackets ([ ]) (e.g. the respective Example of which the title compound is used as starting material)):
a) 1-[2(S)-(2-furylcarbonyl)oxy-3(S)-(N-(benzyloxycarbonyl)-(L)-valyl)-amino-4-phenylbutyl)]-1-[benzyl]-2-[N-(benzyloxycarbonyl)-(L)-valyl]-hydrazine [from the title compound of Example 2 and furan-2-carboxylic acid chloride];
b) 1-[2(S)-pivaloyloxy-3(S)-(N-(benzyloxycarbonyl)-(L)-valyl)-amino-4-phenyl-butyl]-1-[cyclohexylmethyl]-2-[N-(benzyloxycarbonyl)-(L)-valyl]-hydrazine [from the title compound of Example 11 and pivalic acid anhydride];
c) 1-[2(S)-(N-methylaminoacetyl)oxy-3(S)-(N-(morpholinocarbonyl)-(L)-valyl)-amino-4-phenyl-butyl]-1-[p-fluorophenylmethyl]-2-[N-(benzyloxycarbonyl)-(L)-valyl]-hydrazine [from the title compound of Example 18 and N-benzyloxycarbonyl-N-methlaminoacetic acid with subsequent hydrogenolysis of the resulting 2(S)-(N-benzyloxycarbonyl-N-methylaminoacetyl compound catalysed by Pd/C];
d) 1-[2(S)-(N-benzyloxycarbonyl-N-methylaminoacetyl)oxy-3(S)-(N-(benzyloxycarbonyl)-(L)-valyl)-amino-4-phenyl-butyl]-1-[p-fluorophenylmethyl]-2-[N-(N-(morpholinocarbonyl)-glycyl)-(L)-valyl]-hydrazine [from the title compound of Example 24 and N-benzyloxycarbonyl-N-methylaminoacetic acid chloride];
e) 1-[2(S)-(N,N-dimethylaminoacetyl)oxy-3(S)-(N-(quinolin-2-ylcarbonyl)-(L)-aspartoyl)-amino-4-phenyl-butyl]-1-[p-fluorophenylmethyl]-2-[tert-butoxycarbonyl]-hydrazine [from the title compound of Example 27 and dimethylaminoacetic acid chloride];
f) 1-[2(S)-(2-pyridylcarbonyl)oxy-3(S)-(N-(benzyloxycarbonyl)-(L)-aspartoyl)-amino-4-phenyl-butyl]-1-[p-fluorophenylmethyl]-2-[N-(benzyloxycarbonyl)-(L)-valyl]-hydrazine [from the title compound of Example 28 and 2-pyridinecarboxylic acid chloride);
g) 1-[2(S)-(4-(morpholinomethyl)benzoyl)oxy-3(S)-(N-(benzyloxycarbonyl)-(L)-valyl)-amino-4-(p-fluorophenyl)-butyl]-1-[p-fluorophenylmethyl]-2-[tert-butoxycarbonyl]-hydrazine [from the title compound of Example 31 and 4-morpholinomethylbenzoic acid by way of the acid chloride in the presence of N,N,2-trimethyl-1-chloropropen-(1)-amine];
h) 1-[2(S)-benzoyloxy-3(S)-(N-(benzyloxycarbonyl)-(L)-valyl)-amino-4-(p-fluorophenyl)-butyl]-1-[p-fluorophenylmethyl]-2-[N-(N-(2-pyridylmethyl)-N-methylaminocarbonyl)-(L)-valyl]-hydrazine [from the title compound of Example 33 and benzoyl chloride];
i) 1-[2(S)-(4-chloromethylbenzoyl)oxy-3(S)-(N-(benzyloxycarbonyl)-(L)-valyl)-amino-4-(p-fluorophenyl)-butyl]-1-[p-fluorophenylmethyl]-2-[N-(2(R,S)-carbamoyl-3-phenylpropionyl)-(L)-valyl]-hydrazine [from the title compound of Example 34 and 4-chloromethylbenzoic acid in the presence of N,N,2-trimethyl-1-chloropropen-(1)-amine];
j) 1-[2(S)-(imidazol-4-ylacetyl)oxy-3(S)-(N-acetyl-valyl)-amino-4-phenyl-butyl]-1-[benzyl]-2-[N-acetyl-valyl]-hydrazine [from the title compound of Example 35 and 1-tritylimidazolyl-4-acetic acid (prepared from trityl chloride and 4-imidazolylacetic acid in the presence of pyridine) in the presence of N,N,2-trimethyl-1-chloropropen-(1)-amine by way of the trityl-protected intermediate with subsequent acidolytic removal of the trityl protecting group, e.g. using trifluoroacetic acid];
k) 1-[2(S)-(2-pyridylacetyl)oxy-3(S)-(N-(quinolin-2-ylcarbonyl)-valyl)-amino-4-phenyl-butyl]-1-[benzyl]-2-[N-quinolin-2-yl-carbonyl-valyl]-hydrazine [from the title compound of Example 37 and 2-pyridineacetic acid in the presence of N,N,2-trimethyl-1-chloropropen-(1)-amine];
l) 1-[2(S)-(3-pyridylacetyl)oxy-3(S)-(N-acetyl-(L)-valyl)-amino-4-phenyl-butyl]-1-[cyclohexylmethyl]-2-[N-acetyl-(L)-valyl]-hydrazine [from the title compound of Example 38 and 3-pyridineacetic acid in the presence of N,N,2-trimethyl-1-chloropropen-(1)-amine];
m) 1-[2(S)-(4-pyridylacetyl)oxy-3(S)-(N-(3-pyridylacetyl)-(L)-valyl)-amino-4-phenyl-butyl]-1-[cyclohexylmethyl]-2-[N-(3-pyridylacetyl)-(L)-valyl]-hydrazine [from the title compound of Example 39 and 4-pyridineacetic acid in the presence of N,N,2-trimethyl-1-chloropropen-(1)-amine];
n) 1-[2(S)-(quinolin-2-ylcarbonyl)oxy-3(S)-(N-(N-(2-pyridylmethyl)-N-methylamino-carbonyl)-(L)-valyl)-amino-4-phenyl-butyl]-1-[p-fluorophenylmethyl]-2-[N-(N-(2-pyridylmethyl)-N-methylaminocarbonyl)-(L)-valyl]-hydrazine [from the title compound of Example 43 and quinoline-2-carboxylic acid in the presence of N,N,2-trimethyl-1-chloropropen-(1)-amine];
o) 1-[2(S)-(2-pyrrolidinylcarbonyl)oxy-3(S)-(N-(benzyloxycarbonyl)-(L)-valyl)-amino-4-phenyl-butyl]-1-[p-fluorophenylmethyl]-2-[N-(benzyloxycarbonyl)-(L)-valyl]-hydrazine [from the title compound of Example 45 and proline in the presence of N,N,2-trimethyl-1-chloropropen-(1)-amine];
p) 1-[2(S)-propionyloxy-3(S)-(N-(benzyloxycarbonyl)-(L)-valyl)-amino-4-phenyl-butyl]-1-[p-cyanophenylmethyl]-2-[N-(benzyloxycarbonyl)-(L)-valyl]-hydrazine [from the title compound of Example 48 and propanoic acid anhydride];
q) 1-[2(S)-butyryloxy-3(S)-(N-(benzyloxycarbonyl)-(L)-isoleucyl)-amino-4-phenyl-butyl]-1-[isobutyl]-2-[N-(benzyloxycarbonyl)-(L)-isoleucyl]-hydrazine [from the title compound of Example 49 and butyric acid anhydride];
r) 1-[2(S)-pentanoyloxy-3(S)-(N-(isobutoxycarbonyl)-(L)-valyl)-amino-4-phenyl-butyl]-1-[isobutyl]-2-[N-(isobutoxycarbonyl)-(L)-valyl]-hydrazine [from the title compound of Example 50 and pentanoic acid chloride];
s) 1-[2(S)-decanoyloxy-3(S)-(N-acetyl-valyl)-amino-4-phenyl-butyl]-1-[isobutyl]-2-[N-acetyl-valyl]-hydrazine [from the title compound of Example 52 and decanoic acid in the presence of N,N,2-trimethyl-1-chloropropen-(1)-amine];
t) 1-[2(S)-dodecanoyloxy-3(S)-N-valyl-amino-4-phenyl-butyl]-1-[isobutyl]-2-[N-valyl]-hydrazine [from the title compound of Example 54, protected at the two free valylamino groups by benzyloxycarbonyl, in the presence of N,N,2-trimethyl-1-chloropropen-(1)-amine with subsequent hydrogenolytic removal of the benzyloxycarbonyl protecting groups from the obtainable intermediate];
u) 1-[2(S)-(3-carboxypropionyl)oxy-3(S)-(N-(thiomorpholinocarbonyl)-(L)-valyl)-amino-4-phenyl-butyl]-1-[isobutyl]-2-[N-(thiomorpholinocarbonyl)-(L)-valyl]-hydrazine [prepared from the title compound of Example 55 and succinic acid anhydride in the presence of pyridine];
v) 1-[2(S)-(4-imidazolylacetyl)oxy-3(S)-(N-(benzyloxycarbonyl)-(L)-valyl)amino-4-phenyl-butyl]-1-[isobutyl]-2-[N-(3-(tetrazol-1-yl)propionyl)-(L)-valyl]-hydrazine [prepared from the title compound of Example 57 and 1-trityl-4-imidazolylacetic acid analogously to Example 77j];
w) 1-[2(S)-(furan-2-ylcarbonyl)oxy-3(S)-(N-acetyl-(L)-valyl)amino-4-phenyl-butyl]-1-[isobutyl]-2-[N-(2(R,S)-carbamoyl-3-phenyl-propionyl)-(L)-valyl]-hydrazine [prepared from the title compound of Example 58 and furan-2-carboxylic acid chloride];
x) 1-[2(S)-pivaloyloxy-3(S)-(N-(benzyloxycarbonyl)-(L)-valyl)amino-4phenyl-butyl]-1-[n-butyl]-2-[2(R,S)-(N-(2-morpholinoethyl)carbamoyl)-3-methylbutyryl]-hydrazine [prepared from the title compound of Example 61 and pivalic acid anhydride];
y) 1-[2(S)-(N-benzyloxycarbonyl-N-methylaminoacetyl)oxy-3(S)-(N-(benzyloxycarbonyl)-(L)-valyl)amino-4-phenylbutyl]-1-[n-butyl]-2-[N-(3-(tetrazol-1-yl)-propionyl)valyl]-hydrazine [from the title compound of Example 62 analogously to Example 77d];
z) 1-[2(S)-(N-methylaminoacetyl)oxy-3(S)-(N-(benzyloxycarbonyl)-(L)-valyl)amino-4-phenyl-butyl]-1-[n-butyl]-2-[N-(2(R,S)-(N-(2-pyridylmethyl)-carbamoyl)-3-methyl)butyryl]-hydrazine [from the title compound of Example 63 by way of the 2(S)-N-benzyloxycarbonyl-N-methylaminoacetoxy analogue by hydrogenolysis in accordance with Example 77c).

### Example 78

The following compounds are prepared in accordance with one of the above-mentioned processes (the starting materials are given in square brackets ([ ]) (e.g. the respective Example of which the title compound is used as starting material)):
a) 1-[2(S)-(N,N-dimethylaminoacetyl)oxy-3(S)-((N-benzyloxycarbonyl)-(L)-valyl)-amino-4-phenyl-butyl]-1-[p-fluorophenylmethyl]-2-[N-(3-(tetrazol-1-yl)-propionyl)valyl]-hydrazine [from the title compound of Example 64 and dimethylaminoacetic acid chloride];
b) 1-[2(S)-(pyridin-2-ylcarbonyl)oxy-3(S)-(N-methoxycarbonyl)-(L)-valyl)-amino-4-phenyl-butyl]-1-[isobutyl]-2-[N-(methoxycarbonyl)-(L)-valyl]-hydrazine [from the title compound of Example 66 and 2-pyridinecarboxylic acid chloride].
c) 1-[2(S)-(4-morpholinomethylbenzoyl)oxy-3(S)-(N-methoxycarbonyl)-(L)-valyl)-amino-4-phenyl-butyl]-1-[p-fluorophenylmethyl]-2-[N-(methoxycarbonyl)-(L)-valyl]-hydrazine [from the title compound of Example 67 and 4-morpholinomethyl benzoic acid (by way of the acid chloride in the presence of N,N,2-trimethyl-1-chloropropen-(1)-amine)];
d) 1-[2(S)-benzoyloxy-3(S)-(N-methoxycarbonyl)-(L)-valyl)-amino-4-phenyl-butyl]-1-[p-cyanophenylmethyl]-2-[N-(methoxycarbonyl)-(L)-valyl]-hydrazine [from the title compound of Example 68 and benzoic acid anhydride].

### Example 79

### 1-[2(S)-Hydroxy-3(S)-(N-allyloxycarbonyl-(L)-valyl)amino-4-phenylbutyl]-1-[cyclohexylmethyl]-2-[N-allyloxycarbonyl-(L)-valyl]-hydrazine

Analogously to Example 2, the title compound is obtained from 500 mg (1.25 mmol) of H-[Phe^{NN}Cha]-H.3HCl from Example 10a, 753 mg (3.74 mmol) of N-allyloxycarbonyl-(L)-valine, 1.65 g (3.74 mmol) of BOP, 505 mg (3.74 mmol) of HOBt and 24.7 mL of 0.3M N-methylmorpholine in DMF after precipitation twice from methylene chloride by the addition of DIPE and lyophilisation from dioxane. FAB-MS: (M+H)⁺=658, t_{Ret} (I)=25.5 min, R_{f} (H)=0.44.

The starting compound is prepared in the following manner:

### a) Allyloxycarbonyl-(L)-valine

The title compound is obtained in the form of a colorless oil analogously to Example 1c) starting from 10 g (85.3 mmol) of (L)-valine and 10.3 g (85.3 mmol) of allyl chloroformate. ¹H-NMR (200 MHz, CDCl₃): 6.05-5.8 (m, 1H), 5.35 (s, broad, 1H), 5.20 (m, 2H), 4.60 (d, broad, J=6 Hz, 2H), 4.33 (m, 1H), 2.25 (m, 1H), 1.00 (d, J=7 Hz, 3H), 0.93 (d, J=7 Hz, 3H).

### Example 80

### 1-[2(S)-Hydroxy-3(S)-(N-ethoxycarbonyl-(L)-valyl)amino-4-phenylbutyl]-1-cyclohexylmethyl]-2-[N-ethoxycarbonyl-(L)-valyl]-hydrazine

Analogously to Example 2, the title compound is obtained from 500 mg (1.25 mmol) of H-[Phe^{NN}Cha]-H.3 HCl from Example 10a, 708 mg (3.74 mmol) of N-ethoxycarbonyl-(L)-valine, 1.65 g (3.74 mmol) of BOP, 505 mg (3.74 mmol) of HOBt and 24.7 mL of 0.3M N-methylmorpholine in DMF after chromatographic purification (SiO₂, methylene chloride/diethyl ether (1:1)), precipitation from methylene chloride by the addition of DIPE and lyophilization from dioxane. FAB-MS: (M+H)⁺=634, t_{Ret} (I)=24.2 min., R_{f} (H)=0.3.

The starting compound is prepared in the following manner:

### a) N-Ethoxycarbonyl-(L)-valine

Analogously to Example 1c, the title compound is obtained in the form of a colorless oil starting from 10 g (85.3 mmol) of (L)-valine and 9.3 g (85.3 mmol) of ethyl chloroformate. ¹H-NMR (200 MHz, CDCl₃): 5.15 (d, broad, 1H), 4.32 (m, 1H), 4.15 (q, J=7 Hz, 2H), 2.25 (m, 1H), 1.25 (t, J=7 Hz, 3H) 0.98 (d, J=7 Hz, 3H), 0.92 (d, J=7 Hz, 3H).

### Example 81

### 1-[2(S)-Hydroxy-3(S)-(N-trifluoroacetyl-(L)-valyl)amino-4-phenylbutyl]-1-[cyclohexylmethyl]-2-[N-trifluoroacetyl-(L)-valyl]-hydrazine

Analogously to Example 2, the title compound is obtained from 500 mg (1.25 mmol) of H-[Phe^{NN}Cha]-H.3HCl from Example 10a, 798 mg (3.74 mmol) of N-trifluoroacetyl-(L)-valine, 1.65 g (3.74 mmol) of BOP, 505 mg (3.74 mmol) of HOBt and 24.7 mL of 0.3M N-methylmorpholine in DMF after chromatographic purification (SiO₂, methylene chloride/diethyl ether (1:1)), precipitation from methylene chloride by the addition of hexane and lyophilisation from dioxane. FAB-MS: (M+H)⁺=682, t_{Ret} (I)=26.1 min. R_{f} (H)=0.65.

The starting compound is prepared in the following manner:

### a) N-Trifluoroacetyl-(L)-valine

At 0° C., 6 mL (42.8 mmol) of trifluoroacetic anhydride are added dropwise to a solution of 8.16 g (38.9 mmol) of (L)-valine-tert-butyl ester and 17.3 mL (124.5 mmol) of triethylamine in 100 mL of methylene chloride and the reaction mixture is stirred overnight at RT. After washing with 10% citric acid and saturated sodium chloride solution, the organic phase is concentrated by evaporation and yields N-trifluoroacetyl-(L)-valine tert-butyl ester in the form of a yellow oil, which is dissolved in 40 mL of a (1:1) mixture of methylene chloride and TFA and stirred for 5 h at RT. Concentration by evaporation of the reaction solution and digestion of the residue with hexane yields the title compound in the form of a white solid. ¹H-NMR (200 MHz, CD₃OD): 4.32 (d, J=6 Hz, 1H), 2.23 (m, 1H), 0.98 (d, J=7 Hz, 3H), 0.96 (d, J=7 Hz, 3H).

### Example 82

### 1-[2(S)-Hydroxy-3(S)-(N-(2-(2-methoxyethoxy)ethoxy)carbonyl-(L)-valyl)-amino-4-phenylbutyl]-1-[cyclohexylmethyl]-2-[N-(2-(2-methoxyethoxy)ethoxy)carbonyl-(L)-valyl]-hydrazine

Analogously to Example 2, the title compound is obtained from 500 mg (1.25 mmol) of H-[Phe^{NN}Cha]-H.3 HCl from Example 10a, 985 mg (3.74 mmol) of N-methoxyethoxy-ethoxycarbonyl-(L)-valine, 1.65 g (3.74 mmol) of BOP, 505 mg (3.74 mmol) of HOBt and 24.7 mL of 0.3M N-methylmorpholine in DMF after chromatographic purification (SiO₂, chloroform/methanol (30:1)), precipitation from methylene chloride by the addition of hexane and lyophilization from dioxane. FAB-MS: (M+H)⁺=782, t_{Ret} (I)=21.2 min, R_{f} (T)=0.26.

The starting compound is prepared in the following manner:

### a) N-(2-(2-Methoxyethoxy)-ethoxy)carbonyl-(L)-valine

19.8 mL (168 mmol) of diethylene glycol monomethyl ether are added dropwise at 0° C. to 100 mL of a 20% solution of phosgene in toluene and the mixture is stirred overnight at RT. Excess phosgene is expelled with nitrogen and the reaction solution is washed with water and concentrated by evaporation. Analogously to Example 1c, 10 g (85.4 mmol) of (L)-valine are added to the crude 2-(2-methoxyethoxy)-ethylchloroformate (15.6 g, 85.4 mmol) yielding the title compound in the form of a colorless resin. ¹H-NMR (200 MHz, CD₃OD): 4.18 (m, 2H), 4.05 d, J=6 Hz, 1H), 3.72-3.40 (m, 8H), 3.35 (s, 3H), 2.18 (m, 1H), 0.95 (t, J=7H, 6H).

### Example 83

### 1-[2(S)-Hydroxy-3(S)-(N-(2-methoxyethoxy)acetyl-(L)-valyl)amino-4-phenylbutyl]-1-[cyclohexylmethyl]-2-[N-(2-methoxyethoxy)acetyl-(L)-valyl]-hydrazine

Analogously to Example 2, the title compound is obtained from 500 mg (1.25 mmol) of H-[Phe^{NN}Cha]-H-3.HCl from Example 10a, 873 mg (3.74 mmol) of N-(2)-methoxyethoxyacetyl-(L)-valine, 1.65 g (3.74 mmol) of BOP, 505 mg (3.74 mmol) of HOBt and 24.7 mL of 0.3M N-methylmorpholine in DMF after chromatographic purification (SiO₂, chloroform/methanol (30:1)), precipitation from methylene chloride by the addition of hexane and lyophilization from dioxane. FAB-MS: (M+H)⁺=722, t_{Ret} (I)=21.3 min, R_{f} (T)=0.23.

The starting compound is prepared in the following manner:

### a) N-(2-Methoxyethoxy)-acetyl-(L)-valine

At 0° C., 6.4 mL (45.7 mmol) of triethylamine are added to a solution of 5 g (23.9 mmol) of (L)-valine tert-butyl ester, 2.91 g (21.7 mmol) of 2-(methoxyethoxy)-acetic acid and 3.55 g (21.7 mmol) of cyanophosphonic acid diethyl ester in 30 mL of DMF and the mixture is then stirred overnight at RT. The reaction mixture is diluted with methylene chloride, washed in succession with 10% citric acid, saturated sodium hydrogen carbonate solution and saturated sodium chloride solution and, after concentration by evaporation of the organic phase, yields 5.1 g of N-methoxyethoxyacetyl-(L)-valine tert-butyl ester, which is stirred for 1 h at RT in 22 mL of a (1:1) mixture of methylene chloride and TFA. Concentration by evaporation of the reaction solution yields the title compound in the form of a colourless oil. ¹H-NMR (200 MHz, CDCl₃): 10.0 (s, broad, 1H), 7.62 d, broad, 1H), 4.55 (m, 1H), 4.10 (s, 2H), 3.70 (m, 2H), 3.58 (m, 2H), 3.40 (s, 3H), 2.28 (m, 1H), 0.98 (d, J=7 Hz, 0.3H), 0.95 (d, J=7 Hz, 3H).

### Example 84

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[thien-2-ylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[thien-2-ylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[thien-2-ylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[thien-2-ylmethyl]-2-[N-(N,N-dimethylaminocarbonyl)-(L)-valyl]-hydrazine;
E) 1-[2(S)-hydroxy-3(S)-(N-(N-(2-methoxyethyl)aminocarbonyl)-(L)-valyl)amino-4-phenylbutyl]-1-[thien-2-ylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)-amino-4-phenylbutyl]-1-[thien-2-ylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methyl-aminocarbonyl)-(L)-valyl)amino-4-phenylbutyl]-1-[thien-2-ylmethyl]-2-[N-(N-(2-(morpholin-4-yl)ethyl)-N-methylaminocarbonyl)-(L)-valyl]-hydrazine.

### Example 85

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[2,3,5,6-tetrahydropyran-4-ylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[2,3,5,6-tetrahydropyran-4-ylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[2,3,5,6-tetrahydropyran-4-ylmethyl]-2-[N-ethoxycarbonyl-(L)-valyl]-hydrazine
D) 1-[2(S)-hydroxy-3(S)-(N-(N,N-dimethylaminocarbonyl)-(L)-valyl)amino-4-phenylbutyl]-1-[2,3,5,6-tetrahydropyran-4-ylmethyl]-2-[N-(N,N-dimethylaminocarbonyl)-(L)-valyl]-hydrazine;
E) 1-[2(S)-hydroxy-3(S)-(N-(N-(2-methoxyethyl)aminocarbonyl)-(L)-valyl)amino-4-phenylbutyl]-1-[2,3,5,6-tetrahydropyran-4-ylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)-amino-4-phenylbutyl]-1-[2,3,5,6-tetrahydropyran-4-ylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[2,3,5,6-tetrahydropyran-4-ylmethyl]-2-[N-(N-(2-(morpholin-4-yl)ethyl)-N-methylaminocarbonyl)-(L)-valyl]-hydrazine.

### Example 86

The following compounds are obtained analogously to the Examples and processes described hereinbefore and hereinafter:
A) 1-[2(S)-Hydroxy-3(S)-(N-acetyl-(L)-valyl)amino-4-phenylbutyl]-1-[4-hydroxyphenylmethyl]-2-[N-acetyl-(L)-valyl]-hydrazine:

Analogously to Example 37, the title compound is obtained from 200 mg (0.48 mmol) of 1-[2(S)-hydroxy-3(S)-amino-4-phenylbutyl]-1-[4-hydroxyphenylmethyl]-hydrazine.3HCl, 175 mg (1.07 mmol) of N-methoxycarbonyl-(L)-valine from Example 73b, 473 mg (1.07 mmol) of BOP, 145 mg (1.07 mmol) of HOBt and 8.4 mL of 0.3M N-methylmorpholine in DMF after chromatographic purification (SiO₂, methylene chloride/diethyl ether/methanol (10:1:1)), precipitation from methylene chloride by the addition of DIPE and lyophilization from dioxane. FAB-MS: (M+H)⁺=616, t_{Ret} (V)=11.6 min., R_{f} (K′)=0.56.

The starting compounds are prepared in the following manner:

### a) 1-[2(S)-Hydroxy-3(S)-amino-4-phenylbutyl]-1-[4-hydroxyphenylmethyl]-hydrazine.3HCl

Analogously to Example 2a, the title compound is obtained from 470 mg (0.94 mmol) of 1-[2(S)-hydroxy-3(S)-tert-butoxycarbonylamino-4-phenylbutyl]-1-[4-hydroxyphenylmethyl]-2-[tert-butoxycarbonyl]-hydrazine after complete concentration by evaporation and digestion of the residue with methylene chloride. ¹H-NMR (200 MHz, CD₃OD): 7.42-7.15 (m, 7H), 6.92 (d, J=8 Hz, 2H), 4.1-3.85 (m, 3H), 3.55 (m, 1H), 3.1 (m, 2H), 2.8 (m, 2H).

### b) 1-[2(S)-Hydroxy-3(S)-tert-butoxycarbonylamino-4-phenylbutyl]-1-[4-hydroxyphenyl-methyl]-2-[tert-butoxycarbonyl]-hydrazine

Analogously to Example 1, the title compound is obtained from 26.2 g (23.6 mmol) of (2R,3S)-1-[3-Boc-amino-2-phenylethyl]oxirane and 5.63 g (23.6 mmol) of tert-butyl-3-(4-hydroxyphenyl-methyl)-carbazate after crystallization from ethyl acetate/DIPE. ¹H-NMR (200 MHz, CD₃OD): 7.28-7.10 (m, 7H), 6.70 (d, J=8 Hz, 2H), 4.7 (m, 4H), 2.95-2.45 (m, 4H), 1.31 (s, 9H), 1.28 (s, 9H). t_{Ret} (V)=15.0 min.

### c) tert-Butyl-3-(4-hydroxyphenyl-methyl)-carbazate

Analogously to Example 4a, 14 g (106 mmol) of 4-hydroxybenzaldehyde and 14 g (117 mmol) of tert-butylcarbazate in 125 mL of ethanol are reacted to form 4-hydroxybenzaldehyde-tert-butoxycarbonylhydrazone. (19.8 g, 80%). 9.73 g thereof are hydrogenated in 200 ml of THF in the presence of 0.6 g of 5% palladium on carbon at 1 atm hydrogen pressure to yield the title compound, which is crystallised from hot methanol. ¹H-NMR (200 MHz, CD₃OD): 7.18 (d, J=8 Hz, 2H), 6.73 (d, J=8 Hz, 2H), 3.80 (s, 2H), 1.45 (s, 9H).
B) 1-[2(S)-Hydroxy-3(S)-(N-methoxycarbonyl-(L)-valyl)amino-4-phenylbutyl]-1-[4-hydroxyphenylmethyl]-2-[N-methoxycarbonyl-(L)-valyl]-hydrazine
C) 1-[2(S)-Hydroxy-3(S)-(N-ethoxycarbonyl-(L)-valyl)amino-4-phenylbutyl]-1-[4-hydroxyphenylmethyl]-2-[N-ethoxycarbonyl-(L)-valyl]-hydrazine

A solution of 2.0 g (4.86 mmol) of 1-[2(S)-hydroxy-3(S)-amino-4-phenylbutyl]-1-[4-methoxyphenylmethyl]-hydrazine .3HCl (Example 86A) a)), 2.54 g (13.8 mmol) of N-ethoxycarbonyl-(L)-valine from Example 9a and 2.13 mL (13.8 mmol) of cyanophosphonic acid diethyl ester in 45 mL of DMF is cooled to 0° C. and 4.0 mL (29.2 mmol) of triethylamine are added. The reaction mixture is stirred for 6 h at RT under a nitrogen atmosphere and fully concentrated by evaporation in vacuo. The residue is dissolved in methylene chloride, washed with saturated sodium carbonate solution, 10% aqueous citric acid and saturated sodium chloride solution, filtered through cotton wadding and concentrated by evaporation. Chromatographic purification (SiO₂, methylene chloride/diethyl ether/methanol (10:10:1)) and precipitation twice from methanol/methylene chloride by the addition of DIPE yields the title compound. FAB-MS: (M+H)⁺=644, t_{Ret} (V)=12.8 min, R_{f} (B)=0.41.
D) 1-[2(S)-Hydroxy-3(S)-(N-(N,N-dimethylaminocarbonyl)-(L)-valyl)amino-4-phenylbutyl]-1-[4-hydroxyphenylmethyl]-2-[N-(N,N-dimethylaminocarbonyl)-(L)-valyl]-hydrazine.
E) 1-[2(S)-Hydroxy-3(S)-(N-(N-(2-methoxyethyl)aminocarbonyl)-(L)-valyl)amino-4-phenylbutyl]-1-[4-hydroxyphenylmethyl]-2-[N-(N-(2-methoxyethyl)aminocarbonyl)-(L)-valyl]-hydrazine.
F) 1-[2(S)-Hydroxy-3(S)-(N-(N-(2-(morpholin-4-yl)ethyl)aminocarbonyl)-(L)-valyl)-amino-4-phenylbutyl]-1-[4-hydroxyphenylmethyl]-2-[N-(N-(2-(morpholin-4-yl)ethyl)-aminocarbonyl)-(L)-valyl]-hydrazine.
G) 1-[2(S)-Hydroxy-3(S)-(N-(N-(2-(morpholin-4-yl)ethyl)-N-methyl-aminocarbonyl)-(L)-valyl)amino-4-phenylbutyl]-1-[4-hydroxyphenylmethyl]-2-[N-(N-(2-(morpholin-4-yl)ethyl)-N-methylaminocarbonyl)-(L)-valyl]-hydrazine.

### Example 87

The following compounds are obtained analogously to the Examples and processes described hereinbefore and hereinafter:
A) 1-[2(S)-Hydroxy-3(S)-(N-acetyl-(L)-valyl)amino-4-phenylbutyl]-1-[4-methoxyphenylmethyl]-2-[N-acetyl-(L)-valyl]-hydrazine:

Analogously to Example 37, the title compound is obtained from 200 mg (0.47 mmol) of 1-[2(S)-hydroxy-3(S)-amino-4-phenylbutyl]-1-[4-methoxyphenylmethyl]-hydrazine-3HCl, 225 mg (1.41 mmol) of N-acetyl-(L)-valine, 624 mg (1.41 mmol) of BOP, 191 mg (1.41 mmol) of HOBt and 9.4 mL of 0.3M N-methylmorpholine in DMF after chromatographic purification (SiO₂, methylene chloride/methanol (12:1)) and lyophilisation from dioxane/water/tert-butanol. FAB-MS: (M+H)⁺=598, t_{Ret} (V)=11.2 min, R_{f} (I′)=0.25.

The starting materials are prepared in the following manner:

### a) 1-[2(S)-Hydroxy-3(S)-amino-4-phenylbutyl]-1-[4-methoxyphenylmethyl]-hydrazine.3HCl

Analogously to Example 2a, the title compound is obtained from 2.65 g (5.14 mmol) of 1-[2(S)-hydroxy-3(S)-tert-butoxycarbonylamino-4-phenylbutyl]-1-[4-methoxyphenylmethyl]-2-[tert-butoxycarbonyl]-hydrazine after lyophilization. ¹H-NMR (200 MHz, CD₃ OD): 7.42-7.15 (m, 7H), 6.92 (d, J=8 Hz, 2H), 4.1-3.8 (m, 3H), 3.75 (s, 3H), 3.55 (m, 1H), 3.1 (m, br, 2H), 2.75 (m, br, 2H).

### b) 1-[2(S)-Hydroxy-3(S)-tert-butoxycarbonylamino-4-phenylbutyl]-1-[4-methoxyphenylmethyl]-2-[tert-butoxycarbonyl]-hydrazine

Analogously to Example 1, the title compound is obtained from 3.13 g (11.9 mmol) of (2R,3S)-1-[3-Boc-amino-2-phenylethyl]oxirane and 3.0 g (11.9 mmol) of tert-butyl-3-(4-methoxyphenyl-methyl)-carbazate from Example 100 C) b) (see below) after crystallization from methanol/DIPE. ¹H-NMR (200 MHz, CD₃OD): 7.3-7.1 (m, 7H), 6.85 (d, J=8 Hz, 2H), 3.78 (s, 3H), 3.65 (m, 4H), 2.9-2.5 (m, 4H), 1.25 (s, 9H), 1.20 (s, 9H), t_{Ret} (V)=16.6 min.
B) 1-[2(S)-Hydroxy-3(S)-(N-methoxycarbonyl-(L)-valyl)amino-4-phenylbutyl]-1-[4-methoxyphenylmethyl]-2-[N-methoxycarbonyl-(L)-valyl]-hydrazine:

Analogously to Example 37, the title compound is obtained from 200 mg (0.47 mmol) of the title compound of Example 87A) a), 247 mg (1.41 mmol) of N-methoxycarbonyl-(L)-valine from Example 73b, 624 mg (1.41 mmol) of BOP, 191 mg (1.41 mmol) of HOBt and 9.4 mL of 0.3M N-methylmorpholine in DMF after chromatographic purification (SiO₂, methylene chloride/methanol (19:1)) and lyophilization from dioxane. FAB-MS: (M+H)⁺=630, t_{Ret} (V)=13.5 min. R_{f} (A′)=0.27.
C) 1-[2(S)-Hydroxy-3(S)-(N-ethoxycarbonyl-(L)-valyl)amino-4-phenylbutyl]-1-[4-methoxyphenylmethyl]-2-[N-methoxycarbonyl-(L)-valyl]-hydrazine:

Analogously to Example 37, the title compound is obtained from 200 mg (0.47 mmol) of the title compound of Example 87A) a), 267 mg (1.41 mmol) of N-ethoxycarbonyl-(L)-valine from Example 80a, 624 mg (1.41 mmol) of BOP, 191 mg (1.41 mmol) of HOBt and 9.4 mL of 0.3M N-methylmorpholine in DMF after chromatographic purification (SiO₂, methylene chloride/methanol (12:1)), precipitation from methylene chloride by the addition of DIPE and lyophilization from dioxane. FAB-MS: (M+H)⁺=658, t_{Ret} (V)=15.0 min, R_{f} (I′)=0.55.
D) 1-[2(S)-Hydroxy-3(S)-(N-(N,N-dimethylaminocarbonyl)-(L)-valyl)amino-4-phenylbutyl]-1-[4-methoxyphenylmethyl]-2-[N-(N,N-dimethylaminocarbonyl)-(L)-valyl]-hydrazine.
E) 1-[2(S)-Hydroxy-3(S)-(N-(N-(2-methoxyethyl)aminocarbonyl)-(L)-valyl)amino-4-phenylbutyl]-1-[4-methoxyphenylmethyl]-2-[N-(N-(2-methoxyethyl)aminocarbonyl)-(L)-valyl]-hydrazine.
F) 1-[2(S)-Hydroxy-3(S)-(N-(N-(2-(morpholin-4-yl)ethyl)aminocarbonyl)-(L)-valyl)-amino-4-phenylbutyl]-1-[4-methoxyphenylmethyl]-2-[N-(N-(2-(morpholin-4-yl)ethyl)-aminocarbonyl)-(L)-valyl]-hydrazine.
G) 1-[2(S)-Hydroxy-3(S)-(N-(N-(2-(morpholin-4-yl)ethyl)-N-methyl-aminocarbonyl)-(L)-valyl)amino-4-phenylbutyl]-1-[4-methoxyphenylmethyl]-2-[N-(N-(2-(morpholin-4-yl)ethyl)-N-methylaminocarbonyl)-(L)-valyl]-hydrazine.

### Example 88

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[4-benzyloxyphenylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[4-benzyloxyphenylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[4-benzyloxyphenylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[4-benzyloxyphenylmethyl]-2-[N-(N,N-dimethylaminocarbonyl)-(L)-valyl]-hydrazine;
E) 1-[2(S)-hydroxy-3(S)-(N-(N-(2-methoxyethyl)aminocarbonyl)-(L)-valyl)amino-4-phenylbutyl]-1-[4-benzyloxyphenylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)amino-4-phenylbutyl]-1-[4-benzyloxyphenylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methyl-aminocarbonyl)-(L)-valyl)amino-4-phenylbutyl]-1-[4-benzyloxyphenylmethyl]-2-[N (N-(2-(morpholin-4-yl)ethyl)-N-methylaminocarbonyl)-(L)-valyl]-hydrazine.

### Example 89

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[4-(3,4-dimethoxybenzyl)oxyphenylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[4-(3,4-dimethoxybenzyl)oxyphenylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[4-(3,4-dimethoxybenzyl)oxyphenylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[4-(3,4-dimethoxybenzyl)oxyphenylmethyl]-2-[N-(N,N-dimethylamino carbonyl)-(L)-valyl]-hydrazine;
E) 1-[2(S)-hydroxy-3(S)-(N-(N-(2-methoxyethyl)aminocarbonyl)-(L)-valyl) amino-4-phenylbutyl]-1-[4-(3,4-dimethoxybenzyl)oxyphenylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)-N-methylaminocarbonyl)-(L)-valyl]-hydrazine.

### Example 90

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[4-isobutoxyphenylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[4-isobutoxyphenylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[4-isobutoxyphenylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[4-isobutoxyphenylmethyl]-2-[N-(N,N-dimethylaminocarbonyl)-(L)-valyl]-hydrazine;
E) 1-[2(S)-hydroxy-3(S)-(N-(N-(2-methoxyethyl)aminocarbonyl)-(L)-valyl)amino-4-phenylbutyl]-1-[4-isobutoxyphenylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)amino-4-phenylbutyl]-1-[4-isobutoxyphenylmethyl]-2-[N-(N-(2-(morpholin-4-yl)ethyl)-aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[4-isobutoxyphenylmethyl]-2-[N-(N-(2-(morpholin-4-yl)-ethyl)-N-methylaminocarbonyl)-(L)-valyl]-hydrazine.

### Example 91

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[4-(2-methoxyethoxy)phenylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[4-(2-methoxyethoxy)phenylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[4-(2-methoxyethoxy)phenylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[4-(2-methoxyethoxy)phenylmethyl]-2-[N-(N,N-dimethylaminocarbonyl)-(L)-valyl]-hydrazine;
E) 1-[2(S)-hydroxy-3 (S)-(N-(N-(2-methoxyethyl)aminocarbonyl)-(L)-valyl)amino-4-phenylbutyl]-1-[4-(2-methoxyethoxy)phenylmethyl]-2-[N— (N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)-amino-4-phenylbutyl]-1-[4-(2-methoxyethoxy)phenylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[4-(2-methoxyethoxy)phenylmethyl]-2-[N-(N-(2-(morpholin-4-yl)ethyl)-N-methylaminocarbonyl)-(L)-valyl]-hydrazine.

### Example 92

The following compounds are obtained analogously to the Examples and processes described hereinbefore and hereinafter:
A) 1-[2(S)-hydroxy-3(S)-(N-acetyl-(L)-valyl)amino-4-phenylbutyl]-1-[methylene-3,4-dioxyphenylmethyl]-2-[N-acetyl-(L)-valyl]-hydrazine;
B) 1-[2(S)-hydroxy-3(S)-(N-methoxycarbonyl-(L)-valyl)amino-4-phenylbutyl]-1-[methylene-3,4-dioxyphenylmethyl]-2-[N-methoxycarbonyl-(L)-valyl]-hydrazine;
C) 1-[2(S)-hydroxy-3(S)-(N-ethoxycarbonyl-(L)-valyl)amino-4-phenylbutyl]-1-[methylene-3,4-dioxyphenylmethyl]-2-[N-ethoxycarbonyl-(L)-valyl]-hydrazine;
D) 1-[2(S)-hydroxy-3(S)-(N-(N,N-dimethylaminocarbonyl)-(L)-valyl)amino-4-phenylbutyl]-1-[methylene-3,4-dioxyphenylmethyl]-2-[N-(N,N-dimethylaminocarbonyl)-(L)-valyl]-hydrazine;
E) 1-[2(S)-hydroxy-3 (S)-(N-(N-(2-methoxyethyl)aminocarbonyl)-(L)-valyl) amino-4-phenylbutyl]-1-[methylene-3,4-dioxyphenylmethyl]-2-[N-(N-(2-methoxyethyl) aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)amino-4-phenylbutyl]-1-[methylene-3,4-dioxyphenylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[methylene-3,4-dioxyphenylmethyl]-2-[N— (N-(2-(morpholin-4-yl)ethyl)-N-methylaminocarbonyl)-(L)-valyl]-hydrazine;
H) 1-[2(S)-hydroxy-3(S)-(N-acetyl-(L)-valyl)amino-4-phenylbutyl]-1-[3,4-dimethoxyphenylmethyl]-2-[N-acetyl-(L)-valyl]-hydrazine;
I) 1-[2(S)-hydroxy-3(S)-(N-methoxycarbonyl-(L)-valyl)amino-4-phenylbutyl]-1-[3,4-dimethoxyphenylmethyl]-2-[N-methoxycarbonyl-(L)-valyl]-hydrazine;
J) 1-[2(S)-hydroxy-3(S)-(N-ethoxycarbonyl-(L)-valyl)amino-4-phenylbutyl]-1-[3,4-dimethoxyphenylmethyl]-2-[N-ethoxycarbonyl-(L)-valyl]-hydrazine;
K) 1-[2(S)-hydroxy-3(S)-(N-(N,N-dimethylaminocarbonyl)-(L)-valyl)amino-4-phenylbutyl]-1-[3,4-dimethoxyphenylmethyl]-2-[N—(N,N-dimethylaminocarbonyl)-(L)-valyl]-hydrazine;
L) 1-[2(S)-hydroxy-3(S)-(N-(N-(2-methoxyethyl)aminocarbonyl)-(L)-valyl) amino-4-phenylbutyl]-1-[3,4-dimethoxyphenylmethyl]-2-[N-(N-(2-methoxyethyl)aminocarbonyl)-(L)-valyl]-hydrazine;
M) 1-[2(S)-hydroxy-3(S)-(N-(N-(2-(morpholin-4-yl)ethyl)aminocarbonyl)-(L)-valyl)-amino-4-phenylbutyl]-1-[3,4-dimethoxyphenylmethyl]-2-[N-(N-(2-(morpholin-4-yl)ethyl)aminocarbonyl)-(L)-valyl]-hydrazine;
N) 1-[2(S)-hydroxy-3(S)-(N-(N-(2-(morpholin-4-yl)ethyl)-N-methylaminocarbonyl)-(L)-valyl)amino-4-phenylbutyl]-1-[3,4-dimethoxyphenylmethyl]-2-[N-(N-(2-(morpholin-4-yl)ethyl)-N-methylaminocarbonyl)-(L)-valyl]-hydrazine.

### Example 93

The following compounds are obtained analogously to the Examples and processes described hereinbefore and hereinafter:
A) 1-[2(S)-Hydroxy-3(S)-(N-acetyl-(L)-valyl)amino-4-phenylbutyl]-1-[4-biphenylylmethyl]-2-[N-acetyl-(L)-valyl]-hydrazine

Preparation analogously as described in Example 93B) with acetyl-(L)-valine instead of the methoxycarbonyl-(L)-valine. After purification by column chromatography on silica gel (eluent: 15:1 dichloromethane:methanol), the title compound is obtained: FAB MS (M+H)⁺=644. R_{f} (15:1 dichloromethane: methanol)=0.40.
B)1-[²(S)-Hydroxy-3(S)-(N-methoxycarbonyl-(L)-valyl)amino-4-phenylbutyl]-1-[4-biphenylylmethyl]-2-[N-methoxycarbonyl-(L)-valyl]-hydrazine

82.9 mL (594.6 mmol) of triethylamine are added to a stirred and cooled (0° C.) suspension of 48.4 g (276.1 mmol) of methoxycarbonyl-(L)-valine, 101.8 g (530.9 mmol) of dimethyl-3-[3-dimethylamino)propyl]carbodiimide hydrochloride and 43 g (318.5 mmol) of 1-hydroxybenzotriazole hydrate in 1 L of DMF. After 30 min, 50 g (106.2 mmol) of 1-(4-biphenylyl)-5(S)-2,5-diamino-4(S)-hydroxy-6-phenyl-2-azahexane hydrochloride are added in one portion. After 10 min, the cooling bath is removed and the mixture is allowed to stir at room temperature for 16 h. The solvent is evaporated under reduced pressure, and the residue is dissolved in 500 mL of dichloromethane. This solution is subsequently washed with 1 L of 10% aqueous citric acid, 1 L of saturated NaHCO₃ solution and 1L of brine. The aqueous layers are re-extracted three times with 500 mL of dichloromethane. The combined organic layers are filtered through a pad of cotton wool and evaporated under reduced pressure. The residue is crystallized by dissolving it in 100 mL of dichloromethane and adding diisopropylether. Crystallization is completed upon cooling to 5° C. for 16 h. Filtration of the solid and drying at 60° C. gives the title compound as a white, amorphous solid which is homogenous by thin layer chromatography (15:1 dichloromethane:methanol):

m.p. 214°-216° C., ¹H-NMR (CD₃OD) δ=7.58-7.15 (m, 14H), 4.13 (t, br, 1H), 4.0 and 3.90 (AB-system, J=8, 2H), 3.81 (d, J=4, 1H), 3.75/d, br, 1H), 3.63-3.60 (m, 1H), 3.6 (s, 3H), 3.60 (s, 3H), 2.96-2.80 (m, 3H), 2.65 (dd, J=8, 1, 1H), 1.83 (sept, J=7, 1H), 1.64 (sept, J=7, 1H), 0.76 (apparent t, J=7, 6H), 0.63 (d, J=7, 3H), 0.61 (J=7, 3H); FAB-MS (M+H)⁺=676.

The starting material is prepared as follows:

### a) N¹-(4-Biphenylylcarbaldehyde)-N²-(tert-butoxycarbonyl)-hydrazone

75.8 g (0.416 mol) of biphenylyl-4-carbaldehyde (Fluka, Buchs, Schweiz) are added at ambient temperature to a solution of 50 g (0.378 mol) of N-(tert-butoxycarbonyl)-hydrazine (tert-butylcarbazate) in 750 mL of absolute ethanol. Upon completion of the addition, the mixture is heated to reflux during 16 h. Then about 200 mL of solvent are removed by distillation, and the remainder is diluted with 500 mL of water. Spontaneous crystallisation of the product is completed by cooling the mixture to 0° C. The resulting solid is separated by filtration, washed with 1 L of water and dried at 60° C. to give the title compound:

m.p. 188°-189° C., ¹H-NMR (CH₃OD): δ=7.92 (s, 1H), 7.78 and 7.65 (AB-system, J=8, 4H), 7.63 (m, 2H), 7.5-7.3 (m, 3H), 1.52 (s, 9H).

### b) N¹-(tert-Butoxycarbonyl)-N²-(4-biphenylylmethyl)-hydrazine

A suspension of 9.4 g 5% Pd on carbon is added to a solution of 94.4 g (0.318 mol) of N¹-(4-biphenylylcarbaldehyde)-N²-(tert-butoxycarbonyl)-hydrazone in 2.5 L of methanol, and the mixture is hydrogenated during 2 h at room temperature using hydrogen at atmospheric pressure. After uptake of the theoretically required amount of hydrogen the catalyst is filtered off, and the filtrate is concentrated under reduced pressure. The resulting solid is suspended in 1 L of hexane to give the title compound as a solid which is separated by filtration. The mother liquors are allowed to stand for several days in order to obtain a further amount of the title compound.

Physical properties of the title compound: m.p. 84°-85° C., ¹H-NMR (CD₃OD) δ=7.55 (m, 4H), 7.50-7.38 (m, 5H), 3.95 (s, 2H), 1.45, (s, 9H).

### c) 1-[2 (S)-hydroxy-3 (S) (tert-butoxycarbonylamino)-4-phenylbutyl]-1-[4-biphenylylmethyl]-2-[tert-butoxycarbonyl)hydrazine

A solution of 58.7 g (197-mmol) of N¹-(tert-butoxycarbonyl)-N²-(4-biphenylylmethyl)hydrazine and 51.8 g (197 mmol) of N-(tert-butoxycarbonyl)-2(S)-amino-1-phenyl-3(R)-3,4-epoxybutane (=(2R,3S)-1-[3-Boc-amino-2-phenylethyl]-oxirane) in 700 mL of absolute methanol is heated to reflux temperature during 16 h. The mixture is then cooled to 0° C. and diluted with 1.5 L of water. The solid is filtered and dried at 70° C. for 6 h to give the title compound:

¹H-NMR (CD₃OD) δ=7.55 (m, 5H), 7.45-7.18 (m, 9H), 3.95 (br, 1H), 3.88 (br, 1H), 3.70 (m, 2H), 2.90-2.55 (m, 4H), 1.33 (s, 9H), 1.28 (s, 9H); R_{f} (30:1 dichloromethane:methanol)=0.75.

### d) 1-(4-Biphenylyl)-5(S)-2,5-diamino-4(S)-hydroxy-6-phenyl-2-azahexane Hydrochloride

A solution of 95.19 g (169.5 mmol) of 1-[2(S)-hydroxy-3(S)-(tert-butoxycarbonylamino)-4-phenylbutyl]-1-[4-biphenylylmethyl]-2-[tert-butoxycarbonyl)hydrazine in 300 mL of 4N HCl in dioxane is stirred at room temperature. Thin layer chromatography (5:1 chloroform:methanol) indicates completion of the reaction after 3 h at ambient temperature. The mixture is evaporated under reduced pressure, and the residue is suspended in 300 mL dioxane and evaporated to dryness to give the title compound as an amorphous white solid:

¹H-NMR (CD₃OD) δ=7.70-7.15 (m, 15H), 4.22-3.92 (m, 3H), 3.55 (apparent q, 1H), 3.10 (m, 2H), 2.75 (apparent d, J=4, 2H); R_{f} (5:1dichloromethane:methanol)=0.27.
C) 1-[2(S)-Hydroxy-3(S)-(N-ethoxycarbonyl-(L)-valyl)amino-4-phenylbutyl]-1-[4-biphenylylmethyl]-2-[N-ethoxycarbonyl-(L)-valyl]-hydrazine

Preparation analogously as described in Example 93B) with ethoxycarbonyl-(L)-valine (Example 80a)) instead of the methoxycarbonyl-(L)-valine. After purification by column chromatography on silica gel (eluent: 30:1 dichloromethane: methanol), the title compound is obtained:

¹H-NMR (CD₃OD) δ=7.6-7.1 (m, 14H), 4.18-3.95 (m, 6H), 3.9 (d, J=8, 1H), 3.77 (d, J=4, 1H), 3.72 (d, br, 1H), 3,69 (2 s, 2.times.CH₃), 3.65 (d, J=4, 1H), 2.95-2.75 (m, 3H), 2.65 (dd, J=8, 1, 1H), 1.84 (sept, J=7, 1H), 1.66 (sept, J=7, 1H), 1.22 (m, 6H), 0.75 (apparent t, J=7, 6H), 0.62 (d, J=7, 3H), 0.60 (d, J=7, 3H); FAB MS (M+H)⁺=644, R_{f} (15:1 dichloromethane:methanol)=0.53.
D) 1-[2(S)-hydroxy-3(S)-(N-(N,N-dimethylaminocarbonyl)-(L)-valyl)amino-4-phenylbutyl]-1-[4-biphenylylmethyl]-2-[N-(N,N-dimethylaminocarbonyl)-(L)-valyl]-hydrazine;
E) 1-[2(S)-hydroxy-3(S)-(N-(N-(2-methoxyethyl)aminocarbonyl)-(L)-valyl)amino-4-phenylbutyl]-1-[4-biphenylylmethyl]-2-[N-(N-(2-methoxyethyl)aminocarbonyl)-(L)-valyl]-hydrazine;
F) 1-[2(S)-hydroxy-3(S)-(N-(N-(2-(morpholin-4-yl)ethyl)aminocarbonyl)-(L)-valyl)-amino-4-phenylbutyl]-1-[4-biphenylylmethyl]-2-[N-(N-(2-(morpholin-4-yl)ethyl)aminocarbonyl)-(L)-valyl]-hydrazine;
G) 1-[2(S)-hydroxy-3(S)-(N-(N-(2-(morpholin-4-yl)ethyl)-N-methylaminocarbonyl)-(L)-valyl)amino-4-phenylbutyl]-1-[4-biphenylylmethyl]-2-[N-(N-(2-(morpholin-4-yl)ethyl)-N-methylaminocarbonyl)-(L)-valyl]-hydrazine.

### Example 94

### 1-[2(S)-Hydroxy-3(S)-(N-(methoxycarbonyl)-(L)-valyl)amino-4-cyclohexyl-butyl]-1-[cyclohexylmethyl]-2-[N-methoxycarbonyl-(L)-valyl]-hydrazine

In the presence of 20 mg of Nishimura catalyst (Rh(III)- and Pt(VI)-oxide monohydrate, Degussa) 100 mg (0.165 mmol) of 1-[2(S)-hydroxy-3(S)-(N-(methoxycarbonyl)-(L)-valyl)amino-4-phenyl-butyl]-1-[cyclohexylmethyl]-2-[N-methoxycarbonyl-(L)-valyl]-hydrazine (Example 73a) in the form of a solution in 8 mL of methanol are hydrogenated under low pressure at RT. Removal of the catalyst by filtration through Celite and concentration of the filtrate by evaporation yields the title compound: FAB-MS (M+H)⁺=612.

### Example 95

### 1-[2(S)-Hydroxy-3(S)-(N-(n-propoxycarbonyl)-(L)-valyl)amino-4-phenylbutyl]-1-[cyclohexylmethyl]-2-[N-(n-propyl)oxycarbonyl-(L)-valyl]-hydrazine

Hydrogenation under low pressure at RT of a solution of 100 mg (0.152 mmol) of 1-[2(S)-hydroxy-3(S)-(N-(allyloxycarbonyl)-(L)-valyl)amino-4-phenyl-butyl]-1-[cyclohexylmethyl]-2-[N-allyloxycarbonyl-(L)-valyl]-hydrazine (Example 79) in 4 mL of methanol using 50 mg of 5% Pd/C as catalyst, followed by filtration through Celite (siliceous earth; Fluka, Switzerland), concentration of the filtrate by evaporation and digestion from DIPE, yields the pure title compound: FAB-MS (M+H)⁺=662.

### Example 96

### 1-[2(R)-Hydroxy-3(S)-(N-(methoxycarbonyl)-(L)-valyl)amino-4-phenylbutyl]-1-[cyclohexylmethyl]-2-[N-methoxycarbonyl-(L)-valyl]-hydrazine

Under a protective gas atmosphere, 33 mg (0.186 mmol) of N-methoxycarbonyl-(L)-valine (Example 73b) are activated with 82 mg (0.186 mmol) of BOP and 25 mg (0.186 mmol) of HOBT in 1.24 mL of a 0.3M solution of NMM in DMF and, after 15 min, reacted with 25 mg (0.062 mmol) of 1-[2(R)-hydroxy-3(S)-amino-4-phenyl-butyl]-1-[cyclohexylmethyl]-hydrazine (in the form of the hydrochloride salt) for 18 h. The reaction mixture is concentrated by evaporation under HV and the residue is partitioned between 3 portions of methylene chloride, 2 portions of saturated NaHCO₃ solution and brine. Column chromatography (Si₂, methylene chloride/methanol 99:1→15:1) and precipitation with DIPE from a concentrated solution in DMF yields the pure title compound: t_{Ret} (V)=14.7 min; FAB-MS (M+H)⁺=606.

The starting material is prepared as follows:

### a) 2(R)-[1′(S)-(Trifluoroacetylamino)-2′-phenylethyl]-oxirane (Alternative to Example 16d)

54.28 g (314 mmol) of m-chloroperbenzoic acid are added to a solution of 14.5 g (60 mmol) of N-trifluoroacetyl-1-phenyl-3-buten-2(S)-amine (Example 16c) in 600 mL of chloroform and the mixture is stirred for 16 h at RT to complete the reaction. The reaction mixture is washed twice with 10% sodium sulfite solution, twice with saturated sodium carbonate solution, with water and finally with brine. The aqueous phases are extracted a further twice with methylene chloride, and the combined organic phases are dried with sodium sulfate and concentrated by evaporation. Column chromatography (SiO₂, hexane/ethyl acetate 2:1) yields the title compound in the form of a 6:1 mixture of the (2R)- and (2S)-epimers: TLC R_{f} (F)=0.41, TLC R_{f} (N)=0.6; t_{Ret} (V)=12.6 min; .sup. ¹H-NMR (200 MHz, CDCl₃): inter alia 4.08 (m, 1/7H, H—C(2(S))), 4.47 (m, 6/7H, H—C(2(R))).

### b) 1-[2(S)-Hydroxy-3(S)-(trifluoroacetylamino)-4-phenyl-butyl]-1-[cyclohexylmethyl]-2-[tert-butoxycarbonyl]-hydrazine and 1-[2(R)-hydroxy-3(S)-(trifluoroacetylamino)-4-phenyl-butyl]-1-[cyclohexylmethyl]-2-[tert-butoxycarbonyl]-hydrazine

Under a nitrogen atmosphere, 2.5 g (9.65 mmol) of 2(R)-[1′(S)-(trifluoroacetylamino)-2′-phenylethyl]-oxirane (contains 14% of the (2S)-epimer) and 2.2 g (9.65 mmol) of tert-butyl-3-cyclohexylmethyl-carbazate (Example 4a) dissolved in 31 mL of methanol are reacted for 18 h at 75° C. The reaction mixture is concentrated by evaporation and the residue is chromatographed (SiO₂, toluene/ethyl acetate 10:1). The (2S)-epimer is eluted first as the main product, followed by the (2R)-epimer: (2S)-epimer TLC R_{f} (I)=0.7; t_{Ret} (V)=18.5 min; Anal: calc. C 59.12%, H 7.44%, N 8.62%; found C 59.10%, H 7.09%, N 8.81%. (2R)-epimer TLC R_{f} (I)=0.6; t_{Ret} (V)=18.5 min; FAB-MS (M+H)⁺=488.

### c) 1-[2(R)-Hydroxy-3(S)-amino-4-phenyl-butyl]-1-[cyclohexylmethyl]-2-[tert-butoxycarbonyl]-hydrazine

Under a nitrogen atmosphere, 326 mg (0.669 mmol) of 1-[2(R)-hydroxy-3(S)-(trifluoroacetylamino)-4-phenyl-butyl]-1-[cyclohexylmethyl]-2-[tert-butoxycarbonyl]-hydrazine dissolved in 52 mL of MeOH are heated to 70° C., 17 mL of 1M aqueous K₂ CO₃ solution are added dropwise and the mixture is stirred for 16 h at 70° C. The reaction mixture is concentrated by evaporation and the residue is partitioned between 3 portions of methylene chloride, 2 portions of water and brine. Concentration by evaporation of the organic phases which have been dried with Na₂SO₄ yields the title compound, which is used directly in the next step: ¹H-NMR (200 MHz, CD₃OD): 1.42 (s, 9H, Boc), 0.8-2.1 (m, 11H, cyclohexyl), 2.4-3.0 (m, 6H), 3.1 (m, 1H), 3.54 (m, 1H), 7.28 (m, 5H).

### d) 1-[2(R)-Hydroxy-3(S)-amino-4-phenyl-butyl]-1-[cyclohexylmethyl]-hydrazine (Hydrochloride Salt)

Under a nitrogen atmosphere, 2 mL of 4N HCl/dioxane are added to a solution of 80 mg (0.204 mmol) of 1-[2(R)-hydroxy-3(S)-amino-4-phenyl-butyl]-1-[cyclohexylmethyl]-2-[tert-butoxycarbonyl]-hydrazine in 2 mL of dioxane and the mixture is stirred for 7 h at RT and then lyophilized. The crude product is used directly in the above reaction.

### Example 97

### 1-[2 (R)-Hydroxy-3(R)— (N-(methoxycarbonyl)-(L)-valyl)amino-4-phenylbutyl]-1-(cyclohexylmethyl]-2-[N-methoxycarbonyl-(L)-valyl]-hydrazine

Under a nitrogen atmosphere, 315 mg (1.80 mmol) of N-methoxycarbonyl-(L)-valine (Example 73b) are activated with 795 mg (1.80 mmol) of BOP and 267 mg (1.80 mmol) of HOBT in 12 mL of a 0.3M solution of NMM in DMF and, after 15 min, reacted for 48 h with 240 mg (0.60 mmol) of 1-[2(R)-hydroxy-3(R)-amino-4-phenyl-butyl]-1-[cyclohexylmethyl]-hydrazine (in the form of the hydrochloride salt) (can be prepared analogously to 1-[2(S)-hydroxy-3(S)-amino-4-phenyl-butyl]-1-[cyclohexylmethyl]-hydrazine hydrochloride salt (Example 10a, ═H-[Phe^{NN}Cha]-H.3HCl) from (D)-N-Boc-phenylalanine). The reaction mixture is concentrated by evaporation under HV and the residue is partitioned between 3 portions of methylene chloride, 2 portions of saturated NaHCO₃ solution and brine. Column chromatography (SiO₂, methylene chloride/methanol 19:1) and digestion with methylene chloride/DIPE yields the pure title compound: TLC R_{f} (B′)=0.60; t_{Ret} (V)=14.7 min; FAB-MS (M+H)⁺=606.

### Example 98

### 1-[2(S)-Hydroxy-3(S)-(benzyloxycarbonylamino)-4-phenyl-butyl]-1-[phenylmethyl]-2-[N-methoxycarbonyl-(L)-valyl]-hydrazine

Under a nitrogen atmosphere, 0.28 mmol of 1-[2(S)-hydroxy-3(S)-(benzyloxycarbonylamino)-4-phenyl-butyl]-1-[phenylmethyl]-hydrazine and 54.3 mg (0.31 mmol) of N-methoxycarbonyl-(L)-valine (Example 73b) are dissolved in 3.36 mL of 0.25M NMM/CH₃CN, 117.6 mg (0.31 mmol) of HBTU are added and the mixture is stirred for 3 h at RT. During that time the pure title compound separates from the initially clear solution in the form of a precipitate, which is filtered off and washed with 3 mL of CH₃CN/DIPE 1:2: TLC R_{f} (I)=0.22; t_{Ret} (V)=15.4 min; FAB-MS (M+H)⁺=577. Further product can be recovered by concentrating the filtrate by evaporation, partitioning the residue between 3 portions of ethyl acetate, 2 portions of saturated NaHCO₃ solution, water and brine, drying the organic phases with Na₂SO₄, concentrating by evaporation and digesting from DIPE. The starting material is prepared as follows:

### a) 1-[2(S)-Hydroxy-3(S)-(benzyloxycarbonylamino)-4-phenyl-butyl]-1-[phenylmethyl]-2-[tert-butoxycarbonyl]-hydrazine

Under a nitrogen atmosphere, 220 mg (0.57 mmol) of 1-[2(S)-hydroxy-3(S)-amino-4-phenyl-butyl]-1-[phenylmethyl]-2-[tert-butoxy carbonyl]-hydrazine (═H-[Phe^{NN}Phe]-Boc, Example 30b) in 15 mL of dioxane/water 1:1 and 408 mg (2.9 mmol) of K₂CO₃ are reacted with 120 mg (0.69 mmol) of chloroformic acid benzyl ester for 16 h at RT. KHSO₄ solution is added, the mixture is extracted with 3 portions of ethyl acetate, and the organic phases are washed with water and brine, dried with Na₂SO₄ and concentrated by evaporation. Digestion from hexane yields the pure title compound: TLC R_{f} (I)=0.52; t_{Ret} (V)=17.1 min; FAB-MS (M+H)⁺=520.

### b) 1-[2(S)-Hydroxy-3(S)-(benzyloxycarbonylamino)-4-phenyl-butyl]-1-[phenylmethyl]hydrazine

148 mg (0.28 mmol) of 1-[2(S)-hydroxy-3(S)-(benzyloxycarbonylamino)-4-phenylbutyl]-1-[(phenylmethyl]-2-[tert-butoxycarbonyl]-hydrazine dissolved in 7 mL of formic acid are stirred overnight at RT. The reaction mixture is concentrated by evaporation under HV, the residue is partitioned between 3 portions of ethyl acetate, 2 portions of NaHCO₃ solution and brine, and the organic phases are dried with Na₂SO₄ and concentrated by evaporation: TLC R_{f} (I)=0.50; t_{Ret} (V)=11.8 min.

### Example 99

### 1-[2(S)-Hydroxy-3(S)-(N-(methoxycarbonyl)-(L)-valyl)amino-4-cyclohexyl-butyl]-1-[phenylmethyl]-2-[N-methoxycarbonyl)-L-valyl]-hydrazine

Analogously to Example 87B, 91.7 mg (0.52 mmol) of N-methoxycarbonyl-(L)-valine (Example 73b) are activated with 232 mg (0.52 mmol) of BOP and 70.7 mg (0.53 mmol) of HOBT in 3.5 mL of a 0.3M solution of NMM in DMF and reacted with 70 mg (0.174 mmol) of 1-[2(S)-hydroxy-3(S)-amino-4-cyclohexyl-butyl]-1-[phenylmethyl]hydrazine (in the form of the hydrochloride salt) for 18 h. The reaction mixture is concentrated by evaporation under HV, and the residue is partitioned between 3 portions of methylene chloride, 2 portions of saturated NaHCO₃ solution and brine. Precipitation with DIPE from a concentrated solution in DMF yields the pure title compound: TLC R_{f} (G′)=0.61; t_{Ret} (V)=14.7 min; FAB-MS (M+H)⁺=606.

The starting material is prepared as follows:

### a) N-3(S)-(Boc-amino)-2(R,S)-hydroxy-4-cyclohexyl-1-trimethylsilyl-butane

Using 2.94 g of Nishimura catalyst (Rh(III)- and Pt(VI)-oxide monohydrate, Degussa, Germany) 25 g (81.3 mmol) of N-3(S)-(Boc-amino)-2(R,S)-hydroxy-4-phenyl-1-trimethylsilyl-butane (for preparation see Eβ-532 466-A2, page 42) in 882 mL of methanol are hydrogenated under low pressure at RT. Removal of the catalyst by filtration through Celite and concentration by evaporation of the filtrate yields the title compound: TLC R_{f} (I)=0.7; FAB-MS (M+H)⁺=344.

### b) 1-Cyclohexyl-3-buten-2(S)-amine

42.2 mL (336 mmol) of an approximately 48% solution of boron trifluoride in ether are added at 5° C. to a solution of 23.1 g (67.2 mmol) of 3(S)-(Boc-amino)-2(R,S)-hydroxy-4-cyclohexyl-1-trimethylsilyl-butane in 460 mL of methylene chloride. The reaction mixture is then stirred for 6 h at RT and 3M sodium carbonate solution is added. The aqueous phase is removed and extracted twice with methylene chloride. The organic extracts are washed with brine, dried over sodium sulfate and concentrated by evaporation. The title product is further used without additional purification.

### c) N-Boc-1-cyclohexyl-3-buten-2(S)-amine

15.2 g (69.5 mmol) of Boc-anhydride are added at RT to 8.2 g (53.5 mmol) of 1-cyclohexyl-3-buten-2(S)-amine in the form of a solution in 110 mL of methylene chloride. The mixture is stirred for 17 h at RT and then extracted twice with 10% citric acid, water and brine. The aqueous phases are washed a further twice with methylene chloride, dried with sodium sulfate and concentrated by evaporation. Column chromatography (SiO₂, hexane/ethyl acetate 4:1) yields the title compound: TLC R_{f} (I)=0.8.

### d) 2(R)-[1′(S)-(Boc-amino)-2′-cyclohexylethyl]-oxirane

8.5 g (172 mmol) of m-chloroperbenzoic acid are added to a solution of 2.5 g (9.86 mmol) of N-Boc-1-cyclohexyl-3-buten-2(S)-amine in 65 mL of chloroform and the mixture is stirred for 18 h at RT to complete the reaction. The reaction mixture is washed with 10% sodium sulfite solution, saturated sodium carbonate solution, water and brine. The aqueous phases are extracted a further twice with methylene chloride, and the combined organic phases are dried with sodium sulfate and concentrated by evaporation. Column chromatography (SiO₂, hexane/ethyl acetate 5:1) finally yields the pure title compound: TLC R_{f} (L)=0.22.

### e) 1-[2(S)-Hydroxy-3(S)-(tert-butoxycarbonylamino)-4-cyclohexyl-butyl]-1-[phenylmethyl]-2-[tert-butoxycarbonyl]-hydrazine

Under a nitrogen atmosphere, 200 mg (0.745 mmol) of 2(R)-[1′(S)-(Boc-amino)-2′-cyclohexylethyl)-oxirane and 165 mg (0.745 mmol) of tert-butyl-3-benzyl-carbazate (J. Chem. Soc., Perkin I, 1712 (1975)) in 6 mL of methanol are stirred for 2 days at 75.degree. C. Concentration of the reaction mixture by evaporation and precipitation with hexane from a concentrated solution in methylene chloride yields the title compound: TLC R_{f} (I)=0.54.

### f) 1-[2(S)-Hydroxy-3(S)-amino-4-cyclohexyl-butyl]-1-[phenylmethyl]-hydrazine (Hydrochloride Salt)

0.5 mL of 4N HCl/dioxane are added, with the exclusion of moisture, to a solution of 90 mg (0.183 mmol) of 1-[2(S)-hydroxy-3(S)-(tert-butoxycarbonylamino)-4-cyclohexylbutyl]-1-[phenylmethyl]-2-[tert-butoxycarbonyl]-hydrazine in 0.5 mL of dioxane. After 5 h at RT the reaction mixture is lyophilized and the lyophilisate is directly further used.

### Example 100

### A) 1-[2(S)-Hydroxy-3(S)-(N-(quinoline-2-carbonyl)-(L)-asparaginyl)amino-4-phenyl-butyl]-1-[cyclohexylmethyl]-2-[tert-butoxycarbonyl]-hydrazine

At 5° C., 940 mg (3.27 mmol) of quinoline-2-carbonyl-(L)-asparagine (in the form of the hydrochloride salt) are dissolved in 35 mL of THF, and 736 mg (3.57 mmol) of DCC are added. After 10 min, 482 mg (3.57 mmol) of HOBT, 0.82 mL (7.44 mmol) of NMM and 1.165 g (2.98 mmol) of 1-[2(S)-hydroxy-3(S)-amino-4-phenyl-butyl]-1-[cyclohexylmethyl]-2-[tert-butoxycarbonyl]-hydrazine are added and the mixture is stirred at RT for 18 h. The reaction mixture is filtered and the filtrate is concentrated by evaporation. The residue is partitioned between 3 portions of ethyl acetate, saturated NaHCO₃ solution, water and brine, and the organic phases, which have been dried with Na₂SO₄, are concentrated by evaporation and subjected to column chromatography (SiO2, ethyl acetate) to yield the title compound: TLC R_{f} (O)=0.16; t_{Ret} (V)=16.5 min; FAB-MS (M+H)⁺=661.

The starting compound is prepared in the following manner:

### a) Quinoline-2-carbonyl-(L)-asparagine tert-butyl Ester

Analogously to Example 100, 5.45 g (31.4 mmol) of quinaldic acid in 161 mL of THF are reacted with 7.08 g (34.3 mmol) of DCC, 4.63 g (34.3 mmol) of HOBT and 5.38 g (28.6 mmol) of (L)-asparagine tert-butyl ester (Bachem, Bubendorf, Switzerland). Filtration, extraction and column chromatography (SiO₂, ethyl acetate/hexane 3:1) yields the pure title compound: TLC R_{f} (C′)=0.15; t_{Ret} (V)=12.2 min; FAB-MS (M+H)⁺=344.

### b) Quinoline-2-carbonyl-(L)-asparagine (Hydrochloride Salt)

Under a nitrogen atmosphere, 4.0 g (11.6 mmol) of quinoline-2-carbonyl-(L)-asparagine tert-butyl ester are dissolved in 40 mL of dioxane, and 40 mL of 4N HCl/dioxane are added. On stirring for 17 h at RT, the product is precipitated in the form of a solid. Filtration and washing with DIPE yields the pure title compound: ¹H-NMR (200 MHz, CD₃OD): 3.02 (d, J=6 Hz, 2H), 5.09 (t, J=6 Hz, 1H), 7.92 (m, 1H), 8.12 (m, 1H), 8.26 (m, 1H), 8.4 (m, 2H), 9.03 (m, 1H).

### c) 1-[2(S)-Hydroxy-3(S)-amino-4-phenyl-butyl]-1-[cyclohexylmethyl]-2-[tert-butoxycarbonyl]-hydrazine

Under a nitrogen atmosphere, 2.0 g (4.10 mmol) of 1-[2(S)-hydroxy-3(S)-(trifluoroacetylamino)-4-phenyl-butyl]-1-[cyclohexylmethyl]-2-[tert-butoxycarbonyl]-hydrazine (Example 96b) dissolved in 316 mL of MeOH are heated to 70° C., 103 mL of 1M aqueous K₂CO₃ solution are added dropwise, and the mixture is stirred for 18 h at 70° C. The reaction mixture is concentrated by evaporation under HV and the residue is partitioned between 3 portions of methylene chloride, 2 portions of water and brine. Concentration by evaporation of the organic phases, which have been dried with Na₂SO₄, yields the title compound: ¹H-NMR (200 MHz, CD₃OD): 1.42 (s, 9H, Boc), 0.8-2.1 (m, 11H, cyclohexyl), 2.35-3.0 (m, 7H), 3.51 (m, 1H), 7.25 (m, 5H).

### B) 1-[2(S)-Hydroxy-3(S)-(N-(quinoline-2-carbonyl)-(L)-asparaginyl)amino-4-phenyl-butyl]1-[phenylmethyl]-2-[tert-butoxycarbonyl]-hydrazine

At 0° C., 3.84 g (13.4 mmol) of quinoline-2-carbonyl-(L)-asparagine (hydrochloride salt) (Example 100A) b)) are added to 4.69 g (12.2 mmol) of 1-[2(S)-hydroxy-3(S)-amino-4-phenyl-butyl]-1-[phenylmethyl]-2-[tert-butoxy carbonyl]-hydrazine in 250 mL of THF. 2.18 g (13.4 mmol) of HOBT, 2.76 g (13.4 mmol) of DCC and 2.14 mL (19.5 mmol) of NMM are added to the suspension which is then stirred for 30 min at 0° C. and for 17 h at RT. The reaction mixture is filtered and the filtrate is concentrated by evaporation to a residual volume of approximately 50 mL. The fine suspension is taken up in methylene chloride and washed with NaHCO₃ solution and brine, and the aqueous phases are extracted with 2 portions of methylene chloride. Filtration of the combined organic phases through cotton wadding, concentration by evaporation and precipitation from a concentrated solution in methanol/methylene chloride with DIPE and finally hexane yields the pure title compound: TLC R_{f} (P)=0.41; t_{Ret} (V)=14.8 min; FAB-MS (M+H)⁺=655.

The starting material is prepared as follows:

### a) 1-[2(S)-Hydroxy-3(S)-(trifluoroacetylamino)-4-phenyl-butyl]-1-[phenylmethyl)-2-[tert-butoxycarbonyl]-hydrazine: (Improved Version for Example 30a))

Under a nitrogen atmosphere, 20.49 g (79 mmol) of 2(R)-[1′(S)-(trifluoroacetylamino)-2′-phenylethyl]-oxirane (Example 96a) and 17.56 g (79 mmol) of tert-butyl-3-benzylcarbazate (J. Chem., Perkin I, 1712 (1975)) in 300 mL of ethanol are heated at 80° C. for 20 h. Cooling and partial concentration by evaporation until crystallization commences, filtration and washing with a small amount of ethanol yields the pure title compound: t_{Ret} (V)=16.1 min; FAB-MS (M+H)⁺=482; ¹H-NMR (200 MHz, CD₃OD): 1.30 (s, 9H), 2.70 (m, 2H), 2.83-3.08 (m, 2H), 3.76 (m, 1H), 3.85 (s, 2H), 4.21 (m, 1H 7.2-7.4 (m, 10H).

### b) 1-[2(S)-Hydroxy-3(S)-amino-4-phenyl-butyl]-1-[phenylmethyl]-2-[tert-butoxycarbonyl]-hydrazine: (Improved Version for Example 30b))

Under a nitrogen atmosphere, 6.0 g (12.5 mmol) of 1-[2(S)-hydroxy-3(S)-(trifluoroacetylamino)-4-phenyl-butyl]-1-[phenylmethyl]-2-[tert-butoxycarbonyl]-hydrazine dissolved in 420 mL of MeOH are heated to 80° C., 125 mL of 1M aqueous K₂CO₃ solution are added dropwise (15 min) and the mixture is stirred for 18 h at 80° C. The reaction mixture is concentrated by evaporation and the residue is partitioned between 3 portions of methylene chloride, water and brine. Concentration by evaporation of the organic phases, which have been filtered through cotton wadding, yields the title compound: t_{Ret} (V)=11.5 min; ¹H-NMR (200 MHz, CD₃OD): 1.29 (s, 9H), 2.5-3.05 (m, 5H), 3.56 (m, 1H), 3.8-3.95 (AB, 2H), 7.1-7.4 (m, 10H).

### C): 1-[2(S)-Hydroxy-3(S)-(N-(quinoline-2-carbonyl)-(L)-asparaginyl)amino-4-phenylbutyl]-1-[p-(methoxyphenyl)methyl]-2-[tert-butoxycarbonyl]-hydrazine

Analogously to Example 100B), 7.00 g (16.8 mmol) of 1-[2(S)-hydroxy-3(S)-amino-4-phenyl-butyl]-1-[p-(methoxy-phenyl)methyl]-2-[tert-butoxycarbonyl]-hydrazine in 420 mL of THF are reacted with 5.3 g (18.5 mmol) of quinoline-2-carbonyl-(L)-asparagine (hydrochloride salt) (Example 100A) b)), 3.0 g (18.5 mmol) of HOBT, 3.8 g (18.5 mmol) of DCC and 5 mL of NMM. The reaction mixture is filtered, the filtrate is concentrated by evaporation, and the evaporation residue is taken up in ethyl acetate and washed twice in each case with NaHCO₃ solution and brine. The aqueous phases are extracted with 2 portions of ethyl acetate and the organic phases are dried with Na₂SO₄ and concentrated by evaporation. Column chromatography (SiO₂, ethyl acetate) yields the pure title compound: TLC R_{f} (O)=0.59; t_{Ret} (V)=14.5 min; FAB-MS (M+H)⁺=685.

The starting material is prepared as follows:

### a) p-(Methoxyphenyl)-carbaldehyde tert-butoxycarbonylhydrazone

Under protective gas, 65 mL (534 mmol) of freshly distilled anisaldehyde are dissolved in 850 mL of ethanol, 70.6 g (534 mmol) of tert-butylcarbazate are added and the mixture is heated for 3 h at 80.degree. C. Concentration of the reaction mixture by evaporation yields the title compound: ¹H-NMR (200 MHz, CD₃OD): 1.53 (s, 9H), 3.82 (s, 3H), 6.94 and 7.64 (2 d, J=9 Hz, each 2H), 7.86 (s, 1H).

### b) tert-Butyl-3-(p-methoxyphenyl-methyl)-carbazate

130 g (520 mmol) of p-(methoxyphenyl)-carbaldehyde tert-butoxycarbonylhydrazone are hydrogenated in 1.3 L of THF in the presence of 11.5 g of 5% Pd/C. Removal of the catalyst by filtration through Celite and concentration by evaporation of the filtrate yields the title compound: TLC R_{f} (F)=0.3; t_{Ret} (V)=8.9 min; ¹H-NMR (200 MHz, CD₃ OD): 1.44 (s, 9H), 3.77 (s, 3H), 3.83 (s, 2H), 6.87 and 7.26 (2 d, J=8 Hz, each 2H).

### c) 1-[2(S)-Hydroxy-3(S)-(trifluoroacetylamino)-4-phenyl-butyl]-1-[p-(methoxyphenyl)methyl]-2-[tert-butoxycarbonyl]-hydrazine

Under protective gas, 15 g (57.9 mmol) of 2(R)-[1′(S)-(trifluoroacetylamino)-2′-phenylethyl]-oxirane (Example 96a) and 14.6 g (57.9 mmol) of tert-butyl-3-(p-methoxyphenylmethyl)-carbazate in 220 mL of ethanol are heated for 18 h at 80° C. Cooling, concentration by evaporation and digestion in DIPE yields the title compound: TLC R_{f} (F)=0.42; t_{Ret} (V)=15.8 min.

### d) 1-[2(S)-Hydroxy-3(S)-amino-4-phenyl-butyl]-1-[p-(methoxyphenyl)methyl]-2-[tert-butoxycarbonyl]-hydrazine

Analogously to Example 100B) b), 19.7 g (38.4 mmol) of 1-[2(S)-hydroxy-3(S)-(trifluoroacetylamino)-4-phenyl-butyl]-1-[p-(methoxy-phenyl)methyl]-2-[tert-butoxycarbonyl]-hydrazine in 1 l of methanol are hydrolyzed with 384 mL of 1M K₂CO₃ solution to yield the title compound: TLC R_{f} (G′)=0.4.

### Example 101

### 1-[2(S)-Hydroxy-3(S)-(N-(quinoline-2-carbonyl)-(L)-asparaginyl)amino-4-phenyl-butyl]-1-[cyclohexylmethyl]-2-[N-methoxycarbonyl-(L)-valyl]-hydrazine

Under a nitrogen atmosphere, 100 mg (0.178 mmol) of 1-[2(S)-hydroxy-3(S)-(N-(quinoline-2-carbonyl)-(L)-asparaginyl)amino-4-phenyl-butyl]-1-[cyclohexylmethyl]hydrazine (Example 71e) i)) and 34 mg (0.196 mmol) of N-methoxycarbonyl-(L)-valine (Example 73b) are dissolved in 1.75 mL of a 0.3M solution of NMM in DMF, 74 mg (0.196 mmol) of HBTU are added and the mixture is stirred at RT. Since, after 3 days, there is still unreacted hydrazine present, a further 0.3 equivalents in each case of N-methoxycarbonyl-(L)-valine and HBTU in 0.48 mL of 0.3M NMM/DMF is added. After 18 h the reaction mixture is concentrated by evaporation under HV, the residue is dissolved in methylene chloride and washed with saturated NaHCO₃ solution, water and brine, the aqueous phases are extracted twice with methylene chloride, and the organic phases are dried with Na₂SO₄ and concentrated by evaporation. Column chromatography (SiO₂, methylene chloride/methanol 15:1) and digestion from DIPE yields the title compound: TLC R_{f} (A′)=0.17; t_{Ret} (V)=14.6 min; FAB-MS (M+H)⁺=718.

### Example 102

### 1-[2(S)-Hydroxy-3(S)-(N-(quinoline-2-carbonyl)-(L)-asparaginyl)amino-4-phenyl-butyl]-1-[cyclohexylmethyl]-2-[3,3-dimethylbutylyl]-hydrazine

Under a protective gas atmosphere, 100 mg (0.178 mmol) of 1-[2(S)-hydroxy-3(S)-(N-(quinoline-2-carbonyl)-(L)-asparaginyl)amino-4-phenyl-butyl]-1-[cyclohexylmethyl]hydrazine (Example 71E) i)) and 25 μL (0.196 mmol) of 3,3-dimethylbutyric acid are dissolved in 1.75 mL of a 0.3M solution of NMM in DMF, 74 mg (0.196 mmol) of HBTU are added and the mixture is stirred at RT. Since, after 18 h, there is still unreacted hydrazine present, a further 0.3 equivalents in each case of 3,3-dimethylbutyric acid and HBTU in 0.5 mL of 0.3N NMM/DMF is added. Working up analogously to Example 101, column chromatography (SiO₂, ethyl acetate/ethanol 20:1) and digestion from DIPE yields the title compound: TLC R_{f} (E′)=0.23; t_{Ret} (V)=15.6 min; FAB-MS (M+H)⁺=659.

### Example 103

### 1-[2(S)-Hydroxy-3(S)-(N-(quinoline-2-carbonyl)-(L)-asparaginyl)amino-4-phenyl-butyl]-1-[cyclohexylmethyl]-2-[tert-butylaminocarbonyl]-hydrazine

Under a protective gas atmosphere, 100 mg (0.178 mmol) of 1-[2(S)-hydroxy-3(S)-(N-(quinoline-2-carbonyl)-(L)-asparaginyl)amino-4-phenyl-butyl]-1-[cyclohexylmethyl)hydrazine (Example 71E) i)) are dissolved in 0.7 mL of THF and reacted with 19 μL (0.169 mmol) of tert-butyl-isocyanate for 17 h at RT. The reaction mixture is concentrated by evaporation, the residue is dissolved in ethyl acetate and washed with 5% citric acid solution, water and brine, and the inorganic phases are extracted twice with ethyl acetate, dried with Na₂SO₄ and concentrated by evaporation. Column chromatography (SiO₂, ethyl acetate/ethanol 10:1) yields the title compound: TLC R_{f} (F′)=0.16; t_{Ret} (V)=15.3 min; FAB-MS (M+H)⁺=660.

### Example 104

### 1-[2(S)-Hydroxy-3(S)-(N-(quinoline-2-carbonyl)-(L)-asparaginyl)amino-4-phenyl-butyl]-1-[cyclohexylmethyl]-2-[benzylaminocarbonyl]-hydrazine

Analogously to Example 103, 100 mg (0.178 mmol) of 1-[2(S)-hydroxy-3(S)-(N-(quinoline-2-carbonyl)-(L)-asparaginyl)amino-4-phenyl-butyl]-1-[cyclohexylmethyl]hydrazine (Example 71E) i) in 0.7 mL of THF are reacted with 21 μL (0.169 mmol) of benzyl isocyanate. Column chromatography (SiO₂, methylene chloride/methanol 15:1) and digestion from DIPE yields the title compound: TLC R_{f} (A′)=0.12; t_{Ret} (V)=15.5 min; FAB-MS (M+H)⁺=694.

### Example 105

### 1-[2(S)-Butyryloxy-3(S)-(N-(methoxycarbonyl)-(L)-valyl)amino-4-phenylbutyl]-1-[cyclohexylmethyl]-2-[N-methoxycarbonyl-(L)-valyl]-hydrazine

50 μL (0.495 mmol) of butyric acid chloride and 2 mg (0.017 mmol) of DMAP are added to an ice-cooled mixture of 200 mg (0.33 mmol) of 1-[2(S)-hydroxy-3(S)-(N-(methoxy-carbonyl)-(L)-valyl)amino-4-phenyl-butyl]-1-[cyclohexylmethyl]-2-[N-methoxycarbonyl-(L)-valyl]-hydrazine (Example 73a) in 2.6 mL of dioxane and 0.4 mL of pyridine. After 18 h at RT, according to HPLC there is still 1-[2(S)-hydroxy-3(S)-(N-(methoxycarbonyl)-(L)-valyl)amino-4-phenyl-butyl]-1-[cyclohexylmethyl]-2-[N-methoxycarbonyl-(L)-valyl]-hydrazine present in the reaction mixture, and therefore a further 0.75 equivalents of butyric acid chloride and a few granules of DMAP are added. After a further 18 h, a 3rd portion of 1.5 equivalents of butyric acid chloride is added and the mixture is further stirred for one night at RT (.fwdarw.HPLC: fully reacted). The reaction mixture is diluted with ethyl acetate and washed with 2 portions of saturated NaHCO₃ solution, water and brine, the aqueous phases are extracted twice with ethyl acetate, and the organic phases are dried with Na₂SO₄ and concentrated by evaporation. Digestion of the oily residue from hexane in an ultrasound bath yields the pure title compound: TLC R_{f} (O)=0.67; t_{Ret} (V)=17.2 min; FAB-MS (M+H)⁺=676.

### Example 106

### 1-[2(S)-Palmitoyloxy-3(S)-(N-(methoxycarbonyl)-(L)-valyl)amino-4-phenyl-butyl]-1-[cyclohexylmethyl]-2-[N-methoxycarbonyl-(L)-valyl]-hydrazine

Analogously to Example 105, 200 mg (0.33 mmol) of 1-[2(S)-hydroxy-3(S)-(N-(methoxy-carbonyl)-(L)-valyl)amino-4-phenyl-butyl]-1-[cyclohexylmethyl]-2-[N-methoxycarbonyl-(L)-valyl]-hydrazine (Example 73a) in 2.6 mL of dioxane and 0.4 mL of pyridine are reacted with 0.15 mL (0.495 mmol) of palmitic acid chloride and 2 mg (0.017 mmol) of DMAP. To complete the reaction a further 0.3 mL of palmitic acid chloride and a small amount of DMAP are added and the mixture is stirred. Extraction and column chromatography (SiO₂, ethyl acetate/hexane 3:2) yields the pure title compound: TLC R_{f} (H′)=0.47; t_{Ret} (V)=25.2 min; FAB-MS (M+H)⁺=844.

### Example 107

### 1-[2 (S)— (Methoxy-acetoxy)-3 (S)-(N-(methoxycarbonyl)-(L)-valyl)amino-4-phenyl-butyl]-1-[cyclohexylmethyl]-2-[N-methoxycarbonyl-(L)-valyl]-hydrazine

Analogously to Example 105, 200 mg (0.33 mmol) of 1-[2(S)-hydroxy-3(S)-(N-(methoxycarbonyl)-(L)-valyl)amino-4-phenyl-butyl]-1-[cyclohexylmethyl]-2-[N-methoxycarbonyl-(L)-valyl]-hydrazine (Example 73a) in 2.6 mL of dioxane and 0.4 mL of pyridine are reacted with 50. mu.l (0.495 mmol) of methoxyacetic acid chloride and 2 mg (0.017 mmol) of DMAP. To complete the reaction a further 0.5 equivalents of methoxyacetic acid chloride and a few granules of DMAP are added and the mixture is stirred. Extraction and digestion from DIPE/hexane in an ultrasound bath yields the pure title compound: TLC R_{f} (O)=0.48; t_{Ret} (V)=15.6 min; FAB-MS (M+H)⁺=678.

### Example 108

### 1-[2(S)-(2-Pyridyl-carbonyl)oxy-3(S)-(tert-butoxycarbonyl)amino-4-phenyl-butyl]-1-[cyclohexylmethyl]-2-[tert-butoxycarbonyl]-hydrazine

At 0° C. under a nitrogen atmosphere, 500 mg (4.07 mmol) of picolinic acid (Fluka; Buchs/Switzerland) in 25 mL of methylene chloride are converted into the acid chloride with 0.57 mL (4.07 mmol) of 1-chloro-N,N,2-trimethyl-1-propenamine [B. Haveaux, A. Dekoker, M. Rens, A. R. Sidani, J. Toye, L. Ghosez, M. Murakami, M. Yoshioka, and W. Nagata, Organic Syntheses 59, 26 (1980)]. After 45 min, 10 mL of THF, 8.3 mL of pyridine, 10 mg of DMAP and 1.00 g (2.03 mmol) of 1-[2(S)-hydroxy-3(S)-(tert-butoxycarbonyl)amino-4-phenyl-butyl]-1-[cyclohexylmethyl]-2-(tert-butoxycarbonyl)-hydrazine(=Boc-[Phe^{NN}Cha]-Boc, Example 4) are added and the mixture is stirred for 16 h at RT. Since according to HPLC there is still 1-[2(S)-hydroxy-3(S)-(tert-butoxycarbonyl)amino-4-phenyl-butyl]-1-[cyclohexylmethyl]-2-[tert-butoxycarbonyl]-hydrazine present in the reaction mixture, a further 2 equivalents of picolinic acid chloride (prepared as described above) dissolved in 20 mL of methylene chloride are added. After 18 h at RT the mixture is diluted with methylene chloride and washed twice with saturated NaHCO₃ solution, water and brine, and the organic phases are extracted with 2 portions of methylene chloride, dried with Na₂SO₄ and concentrated by evaporation. The brown residue is dissolved in methylene chloride/ethyl acetate, 10 g of silica gel are added and the mixture is concentrated by evaporation. The resulting powder is applied to a silica gel column (hexane/ethyl acetate 1:1). Elution with hexane/ethyl acetate 1:1 yields the title compound: TLC R_{f} (N)=0.17; t_{Ret} (V)=19.9 min; ¹H-NMR (200 MHz, CD₃OD): 0.6-1.85 (m, 11H, cyclohexyl), 1.36 and 1.40 (2 s, 18H, 2 Boc), 2.4-3.1 (m, 6H), 4.28 (m, 1H), 5.28 (m, 2H), 7.1-7.3 (m, 5H), 7.72 (m, 1H), 8.08 (m, 1H), 8.28 (d, J=7 Hz, 1H), 8.75 (d, J=5 Hz, 1H).

### a) 1-[2(S)-Hydroxy-3(S)-(N-(methoxycarbonyl)-(L)-valyl)amino-4-phenyl-butyl]-1-[cyclohexylmethyl]-2-[pyridin-2-yl-carbonyl]-hydrazine

i. Under a nitrogen atmosphere, 200 mg (0.335 mmol) of 1-[2(S)-(pyridin-2-yl-carbonyl)-oxy-3(S)-(tert-butoxycarbonyl)amino-4-phenyl-butyl]-1-[cyclohexylmethyl]-2-[tert-butoxycarbonyl]-hydrazine are dissolved in 200 mL of formic acid and the solution is stirred for 16 h at RT and then concentrated by evaporation under HV. The residue is partitioned between 3 portions of methylene chloride, saturated NaHCO₃ solution and brine and the organic phases are dried with Na₂SO₄ and concentrated by evaporation (t_{Ret} (V)=11.9 min).

ii. The above residue is dissolved in 5.3 mL of 0.25M NMM in acetonitrile, and 134 mg (0.763 mmol) of N-(methoxycarbonyl)-(L)-valine (Example 73b) and 313 mg (0.826 mmol) of HBTU are added. On stirring for 18 h at RT the title compound separates out in the form of a precipitate and can then be filtered off and washed with a small amount of acetonitrile. t_{Ret} (V)=15.9 min; FAB-MS (M+H)⁺=554, ¹H-NMR (500 MHz, DMSO-d₆): 0.64 and 0.70 (2s, J=7 Hz, (H₃C)₂C), 0.81 (m, 2 Hₐₓ—C₆H₁₁), 1.08 (m, 3 Hₐₓ—C₆H₁₁), 1.30 (m, H_{1-az}—C₆H₁₁), 1.57 (m, 3 H_{eq}—C₆H₁₁), 1.74 (m, 1 H_{eq}—C₆H₁₁), 1.79 (octet, J=7 Hz, HC(CH₃)₂), 1.88 (m, 1 H_{eq}—-C₆H₁₁), 2.47-2.60 (m, H—C₁, HCH—C₆H₁₁), 2.66-2.84 (m, H—C₁, HCH—C₆H₁₁, H₂C-phenyl), 3.44 (m, HC₂), 3.50 (s, H₃C—O), 3.73 (m, HC_{α}-Val), 3.94 (m, HC₃), 4.94 (s, HO), 6.97 (d, J=9 Hz, HN-Val), 7.05 (m, HC_{phenyl}), 7.12 (m, 4 HC_{phenyl}), 7.51 (d, J=9 Hz, HN—C₃), 7.61 (m, H₅-Py), 8.00 (m, H₃-Py, H₄ Py), 8.61 (d, J=5 Hz, H₆-Py, 9.70 (s, HN—N).

### Example 109

### 1-[2(S)-Hydroxy-3(S)-(N-(quinoline-2-carbonyl)-(L)-asparaginyl)amino-4-phenyl-butyl]-1-[benzyl]-2-[N-methoxycarbonyl)-(L)-valyl]-hydrazine

Analogously to Example 111, 157.4 mg (0.898 mmol) of N-(methoxycarbonyl)-(L)-valine (Example 73b) and 453 mg (0.817 mmol) of 1-[2(S)-hydroxy-3(S)-(N-(quinoline-2-carbonyl)-(L)-asparaginyl)amino-4-phenyl-butyl]-1-[phenylmethyl]-hydrazine in 8 mL of 0.3M NMM/DMF are reacted with 340 mg (0.898 mmol) of HBTU. Column chromatography (SiO₂, methylene chloride→methylene chloride/methanol 50:1→25:1) yields the title compound: TLC R_{f} (J′)=0.16; t_{Ret} (V)=13.6 min; FAB-MS (M+H)⁺=712.

### Example 110

### 1-[2(S)-Hydroxy-3(S)-(N-(quinoline-2-carbonyl)-(L)-asparaginyl)amino-4-phenyl-butyl]-1-[benzyl]-2-[N-ethoxycarbonyl)-(L)-valyl]-hydrazine

Under protective gas, 36 mg (0.189 mmol) of N-(ethoxycarbonyl)-(L)-valine (Example 80a), 35 mg (0.259 mmol) of HOBT and 38 mg (0.198 mmol) of EDC are dissolved in 0.7 mL of 0.3M NMM/DMF and the solution is stirred for 10 min at RT. 100 mg (0.180 mmol) of 1-[2(S)-hydroxy-3(S)-(N-(quinoline-2-carbonyl)-(L)-asparaginyl)amino-4-phenyl-butyl]-1-[phenylmethyl]-hydrazine (Example 111a) are added and the mixture is then stirred for 18 h at RT. The reaction mixture is concentrated by evaporation under HV, and the residue is taken up in ethyl acetate and washed with 2 portions of 10% citric acid solution; water, saturated NaHCO₃ solution and brine. The aqueous phases are extracted with ethyl acetate and the combined organic phases are dried with Na₂SO₄ and concentrated by evaporation. Column chromatography (SiO₂, methylene chloride/methanol 12:1) yields the title compound: TLC R_{f} (I′)=0.25; t_{Ret} (V)=14.3 min.

### Example 111

### 1-[2(S)-Hydroxy-3(S)-(N-(quinoline-2-carbonyl)-(L)-asparaginyl)amino-4-phenyl-butyl]-1-[benzyl]-2-[N-benzyloxycarbonyl)-(L)-valyl]-hydrazine

Under a nitrogen atmosphere, 50 mg (0.198 mmol) of Z-(L)-valine and 100 mg (0.18 mmol) of 1-[2(S)-hydroxy-3(S)-(N-(quinoline-2-carbonyl)-(L)-asparaginyl)amino-4-phenyl-butyl]-1-[phenylmethyl]-hydrazine are dissolved in 1.8 mL of a 0.3M solution of NMM in DMF, 75.1 mg (0.198 mmol) of HBTU are added and the mixture is stirred for 18 h at RT. The reaction mixture is concentrated by evaporation under HV, and the residue is taken up in ethyl acetate and washed with 2 portions of 10% citric acid solution, water, saturated NaHCO₃ solution and brine. The aqueous phases are extracted a further twice with ethyl acetate and the combined organic phases are dried with Na₂SO₄ and concentrated by evaporation. Column chromatography (SiO₂, methylene chloride/methanol 19:1) yields the title compound: TLC R_{f} (J′)=0.27; t_{Ret} (V)=15.7 min; FAB-MS (M+H)⁺=788.

The starting material is prepared as follows:

### a) 1-[2(S)-Hydroxy-3(S)-(N-(quinoline-2-carbonyl)-(L)-asparaginyl)amino-4-phenylbutyl]-1-[phenylmethyl]-hydrazine

1.0 g (1.53 mmol) of 1-[2(S)-hydroxy-3(S)-(N-(quinoline-2-carbonyl)-(L)-asparaginyl)amino-4-phenyl-butyl]-1-[phenylmethyl]-2-[tert-butoxycarbonyl]-hydrazine (Example 100 B) is dissolved in 10 mL of formic acid under protective gas and stirred for 16 h at RT. The formic acid is removed by evaporation under HV, the residue is partitioned between 3 portions of ethyl acetate, saturated NaHCO₃ solution and brine, and the organic phases are dried with Na₂SO₄ and concentrated by evaporation to yield the title compound: t_{Ret} (V)=10.7 min.

### Example 112

### 1-[2(S)-Hydroxy-3(S)-(N-(quinoline-2-carbonyl)-(L)-asparaginyl)amino-4-phenyl-butyl]-1-[benzyl]-2-[N-allyloxycarbonyl)-(L)-valyl]-hydrazine is Prepared in Accordance with One of the Processes Mentioned Hereinbefore or Hereinafter

### Example 113

### 1-[2(S)-Hydroxy-3(S)-(N-(quinoline-2-carbonyl)-(L)-asparaginyl)amino-4-phenyl-butyl]-1-[4-methoxyphenylmethyl]-2-[N-methoxycarbonyl)-(L)-valyl]-hydrazine is Prepared in Accordance with One of the Processes Mentioned Hereinbefore or Hereinafter

### Example 114

### 1-[2(S)-Hydroxy-3(S)-(N-(quinoline-2-carbonyl)-(L)-asparaginyl)amino-4-phenyl-butyl]-1-[p-(methoxyphenyl)methyl]-2-[N-(benzyloxycarbonyl)-(L)-valyl]hydrazine

Under a nitrogen atmosphere, 330 mg (1.32 mmol) of Z-(L)-valine and 700 mg (1.197 mmol) of 1-[2(S)-hydroxy-3(S)-(N-(quinoline-2-carbonyl)-(L)-asparaginyl)amino-4-phenyl-butyl]-1-[p-(methoxyphenyl)methyl]-hydrazine (Example 120B) a)) are dissolved in 11.6 mL of a 0.3M solution of NMM in DMF, 0.50 g (1.32 mmol) of HBTU is added and the mixture is stirred for 18 h at RT. The reaction mixture is concentrated by evaporation under HV and the residue is taken up in ethyl acetate and washed with saturated NaHCO₃ solution, water and brine. The aqueous phases are extracted a further twice with ethyl acetate and the combined organic phases are dried with Na₂SO₄ and concentrated by evaporation. Column chromatography (SiO₂, ethyl acetate) yields the title compound after crystallization from DIPE. TLC R_{f} (G′)=0.50; t_{Ret} (V)=15.4 min.

### Example 115

### 1-[2(S)-Hydroxy-3(S)-(N-(quinoline-2-carbonyl)-(L)-asparaginyl)amino-4-phenyl-butyl]-1-[4-methoxyphenylmethyl]-2-[N-allyloxycarbonyl)-(L)-valyl]-hydrazine is Prepared in Accordance with One of the Processes Mentioned Hereinbefore or Hereinafter

### Example 116

### 1-[2(S)-Hydroxy-3(S)-(N-(quinoline-2-carbonyl)-(L)-asparaginyl)amino-4-(4-methoxyphenyl)-butyl]-1-[benzyl]-2-[N-methoxycarbonyl)-(L)-valyl]-hydrazine is Prepared in Accordance with One of the Processes Mentioned Hereinbefore or Hereinafter

### Example 117

### 1-[2(S)-Hydroxy-3(S)-(N-(quinoline-2-carbonyl)-(L)-asparaginyl)amino-4-(4-methoxyphenyl)-butyl]-1-[benzyl]-2-[N-benzyloxycarbonyl)-(L)-valyl]-hydrazine is Prepared in Accordance with One of the Processes Mentioned Hereinbefore or Hereinafter

### Example 118

### 1-[2(S)-Hydroxy-3(S)-(N-(quinoline-2-carbonyl)-(L)-asparaginyl)amino-4-(4-benzyloxyphenyl)-butyl]-1-[benzyl]-2-[N-methoxycarbonyl)-(L)-valyl]-hydrazine is Prepared in Accordance with One of the Processes Mentioned Hereinbefore or Hereinafter

### Example 119

### 1-[2(S)-Hydroxy-3(S)-(N-(quinoline-2-carbonyl)-(L)-asparaginyl)amino-4-(4-benzyloxyphenyl)-butyl]-1-[benzyl]-2-[N-allyloxycarbonyl)-(L)-valyl]-hydrazine is Prepared in Accordance with One of the Processes Mentioned Hereinbefore or Hereinafter

### Example 120

### 1-[2(S)-Hydroxy-3(S)-(N-(guinoline-2-carbonyl)-(L)-asparaginyl)amino-4-phenyl-butyl]-1-[p-(methoxyphenyl)methyl]-2-[N-(ethoxycarbonyl)-(L)-valyl]-hydr

Analogously to Example 110, 71.2 mg (0.376 mmol) of N-(ethoxycarbonyl)-(L)-valine (Example 80a), 66 mg (0.487 mmol) of HOBT, 72.1 mg (0.376 mmol) of EDC and 200 mg (0.34 mmol) of 1-[2(S)-hydroxy-3(S)-(N-(quinoline-2-carbonyl)-(L)-asparaginyl)amino-4-phenyl-butyl]-1-[p-(methoxyphenyl)methyl]-hydrazine are reacted in 1.33 mL of 0.3M NMM/DMF. Column chromatography (SiO₂, methylene chloride→methylene chloride/methanol 50:1→20:1) yields the title compound: t_{Ret} (V)=13.9 min.

The starting material is prepared as follows:

### a) 1-[2(S)-Hydroxy-3(S)-(N-(quinoline-2-carbonyl)-(L)-asparaginyl)amino-4-phenylbutyl]-1-[p-(methoxyphenyl)methyl]-hydrazine

Analogously to Example 111a), 4.6 g (6.71 mmol) of 1-[2(S)-hydroxy-3(S)-(N-(quinoline-2-carbonyl)-(L)-asparaginyl)amino-4-phenyl-butyl]-1-[p-(methoxyphenyl)methyl]-2-[tert-butoxycarbonyl]-hydrazine (Example 100C) are reacted in 168 mL of formic acid to form the title compound: t_{Ret} (V)=10.8 min.

### Example 121

### 1-[2(S)-Hydroxy-3(S)-(N-(quinoline-2-carbonyl)-(L)-asparaginyl)amino-4-phenyl-butyl]-1-[4-methoxyphenylmethyl]-2-[N-allyloxycarbonyl)-(L)-valyl]-hydrazine is Prepared in Accordance with One of the Processes Mentioned Hereinbefore or Hereinafter

### Example 122

### 1-[2(S)-hydroxy-3(S)-(N-(quinoline-2-carbonyl)-(L)-asparaginyl)amino-4-phenyl-butyl]-1-[4-benzyloxyphenylmethyl]-2-[N-methoxycarbonyl)-(L)-valyl]-hydrazine is Prepared in Accordance with One of the Processes Mentioned Hereinbefore or Hereinafter

### Example 123

### 1-[2(S)-Hydroxy-3(S)-(N-(quinoline-2-carbonyl)-(L)-asparaginyl)amino-4-phenyl-butyl]-1-[4-benzyloxyphenylmethyl]-2-[N-allyloxycarbonyl)-(L)-valyl]-hydrazine is Prepared in Accordance with One of the Processes Mentioned Hereinbefore or Hereinafter

### Example 124

The following are prepared analogously to one of the processes mentioned herein before:
A) 1-[2(S)-hydroxy-3(S)-(N-methoxycarbonyl-(L)-valyl)amino-4-cyclohexyl-butyl]-1-[4-methoxyphenylmethyl]-2-[N-methoxycarbonyl)-(L)-valyl]-hydrazine;
B) 1-[2(S)-hydroxy-3(S)-(N-methoxycarbonyl-(L)-valyl)amino-4-cyclohexyl-butyl]-1-[4-isobutoxyphenylmethyl]-2-[N-methoxycarbonyl)-(L)-valyl]-hydrazine;
C) 1-[2(S)-hydroxy-3(S)-(N-methoxycarbonyl-(L)-valyl)amino-4-cyclohexyl-butyl]-1-[4-ethoxyphenylmethyl]-2-[N-methoxycarbonyl)-(L)-valyl]-hydrazine;
D) 1-[2(S)-hydroxy-3(S)-(N-methoxycarbonyl-(L)-valyl)amino-4-cyclohexyl-butyl]-1-[4-benzyloxyphenylmethyl]-2-[N-methoxycarbonyl)-(L)-valyl]-hydrazine.

### Example 125

### 1-[2 (S)-Hydroxy-3 (S)-(N-methoxycarbonyl-(L)-valyl) amino-4-phenylbutyl]-1-[cyclohexylmethyl]-2-[2-pyridylcarbonyl]-hydrazine is Prepared Analogously to One of the Processes Mentioned Hereinbefore

### Example 126

### 1-[2 (S)-Hydroxy-3 (S)-(N-(quinoline-2-carbonyl)-(L)-asparaginyl)amino-4-phenyl-butyl]-1-[phenylmethyl]-2-[N-(methoxy-ethoxy-ethoxycarbonyl)-(L)-valyl]-hydrazine

Analogously to Example 111, 52 mg (0.198 mmol) of N-(2-(2-methoxy-ethoxy)-ethoxy)carbonyl-(L)-valine (Example 82a)) and 100 mg (0.180 mmol) of 1-[2(S)-hydroxy-3(S)-(N-(quinoline-2-carbonyl)-(L)-asparaginyl)amino-4-phenyl-butyl]-1-[phenylmethyl]-hydrazine in 1.8 mL of 0.3M NMM/DMF are reacted with 75.1 mg (0.198 mmol) of HBTU. The evaporation residue is taken up in methylene chloride and washed with 2 portions of 10% citric acid solution, water, saturated NaHCO₃ solution and brine. The aqueous phases are extracted a further twice with methylene chloride and the combined organic phases are dried with Na₂SO₄ and concentrated by evaporation. Column chromatography (SiO₂, methylene chloride/methanol 19:1) yields the title compound: TLC R_{f} (J′)=0.08; t_{Ret} (V)=13.5 min; FAB-MS (M+H)⁺+800.

### Example 127

### 1-[2(S)-(2-Pyridyl-carbonyl)oxy-3(S)-(N-(quinoline-2-carbonyl)-(L)-asparaginyl)amino-4-phenyl-butyl]-1-[phenylmethyl]-2-[N-(methoxycarbonyl)-(L)-valyl]-hydrazine

Analogously to Example 108, 51 mg (0.416 mmol) of 2-picolinic acid in 0.8 mL of methylene chloride are converted with 59 μL (0.416 mmol) of 1-chloro-N,N,2-trimethyl-1-propenamine into the acid chloride. After the addition of 0.5 mL of dioxane and 0.4 mL of pyridine to the latter, the mixture is reacted with 148 mg (0.208 mmol) of 1-[2(S)-hydroxy-3(S)-(N-(quinoline-2-carbonyl)-(L)-asparaginyl)amino-4-phenyl-butyl]-1-[phenylmethyl]-2-[N-(methoxycarbonyl)-(L)-valyl]-hydrazine (Example 109) in 5 mL of dioxane in the presence of 0.5 mg of DMAP. Since, after 18 h, not all of the educt has been acylated according to HPLC, further acid chloride is added. Column chromatography (SiO₂, methylene chloride→methylene chloride/methanol 15:1) yields the title compound: FAB-MS (M+H)⁺+817.

### Example 128

### 1-[2(S)-Butyroxy-3(S)-(N-(quinoline-2-carbonyl)-(L)-asparaginyl) amino-4-phenyl-butyl]-1-[phenylmethyl]2-[N-(methoxycarbonyl)-(L)-valyl]-hydrazine

Under a nitrogen atmosphere, a small amount of DMAP and 0.2 mL of butyric acid chloride are added to a solution of 121 mg (0.17 mmol) of 1-[2(S)-hydroxy-3(S)-(N-(quinoline-2-carbonyl)-(L)-asparaginyl)amino-4-phenyl-butyl]-1-[phenylmethyl]-2-[N-(methoxycarbonyl)-(L)-valyl]-hydrazine (Example 109) in 3.5 mL of dioxane, 2 mL of methylene chloride and 0.4 mL of pyridine. Dilution of the reaction mixture with methylene chloride, washing with 2 portions of saturated NaHCO₃ solution, water and brine, extraction of the aqueous phases with 2 portions of methylene chloride, drying of the organic phases with Na₂SO₄, concentration by evaporation and column chromatography (SiO₂, methylene chloride/methanol 50:1→19:1) yields the title compound: TLC R_{f} (I′)=0.5; t_{Ret} (V)=15.6 min.

### Example 129

### 1-[2(S)-(2-Pyridyl-carbonyl)oxy-3(S)-(N-(guinoline-2-carbonyl)-(L)-asparaginyl)amino-4-phenyl-butyl]-1-[p-(methoxyphenyl)methyl]-2-[N-(benzyloxycarbonyl)-(L)-valyl]-hydrazine

Analogously to Example 108, 72.7 mg (0.591 mmol) of 2-picolinic acid in 2 mL of methylene chloride are converted with 87 μL (0.614 mmol) of 1-chloro-N,N,2-trimethyl-1-propenamine into the acid chloride. After the addition of 1.36 mL of pyridine to the latter, the mixture is reacted with 100 mg (0.118 mmol) of 1-[2(S)-hydroxy-3(S)-(N-(quinoline-2-carbonyl)-(L)-asparaginyl)amino-4-phenyl-butyl]-1-[p-(methoxyphenyl)-methyl]-2-[N-(benzyloxycarbonyl)-(L)-valyl]-hydrazine (Example 114)) in the presence of 1 mg of DMAP. Further acid chloride is added in portions until according to HPLC all of the educt has been acylated to the title compound.

### Example 130

### 1-[2(S)-(Methoxy-acetoxy)-3(S)-(N-(quinoline-2-carbonyl)-L)-asparaginyl)amino-4-phenyl-butyl]-1-[p-(methoxyphenyl)methyl]-2-[N-(benzyloxycarbonyl)-(L)-valyl]-hydrazine

Analogously to Example 105, 100 mg (0.118 mmol) of 1-[2(S)-hydroxy-3(S)-(N-(quinoline-2-carbonyl)-(L)-asparaginyl)amino-4-phenyl-butyl]-1-[p-(methoxyphenyl)-methyl]-2-[N-(benzyloxycarbonyl)-(L)-valyl]-hydrazine (Example 114)) in 2.4 mL of dioxane and 0.14 mL of pyridine are acylated in the presence of 0.7 mg of DMAP with 38.5 μL (0.35 mmol) of methoxyacetic acid chloride to form the title compound: t_{Ret} (V)=17.8 min.

### Example 131

Gelatine Solution

A sterile-filtered aqueous solution, with 20% cyclodextrins as solubilizers, of one of the compounds of formula I or I-A mentioned in the preceding Examples as active ingredient, is so mixed under aseptic conditions, with heating, with a sterile gelatine solution containing phenyl as preservative, that 1.0 mL of solution has the following composition:

| | |
|---|---|
| active ingredient | 3 mg |
| gelatine | 150.0 mg |
| phenol | 4.7 mg |
| dist. water with 20% | 1.0 mL |
| cyclodextrins (as solubilizer) | |
| as solubilisers | |

### Example 132

Sterile Dry Substance for Injection

5 mg of one of the compounds of formula I or I-A mentioned in the preceding Examples as active ingredient are dissolved in 1 mL of an aqueous solution with 20 mg of mannitol and 20% cyclodextrins as solubilisers. The solution is sterile-filtered and introduced under aseptic conditions into a 2 mL ampoule, deep-frozen and lyophilized. Before use, the lyophilisate is dissolved in 1 mL of distilled water or 1 mL of a physiological saline solution. The solution is administered intramuscularly or intravenously. This formulation can also be introduced into a twin-chambered injection ampoule.

### Example 133

Nasal Spray

500 mg of finely ground (<5.0 μm) powder of one of the compounds of formula I or I-A mentioned in the preceding Examples is suspended as active ingredient in a mixture of 3.5 mL of Myglyol 812 and 0.08 g of benzyl alcohol. The suspension is introduced into a container having a metering valve. 5.0 g of Freon 12 are introduced under pressure into the container through the valve. The “Freon” is dissolved in the Myglyol/benzyl alcohol mixture by shaking. The spray container contains approximately 100 single doses which can be administered individually.

### Example 134

Film-Coated Tablets

The following ingredients are used for the preparation of 10,000 tablets each containing 100 mg of active ingredient:

| | |
|---|---|
| active ingredient | 1000 g |
| corn starch | 680 g |
| colloidal silica | 200 g |
| magnesium stearate | 20 g |
| stearic acid | 50 g |
| sodium carboxymethyl starch | 250 g |
| water | quantum satis |

A mixture of one of the compounds of formula I or I-A mentioned in the preceding Examples as active ingredient, 50 g of corn starch and the colloidal silica is processed with a starch paste, made from 250 g of corn starch and 2.2 kg of demineralized water, to form a moist mass. This is forced through a sieve having a mesh size of 3 mm and dried at 45° C. for 30 min in a fluidized bed drier. The dry granules are pressed through a sieve having a mesh size of 1 mm, mixed with a pre-sieved mixture (1 mm sieve) of 330 g of corn starch, the magnesium stearate, the stearic acid and the sodium carboxymethyl starch, and compressed to form slightly biconvex tablets.

### Example 135

Certain compounds of the invention can generally be prepared by one skilled in the art using the processes disclosed in: Tetrahedron Letters, 39, 4925-4928 (1998); Journal of Medicinal Chemistry, 41, 3387-3401 (1998); Journal of Medicinal Chemistry, 39, 3203-3216 (1996); and/or Journal of the Chemical Society Chemical Communications, 1052-1053 (1993).

As generally shown below in Scheme 1a, Scheme 2a, Scheme 3a and Scheme 4a, the chemistry is straight forward. Specific exemplary descriptions of the routes depicted in Schemes 1a-4a are found in the synthetic examples given for Intermediates A-D, general compound couplings E-G, and examples 1b-6b below.

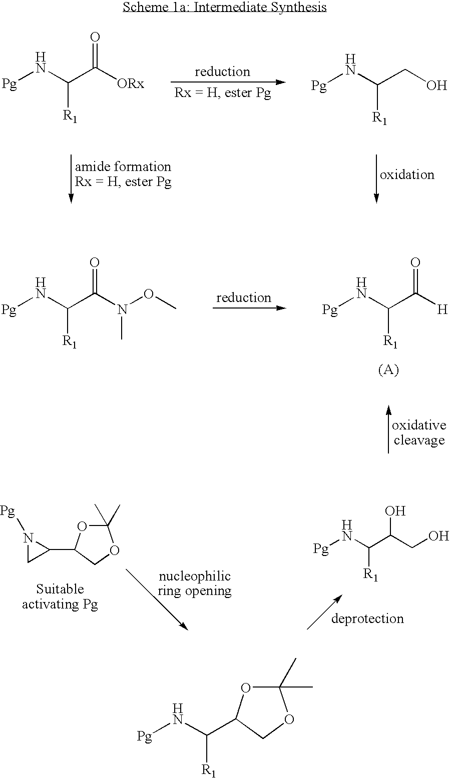

In accordance with this intermediate synthesis route, Pg means protecting group; for examples see Wuts & Green, Protective Groups in Organic Synthesis. 1st-3rd Ed.; for exemplary synthesis of N-protected α-amino aldehydes see: Chem. Rev. 1989, 89, 149. 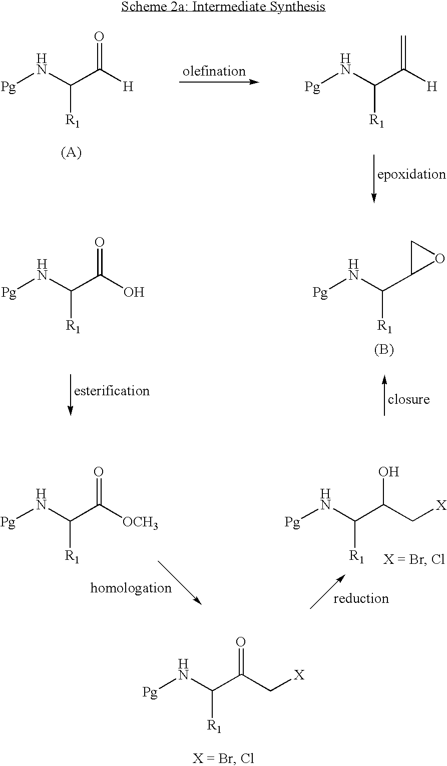 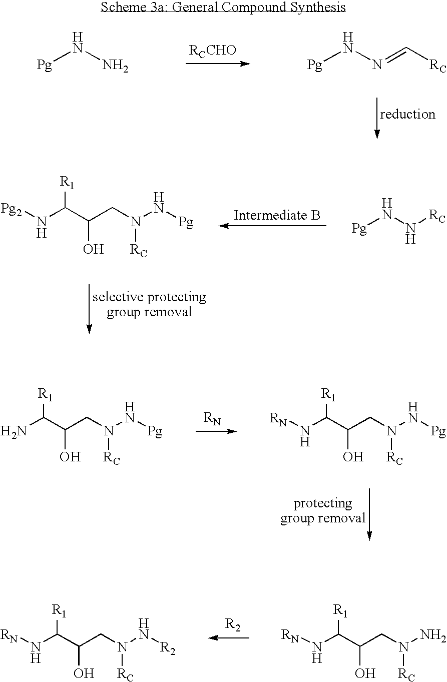
Intermediate Synthesis
A. 2-Methanesulfonylamino-thiazole-4-carboxylic Acid

2-Methanesulfonylamino-thiazole-4-carboxylic acid methyl ester: To a well stirred solution of methyl 2-amino-thiazole-4-carboxylate hydrobromide (Justus Liebigs Ann. Chem. 1951, 571, 44, Heterocycles 1997, 45, 1299) (13 g, 51 mmol) and TEA (40 mL, 290 mmol) in DCM (130 mL) at room temperature is added methanesulfonyl chloride (11 mL, 140 mmol) dropwise. After stirring for 1 h, the reaction mixture is filtered and the filtrate is concentrated. The residue is re-suspended in ethyl acetate, filtered and chromatographed on silica gel (ethyl acetate/ethanol, 97:3) to provide 2-methanesulfonylamino-thiazole-4-carboxylic acid methyl ester (5.5 g). MS (ESI−) for C₆H₈N₂O₄S₂ m/z 234.9 (M−H)⁻.

2-Methanesulfonylamino-thiazole-4-carboxylic acid: A mixture of 2-methanesulfonylamino-thiazole-4-carboxylic acid methyl ester (5.5 g, 22 mmol) in 1M NaOH (50 mL) is stirred at room temperature for 1 h. The reaction mixture is extracted with ethyl acetate, concentrated and chromatographed on silica gel (ethyl acetate/ethanol, 97:3) to provide 2-methanesulfonylamino-thiazole-4-carboxylic acid (3.7 g) as a solid. MS (ESI−) for C₅H₆N₂O₄S₂ m/z 220.9 (M−H)⁻.

B. tert-Butyl 2-((2R,3S)-3-amino-2-hydroxy-4-phenylbutyl)-2-ethylhydrazinecarboxylate

N′-Ethylidene-hydrazinecarboxylic acid tert-butyl ester: A solution of tert-butyl carbazate (50.0 g, 0.38 mole) and acetaldehyde (17.5 g, 0.4 mole) in ethanol (250 mL) is refluxed for 72 h. The solution is concentrated in vacuo and dissolved in ethyl acetate. The solution is washed with water, dried (anhydrous sodium sulfate), filtered and concentrated to afford N′-ethylidene-hydrazinecarboxylic acid tert-butyl ester (56 g) as a light yellow oil that solidified on standing. MS (ESI+) for C₇H₁₄N₂O₂ m/z 159.0 (M+H)⁺.

N′-Ethylhydrazinecarboxylic acid tert-butyl ester: A slurry of N′-ethylidene-hydrazinecarboxylic acid tert-butyl ester (5.0 g, 32 mmol), acetic acid (48 μL) and PtO₂ (250 mg) in ethanol (50 mL) is shaken under a 40 psi pressure of H₂ in a Parr hydrogenator for 24 h. The mixture is filtered through Celite to provide N′-ethylhydrazinecarboxylic acid tert-butyl ester (5 g) as a clear oil. MS (ESI+) for C₇H₁₆N₂O₂ m/z 161.0 (M+H)⁺.

tert-Butyl 2-((2R,3S)-3-{[(benzyloxy)carbonyl]amino}-2-hydroxy-4-phenylbutyl)-2-ethylhydrazinecarboxylate (J. Med. Chem. 1998, 41, 3387-3401): To a well stirred solution of [(1S)-1-(2S)-oxiranyl-2-phenylethyl]-carbamic acid phenylmethyl ester (6.07 g, 20.4 mmol) in isopropanol (100 mL) is added N′-ethylhydrazinecarboxylic acid tert-butyl ester (3.93 g, 24.5 mmol) and the reaction refluxed for 67 h. The reaction is concentrated and chromatographed on silica gel (eluting with a gradient from hexanes to dichloromethane to 1% methanol saturated with ammonia in dichloromethane) to give tert-butyl 2-((2R,3S)-3-{[(benzyloxy)carbonyl]amino}-2-hydroxy-4-phenylbutyl)-2-ethylhydrazinecarboxylate (7.9 g) as a white solid. MS (ESI+) for C₂₅H₃₅N₃O₅ m/z 458.3 (M+H)⁺.

tert-Butyl 2-((2R,3S)-3-amino-2-hydroxy-4-phenylbutyl)-2-ethylhydrazinecarboxylate: A solution of tert-butyl 2-((2R,3S)-3-{[(benzyloxy)carbonyl]amino}-2-hydroxy-4-phenylbutyl)-2-ethylhydrazinecarboxylate (3.11 g, 6.8 mmol) in methanol (50 mL) is added to 10% Pd/C (0.32 g) and stirred in a hydrogen atmosphere (@ ambient pressure) for 5 h. The reaction is filtered through Celite and the filtrate concentrated under reduced pressure to yield tert-butyl 2-((2R,3S)-3-amino-2-hydroxy-4-phenylbutyl)-2-ethylhydrazinecarboxylate (2.2 g) as an oil. MS (ESI+) for C₁₇H₂₉N₃O₃ m/z 324.3 (M+H)⁺.

C. tert-Butyl 2-((2S,3S)-3-amino-2-hydroxy-4-phenylbutyl)-2-ethylhydrazinecarboxylate

tert-Butyl 2-((2S,3S)-3-{[(benzyloxy)carbonyl]amino}-2-hydroxy-4-phenylbutyl)-2-ethylhydrazinecarboxylate: To a well stirred solution of [(1S)-1-(2R)-oxiranyl-2-phenylethyl]-carbamic acid phenylmethyl ester (312 mg, 11.0 mmol) in isopropanol (3 mL) is added N′-ethylhydrazinecarboxylic acid tert-butyl ester (202 mg, 1.3 mmol) and then reaction is refluxed for 24 h. The reaction is concentrated to give crude tert-butyl 2-((2S,3S)-3-{[(benzyloxy)carbonyl]amino]-2-hydroxy-4-phenylbutyl)-2-ethylhydrazinecarboxylate (537 mg). MS (ESI+) for C₂₅H₃₅N₃O₅ m/z 458.1 (M+H)⁺.

tert-Butyl 2-((2S,3S)-3-amino-2-hydroxy-4-phenylbutyl)-2-ethylhydrazinecarboxylate: A solution of crude tert-butyl 2-((2S,3S)-3-{[(benzyloxy)carbonyl]amino}-2-hydroxy-4-phenylbutyl)-2-ethylhydrazinecarboxylate (502 mg, 1.1 mmol) in MeOH (10 mL) is added to 10% Pd/C (5.2 mg) and stirred in a hydrogen atmosphere (@ ambient pressure) for 6 h. The reaction is filtered through Celite and the filtrate concentrated under reduced pressure to yield crude tert-butyl 2-((2S,3S)-3-amino-2-hydroxy-4-phenylbutyl)-2-ethylhydrazinecarboxylate (293 mg). MS (ESI+) for C₁₇H₂₉N₃O₃ m/z 324.2 (M+H)⁺.

### D. tert-Butyl 2-((2S,3S)-3-amino-2-hydroxy-4-(3,5-difluorophenyl)butyl)-2-ethylhydrazinecarboxylate

(Z)-2-Benzyloxycarbonylamino-3-phenyl-acrylate: A well stirred solution of 3,5-difluorobenzaldehyde (1.92 g, 13.5 mmol) and methyl benzyloxycarbonylamino-(dimethoxyphosphoryl)acetate (5.37 g, 16.2 mmol) in dry THF (20 mL), under N₂, is cooled to 0° C. and 1,1,3,3-tetramethylgaunidine (1.38 g, 16.2 mmol) is added via syringe. The reaction is allowed to warm to room temperature and stir for 6 h. The reaction is then quenched with saturated ammonium chloride and extracted with ethyl acetate. After concentration, the residue is chromatographed on silica gel (eluting with 20% ethyl acetate/heptane) to give methyl (Z)-2-benzyloxycarbonylamino-3-phenyl-acrylate (3.55 g) as a white solid. MS (ESI+) for C₁₈H₁₅F₂NO₄ m/z 348.2 (M+H)⁺.

CBZ-L-3,5-Difluorophenylalanine Methyl Ester: A Parr Hastelloy bomb is charged with (Z)-2-benzyloxycarbonylamino-3-phenyl-acrylate (21 g, 48 mmol) in degassed methanol (315 mL) and pressurized with H₂ @ 80 psi. After 1 h, (S)-EtDuPHOSRh(COD)BF₄ (476 mg, 1.5 mol %) in degassed methanol (15mL) is added and the reaction stirred at room temperature under 40 psi H₂ for 16 h. The solution is filtered through Celite, concentrated and chromatographed on silica gel (eluting with 20% ethyl acetate/heptane) to give 20 g of CBZ-L-3,5-difluorophenylalanine methyl ester as a white solid. MS (ESI+) for C₁₈H₁₇F₂NO₄ m/z 350.1 (M+H)⁺.

[1S-(1-(3,5-Difluorobenzyl)-2-hydroxy-ethyl)]-carbamic acid benzyl ester: To a well stirred solution of L-3,5-difluorophenylalanine methyl ester (795 mg, 2.3 mmol) and lithium chloride (97 mg, 2.3 mmol) in THF/ethanol (10 mL, 1:1), under N₂, is added solid sodium borohydride (86 mg, 2.3 mmol.) The reaction is stirred at room temperature overnight. The reaction is quenched with saturated ammonium chloride, extracted with ethyl acetate and the extracts combined, dried (anhydrous sodium sulfate), filtered and concentrated. The residue is chromatographed an silica gel (eluting with 20% ethyl acetate/heptane) to give [1S-(1-(3,5-difluorobenzyl)-2-hydroxy-ethyl)]-carbamic acid benzyl ester (660 mg) as a white solid. MS (ESI+) for C₁₇H₁₇F₂NO₃ m/z 322.2 (M+H)⁺.

[1S-(1-(3,5-difluorobenzyl)-2-oxo-ethyl)]-carbamic acid benzyl ester: To a cold (−78° C.) well-stirred solution of oxalyl chloride (0.16 mL, 1.8 mmol) in methylene chloride (20 mL) is added DMSO (0.13 mL, 1.8 mmol) followed by the slow addition of [1S-(1-(3,5-difluoro-benzyl)-2-hydroxy-ethyl)]-carbamic acid benzyl ester (1.5 mmol)as a solution in CH₂Cl₂ (10 mL). The reaction mixture is stirred at −78° C. for 10 minutes followed by the addition of Et₃N (0.63 mL, 4.5 mmol, 3.0 equiv). The mixture is stirred at −78° C. until no starting material is observed by TLC (2 h). The reaction mixture is then washed with 10% citric acid (aq), dried over MgSO₄, filtered, and condensed. The white solid is purified by silica chromatography (20% to 50% EtOAc/Heptane) to yield [1S-(1-(3,5-difluorobenzyl)-2-oxo-ethyl)]-carbamic acid benzyl ester (445 mg) as a white solid. MS (ESI+) for C₁₇H₁₅F₂NO₃ m/z 320 (M+H)⁺.

[1S-(1-(3,5-Difluorobenzyl)-2-propenyl)]-carbamic acid benzyl ester: To a slurry of diethyl ether (5 mL) and Mg turnings (0.17 g, 7.1 mmol) is added ClCH₂Si(CH₃)₃ (0.97 mL, 7.0 mmol.) The mixture is then heated to reflux. After initiation (0.5 h @ reflux), the reaction mixture is removed from heating and stirred for an additional 1 h, then cooled to rt. To the cooled solution is [1S-(1-(3,5-difluoro-benzyl)-2-oxo-ethyl)]-carbamic acid benzyl ester (0.45 g, 1.4 mmol) as a solution in distilled THF (10 mL). The reaction mixture is stirred at room temperature for 1 h, cooled to 0° C. and BF₃(OEt)₂ (0.97 mL, 7.0 mmol) added. The reaction mixture is further stirred at room temperature for 16 h, quenched with. 10% NaOH (aq) (50 mL) and stirring continued for 1 h. The reaction mixture is extracted with CH₂Cl₂, dried over MgSO₄, filtered and condensed to yield a clear oil. The oil is purified by silica chromatography (20% to 50% EtOAc/heptane) to yield [1S-(1-(3,5-difluorobenzyl)-2-propenyl)]-carbamic acid benzyl ester (270 mg) as a white solid. MS (ESI+) for C₁₈H₁₈F₂NO₂ m/z 318 (M+H)⁺.

[(1S)-1-(2R)-oxiranyl-2-(3,5-difluorophenyl)ethyl]-carbamic acid phenylmethyl ester: To CH₂Cl₂ (20 mL) is added [1S-(1-(3,5-difluoro-benzyl)-2-propenyl)]-carbamic acid benzyl ester (270 mg, 0.85 mmol) followed by MCPBA (77%, 458 mg, 1.0 mmol). The reaction mixture is stirred at room temperature for 24 h. The reaction is then diluted with CH₂Cl₂, washed with 10% sodium thiosulfate (aq), dried over Na₂SO₄, filtered and condensed. Preparative chromatography (5×50 cm Chiralcel OD, 30° C., 70 ml/min. 25% IPA/75% heptane) provides [(1S)-1-(2R)-oxiranyl-2-(3,5-difluorophenyl)ethyl]-carbamic acid phenylmethyl ester. MS (ESI+) for C₁₈H₁₈F₂NO₃ m/z 334.2 (M+H)⁺.

tert-Butyl 2-((2S,3S)-3-{[(benzyloxy)carbonyl]amino}-2-hydroxy-4-(3,5-difluorophenyl)butyl)-2-ethylhydrazinecarboxylate: To a well stirred solution of [(1S)-1-(2R)-oxiranyl-2-(3,5-difluorophenyl)ethyl]-carbamic acid phenylmethyl ester (496 mg, 1.5 mmol) in isopropanol (4.25 mL) is added N′-ethylhydrazinecarboxylic acid tert-butyl ester (575 mg, 3.6 mmol) and then reaction is refluxed for 48 h. The reaction is cooled and concentrated to provide crude tert-butyl 2-((2S,3S)-3-{[(benzyloxy)carbonyl]amino}-2-hydroxy-4-(3,5-difluorophenyl)butyl)-2-ethylhydrazine-carboxylate. MS (ESI+) for C₂₅H₃₃F₂N₃O₅ m/z 516.2 (M+Na)⁺.

tert-Butyl 2-((2S,3S)-3-amino-2-hydroxy-4-(3,5-difluorophenyl)butyl)-2-ethylhydrazinecarboxylate: A solution of crude tert-butyl 2-((2S,3S)-3-{[(benzyloxy)carbonyl]-amino}-2-hydroxy-4-(3,5-difluorophenyl)butyl)-2-ethylhydrazinecarboxylate (906 mg, 1.8 mmol) in MeOH (15 mL) is added to 10% Pd/C (95 mg) and stirred in a hydrogen atmosphere (ambient pressure) for 4 h. The catalyst is removed by filtering the reaction mixture through Celite and the filtrate is concentrated under reduced pressure to yield crude tert-butyl 2-((2S, 3S)-3-amino-2-hydroxy-4-(3,5difluorophenyl)butyl)-2-ethylhydrazinecarboxylate. MS (ESI+) for C₁₇H₂₇F₂N₃O₃ m/z 360.2 (M+H)⁺. 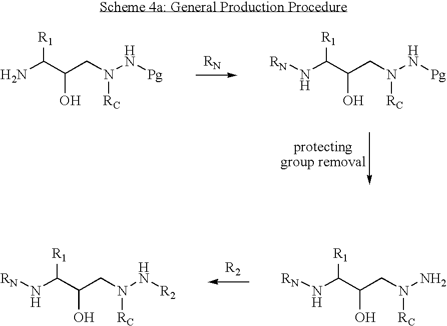
E. N-Terminal Acid Coupling (R_{N})

Solutions of each acid (R_{N}) were prepared (1.08 mmol) in DMF (3 mL) (enough for 6 cartridges per acid). Solutions of PyBOP (3.74 g, 7.2 mmol) and HOBT (0.96 g, 7.2 mmol) in DMF (24 mL) were prepared. To each cartridge on a Bohdan block is added acid solution (0.5niL, 0.18 mmol) according to product layout (6 cartridges of each of the 8 acids). To each cartridge is added the PyBOP/HOBT solution (0.5 mL, 0.078 g PYBOP, 0.15 mmol/0.02 g HOBT, 0.15 mmol) and DIEA (0.052 mL, 0.30 mmol). The Bohdan block is agitated on a Bohdan shaker at 700-800 rpm for 1 h. A solution of amine is prepared (2.30 g, 7.2 mmol) in 24 mL of CH₂Cl₂. To each cartridge is added 0.5 mL of amine solution (0.48 g, 0.15 mmol). The Bohdan block is agitated on the Bohdan shaker at 700-800 rpm for 16 h. The reaction mixtures were drained into 48 well Robbin's blocks. Each cartridge is rinsed with 0.5 mL DMF into another 48 well Robbin's block. The reaction mixtures were concentrated in the Robbin's blocks under reduced pressure at 50° C. for 6 h in a Jouan centrifugal evaporator. Products were carried on crude to next reaction.

F. Deprotection (J. Org. Chem. 1998, 63, 3471)

Add Dowex 50Wx2-400 ion-exchange resin (˜230 mg) to each clean cartridge on a Bohdan block using an Argo Scoop. The previous product is pipetted as a solution in 2 mL MeOH into each cartridge. The Bohdan block is agitated on a Bohdan shaker at 50° C. at 700-800 rpm for 4 h. Each cartridge is rinsed with CH₂Cl₂ (2×2.5 mL) and MeOH (10×2.5 mL). The products were eluted using 3.5M NH₃ in MeOH (2×3 mL) into 2 separate 48 well Robbin's blocks. Reaction mixtures were concentrated in the Robbin's blocks under reduced pressure at 40° C. for 4 h in a Jouan.

G. C-Terminal Acid Coupling (R₂)

Solutions of each acid (R₂) were prepared (1.80 mmol) in DMF (2 mL) (enough for 8 cartridges per acid). A solution of HATU (4.13 g, 10.8 mmol) in DMF (12 mL) is prepared. To each cartridge on a Bohdan block is added acid solution (0.25 mL, 0.225mmol) according to product layout (8 cartridges of each of the 6 acids). To each cartridge is added 0.25 mL of the HATU solution (0.086 g, 0.225 mmol) and DIEA (0.163 mL, 0.94 mmol). The Bohdan block is agitated on a Bohdan shaker at 700-800 rpm for 1 h. Solution of the amines were prepared (0.15 mmol/mL) in DMF. To each cartridge is added the amine solution (0.15 mmol,) according to the product layout. The Bohdan block is agitated on the Bohdan shaker at 700-800 rpm for 16 h. The reaction mixtures were drained into 48 well Robbin's blocks. Each cartridge is rinsed with 0.5 mL DMF into another 48 well Robbin's block. The reaction mixtures are concentrated in the Robbin's blocks under reduced pressure at 50° C. for 6 h in a Jouan. Products were dissolved in 2 mL MeOH and each transferred to a Varian Mega BE-SCX (2 gm, 12 mL) cartridge that had been presoaked with MeOH (5 mL). After products were loaded onto SCX cartridges by gravity, each cartridge is washed with MeOH (9×5 mL) using vacuum filtration. Final products are eluted off by gravity using 3.5M NH₃ in MeOH (3×3 mL) into pre-tared vials.

### Examples

### Example 1b

N-[(1S,2R)-3-(2-benzoyl-1-ethylhydrazino)-1-benzyl-2-hydroxypropyl]-2-[(methylsulfonyl)amino]-1,3-thiazole-4-carboxamide: Synthesis as described above from tert-butyl 2-((2R,3S)-3-amino-2-hydroxy-4-phenylbutyl)-2-ethylhydrazinecarboxylate, benzoic acid and 2-methanesulfonylamino-thiazole-4-carboxylic acid. MS (ESI+) for C₂₄H₂₉N₅O₅S₂ m/z 532.7 (M+H)⁺.

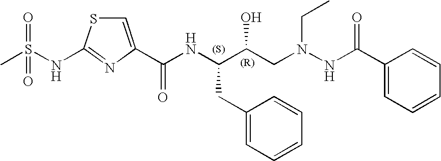

### Example 2b

N-{(1S,2R)-1-benzyl-3-[1-ethyl-2-(4-methylpentanoyl)hydrazino]-2-hydroxypropyl}-2-[(methylsulfonyl)amino]-1,3-thiazole-4-carboxamide: Synthesis as described above from tert-butyl 2-((2R,3S)-3-amino-2-hydroxy-4-phenylbutyl)-2-ethylhydrazinecarboxylate, 4-methylpentanoic acid and 2-methanesulfonylamino-thiazole-4-carboxylic acid. MS (ESI+) for C₂₃H₃₅N₅O₅S₂ m/z 526.7 (M+H)⁺.

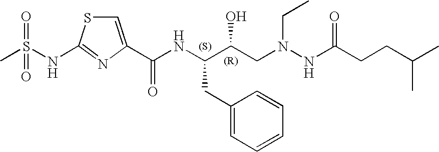

### Example 3b

N-{(1S,2R)-1-benzyl-3-[1-ethyl-2-(4-methylpentanoyl)hydrazino]-2-hydroxypropyl}-4-methylpentanamide: Synthesis as described above from tert-butyl 2-((2R,3S)-3-amino-2-hydroxy-4-phenylbutyl)-2-ethylhydrazinecarboxylate and 4-methylpentanoic acid. MS (ESI+) for C₂₄H₄₁N₃O₃ m/z 420.4 (M+H)⁺.

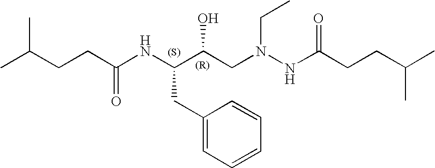

### Example 4b

N¹-{(1S,2S)-1-benzyl-3-[1-ethyl-2-(4-methylpentanoyl)hydrazino]-2-hydroxypropyl}-5-methyl-N³,N³-dipropylisophthalamide: Synthesis as described above from tert-butyl 2-((2S,3S)-3-amino-2-hydroxy-4-phenylbutyl)-2-ethylhydrazinecarboxylate, 4-methylpentanoic acid and 5-methyl-N,N-dipropyl-isophthalamic acid (WO 02/02512). MS (ESI+) for C₃₃H₅₀N₄O₄ M/z 567.3 (M+H)⁺.

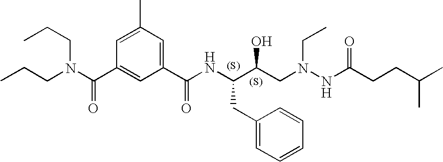

### Example 5b

N¹-{(1S,2S)-1-(3,5-difluorobenzyl)-3-[1-ethyl-2-(4-methylpentanoyl)hydrazino]-2-hydroxypropyl}-5-methyl-N³,N³-dipropylisophthalamide: Synthesis as described above from tert-butyl 2-((2S,3S)-3-amino-2-hydroxy-4-(3,5difluorophenyl)butyl)-2-ethylhydrazinecarboxylate, 3-methylpentanoic acid and 5-methyl-N,N-dipropyl-isophthalamic acid. MS (ESI+) for C₃₃H₄₈F₂N₄O₄ m/z 603.3 (M+H)⁺.

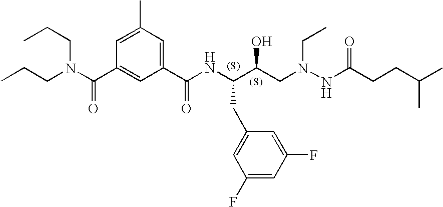

### Example 6b

N¹-{(1S,2S)-1-(3,5-difluorobenzyl)-3-[1-ethyl-2-(4-methylbutanoyl)hydrazino]-2-hydroxypropyl}-5-methyl-N³,N³-dipropylisophthalamide: Synthesis as described above from tert-butyl 2-((2S,3S)-3-amino-2-hydroxy-4-(3,5difluorophenyl)butyl)-2-ethylhydrazinecarboxylate, 3-methylbutanoic acid and 5-methyl-N,N-dipropyl-isophthalamic acid. MS (ESI+) for C₃₂H₄₆F₂N₄O₄ m/z 589.3 (M+H)⁺.

1. A method of treating or preventing Alzheimer's disease in a patient in need of such treatment comprising administering a therapeutically effective amount of a compound of Formula (I) or a pharmaceutically acceptable salt thereof: 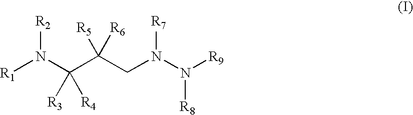
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

2. A method of treating Alzheimer's disease in a patient in need of such treatment comprising administering to the patient a compound disclosed in claim 1, or a pharmaceutically acceptable salt thereof.

3. A method of treating Alzheimer's disease by modulating the activity of beta amyloid converting enzyme, comprising administering to a patient in need of such treatment a compound disclosed in claim 1, or a pharmaceutically acceptable salt thereof.

4. The method according to claim 1, further comprising the administration of a P-gp inhibitor, or a pharmaceutically acceptable salt thereof.

5. A method of treating a patient who has, or in preventing a patient from getting, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I), or a pharmaceutically acceptable salt thereof: 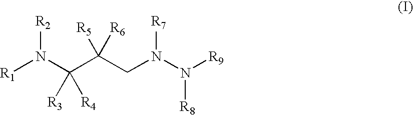
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

6. The method according to claim 5 wherein the compound of formula (I) is selected from the group consisting of:
1-[2(S)-(2-pyridylcarbonyl)oxy-3(S)-(N-quinoline-2-carbonyl)-(L)-asparaginyl)amino4-phenylbutyl-1-[phenylmethyl]-2-[N-(methoxycarbonyl)-(L)-valyl]hydrazine;
1-[2(S)-butyryloxy-3(S)-(N-quinoline-2-carbonyl)-(L)-asparaginyl)amino-4-phenylbutyl-1-[phenylmethyl]-2-[N-(methoxycarbonyl)-(L)-valyl]hydrazine;
1-[2(S)-(2-pyridylcarbonyl)oxy-3(S)-(N-quinoline-2-carbonyl)-(L)-asparaginyl)amino4-phenylbutyl-1-[4-methoxyphenylmethyl]-2-[N(benzyloxycarbonyl)-(L) valyl]hydrazine;
1-[2(S)-(methoxy-acetyl)oxy-3(S)-(N-quinoline-2-carbonyl)-(L)-asparaginyl)amino-4-phenylbutyl-1-[4-methoxyphenylmethyl]-2-[N(benzyloxycarbonyl)-(L)-valyl]hydrazine;
1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-(2(S)-octanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-furylcarbonyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-imidazolylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-pyridylacetyl)oxy-3(S)-(N-(methoxycarbonyl)-(L)-valyl)amino-4-phenylbutyl]-1[cyclohexylmethyl]-2-[N-(methoxycarbonyl)-(L)-valyl]hydrazine;
1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]hydrazine;
1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-benzyloxycarbonyl-N-methyl-aminoacetyl)oxy-3(S)-(N-(methoxycarbonyl)(L)-valyl)amino-4-phenyl-butyl]-1-[cyclohexylmethyl]-2-[N-(methoxycarbonyl) (L)-valyl]hydrazine;
1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-morpholinomethylbenzoyl)oxy-3 (S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(3-carboxypropionyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-(benzyloxycarbonyl)-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butoxy-carbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-methoxy-carbonyl-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[3,3-dimethylbutyryl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butylamino-carbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[benzylamino-carbonyl]hydrazine;
1-[2(S)-hydroxy-3(S)-(N-(quinoline-2-carbonyl)-(L)-asparaginyl)amino-4-phenyl-butyl]-1-[benzyl]-2-[N-methoxy-carbonyl)-(L)-valyl]hydrazine;
1-2(S)-hydroxy-3(S)-(N-(quinoline-2-carbonyl)-(L)-asparaginyl)amino-4-phenyl-butyl]-1-[benzyl]-2-[N-ethoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-allyloxy-carbonyl)-(L)-valyl]hydrazine;
1-[2(S)-hydroxy-3(S)-(N-(quinoline-.sup.2 carbonyl)-(L)-asparaginyl)amino-4-phenyl-butyl]-1-[4-methoxyphenylmethyl]-2-[N-methoxy-carbonyl)-(L) valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-(4-methoxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-methoxyphenyl)-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4methoxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-benzyloxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[2(S)-hydroxy-3(S)—(N-quinoline-2-carbonyl)-(L)-asparaginyl)amino-4-phenylbutyl-1-[phenylmethyl]-2-[N-(2-(2-methoxyethoxy)ethoxycarbonyl)-(L)-valyl]hydrazine 1-[2(S)-hydroxy-3(S)-(N-quinoline-2-carbonyl)-(L)-asparaginyl)amino-4-phenylbutyl-1-[4-methoxyphenylmethyl]-2-[tert-butoxy-carbonyl]hydrazine; and
1-[2(S)-hydroxy-3(S)-(N-quinoline-2-carbonyl)-(L)-asparaginyl)amino-4-phenylbutyl-1-[4-methoxyphenylmethyl]-2-[N-(ethoxycarbonyl)-(L)-valyl]hydrazine;
1-[2(S)-hydroxy-3(S)-(N-(2-methoxyethoxy)acetyl-(L)-valyl)amino-4-phenylbutyl]-1-[cyclohexylmethyl]-2-[N-(2-methoxyethoxy)acetyl-(L)-valyl]hydrazine;
1-[2(S)-hydroxy-3(S)-(N-(2-(2-methoxyethoxy)ethoxy)carbonyl-(L)-valyl)amino-4-phenylbutyl]-1-[cyclohexylmethyl]-2-[N-(2-(2-methoxyethoxy)ethoxy)carbonyl-(L)-valyl]hydrazine;
1-[2(S)-hydroxy-3(S)-(N-acetyl-(L)-valyl)amino-4-phenylbutyl]-1-[thien-2-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[thien-2-yl-methyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[thien-2-yl-methyl]-2-[N-ethoxycarbonyl-(L)-valyl]hydrazine;
1-(2(S)-hydroxy-3(S)-(N-(N,N-dimethylaminocarbonyl)-(L)-valyl)amino-4-phenylbutyl]-1-[thien-2-ylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenyl-butyl]-1-[thien-2-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[thien-2-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[thien-2-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[2,3,5,6-tetrahydropyran-4-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[2,3,5,6-tetrahydropyran-4-ylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[2, 3,5,6-tetrahydropyran-4-ylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[2,3,5,6tetrahydropyran-4-ylmethyl]-2-(N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[2,3,5,6-tetrahydropyran-4-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[2,3,5,6-tetrahydropyran-4-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[2,3,5,6-tetrahydropyran-4-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-hydroxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-hydroxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-hydroxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-hydroxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-hydroxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-hydroxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-hydroxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-methoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-methoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-methoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-methoxyphenylmethyl]-2-[N-(N,N-dimethylaminocarbonyl)-(L)-valyl]hydrazine 1-[2(S)-hydroxy-3(S)-(N-(N-(2-methoxyethyl)aminocarbonyl)-(L)-valyl)amino-4-phenylbutyl]-1-[4-methoxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-methoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-methoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-benzyloxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-benzyloxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-benzyloxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-benzyloxyphenylmethyl)-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-benzyloxyphenylmethyl]-2-[N-(N-(2-methoxyethyl)-aminocarbonyl)(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-benzyloxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-benzyloxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(3,4-dimethoxybenzyl)oxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(3,4-dimethoxybenzyl)oxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(3,4-dimethoxybenzyl)oxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(3,4-dimethoxybenzyl)oxyphenylmethyl]-2-[N-(N,N-dimethylaminocarbonyl)(L)-valyl]hydrazine;
1-[2(S)-hydroxy-3 (S)-(N-(N-(2-methoxyethyl)aminocarbonyl)-(L)-valyl)amino-4-phenylbutyl]-1-[4-(3,4-dimethoxybenzyl)oxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)aminocarbonyl)-(L)-valyl]hydrazine;
1-[2(S-hydroxy-3(S)-(N-(N-(2-(morpholin-4-yl)ethyl)-N-methyl-aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-isobutoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-isobutoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-isobutoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-isobutoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-isobutoxyphenylmethyl]-2-[N-(N-(2-methoxyethyl)aminocarbonyl)-(L)-valyl]-hydrazine;
1-[2(S)-hydroxy-3(S)-(N-(N-(2-(morpholin-4-yl)ethyl)aminocarbonyl)-(L)-valyl)amino-4-phenylbutyl]-1-[4-isobutoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-isobutoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(2-methoxyethoxy)phenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(2-methoxyethoxy)phenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(2-methoxyethoxy)phenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(2-methoxyethoxy)phenylmethyl]-2-[N-(N,N-dimethylaminocarbonyl)-(L)-valyl]hydrazine;
1-[2(S)-hydroxy-3 (S)-(N-(N-(2-methoxyethyl)aminocarbonyl)-(L)-valyl)amino-4-phenylbutyl]-1-[4-(2-methoxyethoxy)phenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(2-methoxyethoxy)phenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(2-methoxyethoxy)phenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[methylene-3,4-dioxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[methylene-3,4-dioxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[methylene-3,4-dioxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[methylene-3,4-dioxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[methylene-3,4-dioxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[methylene-3,4-dioxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[methylene-3,4-dioxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[3,4-dimethoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[3,4-dimethoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[3,4-dimethoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[3,4-dimethoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[3,4-dimethoxyphenylmethyl]-2-[N-(N-(2-methoxyethyl)aminocarbonyl)-(L)-valyl]hydrazine:
1-[2(S)-hydroxy-3(S)-(N-(N-(2-(morpholin-4-yl)ethyl)aminocarbonyl)-(L)-valyl)amino-4-phenylbutyl]-1-[3,4-dimethoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[3,4-dimethoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-biphenylylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-biphenylmethyl]-2-N-methoxycarbonyl-(L)-valyl]hydrazine;
1-[2(S)-hydroxy-3(S)-(N-ethoxycarbonyl-(L)-valyl)amino-4-phenylbutyl]-1-[4-biphenylylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-biphenylylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-biphenylylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-biphenylylmethyl]-2-[N-(N-(2-(morpholin-4-yl)ethyl)aminocarbonyl)-(L)-valyl]hydrazine and 1-[2(S)-hydroxy-3(S)-(N-(N-(2-(morpholin-4-yl)ethyl)-N-methyl-aminocarbonyl)-(L)-valyl)amino-4-phenylbutyl]-1-[4-biphenylylmethyl]-2-[N-(N-(2-(morpholin-4-yl)ethyl)N-methylaminocarbonyl)-(L)-valyl]hydrazine;
1-[2(S)-hydroxy-3(S)-(N-ethoxycarbonyl-(L)-valyl)amino-4-phenylbutyl]-1-[cyclohexylmethyl]-2-[N-ethoxycarbonyl-(L)-valyl]hydrazine;
1-[2(S)-hydroxy-3(S)-(N-trifluoroacetyl-(L)-valyl)amino-4-phenylbutyl]-1-[cyclohexylmethyl]-2-[N-trifluoroacetyl-(L)-valyl]hydrazine;
1-[2(S)-hydroxy-3(S)-(N-(methoxy-carbonyl)-(L)-valyl)amino-4-cyclohexyl-butyl]-1-[cyclohexylmethyl]-2-[N-methoxy-carbonyl-(L)-valyl]hydrazine;
1-[2(S)-hydroxy-3(S)-(N-(n-propoxy-carbonyl)-(L)-valyl)amino-4-phenyl-butyl]-1-[cyclohexylmethyl]-2-[N-(n-propyl)oxy-carbonyl-(L)-valyl]hydrazine;
1-[2(R)-hydroxy-3(S)-(N-(methoxy-carbonyl)-(L)-valyl)amino-4-phenyl-butyl]-1-[cyclohexylmethyl]-2-[N-methoxy-carbonyl-(L)-valyl]hydrazine;
1-[2(R)-hydroxy-3(R)—(N-(methoxy-carbonyl)-(L)-valyl)amino-4-phenyl-butyl]-1-[cyclohexylmethyl]-2-[N-methoxy-carbonyl-(L)-valyl]hydrazine;
1-[2(S)-hydroxy-3(S)-(benzyloxy-carbonyl-amino)-4-phenyl-butyl]-1-[phenylmethyl]-2-[N-methoxy-carbonyl-(L)-valyl]hydrazine;
1-[2(S)-hydroxy-3(S)-(N-(methoxy-carbonyl)-(L)-valyl)amino-4-cyclohexyl-butyl]-1-[phenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-methoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-isobutoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-ethoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-benzyloxyphenylmethyl]-2-[N-methoxy-carbonyl)-(L)-valyl]hydrazine;
1-[2(S)-hydroxy-3(S)-(N-methoxycarbonyl-(L)-valyl)amino-4-phenyl-butyl]-1-[cyclohexylmethyl]-2-[2-pyridylcarbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-(benzyloxycarbonyl)-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butoxy-carbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[N-methoxy-carbonyl-(L)-valyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[3,3-dimethylbutyryl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[tert-butylamino-carbonyl]hydrazine;
1-[2(S)-hydroxy-3(S)-(N-(quinoline-2-carbonyl)-(L)-asparaginyl)amino-4-phenyl-butyl]-1-[cyclohexylmethyl]-2-[benzylamino-carbonyl]hydrazine;
1-[2(S)-hydroxy-3(S)-(N-(quinoline-2-carbonyl)-(L)-asparaginyl)amino-4-phenyl-butyl]-1-[benzyl]-2-[N-methoxy-carbonyl)-(L)-valyl]hydrazine;
1-2(S)-hydroxy-3(S)-(N-(quinoline-2-carbonyl)-(L)-asparaginyl)amino-4-phenyl-butyl]-1-[benzyl]-2-[N-ethoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[benzyl]-2-[N-allyloxy-carbonyl)-(L)-valyl]hydrazine;
1-[2(S)-hydroxy-3(S)-(N-(quinoline-.sup.2 carbonyl)-(L)-asparaginyl)amino-4-phenyl-butyl]-1-[4-methoxyphenylmethyl]-2-[N-methoxy-carbonyl)-(L)valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine
1-[2(S)-hydroxy-3(S)-(N-(quinoline-2-carbonyl)-(L)-asparaginyl)amino-4-(4-methoxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-methoxyphenyl)-butyl]-1-[benzyl]-2-[N-benzyloxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-methoxy-carbonyl)-(L)-valyl]hydrazine;
1-[2(S)-hydroxy-3(S)-(N-(quinoline-2-carbonyl)-(L)-asparaginyl)amino-4-(4-benzyloxyphenyl)-butyl]-1-[benzyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-methoxyphenylmethyl]-2-[N-allyloxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[4-benzyloxyphenylmethyl]-2-[N-methoxy-carbonyl)-(L)-valyl]hydrazine;
1-[2(S)-hydroxy-3(S)-(N-(quinoline-2-carbonyl)-(L)-asparaginyl)amino-4-phenyl-butyl]-1-[2(S)-hydroxy-3(S)—(N-quinoline-2-carbonyl)-(L)-asparaginyl)amino-4-phenylbutyl-1-[phenylmethyl]-2-[N-(2-(2-methoxyethoxy)ethoxycarbonyl)-(L)-valyl]hydrazine;
1-[2(S)-hydroxy-3(S)-(N-quinoline-2-carbonyl)-(L)-asparaginyl)amino-4-phenylbutyl-1-[4-methoxyphenylmethyl]-2-[tert-butoxy-carbonyl]hydrazine; and
1-[2(S)-hydroxy-3(S)-(N-quinoline-2-carbonyl)-(L)-asparaginyl)amino-4-phenylbutyl-1-[4-methoxyphenylmethyl]-2-[N-(ethoxycarbonyl)-(L)-valyl]hydrazine;
1-[2(S)-hydroxy-3(S)-(N-allyloxycarbonyl-(L)-valyl)amino-4-phenylbutyl]-1-[cyclohexylmethyl]-2-[N-allyloxycarbonyl-(L)-valyl]hydrazine;
1-[2(S)-hydroxy-3(S)-(N-(2-(2-methoxyethoxy)ethoxy)carbonyl-(L)-valyl)amino-4-phenylbutyl]-1-[cyclohexylmethyl]-2-[N-(2-(2-methoxyethoxy)ethoxy)carbonyl-(L)-valyl]hydrazine;
1-[2(S)-hydroxy-3(S)-(N-(2-methoxyethoxy)acetyl-(L)-valyl)amino-4-phenylbutyl]-1-[cyclohexylmethyl]-2-[N-(2-methoxyethoxy)acetyl-(L)-valyl]hydrazine;
1-[2(S)-hydroxy-3(S)-(N-acetyl-(L)-valyl)amino-4-phenylbutyl]-1-[thien-2-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[thien-2-yl-methyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[thien-2-yl-methyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[thien-2-ylmethyl]-2-[N—(N,N-dimethylaminocarbonyl)-(L)-valyl]hydrazine;
1-[2(S)-hydroxy-3 (S)-(N-(N-(2-methoxyethyl)aminocarbonyl)-(L)-valyl)amino-4-phenyl-butyl]-1-[thien-2-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[thien-2-ylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[thien-2-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[2,3,5,6-tetrahydropyran4-ylmethyl]-2-[N-acetyl-(L)-valyl]hydrazine;
1-[2(S)-hydroxy-3(S)-(N-methoxycarbonyl-(L)-valyl)amino-4-phenylbutyl]-1-[2,3,5,6-tetrahydropyran-4-ylmethyl]-2-[N-methoxycarbonyl-(L)-valyl]hydrazine
1-[2(S)-hydroxy-3(S)-(N-ethoxycarbonyl-(L)-valyl)amino-4-phenylbutyl]-1-[2,3,5,6-tetrahydropyran-4-ylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[2,3,5,6-tetrahydropyran-4-ylmethyl]-2-[N-(N,N-dimethylaminocarbo nyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[2,3,5,6-tetrahydropyran-4-ylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[2,3,5,6-tetrahydropyran-4-ylmethyl]-2-[N-(N-(2-(morpholin-4-yl)ethyl)aminocarbonyl)-(L)-valyl]hydrazine; and
1-[2(S)-hydroxy-3(S)-(N-(N-(2-(morpholin-4-yl)ethyl)-N-methyl-aminocarbonyl)-(L)-valyl)amino-4-phenylbutyl]-1-[2,3,5,6-tetrahydropyran-4-ylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-hydroxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-hydroxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-hydroxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-hydroxyphenylmethyl]-2-[N-(N,N-dimethylaminocarbonyl)-(L)-valyl]hydrazine;
1-[2(S)-hydroxy-3 (S)-(N-(N-(2-methoxyethyl)aminocarbonyl)-(L)-valyl)amino-4-phenylbutyl]-1-[4-hydroxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-hydroxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-hydroxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-methoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-methoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-methoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-methoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-methoxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-methoxyphenylmethyl]-2-[N-(N-(2-(morpholin-4-yl)ethyl)aminocarbonyl)-(L)-valyl]hydrazine;
1-[2(S)-hydroxy-3 (S)-(N-(N-(2-(morpholin-4-yl)ethyl)-N-methyl-aminocarbonyl)-(L)-valyl)amino-4-phenylbutyl]-1-[4-methoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-benzyloxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-benzyloxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-benzyloxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-benzyloxyphenylmethyl)-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-benzyloxyphenylmethyl]-2-[N-(N-(2-methoxyethyl)-aminocarbonyl)(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-benzyloxyphenylmethyl]-2-(N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-benzyloxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(3,4-dimethoxybenzyl)oxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(3,4-dimethoxybenzyl)oxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(3,4-dimethoxybenzyl)oxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(3,4-dimethoxybenzyl)oxyphenylmethyl]-2-[N-(N,N-dimethylaminocarbonyl)(L)-valyl]hydrazine;
1-[2(S)-hydroxy-3 (S)-(N-(N-(2-methoxyethyl)aminocarbonyl)-(L)-valyl)amino-4-phenylbutyl]-1-[4-(3,4-dimethoxybenzyl)oxyphenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(3,4-dimethoxybenzyl)oxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-isobutoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-isobutoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-isobutoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-isobutoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-isobutoxyphenylmethyl]-2-[N-(N-(2-methoxyethyl)aminocarbonyl)-(L)-valyl]-hydrazine;
1-[2(S)-hydroxy-3(S)-(N-(N-(2-(morpholin-4-yl)ethyl)aminocarbonyl)-(L)-valyl)amino-4-phenylbutyl]-1-[4-isobutoxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-isobutoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-(2-methoxyethoxy)phenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-(2-methoxyethoxy)phenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[4-(2-methoxyethoxy)phenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-(2-methoxyethoxy)phenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[4-(2-methoxyethoxy)phenylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-(2-methoxyethoxy)phenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[4-(2-methoxyethoxy)phenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[methylene-3,4-dioxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[methylene-3,4-dioxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[methylene-3,4-dioxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[methylene-3,4-dioxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[methylene-3,4-dioxyphenylmethyl]-2-[N-(N-(2-methoxyethyl)aminocarbonyl)-(L)-valyl]hydrazine;
1-[2(S)-hydroxy-3 (S)-(N-(N-(2-(morpholin-4-yl)ethyl)aminocarbonyl)-(L)-valyl)amino-4-phenylbutyl]-1-[methylene-3,4-dioxyphenylmethyl]-2-[N-(N-(2-(morpholin-4-yl)ethyl)aminocarbonyl)-(L)-valyl]hydrazine;
1-[2(S)-hydroxy-3(S)-(N-(N-(2-(morpholin-4-yl)ethyl)-N-methyl-aminocarbonyl)-(L)-valyl)amino-4-phenylbutyl]-1-[methylene-3,4-dioxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[3,4-dimethoxyphenylmethyl]-2-[N-acetyl-(L)-valyl]hydrazine;
1-[2(S)-hydroxy-3(S)-(N-methoxycarbonyl-(L)-valyl)amino-4-phenylbutyl]-1-[3,4-dimethoxyphenylmethyl]-2-[N-methoxycarbonyl-(L)-valyl]hydrazine;
1-[2(S)-hydroxy-3(S)-(N-ethoxycarbonyl-(L)-valyl)amino-4-phenylbutyl]-1-[3, 4-dimethoxyphenylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[3,4-dimethoxyphenylmethyl]-2-[N-(N,N-dimethylaminocarbonyl)-(L)-valyl]hydrazine;
1-[2(S)-hydroxy-3(S)-(N-(N-(2-methoxyethyl)aminocarbonyl)-(L)-valyl)amino-4-phenylbutyl]-1-[3,4-dimethoxyphenylmethyl]-2-[N-(N-(2-methoxyethyl)aminocarbonyl)-(L)-valyl]hydrazine:
1-[2(S)-hydroxy-3(S)-(N-(N-(2-(morpholin-4-yl)ethyl)aminocarbonyl)-(L)-valyl)amino-4-phenylbutyl]-1-[3,4-dimethoxyphenylmethyl]-2-[N-(N-(2-(morpholin-4-yl)ethyl)aminocarbonyl)-(L)-valyl]hydrazine;
1-[2(S)-hydroxy-3 (S)-(N-(N-(2-(morpholin-4-yl)ethyl)-N-methyl-aminocarbonyl)-(L)-valyl)amino-4-phenylbutyl]-1-[3,4-dimethoxyphenylmethyl]-2-[N-(N-(2-(morpholin-4-yl)ethyl)-N-methylaminocarbonyl)-(L)-valyl]hydrazine;
1-[2(S)-hydroxy-3(S)-(N-acetyl-(L)-valyl)amino-4-phenylbutyl]-1-[4-biphenylylmethyl]-2-[N-acetyl-(L)-valyl]hydrazine;
1-[2(S)-hydroxy-3(S)-(N-methoxycarbonyl-(L)-valyl)amino-4-phenylbutyl]-1-[4-biphenylmethyl]-2-N-methoxycarbonyl-(L)-valyl]hydrazine;
1-[2(S)-hydroxy-3(S)-(N-ethoxycarbonyl-(L)-valyl)amino-4-phenylbutyl]-1-[4-biphenylylmethyl]-2-[N-ethoxycarbonyl-(L)-valyl]hydrazine;
1-[2(S)-hydroxy-3(S)-(N-(N,N-dimethylaminocarbonyl)-(L)-valyl)amino-4-phenylbutyl]-1-[4-biphenylylmethyl]-2-[N-(N,N-dimethylaminocarbonyl)-(L)-valyl]hydrazine;
1-[2(S)-hydroxy-3 (S)-(N-(N-(2-methoxyethyl)aminocarbonyl)-(L)-valyl)amino-4-phenylbutyl]-1-[4-biphenylylmethyl]-2-[N-(N-(2-methoxyethyl)aminocarbonyl)-(L)-valyl]hydrazine;
1-[2(S)-hydroxy-3(S)-(N-(N-(2-(morpholin-4-yl)ethyl)aminocarbonyl)-(L)-valyl)amino-4-phenylbutyl]-1-[4-biphenylylmethyl]-2-[N-(N-(2-(morpholin-4-yl)ethyl)aminocarbonyl)-(L)-valyl]hydrazine and
1-[2(S)-hydroxy-3(S)-(N-(N-(2-(morpholin-4-yl)ethyl)-N-methyl-aminocarbonyl)-(L)-valyl)amino-4-phenylbutyl]-1-[4-biphenylylmethyl]-2-[N-(N-(2-(morpholin-4-yl)ethyl)N-methylaminocarbonyl)-(L)-valyl]hydrazine;
1-[2(S)-hydroxy-3(S)-(N-ethoxycarbonyl-(L)-valyl)amino-4-phenylbutyl]-1-[cyclohexylmethyl]-2-[N-ethoxycarbonyl-(L)-valyl]hydrazine;
1-[2(S)-hydroxy-3(S)-(N-trifluoroacetyl-(L)-valyl)amino-4-phenylbutyl]-1-[cyclohexylmethyl]-2-[N-trifluoroacetyl-(L)-valyl]hydrazine;
1-[2(S)-hydroxy-3(S)-(N-(methoxy-carbonyl)-(L)-valyl)amino-4-cyclohexyl-butyl]-1-[cyclohexylmethyl]-2-[N-methoxy-carbonyl-(L)-valyl]hydrazine;
1-[2(S)-hydroxy-3(S)-(N-(n-propoxy-carbonyl)-(L)-valyl)amino-4-phenyl-butyl]-1-[cyclohexylmethyl]-2-[N-(n-propyl)oxy-carbonyl-(L)-valyl]hydrazine;
1-[2(R)-hydroxy-3(S)-(N-(methoxy-carbonyl)-(L)-valyl)amino-4-phenyl-butyl]-1-[cyclohexylmethyl]-2-[N-methoxy-carbonyl-(L)-valyl]hydrazine;
1-[2(R)-hydroxy-3(R)-(N-(methoxy-carbonyl)-(L)-valyl)amino-4-phenyl-butyl]-1-[cyclohexylmethyl]-2-[N-methoxy-carbonyl-(L)-valyl]hydrazine;
1-[2(S)-hydroxy-3(S)-(benzyloxy-carbonyl-amino)-4-phenyl-butyl]-1-[phenylmethyl]-2-[N-methoxy-carbonyl-(L)-valyl]hydrazine;
1-[2(S)-hydroxy-3(S)-(N-(methoxy-carbonyl)-(L)-valyl)amino-4-cyclohexyl-butyl]-1-[phenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-methoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-isobutoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-ethoxyphenylmethyl]-2-[N-methoxy-carbonyl)-(L)-valyl]hydrazine;
1-[2(S)-hydroxy-3(S)-(N-methoxycarbonyl-(L)-valyl)amino-4-cyclohexyl-butyl]-1-[4-benzyloxyphenylmethyl]-2-[N-methoxy-carbonyl)-(L)-valyl]hydrazine;
N-[(1S,2R)-3-(2-benzoyl-1-ethylhydrazino)-1-benzyl-2-hydroxypropyl]-2-[(methylsulfonyl)amino]-1,3-thiazole-4-carboxamide;
N-{(1S,2R)-1-benzyl-3-[1-ethyl-2-(4-methylpentanoyl)hydrazino]-2-hydroxypropyl}-2-[(methylsulfonyl)amino]-1,3-thiazole-4-carboxamide;
N-{(1S,2R)-1-benzyl-3-[1-ethyl-2-(4-methylpentanoyl)hydrazino]-2-hydroxypropyl}-4-methylpentanamide;
N¹-{(1S,2S)-1-benzyl-3-[1-ethyl-2-(4-methylpentanoyl)hydrazino]-2-hydroxypropyl}-5-methyl-N³,N³-dipropylisophthalamide;
N¹-{(1S,2S)-1-(3,5-difluorobenzyl)-3-[1-ethyl-2-(4-methylpentanoyl)hydrazino]-2-hydroxypropyl}-5-methyl-N³,N³-dipropylisophthalamide;
N¹-{(1S,2S)-1-(3,5-difluorobenzyl)-3-[1-ethyl-2-(4-methylbutanoyl)hydrazino]-2-hydroxypropyl}-5-methyl-N³,N³-dipropylisophthalamide;
or pharmaceutically acceptable salts thereof.

7. A method of treating or preventing Alzheimer's disease in a patient in need of such treatment comprising administering a therapeutically effective amount of a composition comprising one or more pharmaceutically acceptable carriers and a compound of Formula (I) or a pharmaceutically acceptable salt thereof: 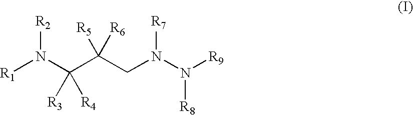
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

8. (canceled)

9. A method for inhibiting beta-secretase activity, comprising contacting an effective amount for inhibition of a compound of formula (I): 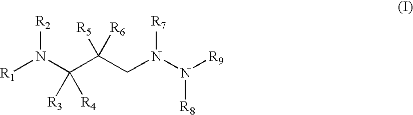
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

10. A method for inhibiting cleavage of an amyloid precursor protein (APP) isotype at a site in the APP isotype that is susceptible to cleavage, comprising contacting said APP isotype with an effective cleavage inhibitory amount of a compound of formula (I): 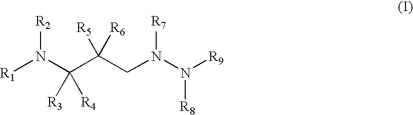
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

11. A method for inhibiting production of amyloid beta peptide (A beta) in a cell, comprising administering to said cell an effective inhibitory amount of a compound of formula (I): 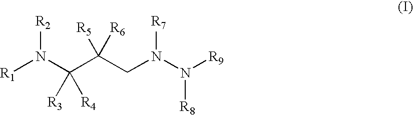
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

12. The method of claim 11, wherein the cell is an animal cell.

13. The method of claim 12, wherein the animal cell is a mammalian cell.

14. The method of claim 13, wherein the mammalian cell is human.

15. A composition comprising beta-secretase complexed with a compound of formula (I), 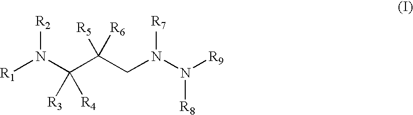
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

16. A method for producing a beta-secretase complex comprising the composition of claim 15.

17. A method for inhibiting the production of beta-amyloid plaque in an animal, comprising administering to said animal an effective inhibiting amount of a compound of formula (I): 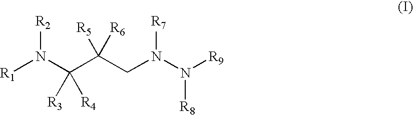
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

18. The method of claim 17, wherein said animal is a human.

19. A method for treating or preventing a disease characterized by beta-amyloid deposits on or in the brain, comprising administering to a patient in need of such treatment or prevention an effective therapeutic amount of a compound of formula (I): 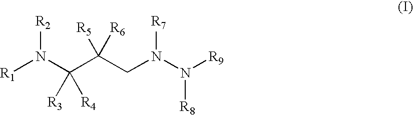
wherein R₁ and R₉ are each independently of the other hydrogen; acyl; unsubstituted or substituted alkyl, alkenyl or alkynyl; heterocyclyl; sulfo; sulfonyl substituted by unsubstituted or substituted alkyl, aryl, heterocyclyl, alkoxy, which is unsubstituted or substituted, or by aryloxy; sulfamoyl that is unsubstituted or substituted at the nitrogen atom; or phosphoryl substituted by one or two radicals, which may be identical or different, selected from unsubstituted or substituted alkyl, from unsubstituted or substituted cycloalkyl, from aryl, from hydroxy, from unsubstituted or substituted alkoxy, from cycloalkoxy and from aryloxy;
with the proviso that not more than one of the radicals R₁ and R₉ is hydrogen;
R₂ and R₈ are each independently of the other hydrogen or one of the radicals mentioned above for R₁ and R₉; or the pairs of substituents R₁ and R₂, and R₈ and R₉, each independently of the other, may form together with the nitrogen atom to which they are bonded a heterocyclic ring consisting of the bonding nitrogen atom together with a radical selected from ethylene, trimethylene, tetramethylene and pentamethylene in which a carbon atom may have been replaced by nitrogen, oxygen, sulfur or by sulfur mono- or di-substituted by oxygen and which may be unsaturated, or one of those radicals with an oxo substituent at each of the two carbon atoms linked to the bonding carbon atom and with or without a fused-on benzene or naphthalene ring;
R₃ and R₄ are each independently of the other hydrogen, unsubstituted or substituted alkyl or cycloalkyl; aryl; heterocyclyl; or unsubstituted or substituted alkenyl; or R₃ and R₄ together form unsubstituted or substituted alkylene, alkylidene or benzo-fused alkylene;
R₅ is hydroxy; R₆ is hydrogen; or R₅ and R₆ together are oxo; and
R₇ is unsubstituted or substituted alkyl or cycloalkyl; aryl; heterocyclyl; or unsubstituted or substituted alkenyl.

20. A method of treatment according to claim 5, further comprising administration of one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta peptide.

21. (canceled)

22. A method of treating or preventing Alzheimer's disease in a patient in need of such treatment comprising administering a therapeutically effective amount of a compound of Formula (I-A) or a pharmaceutically acceptable salt thereof: 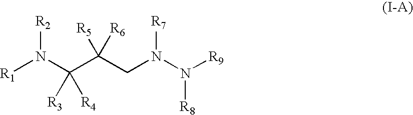
wherein R₁ and R₉ are each independently of the other hydrogen; acyl; unsubstituted or substituted alkyl; sulfo; or sulfonyl substituted by unsubstituted or substituted alkyl, aryl or by heterocyclyl, with the proviso that not more than one of the radicals R₁ and R₉ is hydrogen;
R₂ and R₈ are each independently of the other hydrogen or unsubstituted or substituted alkyl;
R₃ and R₄ are each independently of the other hydrogen, unsubstituted or substituted alkyl, unsubstituted or substituted cycloalkyl or aryl;
R₅ is acyloxy;
R₆ is hydrogen; and
R₇ is unsubstituted or substituted alkyl, unsubstituted or substituted cycloalkyl or aryl; and salts of the mentioned compounds where salt-forming groups are present, with the exception of the compound wherein R₁ and R₉ are each acetyl, R₂, R₃, R₄, R₆ and R₈ are each hydrogen, R₅ is acetoxy and R₇ is 2,2-[N-ethoxycarbonylmethyl)-N-methyl]hydrazin-1-ylcarbonylmethyl.

23. A method of treating a patient who has, or in preventing a patient from getting, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I-A), or a pharmaceutically acceptable salt thereof: 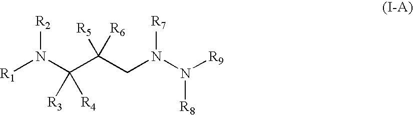
wherein R₁ and R₉ are each independently of the other hydrogen; acyl; unsubstituted or substituted alkyl; sulfo; or sulfonyl substituted by unsubstituted or substituted alkyl, aryl or by heterocyclyl, with the proviso that not more than one of the radicals R₁ and R₉ is hydrogen;
R₂ and R₈ are each independently of the other hydrogen or unsubstituted or substituted alkyl;
R₃ and R₄ are each independently of the other hydrogen, unsubstituted or substituted alkyl, unsubstituted or substituted cycloalkyl or aryl;
R₅ is acyloxy;
R₆ is hydrogen; and
R₇ is unsubstituted or substituted alkyl, unsubstituted or substituted cycloalkyl or aryl; and salts of the mentioned compounds where salt-forming groups are present, with the exception of the compound wherein R₁ and R₉ are each acetyl, R₂, R₃, R₄, R₆ and R₈ are each hydrogen, R₅ is acetoxy and R₇ is 2,2-[N-ethoxycarbonylmethyl)-N-methyl]hydrazin-1-ylcarbonylmethyl.

24. A method according to claim 23, wherein the compound is selected from the group consisting of:
1-[2(S)-palmitoyloxy-3(S)-(N-(methoxy-carbonyl)-(L)-valyl)amino-4-phenyl-butyl]-1-[cyclohexylmethyl]-2-[N-methoxy-carbonyl-(L)-valyl]hydrazine;
1[-2(S)-(methoxy-acetoxy)-3(S)-(N-(methoxy-carbonyl)-(L)-valyl)amino-4-phenylbutyl]-1-[cyclohexylmethyl]-2-[N-methoxy-carbonyl-(L)-valyl]hydrazine;
1-[2(S)-(2-pyridyl-carbonyl)oxy-3(S)-(tert-butoxy-carbonyl)amino-4-phenyl-butyl]-1-[cyclohexylmethyl]-2-[tert-butoxy-carbonyl]hydrazine;
1-[2(S)-(2-pyridylcarbonyl)oxy-3(S)-(N-quinoline-2-carbonyl)-(L)-asparaginyl)amino4-phenylbutyl-1-[phenylmethyl]-2-[N-(methoxycarbonyl)-(L)-valyl]hydrazine;
1-[2(S)-butyryloxy-3(S)-((N-quinoline-2-carbonyl)-(L)-asparaginyl)amino-4-phenylbutyl-1-[phenylmethyl]-2-[N-(methoxycarbonyl)-(L)-valyl]hydrazine;
1-[2(S)-(2-pyridylcarbonyl)oxy-3(S)-(N-quinoline-2-carbonyl)-(L)-asparaginyl)amino4-phenylbutyl-1-[4-methoxyphenylmethyl]-2-[N-(benzyloxycarbonyl)-(L) valyl]hydrazine;
1-[2(S)-(methoxy-acetyl)oxy-3(S)-(N-quinoline-2-carbonyl)-(L)-asparaginyl)amino-4-phenylbutyl-1-[4-methoxyphenylmethyl]-2-[N-(benzyloxycarbonyl)-(L)-valyl]hydrazine;
1-[2(S)-propionyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-butyryloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pentanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-octanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-decanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-dodecanoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-pivaloyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-furylcarbonyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(4-imidazolylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-imidazolylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(4-imidazolyl)-propionyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-benzoyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(2-pyridylacetyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
1-[2(S)-(3-(pyridin-2-yl)-propionyl)oxy-3(S)-(N-(methoxycarbonyl)-(L)-valyl)amino-4-phenylbutyl)]-1-[cyclohexylmethyl]-2-[N-(methoxycarbonyl)-(L)-valyl]hydrazine;
1-[2(S)-(quinolin-2-ylcarbonyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(aminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-methylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N,N-dimethylaminoacetyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(N-benzyloxycarbonyl-N-methyl-aminoacetyl)oxy-3(S)-(N-(methoxycarbonyl)(L)-valyl)amino-4-phenyl-butyl]-1-[cyclohexylmethyl]-2-[N-(methoxycarbonyl)(L)-valyl]hydrazine;
1-[2(S)-prolyloxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-morpholinomethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine;
1-[2(S)-(4-chloromethylbenzoyl)oxy-3(S)-(N-(methoxycarbonyl)-(L)-valyl)amino-4-phenyl-butyl]-1-[cyclohexylmethyl]-2-[N-(methoxycarbonyl)-(L)-valyl]hydrazine; and
1-[2(S)-(3-carboxypropionyl)oxy-3(S)-(N-(methoxycarbonyl)-(L)-valyl)amino-4-phenylbutyl]-1-[cyclohexylmethyl]-2-[N-(methoxycarbonyl)-(L)-valyl]hydrazine;
or a pharmaceutically acceptable salt thereof.
